(19) United States
(12) Patent Application Publication
(10) Pub. No.: US 2007/0249637 A1
(43) Pub. Date: Oct. 25, 2007

(54) Inhibitors Of Hepatitis C Virus Protease, And Compositions And Treatments Using The Same

(76) Inventors: Michael Raymond Collins, San Diego, CA (US); Vijayalakshmi Natarajan, San Diego, CA (US)

Correspondence Address: PFIZER INC, 10555 SCIENCE CENTER DRIVE, SAN DIEGO, CA, 92121, US

(21) Appl. No.: 11/577,593

(22) PCT Filed: Oct. 10, 2005
(86) PCT No.: PCT/IB05/03085

§ 371 Date: Apr. 19, 2007

Related U.S. Application Data
(60) Provisional application No. 60/621,302, filed on Oct. 21, 2004.
(60) Provisional application No. 60/650,150, filed on Feb. 3, 2005.
(60) Provisional application No. 60/705,558, filed on Aug. 3, 2005.

(51) Int. Cl. A61K 31/44; A61K 31/519; A61P 31/12; C07D 495/04; C07D 513/04

(52) U.S. Cl. 514/260.1; 514/301; 544/278; 546/114

(57) ABSTRACT

The present invention provides compounds of formula (I), (II) or (IV), or pharmaceutically acceptable salts and solvates thereof, which are useful as inhibitors of the Hepatitis C virus (HCV) protease enzyme and are also useful for the treatment of HCV infections in HCV-infected mammals, including humans. The present invention also provides pharmaceutical compositions comprising compounds of formula (I), (II) or (IV), their pharmaceutically acceptable salts and solvates. Furthermore, the present invention provides intermediate compounds and methods useful in the preparation of compounds of formulas (I), (II) and (IV).

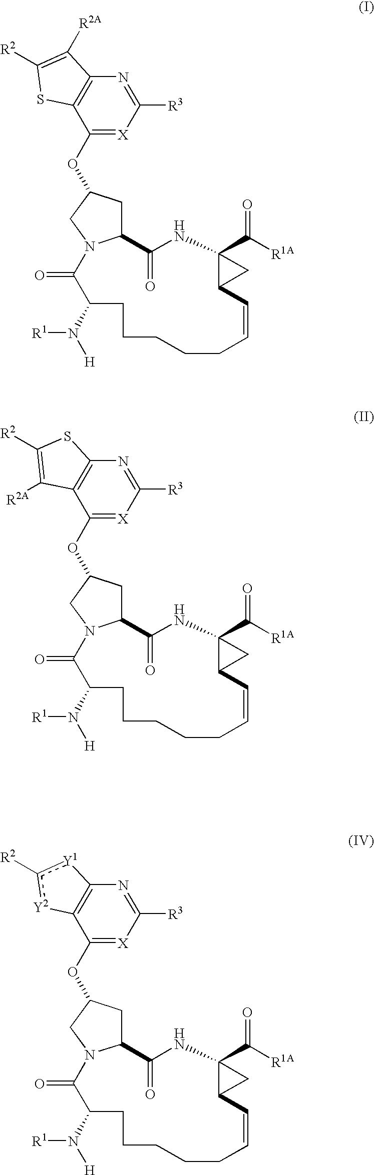

### CROSS-REFERENCE TO RELATED APPLICATIONS

This application claims the benefit of U.S. Provisional Application No. 60/621,302, filed Oct. 21, 2004, U.S. Provisional Application No. 60/650,150, filed Feb. 3, 2005, and U.S. Provisional Application No. 60/705,558, filed Aug. 3, 2005. The disclosure of each of these applications is incorporated herein.

### FIELD OF THE INVENTION

The present invention relates to compounds useful as inhibitors of the Hepatitis C virus (HCV) protease enzyme, pharmaceutical compositions comprising such compounds, methods of using such compounds and formulations in the treatment of HCV-infected mammals, such as humans, and methods and intermediate compounds useful in preparing such compounds.

### BACKGROUND

The invention relates to agents that inhibit hepatitis C virus (HCV) protease. The invention also relates to the use of such compounds in pharmaceutical compositions and therapeutic treatments useful for inhibition of HCV replication.

HCV is an enveloped RNA virus containing a single-stranded positive-sense RNA genome approximately 9.5 kb in length (Choo, et al., Science 244:359-362 (1989)). The RNA genome contains a 5′-nontranslated region (5′ NTR) of 341 nucleotides (Brown, et al., Nucl. Acids Res. 20:5041-5045 (1992); Bukh, et al., Proc. Natl. Acad. Sci. USA 89:4942-4946 (1992)), a large open reading frame (ORF) encoding a single polypeptide of 3,010 to 3,040 amino acids (Choo, et al. (1989), supra;), and a 3′-nontranslated region (3′-NTR) of variable length of about 230 nucleotides (Kolykhalov, et al., J. Virol. 70:3363-3371 (1996); Tanaka, et al., J. Virol. 70:3307-3312 (1996)).

The 5′ NTR is one of the most conserved regions of the viral genome and plays a pivotal role in the initiation of translation of the viral polyprotein. A single ORF encodes a polyprotein that is co- or post-translationally processed into structural (core, E1, and E2) and nonstructural (NS2, NS3, NS4A, NS4B, NS5A, and NS5B) viral proteins by either cellular or viral proteinases (Bartenschlager (1997), supra). The 3′ NTR consists of three distinct regions: a variable region of about 38 nucleotides following the stop codon of the polyprotein, a polyuridine tract of variable length with interspersed substitutions of cystines, and 98 nucleotides (nt) at the very 3′ end which are highly conserved among various HCV isolates. The order of the genes within the genome is: NH₂-C-E1-E2-p7-NS2-NS3-NS4A-NS4B-NS5A-NS5B-COOH (Grakoui, et al., J. Virol. 67:1385-1395 (1993)).

Hepatitis C virus (HCV) is a member of the hepacivirus genus in the family Flaviviridae. It is the major causative agent of non-A, non-B viral hepatitis and is the major cause of transfusion-associated hepatitis and accounts for a significant proportion of hepatitis cases worldwide. Although acute HCV infection is often asymptomatic, nearly 80% of cases resolve to chronic hepatitis. The persistent property of the HCV infection has been explained by its ability to escape from the host immune surveillance through hypermutability of the exposed regions in the envelope protein E2 (Weiner, et al., Virology 180:842-848 (1991); Weiner, et al. Proc. Natl. Acad. Sci. USA 89:3468-3472 (1992).

Processing of the structural proteins core (C), envelope protein 1 and (E1, E2), and the p7 region is mediated by host signal peptidases. In contrast, maturation of the nonstructural (NS) region is accomplished by two viral enzymes. The HCV polyprotein is first cleaved by a host signal peptidase generating the structural proteins C/E1, E1/E2, E2/p7, and p7/NS2 (Hijikata, et al., Proc. Natl. Acad. Sci. USA 88:5547-5551 (1991); Lin, et al., J. Virol. 68:5063-5073 (1994)). The NS2-3 proteinase, which is a metalloprotease, then cleaves at the NS2/NS3 junction. The NS3/4A proteinase complex (NS3 serine protease/NS4A cofactor), then at all the remaining cleavage sites (Bartenschlager, et al., J. Virol. 67:3835-3844 (1993); Bartenschlager, (1997), supra). RNA helicase and NTPase activities have also been identified in the NS3 protein. The N-terminal one-third of the NS3 protein functions as a protease, and the remaining two-thirds of the molecule acts as a helicase/ATPase, which is thought to be involved in HCV replication (Bartenschlager, (1997), supra). NS5A may be phosphorylated and act as a putative cofactor of NS5B. The fourth viral enzyme, NS5B, is an RNA-dependent RNA polymerase (RdRp) and a key component responsible for replication of the viral RNA genome (Lohmann, et al., J. Virol. 71:8416-8428 (1997)).

Replication of HCV is thought to occur in membrane-associated replication complexes. Within these, the genomic plus-strand RNA is transcribed into minus-strand RNA, which in turn can be used as a template for synthesis of progeny genomic plus strands. Two viral proteins appear to be involved in this reaction: (1) the NS3 protein, which carries in the carboxy terminal two-thirds a nucleoside triphosphatase/RNA helicase; and (2) the NS5B protein, which is a membrane-associated phosphoprotein with an RNA-dependent RNA polymerase activity (RdRp) (Hwang et al., J. Virol. 227:439-446 (1997)).

Since persistent infection of HCV is related to chronic hepatitis and eventually to hepatocarcinogenesis, HCV replication is one of the targets to eliminate HCV reproduction and to prevent hepatocellular carcinoma. Some HCV treatment therapies involve alpha-interferon alone or a combination of alpha-interferon with Ribavirin (Schering-Plough Corp.). Unfortunately, present treatment approaches for HCV infection are characterized by relatively poor efficacy and an unfavorable side-effect profile. Therefore, intensive effort is directed at the discovery of molecules to treat this disease, including the discovery of drugs designed to inhibit HCV replication, as there is a persistent need for small-molecule compounds that are HCV protease inhibitors having desirable or improved physical and chemical properties appropriate for pharmaceutical applications.

### SUMMARY OF THE INVENTION

The present invention relates to compounds of formula IV 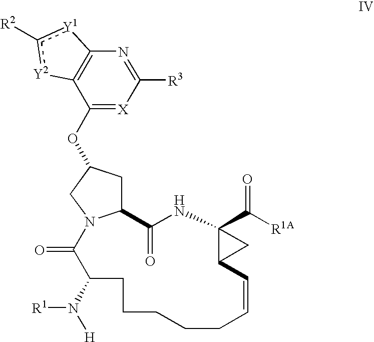
wherein:

R¹ is selected from C₁-C₁₀ alkyl, C₂-C₆ alkenyl, C₂-C₆ alkynyl, —OR⁵, —C(O)R⁵, —C(O)OR⁵, —C(O)NR⁵R⁶, —SO₂NR⁵R⁶, heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² is selected from H, halo, cyano, nitro, azido, C₁-C₆ alkyl, C₂-C₆ alkenyl, C₂-C₆ alkynyl, —C(O)NR⁵R⁶, —OR⁵, —C(O)R⁵, —C(O)OR⁵, —OC(O)R⁵, NR⁵C(O)R⁶, —NR⁵C(O)NR⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), heteroaryl, and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R² groups are optionally substituted with at least one R⁴ group;

R³ is selected from H, halo, cyano, nitro, azido, C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, heteroaryl, —(CR⁷R⁸)ₜ(C₆-C₁₀ aryl), —(CR⁷R⁸)ₜ(4-10 membered heterocyclic), and C₃-C₁₀ cycloalkyl, wherein each of said heteroaryl, —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₃-C₁₀ cycloalkoxy, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, 2, 3, 4, and 5;

X is CH or N; and

Y¹ and Y² are each independently selected from CH, CR¹, O, S, and NR²;

or pharmaceutically acceptable salts or solvates thereof.

The present invention further relates to a compound of Formula IV wherein R¹ is selected from C₁-C₁₀ alkyl, —OR⁵, —C(O)R⁵, —C(O)OR⁵, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said C₆-C₁₀ aryl, 4-10 membered heterocyclic, C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶—SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R³)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² is selected from H, halo, cyano, nitro, azido, C₁-C₆ alkyl, C₂-C₆ alkenyl, C₂-C₆ alkynyl, —C(O)NR⁵R⁶, —OR⁵, —C(O)R⁵, —C(O)OR⁵, —OC(O)R⁵, —NR⁵C(O)R⁶, —NR⁵C(O)NR⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), heteroaryl, and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R² groups are optionally substituted with at least one R⁴ group;

R³ is selected from H, halo, cyano, nitro, azido, C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, 2, 3, 4, and 5;

X is CH or N;

Y¹ and Y² are each independently selected from CH, CR¹, O, S, and NR²;

or pharmaceutically acceptable salts or solvates thereof.

The present invention further relates to a compound of Formula IV wherein R¹ is selected from C₁-C₁₀ alkyl, —OR⁵, C₃-C₁₀ cycloalkoxy, and C₃-C₁₀ cycloalkyl, wherein each of said C₁-C₁₀ alkyl, C₃-C₁₀ cycloalkoxy, and C₃-C₁₀ cycloalkyl moieties of said R¹ groups are optionally substituted with at least one R⁴ group, and wherein at least one carbon in each of said C₁-C₁₀ alkyl, C₃-C₁₀ cycloalkoxy, and C₃-C₁₀ cycloalkyl moieties of said R¹ groups is optionally replaced by —NH—, O or S, with the proviso that said C₁-C₁₀ alkyl, C₃-C₁₀ cycloalkoxy, and C₃-C₁₀ cycloalkyl moieties do not have O—O or S—S bonds;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² is selected from H, halo, cyano, nitro, azido, C₁-C₆ alkyl, C₂-C₆ alkenyl, C₂-C₆ alkynyl, —C(O)NR⁵R⁶, —OR⁵, —C(O)R⁵, —C(O)OR⁵, —OC(O)R⁵, —NR⁵C(O)R⁶, —NR⁵C(O)NR⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), heteroaryl, and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R² groups are optionally substituted with at least one R⁴ group;

R³ is selected from H, halo, cyano, nitro, azido, C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁶)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, 2, 3, 4, and 5;

X is CH or N;

Y¹ and Y² are each independently selected from CH, CR¹, O, S, and NR²;

or pharmaceutically acceptable salts or solvates thereof.

The present invention further relates to a compound of Formula IV wherein R¹ is 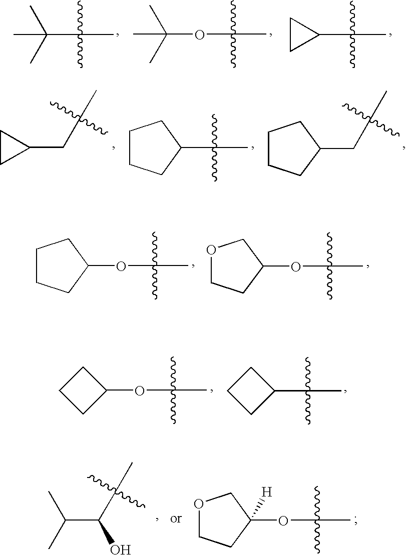
R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² is selected from H, halo, cyano, nitro, azido, C₁-C₆ alkyl, C₂-C₆ alkenyl, C₂-C₆ alkynyl, —C(O)NR⁵R⁶, —OR⁵, —C(O)R⁵, —C(O)OR⁵, —OC(O)R⁵, —NR⁵C(O)R⁶, —NR⁵C(O)NR⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), heteroaryl, and C₃-C₁₀ cycloalkyl, wherein each of said —(CR⁷R⁸)ₜ(C₆-C₁₀ aryl), —(CR⁷R³)ₜ(4-10 membered heterocyclic), and C₃-C₁₀ cycloalkyl moieties of said R² groups are optionally substituted with at least one R⁴ group;

R³ is selected from H, halo, cyano, nitro, azido, C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁵, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, 2, 3, 4, and 5;

X is CH or N;

Y¹ and Y² are each independently selected from CH, CR¹, O, S, and NR²;

or pharmaceutically acceptable salts or solvates thereof.

The present invention further relates to a compound of Formula IV wherein R¹ is selected from C₁-C₁₀ alkyl, C₂-C₆ alkenyl, C₂-C₆ alkynyl, —OR⁵, —C(O)R⁵, —C(O)OR⁵, —C(O)NR⁵R⁶, —SO₂NR⁵R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² is selected from H, —C(O)NR⁵R⁶, —OR⁵, —C(O)R⁵, —C(O)OR⁵, —NR⁵C(O)R⁸, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;

R³ is selected from H, halo, cyano, nitro, azido, C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, 2, 3, 4, and 5;

X is CH or N;

Y¹ and Y² are each independently selected from CH, CR¹, O, S, and NR²;

or pharmaceutically acceptable salts or solvates thereof.

The present invention further relates to a compound of Formula IV wherein R¹ is selected from C₁-C₁₀ alkyl, C₂-C₆ alkenyl, C₂-C₆ alkynyl, —OR⁵, —C(O)R⁵, —C(O)OR⁵, —C(O)NR⁵R⁶, —SO₂NR⁵R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² is selected from H, halo, cyano, nitro, azido, C₁-C₆ alkyl, C₂-C₆ alkenyl, C₂-C₆ alkynyl, —C(O)NR⁵R⁶, —OR⁵, —C(O)R⁵, —C(O)OR⁵, —OC(O)R⁵, —NR⁵C(O)R⁶, —NR⁵C(O)NR⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), heteroaryl, and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R² groups are optionally substituted with at least one R⁴ group;

R³ is selected from H, C₁-C₁₀ alkyl, C₂-C₆ alkenyl, C₂-C₆ alkynyl, —NR⁵C(O)R⁶, —NR⁵C(O)NR⁶, —OR⁵, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, 2, 3, 4, and 5;

X is CH or N;

Y¹ and Y² are each independently selected from CH, CR¹, O, S, and NR²;

or pharmaceutically acceptable salts or solvates thereof.

The present invention further relates to a compound of Formula IV wherein R¹ is selected from C₁-C₁₀ alkyl, C₂-C₆ alkenyl, C₂-C₆ alkynyl, —OR⁵, —C(O)R⁵, —C(O)OR⁵, —C(O)NR⁵R⁶, —SO₂NR⁵R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² is selected from H, halo, cyano, nitro, azido, C₁-C₆ alkyl, C₂-C₆ alkenyl, C₂-C₆ alkynyl, —C(O)NR⁵R⁶, —OR⁵, —C(O)R⁵, —C(O)OR⁵, —OC(O)R⁵, —NR⁵C(O)R⁶, —NR⁵C(O)NR⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), heteroaryl, and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R² groups are optionally substituted with at least one R⁴ group;

R³ is selected from H, halo, cyano, nitro, azido, C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶—NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, and 2;

X is CH or N;

Y¹ and Y² are each independently selected from CH, CR¹, O, S, and NR²;

or pharmaceutically acceptable salts or solvates thereof.

The present invention further relates to a compound of Formula IV wherein R¹ is selected from C₁-C₁₀ alkyl, C₂-C₆ alkenyl, C₂-C₆ alkynyl, —OR⁵, —C(O)R⁵, —C(O)OR⁵, —C(O)NR⁵R⁶, —SO₂NR⁵R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² is selected from H, halo, cyano, nitro, azido, C₁-C₆ alkyl, C₂-C₆ alkenyl, C₂-C₆ alkynyl, —C(O)NR⁵R⁶, —OR⁵, —C(O)R⁵, —C(O)OR⁵, —OC(O)R⁵, —NR⁵C(O)R⁶, —NR⁵C(O)NR⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), heteroaryl, and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R² groups are optionally substituted with at least one R⁴ group;

R³ is selected from H, halo, cyano, nitro, azido, C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R³)ₜ(4-10 membered heterocyclic);

each R⁷ and R⁸, which may be the same or different, is independently selected from H and C₁-C₆ alkyl;

each t is independently selected from 0 and 1;

X is CH or N;

Y¹ and Y² are each independently selected from CH, CR¹, O, S, and NR²;

or pharmaceutically acceptable salts or solvates thereof.

The present invention further relates to a compound of Formula IV wherein R¹ is selected from C₁-C₁₀ alkyl, C₂-C₆ alkenyl, C₂-C₆ alkynyl, —OR⁵, —C(O)R⁵, —C(O)OR⁶, —C(O)NR⁵R⁶, —SO₂NR⁵R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² is selected from H, halo, cyano, nitro, azido, C₁-C₆ alkyl, C₂-C₆ alkenyl, C₂-C₆ alkynyl, —C(O)NR⁵R⁶, —OR⁵, —C(O)R⁵, —C(O)OR⁵, —OC(O)R⁵, —NR⁵C(O)R⁶, —NR⁵C(O)NR⁶, —NR⁵R⁶, —NR⁵OR⁶, —SO₂NR⁵R⁶, NR⁵SO₂R⁶, (CR⁷R⁸)ₜ(C6Co aryl), —(CR⁷R⁸)ₜ(4-10 membered heterocyclic), heteroaryl, and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R² groups are optionally substituted with at least one R⁴ group;

R³ is selected from H, halo, cyano, nitro, azido, C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(C R⁷R⁸)ₜ(4-10 membered heterocyclic);

each R⁷ and R⁸, which may be the same or different, is independently selected from H and C₁-C₆ alkyl;

t is 1;

X is CH or N;

Y¹ and Y² are each independently selected from CH, CR¹, O, S, and NR²;

or pharmaceutically acceptable salts or solvates thereof.

The present invention further relates to a compound of Formula IV wherein R¹ is selected from C₁-C₁₀ alkyl, C₂-C₆ alkenyl, C₂-C₆ alkynyl, —OR⁵, —C(O)R⁵, —C(O)OR⁵, —C(O)NR⁵R⁶, —SO₂NR⁵R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁵, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² is selected from H, halo, cyano, nitro, azido, C₁-C₆ alkyl, C₂-C₆ alkenyl, C₂-C₆ alkynyl, —C(O)NR⁵R⁶, —OR⁵, —C(O)R⁵, —C(O)OR⁶, —OC(O)R⁵, —NR⁵C(O)R⁶, —NR⁵C(O)NR⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), heteroaryl, and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R² groups are optionally substituted with at least one R⁴ group;

R³ is selected from H, halo, cyano, nitro, azido, C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁶)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

t is 0;

X is CH or N;

Y¹ and Y² are each independently selected from CH, CR¹, O, S, and NR²;

or pharmaceutically acceptable salts or solvates thereof.

The present invention further relates to a compound of Formula I 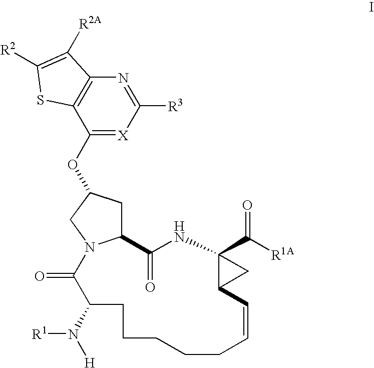
wherein R¹ is selected from C₁-C₁₀ alkyl, C₂-C₆ alkenyl, C₂-C₆ alkynyl, —OR⁵, —C(O)R⁶, —C(O)OR⁵, —C(O)NR⁵R⁶, —SO₂NR⁵R⁶, heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁶C(O)R⁵, —NR⁶C(O)NR⁶, —C(O)NR⁵R⁶, —NR⁵R⁶, —NR⁵OR⁶, —SO₂NR⁵R⁶, —NR⁵SO₂R⁶, heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, halo, cyano, nitro, azido, C₁-C₆ alkyl, C₂-C₆ alkenyl, C₂-C₆ alkynyl, —C(O)NR⁵R⁶, —OR⁵, —C(O)R⁵, —C(O)OR⁵, —OC(O)R⁵, —NR⁵C(O)R⁶, —NR⁵C(O)NR⁶, —NR⁵R⁶, —NR⁵OR⁶—SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), heteroaryl, and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R² groups are optionally substituted with at least one R⁴ group;

R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁵, —NR⁵C(O)NR⁶, —C(O)NR⁵R⁶, —NR⁵R⁶, —NR⁵OR⁶, —SO₂NR⁵R⁶, —NR⁵SO₂R⁶, heteroaryl, —(CR⁷R⁸)ₜ(C₆-C₁₀ aryl), —(CR⁷R⁸)ₜ(4-10 membered heterocyclic), and C₃-C₁₀ cycloalkyl, wherein each of said heteroaryl, —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, 2, 3, 4, and 5; and

X is CH or N;

or pharmaceutically acceptable salts or solvates thereof.

The present invention further relates to a compound of Formula I wherein R¹ is selected from C₁-C₁₀ alkyl, —OR⁵, —C(O)R⁵, —C(O)OR⁵, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁶)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;

R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₅-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, 2, 3, 4, and 5; and

X is CH or N;

or pharmaceutically acceptable salts or solvates thereof.

The present invention further relates to a compound of Formula I wherein R¹ is selected from C₁-C₁₀ alkyl, —OR⁵, C₃-C₁₀ cycloalkoxy, and C₃-C₁₀ cycloalkyl, wherein each of said C₁-C₁₀ alkyl, C₃-C₁₀ cycloalkoxy, and C₃-C₁₀ cycloalkyl moieties of said R¹ groups are optionally substituted with at least one R⁴ group, and wherein at least one carbon in each of said C₁-C₁₀ alkyl, C₃-C₁₀ cycloalkoxy, and C₃-C₁₀ cycloalkyl moieties of said R¹ groups is optionally replaced by —NH—, O or S, with the proviso that said C₁-C₁₀ alkyl, C₃-C₁₀ cycloalkoxy, and C₃-C₁₀ cycloalkyl moieties do not have O—O or S—S bonds;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁵, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;

R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶—NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, 2, 3, 4, and 5; and

X is CH or N;

or pharmaceutically acceptable salts or solvates thereof.

The present invention further relates to a compound of Formula I wherein R¹ is

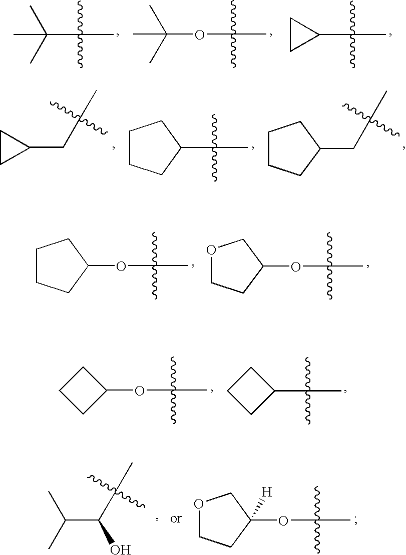

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;

R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁶)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), (CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁵, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, 2, 3, 4, and 5; and

X is CH or N;

or pharmaceutically acceptable salts or solvates thereof.

The present invention further relates to a compound of Formula I wherein R¹ is selected from C₁-C₁₀ alkyl, C₂-C₆ alkenyl, C₂-C₆ alkynyl, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;

R³ is selected from H, C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁵, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, 2, 3, 4, and 5; and

X is CH or N;

or pharmaceutically acceptable salts or solvates thereof.

The present invention further relates to a compound of Formula I wherein R¹ is selected from C₁-C₁₀ alkyl, C₂-C₆ alkenyl, C₂-C₆ alkynyl, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR³, —(CR⁷R⁸)ₜ(C₆-C₁₀ aryl), and heteroaryl, wherein said —(CR⁷R⁸)ₜ(C₆-C₁ aryl) and heteroaryl are optionally substituted with at least one R⁴ group;

R³ is selected from H, C₁-C₁₀ alkyl, C₂-C₆ alkenyl, C₂-C₆ alkynyl, —NR⁵C(O)R⁶, —NR⁵C(O)NR⁶, —OR⁵, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, 2, 3, 4, and 5; and

X is CH or N;

or pharmaceutically acceptable salts or solvates thereof.

The present invention further relates to a compound of Formula I wherein R¹ is selected from C₁-C₁₀ alkyl, C₂-C₆ alkenyl, C₂-C₆ alkynyl, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;

R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁸, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁸, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, and 2; and

X is CH or N;

or pharmaceutically acceptable salts or solvates thereof.

The present invention further relates to a compound of Formula I wherein R¹ is selected from C₁-C₁₀ alkyl, C₂-C₆ alkenyl, C₂-C₆ alkynyl, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;

R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁵, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶—NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0 and 1; and

X is CH or N;

or pharmaceutically acceptable salts or solvates thereof.

The present invention further relates to a compound of Formula I wherein R¹ is selected from C₁-C₁₀ alkyl, C₂-C₆ alkenyl, C₂-C₆ alkynyl, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;

R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶—NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

t is 1; and

X is CH or N;

or pharmaceutically acceptable salts or solvates thereof.

The present invention further relates to a compound of Formula I wherein R¹ is selected from C₁-C₁₀ alkyl, C₂-C₆ alkenyl, C₂-C₆ alkynyl, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R³, —NR⁵OR³, —(CR⁷R⁸)ₜ(C₆-C₁₀ aryl), and heteroaryl, wherein said —(CR⁷R³)ₜ(C₆-C₁₀ aryl) and heteroaryl are optionally substituted with at least one R⁴ group;

R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R³, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R³, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

t is 0; and

X is CH or N;

or pharmaceutically acceptable salts or solvates thereof.

The present invention further relates to a compound of Formula II 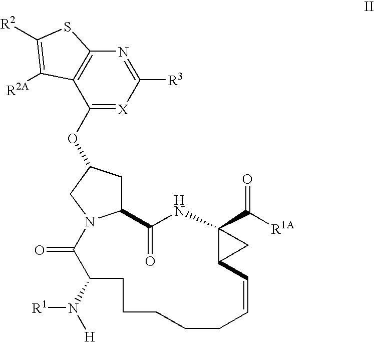
wherein R¹ is selected from C₁-C₁₀ alkyl, C₂-C₆ alkenyl, C₂-C₆ alkynyl, —OR⁵, —C(O)R⁵, —C(O)OR⁵, —C(O)NR⁵R⁶, —SO₂NR⁵R⁶, heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, heteroaryl, —(CR⁷R⁸)ₜ(C₁-C₁₀ aryl), —(CR⁷R⁸)ₜ(4-10 membered heterocyclic), C₃-C₁₀ cycloalkoxy, and C₃-C₁₀ cycloalkyl, wherein each of said heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, halo, cyano, nitro, azido, C₁-C₆ alkyl, C₂-C₆ alkenyl, C₂-C₆ alkynyl, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein each of said —(CR⁷R⁸)ₜ(C₆-C₁₀ aryl) and heteroaryl moieties of said R² and R^{2A} groups are optionally substituted with at least one R⁴ group;

R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, heteroaryl, —(CR⁷R⁸)ₜ(C₆-C₁₀ aryl), —(CR⁷R⁸)ₜ(4-10 membered heterocyclic), and C₃-C₁₀ cycloalkyl, wherein each of said heteroaryl, —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, 2, 3, 4, and 5; and

X is CH or N;

or pharmaceutically acceptable salts or solvates thereof.

The present invention further relates to a compound of Formula II wherein R¹ is selected from C₁-C₁₀ alkyl, —OR⁵, —C(O)R⁵, —C(O)OR⁵, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;

R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, 2, 3, 4, and 5; and

X is CH or N;

or pharmaceutically acceptable salts or solvates thereof.

The present invention further relates to a compound of Formula II wherein R¹ is selected from C₁-C₁₀ alkyl, —OR⁵, C₃-C₁₀ cycloalkoxy, and C₃-C₁₀ cycloalkyl, wherein each of said C₁-C₁₀ alkyl, C₃-C₁₀ cycloalkoxy, and C₃-C₁₀ cycloalkyl moieties of said R¹ groups are optionally substituted with at least one R⁴ group, and wherein at least one carbon in each of said C₁-C₁₀ alkyl, C₃-C₁₀ cycloalkoxy, and C₃-C₁₀ cycloalkyl moieties of said R¹ groups is optionally replaced by —NH—, O or S, with the proviso that said C₁-C₁₀ alkyl, C₃-C₁₀ cycloalkoxy, and C₃-C₁₀ cycloalkyl moieties do not have O—O or S—S bonds;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R³)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said C₆-C₁₀ aryl, 4-10 membered heterocyclic, C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;

R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁵, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, 2, 3, 4, and 5; and

X is CH or N;

or pharmaceutically acceptable salts or solvates thereof.

The present invention further relates to a compound of Formula II wherein R¹ is

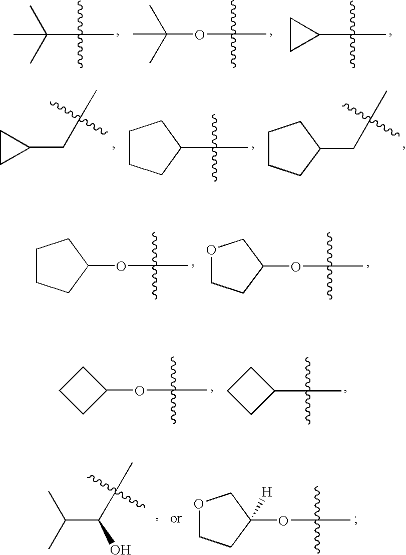

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R³)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, halo, cyano, nitro, azido, C₁-C₆ alkyl, C₂-C₆ alkenyl, C₂-C₆ alkynyl, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein each of said —(CR⁷R⁸)ₜ(C₆-C₁₀ aryl) and heteroaryl moieties of said R² and R^{2A} groups are optionally substituted with at least one R⁴ group;

R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R³)ₜ(4-10 membered heterocyclic), and C₃-C₁₀ cycloalkyl, wherein each of said —(CR⁷R⁸)ₜ(C₆-C₁₀ aryl), —(CR⁷R³)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, 2, 3, 4, and 5; and

X is CH or N;

or pharmaceutically acceptable salts or solvates thereof.

The present invention further relates to a compound of Formula II wherein R¹ is selected from C₁-C₁₀ alkyl, C₂-C₆ alkenyl, C₂-C₆ alkynyl, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, α-NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;

R³ is selected from H, C₁-C₁₀ alkyl, C₂-C₆ alkenyl, C₂-C₆ alkynyl, —OR⁵, —C(O)R⁵, —C(O)OR⁵—OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶—NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, 2, 3, 4, and 5; and

X is CH or N;

or pharmaceutically acceptable salts or solvates thereof.

The present invention further relates to a compound of Formula II wherein R¹ is selected from C₁-C₁₀ alkyl, C₂-C₆ alkenyl, C₂-C₆ alkynyl, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;

R³ is selected from H, C₁-C₁₀ alkyl, C₂-C₆ alkenyl, C₂-C₆ alkynyl, —NR⁵C(O)R⁶, —NR⁵C(O)NR⁶, —OR⁵, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, 2, 3, 4, and 5; and

X is CH or N;

or pharmaceutically acceptable salts or solvates thereof.

The present invention further relates to a compound of Formula II wherein R¹ is selected from C₁-C₁₀ alkyl, C₂-C₆ alkenyl, C₂-C₆ alkynyl, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁸, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁵, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;

R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁶C(O)R⁶—NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0, 1, and 2; and

X is CH or N;

or pharmaceutically acceptable salts or solvates thereof.

The present invention further relates to a compound of Formula II wherein R¹ is selected from C₁-C₁₀ alkyl, C₂-C₆ alkenyl, C₂-C₆ alkynyl, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl; C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;

R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

each t is independently selected from 0 and 1; and

X is CH or N;

or pharmaceutically acceptable salts or solvates thereof.

The present invention further relates to a compound of Formula II wherein R¹ is selected from C₁-C₁₀ alkyl, C₂-C₆ alkenyl, C₂-C₆ alkynyl, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)R⁵—OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;

R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁵, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁵, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

t is 1; and

X is CH or N;

or pharmaceutically acceptable salts or solvates thereof.

The present invention further relates to a compound of Formula II wherein R¹ is selected from C₁-C₁₀ alkyl, C₂-C₆ alkenyl, C₂-C₆ alkynyl, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;

R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;

R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;

R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;

each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶—NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;

each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);

each R⁷ and R⁸, which may be the same or different, is independently selected from H and C₁-C₆ alkyl;

t is 0; and

X is CH or N;

or pharmaceutically acceptable salts or solvates thereof.

In yet another aspect are compounds selected from: 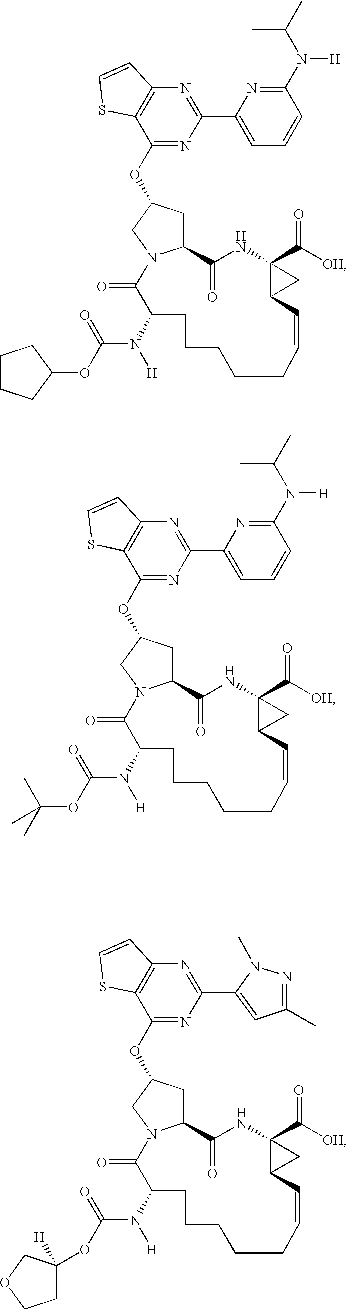 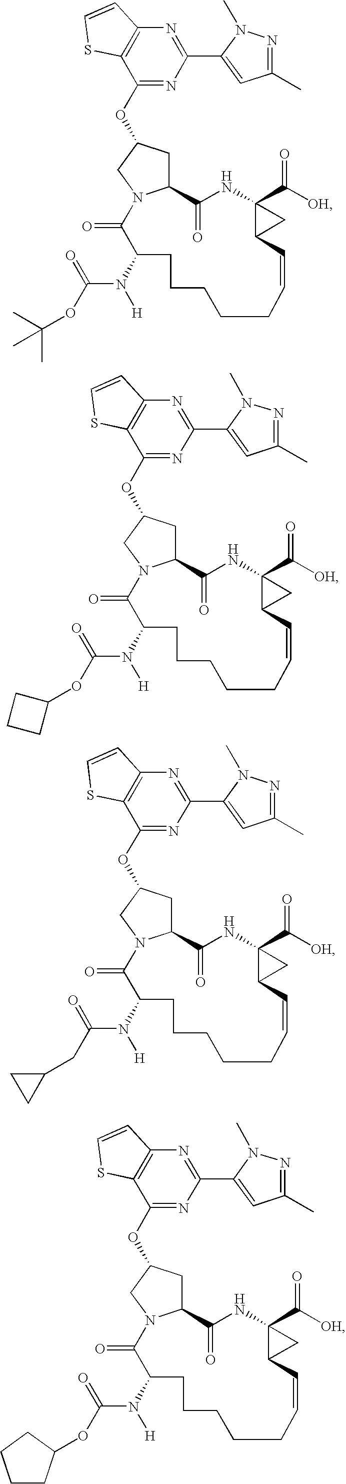 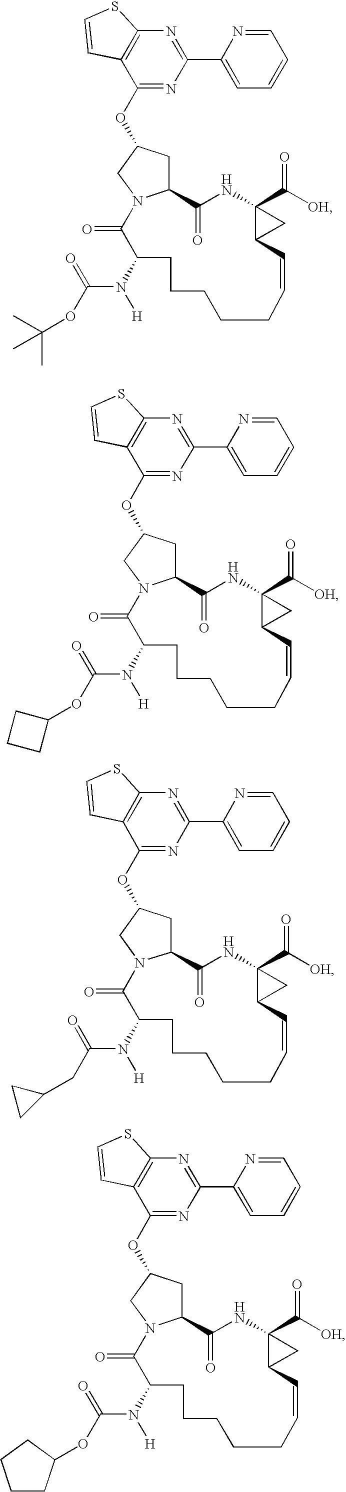 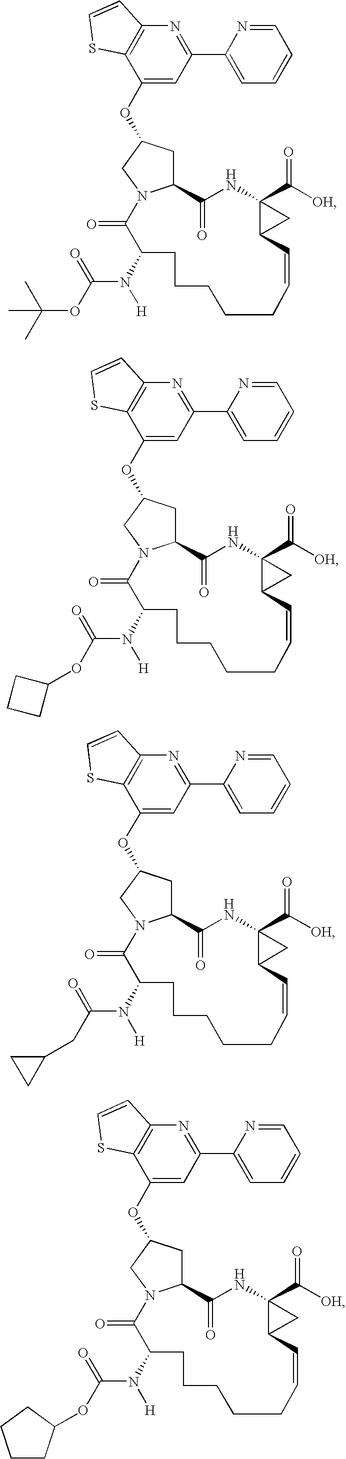 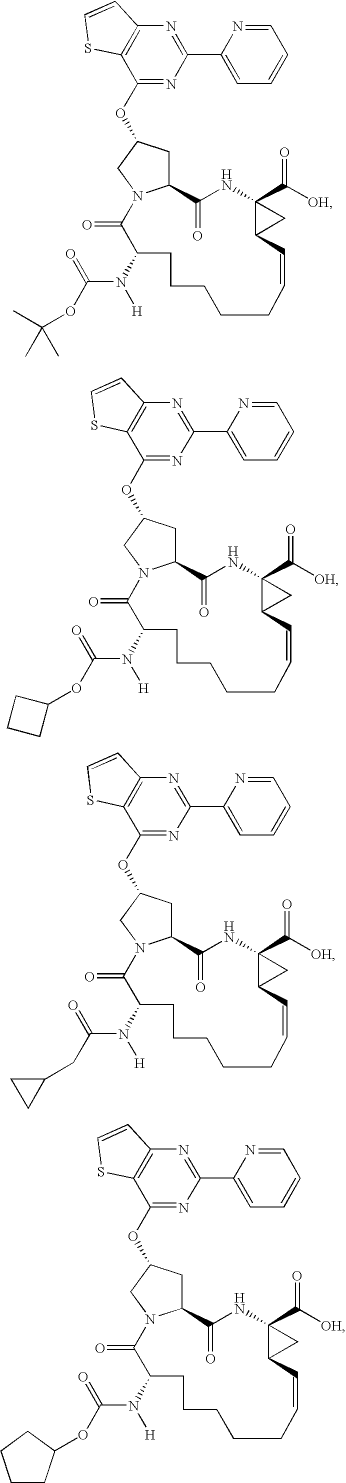 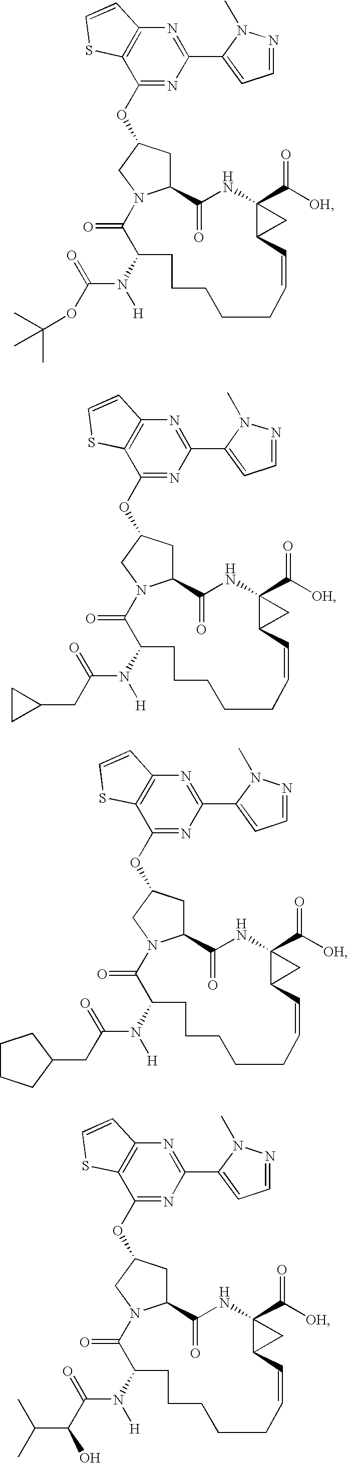 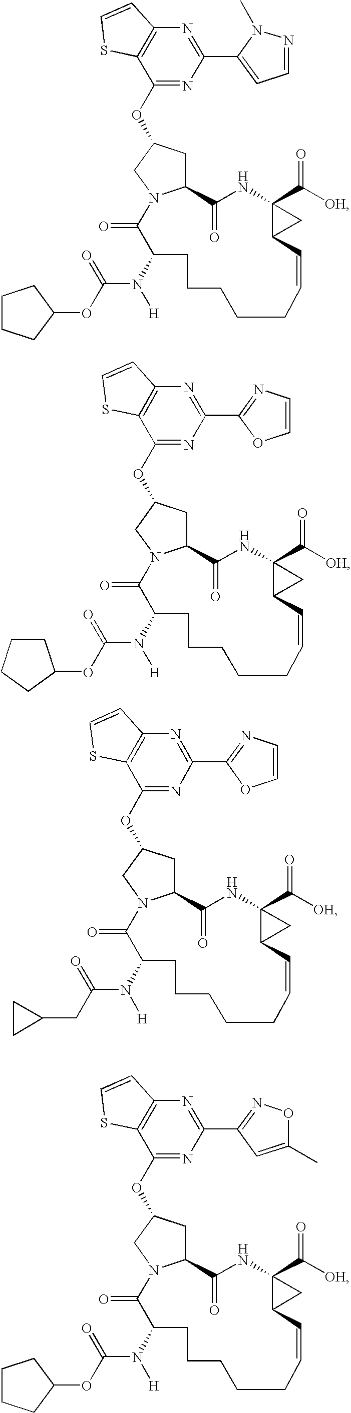 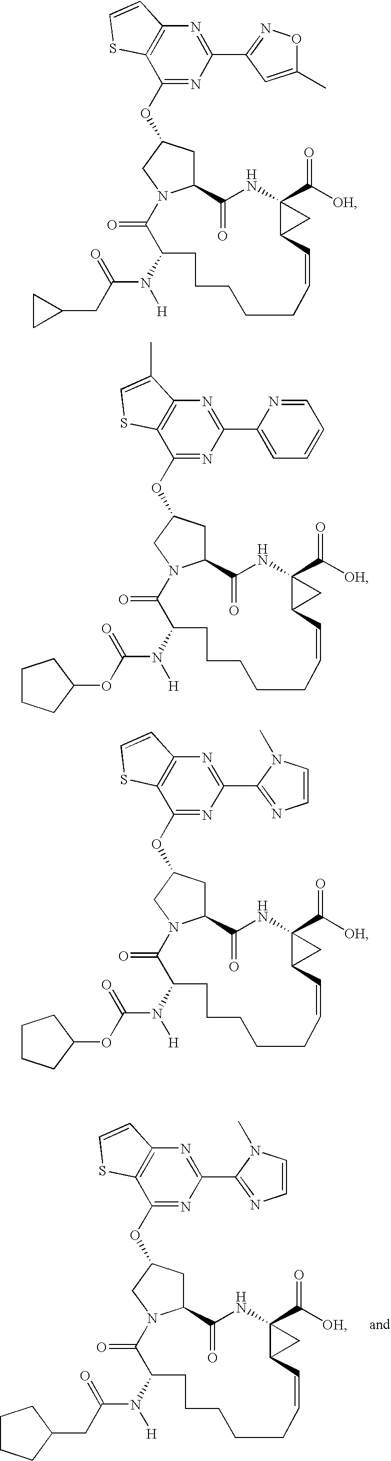 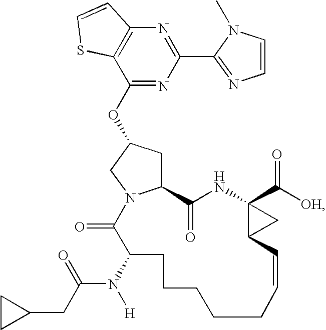
or pharmaceutically acceptable salts or solvates thereof.

In yet another aspect are compounds selected from: 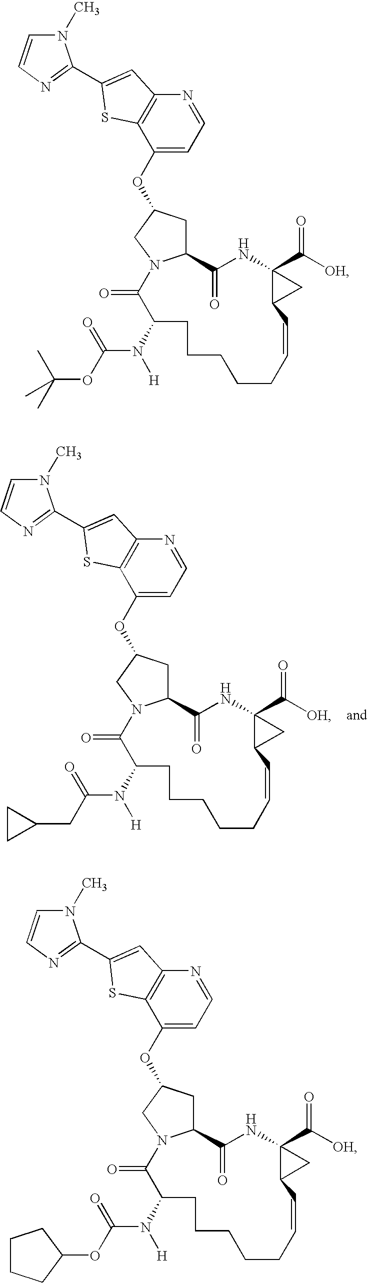
or pharmaceutically acceptable salts or solvates thereof.

The present invention further relates to a method of treating a mammal infected with Hepatitis C virus comprising administering to said mammal a Hepatitis C virus-inhibiting amount of a compound provided herein.

The present invention further relates to a method of inhibiting Hepatitis C protease activity comprising contacting said protease with a protease-inhibiting amount of a compound provided herein.

The present invention further relates to a pharmaceutical composition comprising an amount of a compound provided herein that is effective in treating Hepatitis C virus in an infected mammal, and a pharmaceutically acceptable carrier. For Example, HCV activity may be inhibited in mammalian tissue by administering an HCV-inhibiting agent according to the invention.

The present invention further relates to a method of inhibiting Hepatitis C virus replication comprising contacting said virus with a replication-inhibiting amount of a compound provided herein.

The present invention further relates to a method of inhibiting Hepatitis C virus replication in a mammal comprising administering to said mammal a Hepatitis C virus replication-inhibiting amount of a compound provided herein.

The present invention further relates to a method of inhibiting Hepatitis C virus protein protease activity comprising contacting the protein with an effective amount of a compound provided herein.

The present invention further relates to a use of a compound provided herein in the preparation of a medicament for the treatment of a mammal suffering from infection with Hepatitis C virus. The medicament may comprise a Hepatitis C virus-inhibiting amount of a compound or compounds of the invention and a pharmaceutically acceptable carrier or carriers.

As used herein, the terms “comprising” and “including” are used in their open, non-limiting sense.

The term “C₁-C₆ alkyl”, as used herein, unless otherwise indicated, includes saturated monovalent hydrocarbon radicals having straight, branched, or cyclic moieties (including fused and bridged bicyclic and spirocyclic moieties), or a combination of the foregoing moieties, and containing from 1-6 carbon atoms. For an alkyl group to have cyclic moieties, the group must have at least three carbon atoms.

A “lower alkyl” is intended to mean an alkyl group having from 1 to 4 carbon atoms in its chain. The term “heteroalkyl” refers to a straight- or branched-chain alkyl group having from 2 to 12 atoms in the chain, one or more of which is a heteroatom selected from S, O, and N. Exemplary heteroalkyls include alkyl ethers, secondary and tertiary amines, alkyl sulfides and the like.

The term “C₂-C₆ alkenyl”, as used herein, unless otherwise indicated, includes alkyl moieties having at least one carbon-carbon double bond wherein alkyl is as defined above and including E and Z isomers of said alkenyl moiety, and having from 2 to 6 carbon atoms.

The term “C₂-C₆ alkynyl”, as used herein, unless otherwise indicated, includes alkyl moieties having at least one carbon-carbon triple bond wherein alkyl is as defined above, and containing from 2-6 carbon atoms.

The term “carbocycle” refers to a saturated, partially saturated, unsaturated, or aromatic, monocyclic or fused or non-fused polycyclic, ring structure having only carbon ring atoms (no heteroatoms, i.e., non-carbon ring atoms). Exemplary carbocycles include cycloalkyl, aryl, and cycloalkyl-aryl groups.

A “C₃-C₁₀ cycloalkyl group” is intended to mean a saturated or partially saturated, monocyclic, or fused or spiro polycyclic, ring structure having a total of from 3 to 10 carbon ring atoms (but no heteroatoms). Exemplary cycloalkyls include cyclopropyl, cyclobutyl, cyclopentyl, cyclopentenyl, cyclohexyl, cycloheptyl, adamantyl, and like groups.

A “heterocycloalkyl group” is intended to mean a monocyclic, or fused or spiro polycyclic, ring structure that is saturated or partially saturated, and has a total of from 3 to 18 ring atoms, including 1 to 5 heteroatoms selected from nitrogen, oxygen, and sulfur. Illustrative Examples of heterocycloalkyl groups include pyrrolidinyl, tetrahydrofuryl, piperidinyl, piperazinyl, morpholinyl, thiomorpholinyl, aziridinyl, and like groups.

The term “C₆-C₁₀ aryl”, as used herein, unless otherwise indicated, includes an organic radical derived from an aromatic hydrocarbon by removal of one hydrogen, such as phenyl or naphthyl. The term “phenyl” and the symbol “Ph,” as used herein, refer to a C₆H₅ group.

The term “4-10 membered heterocyclic”, as used herein, unless otherwise indicated, includes aromatic and non-aromatic heterocyclic groups containing one to four heteroatoms each selected from O, S and N, wherein each heterocyclic group has from 4-10 atoms in its ring system, and with the proviso that the ring of said group does not contain two adjacent O or S atoms. Furthermore, the sulfur atoms contained in such heterocyclic groups may be oxidized with one or two sulfur atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, oxadiazolyl, isoxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). An example of a heterocyclic group wherein 2 ring carbon atoms are substituted with oxo (═O) moieties is 1,1-dioxo-thiomorpholinyl.

The term “5-6 membered heterocyclic” means aromatic and non-aromatic heterocyclic groups containing one to four heteroatoms each selected from O, S and N, and wherein each heterocyclic group has a total of from 5 to 6 atoms in its ring system, and with the proviso that the ring of said group does not contain two adjacent O or S atoms. The sulfur atoms contained in such heterocyclic groups may be oxidized with one or two sulfur atoms. Furthermore, any atom in the 5-6 membered heterocyclic group may be substituted with an oxo (═O) group, if such substitution would result in a stable compound. Examples of non-aromatic heterocyclic groups include, but are not limited to, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups include, but are not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, quinazolinyl, and quinoxalinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). An example of a heterocyclic group wherein 2 ring carbon atoms are substituted with oxo (═O) moieties is 1,1-dioxo-thiomorpholinyl.

A “heteroaryl group” is intended to mean a monocyclic or fused or spiro polycyclic, aromatic ring structure having from 4 to 18 ring atoms, including from 1 to 5 heteroatoms selected from nitrogen, oxygen, and sulfur. Illustrative Examples of heteroaryl groups include pyrrolyl, thienyl, oxazolyl, isoxazolyl, pyrazolyl, thiazolyl, furyl, pyridinyl, pyrazinyl, triazolyl, tetrazolyl, indolyl, quinolinyl, quinoxalinyl, benzthiazolyl, benzodioxinyl, benzodioxolyl, benzooxazolyl, oxadiazolyl, and the like.

The term “alkoxy”, as used herein, unless otherwise indicated, includes O-alkyl groups wherein alkyl is as defined above.

The term “amino” is intended to mean the —NH₂ radical.

The terms “halogen” and “halo,” as used herein represent fluorine, chlorine, bromine or iodine.

The term “oxo,” as used herein, means a group (═O). Such a group may be bonded to either a carbon atom or a heteroatom in the compounds of the present invention, if such substitution will result in a stable compound.

The term “trifluoromethyl,” as used herein, is meant to represent a group —CF₃.

The term “trifluoromethoxy,” as used herein, is meant to represent a group —OCF₃.

The term “cyano,” as used herein, is meant to represent a group —CN.

The term “substituted” means that the specified group or moiety bears one or more substituents. The term “unsubstituted” means that the specified group bears no substituents. The term “optionally substituted” means that the specified group is unsubstituted or substituted by one or more substituents.

The term “HCV,” as used herein, refers to Hepatitis C virus.

The terms “inhibiting Hepatitis C virus” and “inhibiting Hepatitis C virus replication” mean inhibiting Hepatitis C virus replication either in vitro or in vivo, such as in a mammal, such as a human, by contacting the Hepatitis C virus with an HCV-replication inhibiting amount of a compound of the present invention, or a pharmaceutically acceptable salt or solvate thereof. Such inhibition may take place in vivo, such as in a mammal, such as a human, by administering to the mammal a Hepatitis C virus-inhibiting amount of a compound of the present invention. The amount of a compound of the present invention necessary to inhibit replication of the HCV virus either in vitro or in vivo, such as in a mammal, such as a human, can be determined using methods known to those of ordinary skill in the art. For example, an amount of a compound of the invention may be administered to a mammal, either alone or as part of a pharmaceutically acceptable formulation. Blood samples may then be withdrawn from the mammal and the amount of Hepatitis C virus in the sample may be quantified using methods known to those of ordinary skill in the art. A reduction in the amount of Hepatitis C virus in the sample compared to the amount found in the blood before administration of a compound of the invention would represent inhibition of the replication of Hepatitis C virus in the mammal. The administration of a compound of the invention to the mammal may be in the form of single dose or a series of doses over successive days.

An “HCV-inhibiting agent” means a compound of the present invention or a pharmaceutically acceptable salt or solvate thereof.

The term “HCV-inhibiting amount,” as used herein, refers to an amount of a compound of the present invention that is sufficient to inhibit the replication of the Hepatitis C virus when administered to a mammal, such as a human.

The term “HCV protease-inhibiting amount,” as used herein, means an amount of a compound of the present invention that is sufficient to inhibit the function of the Hepatitis C virus protease enzyme when the compound is placed in contact with the enzyme.

A “solvate” is intended to mean a pharmaceutically acceptable solvate form of a specified compound that retains the biological effectiveness of such compound. Examples of solvates include compounds of the invention in combination with solvents such as, but not limited to, water, isopropanol, ethanol, methanol, DMSO, ethyl acetate, acetic acid, or ethanolamine. A “pharmaceutically acceptable salt” is intended to mean a salt that retains the biological effectiveness of the free acids and bases of the specified derivative and that is not biologically or otherwise undesirable. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The phrase “pharmaceutically acceptable salt(s)”, as used herein, unless otherwise indicated, includes salts of acidic or basic groups, which may be present in the compounds of the present invention. The compounds of the present invention that are basic in nature are capable of forming a wide variety of salts with various inorganic and organic acids. The acids that may be used to prepare pharmaceutically acceptable acid addition salts of such basic compounds of the present invention are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phosphate/diphosphate, polygalacturonate, salicylate, stearate, subacetate, succinate, tannate, tartrate, teoclate, tosylate, triethiodode, and valerate salts.

The phrases “therapeutically effective amount,” “effective amount,” and “HCV-inhibiting amount,” are intended to mean the amount of an inventive agent that, when administered to a mammal in need of treatment, is sufficient to effect treatment for injury or disease conditions alleviated by the inhibition of HCV RNA replication such as for potentiation of anti-cancer therapies or inhibition of neurotoxicity consequent to stroke, head trauma, and neurodegenerative diseases. The amount of a given HCV-inhibiting agent used in the method of the invention that will be therapeutically effective will vary depending upon factors such as the particular HCV-inhibiting agent, the disease condition and the severity thereof, the identity and characteristics of the mammal in need thereof, which amount may be routinely determined by artisans.

As used herein, the term “catalyst” means a chemical element or compound that increases the rate of a chemical reaction by reducing the activation energy, but which is left unchanged by the reaction. Examples of catalysts include, but are not limited to, palladium (0) and platinum (0). It is specifically contemplated herein that such catalysts may be formed in situ during the course of a chemical reaction, from a so-called “pre-catalyst,” but may never actually be observed or isolated. Such pre-catalysts are chemical compounds that are capable of being converted in situ during the course of a chemical reaction to a chemically and catalytically competent element or compound. Examples of suitable pre-catalysts include, but are not limited to, PdCl₂, PdCl₂(PPh₃)₂, Pd(OH)₂, Pd(PPh₃)₄, Pt(OH)₂, and PtCl₂.

The term “reducing agent,” as used herein, means a chemical element or compound that provides electrons for another chemical element or compound in a reaction mixture. Alternatively, it means a chemical element or compound that is capable of affording a saturated chemical compound from an unsaturated chemical compound by the addition of hydrogen. For example, the addition of hydrogen to an alkene of the present invention to afford a saturated alkane is termed “reduction.” A reducing agent is a chemical element or compound that is capable of affecting such a reduction, usually in the presence of a catalyst. Examples of reducing agents include, but are not limited to hydrogen, formic acid, and formic acid salts, such as ammonium formate.

The term “protecting,” as used herein, refers to a process in which a functional group in a chemical compound is selectively masked by a non-reactive functional group in order to allow a selective reaction(s) to occur elsewhere on said chemical compound. Such non-reactive functional groups are herein termed “protecting groups.” For example, the term “hydroxyl protecting group,” as used herein refers to those groups that are capable of selectively masking the reactivity of a hydroxyl (—OH) group. The term “suitable protecting group,” as used herein refers to those protecting groups that are useful in the preparation of the compounds of the present invention. Such groups are generally able to be selectively introduced and removed using mild reaction conditions that do not interfere with other portions of the subject compounds. Protecting groups that are suitable for use in the processes and methods of the present invention are known to those of ordinary skill in the art. The chemical properties of such protecting groups, methods for their introduction, and their removal can be found, for example, in T. Greene and P. Wuts, Protective Groups in Organic Synthesis (3^{rd} ed.), John Wiley & Sons, NY (1999). The terms “deprotecting,” “deprotected,” or “deprotect,” as used herein, are meant to refer to the process of removing a protecting group from a compound.

The terms “hydrolyze,” “hydrolyzing,” “hydrolysis,” and “hydrolyzed,” as used herein, all mean and refer to a chemical reaction in which an ester, an amide, or both are converted into their corresponding carboxylic acid derivatives, usually through the action of hydroxyl anion (—OH), such as would be present in a basic, aqueous solution.

The term “leaving group,” as used herein, refers to a chemical functional group that generally allows a nucleophilic substitution reaction to take place at the atom to which it is attached. For example, in acid chlorides of the formula Cl—C(O)R, wherein R is alkyl, aryl, or heterocyclic, the —Cl group is generally referred to as a leaving group because it allows nucleophilic substitution reactions to take place at the carbonyl carbon to which it is attached. Suitable leaving groups are known to those of ordinary skill in the art and can include halides, aromatic heterocycles, cyano, amino groups (generally under acidic conditions), ammonium groups, alkoxide groups, carbonate groups, formates, and hydroxy groups that have been activated by reaction with compounds such as carbodiimides. For example, suitable leaving groups can include, but are not limited to, chloride, bromide, iodide, cyano, imidazole, and hydroxy groups that have been allowed to react with a carbodiimide such as dicyclohexylcarbodiimide (optionally in the presence of an additive such as hydroxybenzotriazole) or a carbodiimide derivative.

The term “combination of reagents,” means a chemical reagent, or more than one reagent when necessary, that can be used to affect a desired chemical reaction. The choice of a particular reagent, or combination or reagents, will depend on factors that are familiar to those of ordinary skill in the art and include, but are not limited to, the identity of the reactants, the presence of other functional groups in the reactants, the solvent or solvents used in a particular chemical reaction, the temperature at which the chemical reaction will be performed, and the method or methods of purification of the desired chemical reaction product. The choice of a reagent, or combination of reagents, required to affect a particular chemical reaction are within the knowledge of one of ordinary skill in the art and such a choice can be made without undue experimentation.

The term “base,” as used herein, means a so-called Bronsted-Lowry base. A Bronsted-Lowry base is a reagent that is capable of accepting a proton (H⁺) from an acid present in a reaction mixture. Examples of Bronsted-Lowry bases include, but are not limited to, inorganic bases such as sodium carbonate, sodium bicarbonate, sodium hydroxide, potassium carbonate, potassium bicarbonate, potassium hydroxide, and cesium carbonate, inorganic bases such as triethylamine, diisopropylethylamine, diisopropylamine, dicyclohexylamine, morpholine, pyrrolidone, piperidine, pyridine, 4-N,N-dimethylaminopyridine (DMAP), and imidazole.

The term “chiral, non-racemic base,” as used herein, means a basic compound that can exist in an enantiomeric form and is not present in an equal amount with its corresponding opposite enantiomer. For example, the compound 2-phenylglycinol exists as two enantiomers of opposite configuration, the so-called (R)- and (S)-enantiomers. If the (R)- and the (S)-enantiomers are present in equal amounts, such a mixture is said to be “racemic.” If, however, one enantiomer is present in an amount greater than the other, the mixture is said to be “non-racemic.”

The term “stereoisomers” refers to compounds that have identical chemical constitution, but differ with regard to the arrangement of their atoms or groups in space. In particular, the term “enantiomers” refers to two stereoisomers of a compound that are non-superimposable mirror images of one another. The terms “racemic” or “racemic mixture,” as used herein, refer to a 1:1 mixture of enantiomers of a particular compound. The term “diastereomers”, on the other hand, refers to the relationship between a pair of stereoisomers that comprise two or more asymmetric centers and are not mirror images of one another.

The term “stereochemically-enriched” product, when used herein, refers to a reaction product wherein a particular stereoisomer is present in a statistically significant greater amount relative to the other possible stereoisomeric products. For example, a product that comprises more of one enantiomer than the other would constitute a stereochemically enriched product. Similarly, a product that comprises more of one diastereoisomer than others would also constitute a stereochemically enriched product. The methods and processes contained herein are said to afford a “stereochemically enriched” product. In such cases, the methods and processes contained herein begin with a mixture of stereoisomeric compounds in which all possible stereoisomers are present in about an equal amount and afford a product in which at least one stereoisomer is present in a statistically significant greater amount than the others.

The term “diastereomeric,” as used herein refers to the relationship between a pair of stereoisomers that comprise two or more asymmetric centers and are non-superimposable mirror images of one another. The phrases “diastereomeric salt,” or “diastereomeric salts,” as used herein means a salt of a diastereomeric compound, wherein “diastereomer” is as defined herein.

The term “racemic,” as used herein, means a composition comprising a 1:1 ratio of enantiomers. The term “scalemic,” as used herein, means a composition comprising an unequal amount of enantiomers. For example, a composition comprising a 1:1 mixture of the (R)- and (S)-enantiomers of a compound of the present invention is termed a racemic composition or mixture. As an additional example, a composition comprising a 2:1 mixture of (R)- and (S)-enantiomers of a compound of the present invention is termed a scalemic composition or mixture. It is specifically contemplated that the methods of the present invention may be advantageously used to prepare a scalemic compound of the present invention from a racemic compound of the present invention.

The terms “resolution” and “resolving” mean a method of physically separating stereoisomeric compounds from a mixture of stereoisomers, such as a racemic mixture comprising two enantiomers of a particular compound. As used herein, “resolution” and “resolving” are meant to include both partial and complete resolution.

The terms “separating” or “separated,” as used herein, mean a process of physically isolating at least two different chemical compounds from each other. For example, if a chemical reaction takes place and produces at least two products, (A) and (B), the process of isolating both (A) and (B) from each other is termed “separating” (A) and (B). It is specifically contemplated that the separations of the present invention may be partial or complete as determined by analytical techniques known to those of ordinary skill in the art and those described herein.

The term “converting,” as used herein, means allowing a chemical reaction to take place with a starting material or materials to produce a different chemical product. For example, if chemical reactants (A) and (B) are allowed to react with each other to produce product (C), starting materials (A) and (B) can be said to have “converted” to product (C), or it can be said that (A) was “converted” to (C), or that (B) was “converted” to (C).

The term “substituted,” means that the specified group or moiety bears one or more substituents. The term “unsubstituted,” means that the specified group bears no substituents. The term “optionally substituted” means that the specified group is unsubstituted or substituted by one or more substituents.

### DETAILED DESCRIPTION

In accordance with a convention used in the art, the symbol 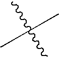
is used in structural formulas herein to depict the bond that is the point of attachment of the moiety or substituent to the core or backbone structure. In accordance with another convention, in some structural formulae herein the carbon atoms and their bound hydrogen atoms are not explicitly depicted, e.g., 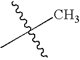
represents a methyl group, 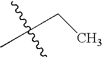
represents an ethyl group, 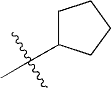
represents a cyclopentyl group, etc.

The compounds of the present invention may have asymmetric carbon atoms. The carbon-carbon bonds of the compounds of the present invention may be depicted herein using a solid line [symbol] a solid wedge [symbol] or a dotted wedge [symbol] The use of a solid line to depict bonds to asymmetric carbon atoms is meant to indicate that all possible stereoisomers at that carbon atom are included. The use of either a solid or dotted wedge to depict bonds to asymmetric carbon atoms is meant to indicate that only the stereoisomer shown is meant to be included. It is possible that compounds of the invention may contain more than one asymmetric carbon atom. In those compounds, the use of a solid line to depict bonds to asymmetric carbon atoms is meant to indicate that all possible stereoisomers are meant to be included. The use of a solid line to depict bonds to one or more asymmetric carbon atoms in a compound of the invention and the use of a solid or dotted wedge to depict bonds to other asymmetric carbon atoms in the same compound is meant to indicate that a mixture of diastereomers is present.

Solutions of individual stereoisomeric compounds of the present invention may rotate plane-polarized light. The use of either a “(+)” or “(−)” symbol in the name of a compound of the invention indicates that a solution of a particular stereoisomer rotates plane-polarized light in the (+) or (−) direction, as measured using techniques known to those of ordinary skill in the art.

Diastereomeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomeric mixtures and pure enantiomers are considered as part of the invention.

Alternatively, individual stereoisomeric compounds of the present invention may be prepared in enantiomerically enriched form by asymmetric synthesis. Asymmetric synthesis may be performed using techniques known to those of skill in the art, such as the use of asymmetric starting materials that are commercially available or readily prepared using methods known to those of ordinary skill in the art, the use of asymmetric auxiliaries that may be removed at the completion of the synthesis, or the resolution of intermediate compounds using enzymatic methods. The choice of such a method will depend on factors that include, but are not limited to, the availability of starting materials, the relative efficiency of a method, and whether such methods are useful for the compounds of the invention containing particular functional groups. Such choices are within the knowledge of one of ordinary skill in the art.

When the compounds of the present invention contain asymmetric carbon atoms, the derivative salts, prodrugs and solvates may exist as single stereoisomers, racemates, and/or mixtures of enantiomers and/or diastereomers. All such single stereoisomers, racemates, and mixtures thereof are intended to be within the scope of the present invention.

As generally understood by those skilled in the art, an optically pure compound is one that is enantiomerically pure. As used herein, the term “optically pure” is intended to mean a compound comprising at least a sufficient activity. Preferably, an optically pure amount of a single enantiomer to yield a compound having the desired pharmacological pure compound of the invention comprises at least 90% of a single isomer (80% enantiomeric excess), more preferably at least 95% (90% e.e.), even more preferably at least 97.5% (95% e.e.), and most preferably at least 99% (98% e.e.).

If a derivative used in the method of the invention is a base, a desired salt may be prepared by any suitable method known to the art, including treatment of the free base with an inorganic acid, such as: hydrochloric acid; hydrobromic acid; sulfuric acid; nitric acid; phosphoric acid; and the like, or with an organic acid, such as: acetic acid; maleic acid; succinic acid; mandelic acid; fumaric acid; malonic acid; pyruvic acid; oxalic acid; glycolic acid; salicylic acid; pyranosidyl acid, such as glucuronic acid or galacturonic acid; alpha-hydroxy acid, such as citric acid or tartaric acid; amino acid, such as aspartic acid or glutamic acid; aromatic acid, such as benzoic acid or cinnamic acid; sulfonic acid, such as p-toluenesulfonic acid or ethanesulfonic acid; and the like.

If a derivative used in the method of the invention is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Illustrative Examples of suitable salts include organic salts derived from amino acids such as glycine and arginine; ammonia; primary, secondary, and tertiary amines; and cyclic amines, such as piperidine, morpholine, and piperazine; as well as inorganic salts derived from sodium, calcium, potassium, magnesium, manganese, iron, copper, zinc, aluminum, and lithium.

In the case of derivatives, prodrugs, salts, or solvates that are solids, it is understood by those skilled in the art that the derivatives, prodrugs, salts, and solvates used in the method of the invention, may exist in different polymorph or crystal forms, all of which are intended to be within the scope of the present invention and specified formulas. In addition, the derivative, salts, prodrugs and solvates used in the method of the invention may exist as tautomers, all of which are intended to be within the broad scope of the present invention.

The compounds of the present invention that are basic in nature are capable of forming a wide variety of different salts with various inorganic and organic acids. Although such salts must be pharmaceutically acceptable for administration to animals, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of the present invention that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of the present invention. Such non-toxic base salts include those derived from such pharmacologically acceptable cations as sodium, potassium calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

The activity of the compounds as inhibitors of HCV activity may be measured by any of the suitable methods available in the art, including in vivo and in vitro assays. An Example of a suitable assay for activity measurements is the HCV replicon assay described herein.

Administration of the compounds and their pharmaceutically acceptable prodrugs, salts, active metabolites, and solvates may be performed according to any of the accepted modes of administration available to those skilled in the art. Illustrative Examples of suitable modes of administration include oral, nasal, parenteral, topical, transdermal, and rectal. Oral and intravenous deliveries are preferred.

An HCV-inhibiting agent of the present invention may be administered as a pharmaceutical composition in any suitable pharmaceutical form. Suitable pharmaceutical forms include solid, semisolid, liquid, or lyophilized formulations, such as tablets, powders, capsules, suppositories, suspensions, liposomes, and aerosols. The HCV-inhibiting agent may be prepared as a solution using any of a variety of methodologies. For Example, the HCV-inhibiting agent can be dissolved with acid (e.g., 1 M HCl) and diluted with a sufficient volume of a solution of 5% dextrose in water (D5W) to yield the desired final concentration of HCV-inhibiting agent (e.g., about 15 mM). Alternatively, a solution of D5W containing about 15 mM HCl can be used to provide a solution of the HCV-inhibiting agent at the appropriate concentration. Further, the HCV-inhibiting agent can be prepared as a suspension using, for example, a 1% solution of carboxymethylcellulose (CMC).

Acceptable methods of preparing suitable pharmaceutical forms of the pharmaceutical compositions are known or may be routinely determined by those skilled in the art. For Example, pharmaceutical preparations may be prepared following conventional techniques of the pharmaceutical chemist involving steps such as mixing, granulating, and compressing when necessary for tablet forms, or mixing, filling, and dissolving the ingredients as appropriate, to give the desired products for oral, parenteral, topical, intravaginal, intranasal, intrabronchial, intraocular, intraaural, and/or rectal administration.

Pharmaceutical compositions of the invention may also include suitable excipients, diluents, vehicles, and carriers, as well as other pharmaceutically active agents, depending upon the intended use. Solid or liquid pharmaceutically acceptable carriers, diluents, vehicles, or excipients may be employed in the pharmaceutical compositions. Illustrative solid carriers include starch, lactose, calcium sulfate dihydrate, terra alba, sucrose, talc, gelatin, pectin, acacia, magnesium stearate, and stearic acid. Illustrative liquid carriers include syrup, peanut oil, olive oil, saline solution, and water. The carrier or diluent may include a suitable prolonged-release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., solution), or a nonaqueous or aqueous liquid suspension.

A dose of the pharmaceutical composition may contain at least a therapeutically effective amount of an HCV-inhibiting agent and preferably is made up of one or more pharmaceutical dosage units. The selected dose may be administered to a mammal, for example, a human, in need of treatment mediated by inhibition of HCV activity, by any known or suitable method of administering the dose, including topically, for example, as an ointment or cream; orally; rectally, for example, as a suppository; parenterally by injection; intravenously; or continuously by intravaginal, intranasal, intrabronchial, intraaural, or intraocular infusion. When the composition is administered in conjunction with a cytotoxic drug, the composition can be administered before, with, and/or after introduction of the cytotoxic drug. However, when the composition is administered in conjunction with radiotherapy, the composition is preferably introduced before radiotherapy is commenced.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15^{th} Edition (1975).

It will be appreciated that the actual dosages of the HCV-inhibiting agents used in the pharmaceutical compositions of this invention will be selected according to the properties of the particular agent being used, the particular composition formulated, the mode of administration and the particular site, and the host and condition being treated. Optimal dosages for a given set of conditions can be ascertained by those skilled in the art using conventional dosage-determination tests. For oral administration, e.g., a dose that may be employed is from about 0.001 to about 1000 mg/kg body weight, or from about 0.1 to about 100 mg/kg body weight, or from about 1 to about 50 mg/kg body weight, or from about 0.1 to about 1 mg/kg body weight, with courses of treatment repeated at appropriate intervals. The dosage forms of the pharmaceutical formulations described herein may contain an amount of a compound of the present invention, or a pharmaceutically acceptable salt of solvate thereof, deemed appropriate by one of ordinary skill in the art. For example, such dosage forms may contain from about 1 mg to about 1500 mg of a compound of the present invention, or may contain from about 5 mg to about 1500 mg, or from about 5 mg to about 1250 mg, or from about 10 mg to about 1250 mg, or from about 25 mg to about 1250 mg, or from about 25 mg to about 1000 mg, or from about 50 mg to about 1000 mg, or from about 50 mg to about 750 mg, or from about 75 mg to about 750 mg, or from about 100 mg to about 750 mg, or from about 125 mg to about 750 mg, or from about 150 mg to about 750 mg, or from about 150 mg to about 500 mg of a compound of the present invention, or a pharmaceutically acceptable salt or solvate thereof.

The subject invention also includes isotopically-labelled compounds, which are identical to those recited in the compounds of the present invention, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures disclosed in the Schemes and/or in the Examples and Preparations below, by substituting a readily available isotopically labeled reagent for a non-isotopically labeled reagent.

The compounds of the present invention are potent inhibitors of Hepatitis C virus, in particular HCV replication, and even in more particular, HCV protease. The compounds are all adapted to therapeutic use as anti-HCV agents in mammals, particularly in humans.

The active compound may be applied as a sole therapy or may involve one or more other antiviral substances, for example those selected from, for example, HCV inhibitors such as interferon alphacon-1, natural interferon, interferon beta-1a, interferon omega, interferon gamma-1b, interleukin-10, BILN 2061 (serine protease), amantadine (Symmetrel), thymozine alpha-1, viramidine; HIV inhibitors such as nelfinavir, delavirdine, indinavir, nevirapine, saquinavir, and tenofovir. Such conjoint treatment may be achieved by way of the simultaneous, sequential or separate dosing of the individual components of the treatment.

In general, the compounds of the present invention may be prepared according to the methods described herein as well as methods known to those of ordinary skill in the art. The methods described herein are not meant to, and should not be construed to, limit the scope of the present invention in any way.

A general scheme for the preparation of a thienopyrimidine isomer is shown below starting from commercially available compound A. 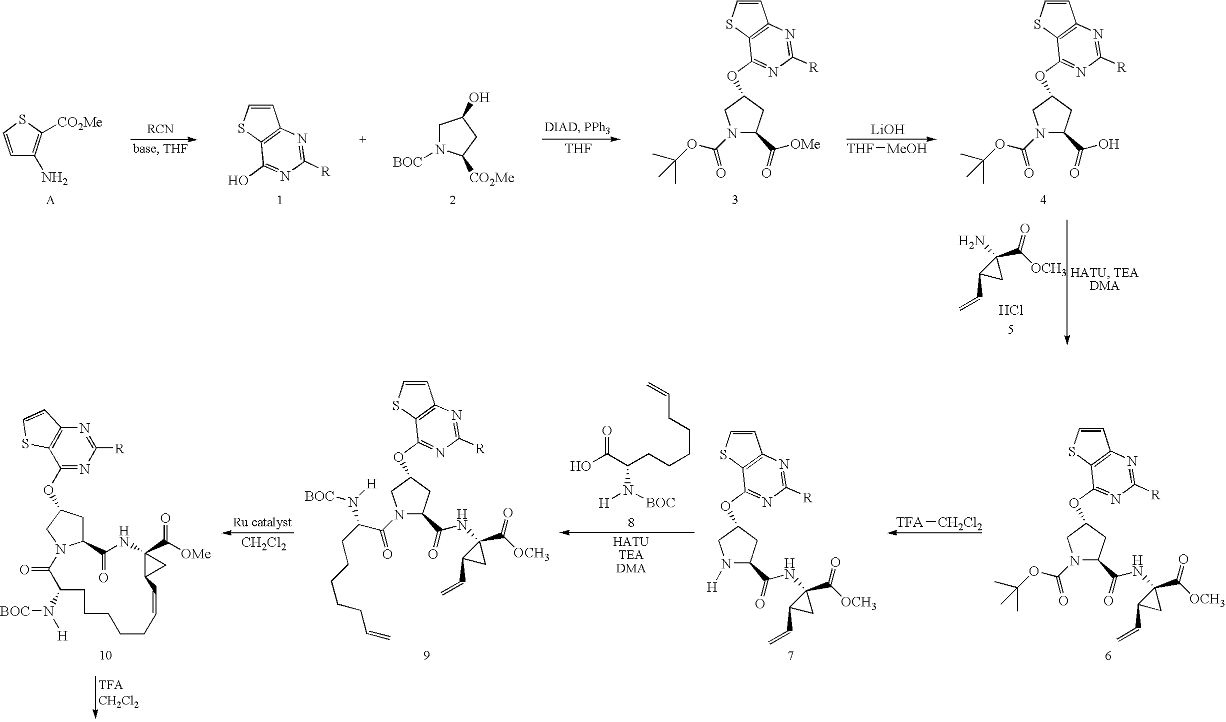 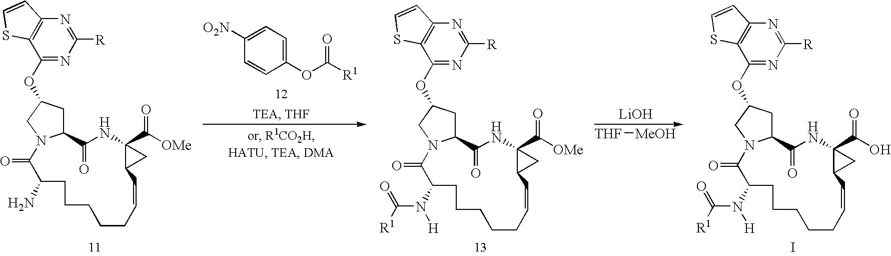
R=aryl or heteroaryl, R¹=O-alkyl, or alkyl. The other thienopyrimidine isomer of type II is made in a similar manner except commercially available compound A is replaced with commercially available compound B.

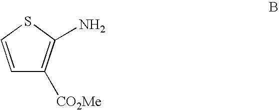

A general scheme for the preparation of a thienopyridine isomer (type I) is shown below starting from commercially available compound A.

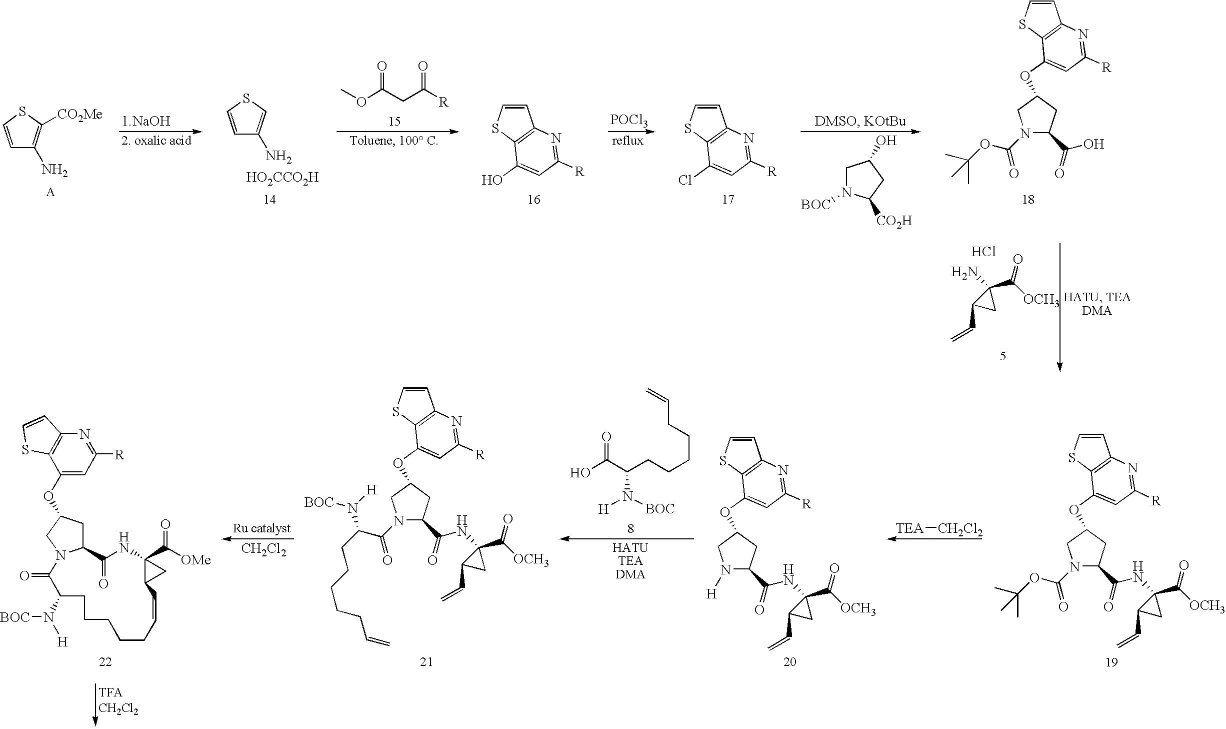

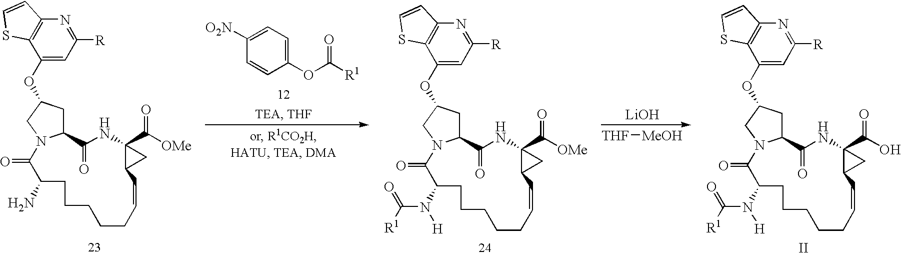

Synthesis of compound 5 utilized the procedure as disclosed in WO 00/09543 and modified as follows:

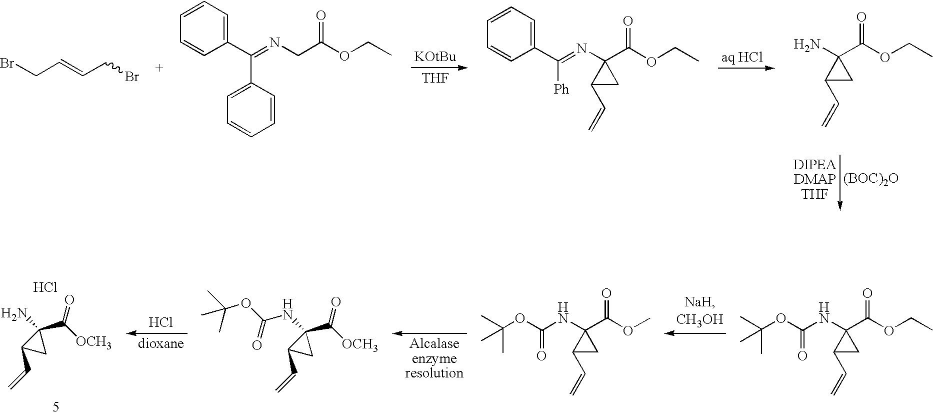

Synthesis of compound 8 utilized the procedure as disclosed in WO 00/59929:

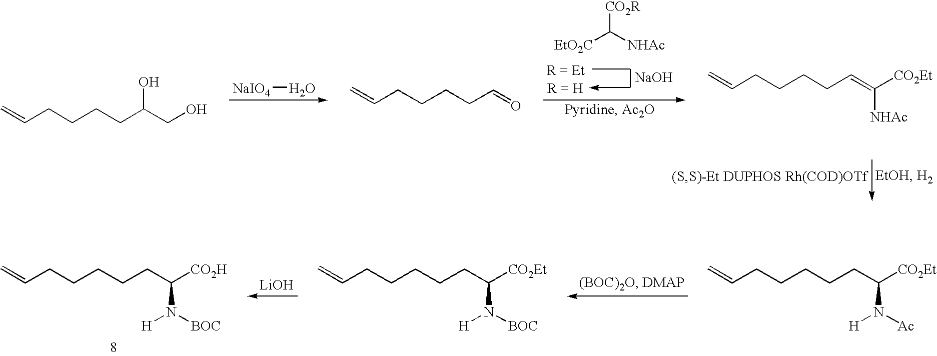

The following is a representative example for the synthesis of compound 12:

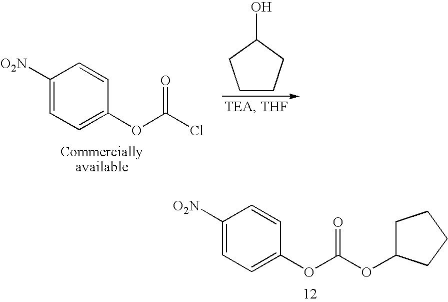

The following is a representative example for the synthesis of thieno pyrimidines:

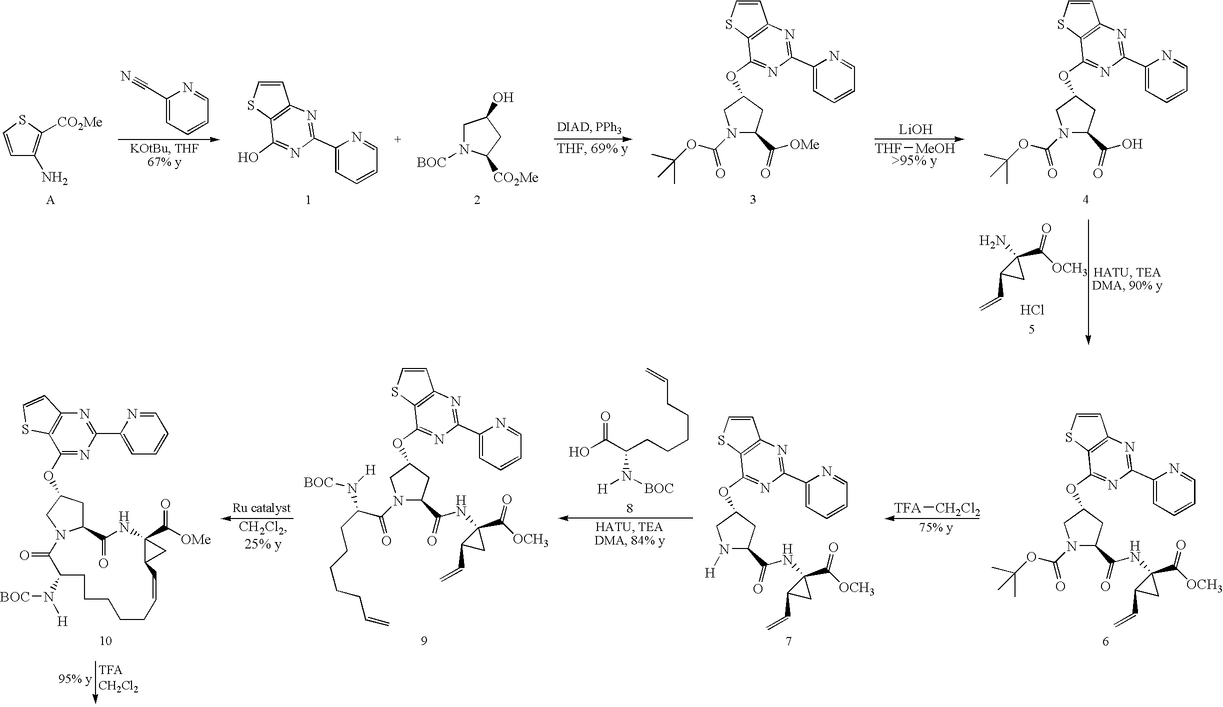

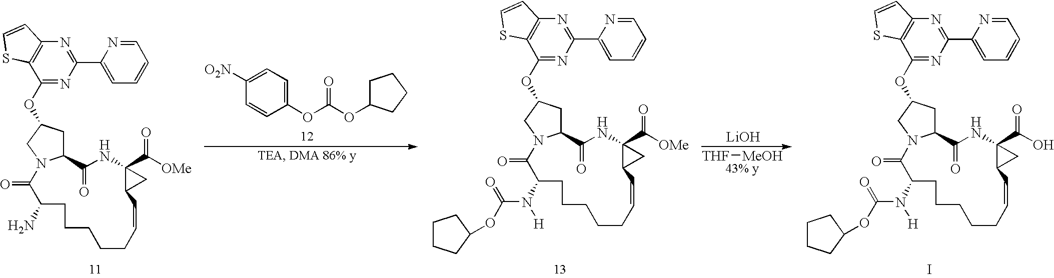

The following is a representative example for the synthesis of compound thienopyridines:

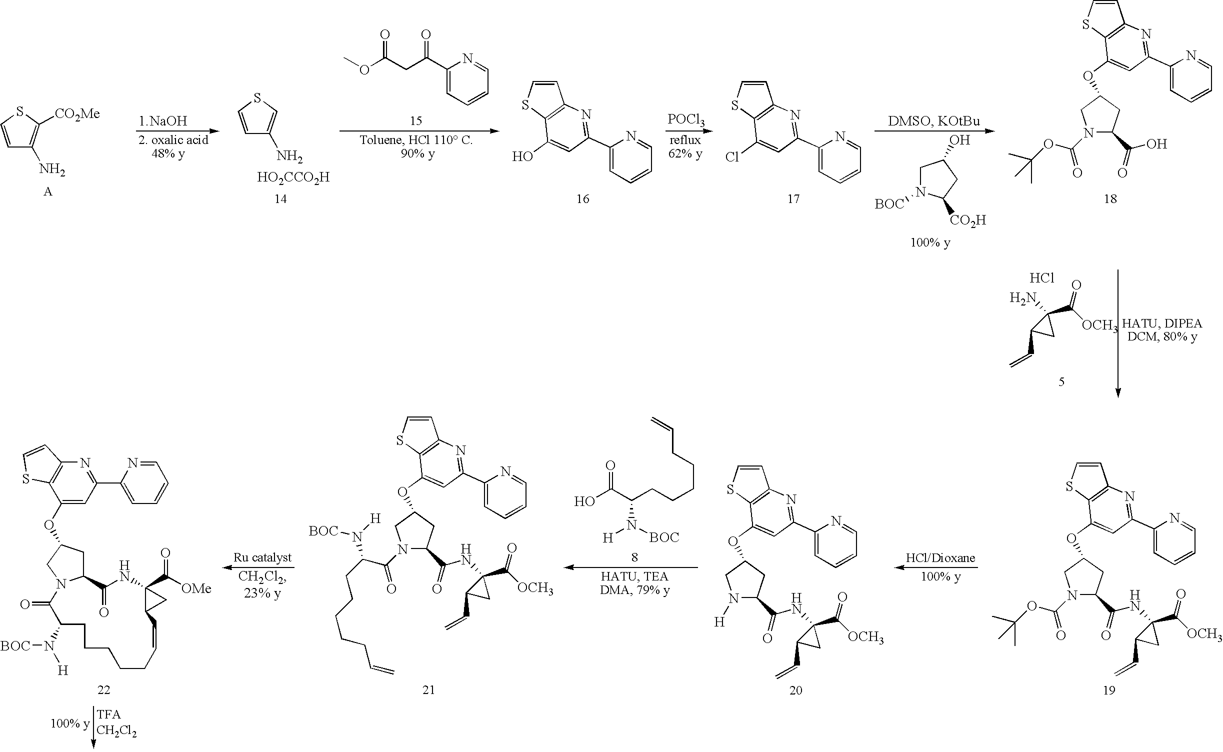

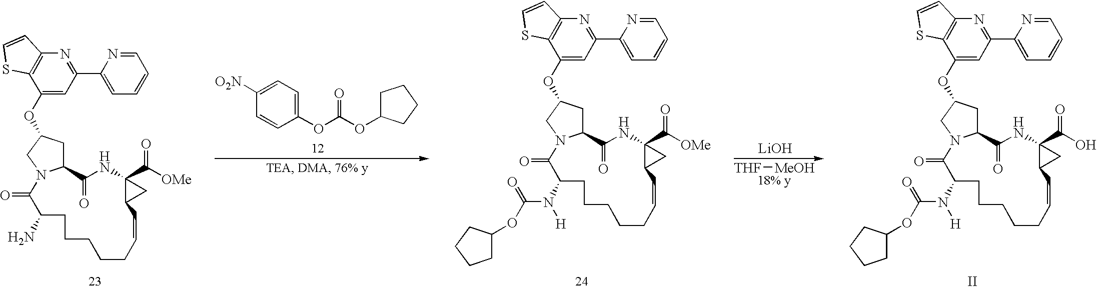

An alternative general scheme to synthesize compounds I or II is outlined below:

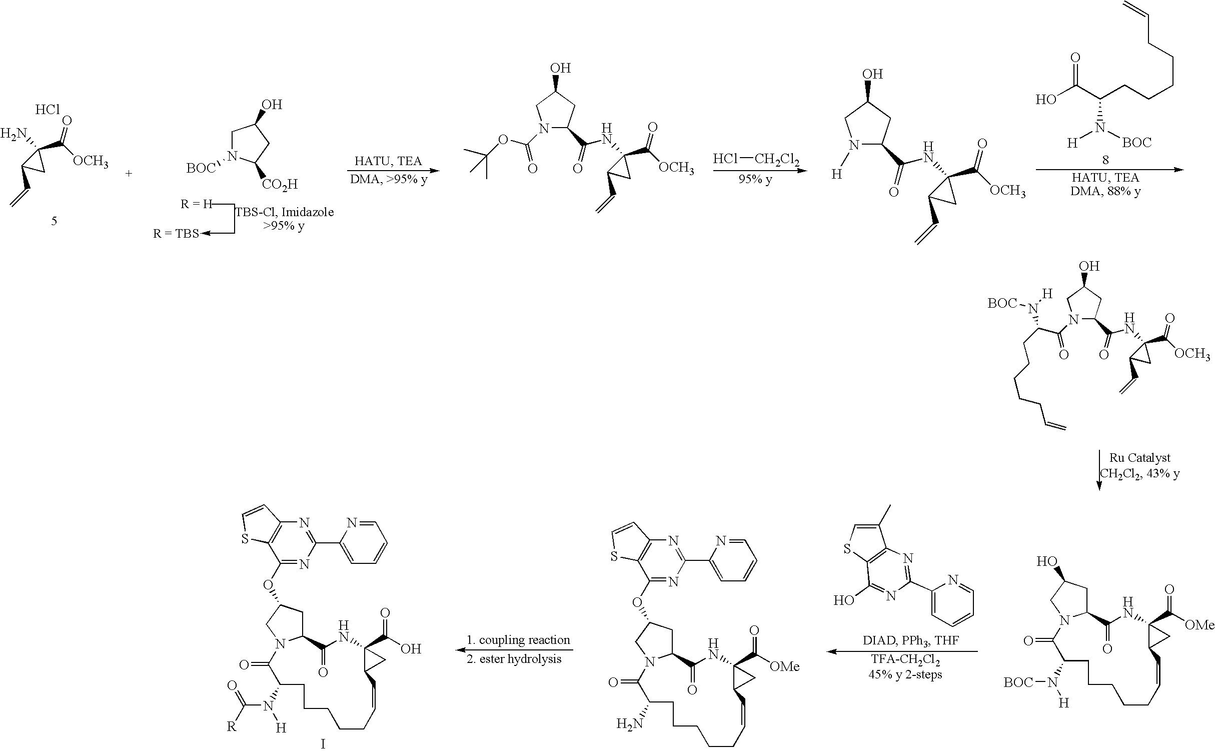

Suitable bases for use in these reactions include inorganic bases and organic bases. Suitable inorganic bases include, but are not limited to, sodium carbonate, sodium bicarbonate, potassium carbonate, potassium bicarbonate, sodium hydroxide, sodium hydride, potassium hydride, and cesium carbonate. Preferably, the base is potassium carbonate. Suitable organic bases include, but are not limited to, pyridine, triethylamine, tributylamine, triethanolamine, N-methylmorpholine, N-ethyl-N,N-diisopropylamine, DBU, and 4-N,N-dimethylaminopyridine. These reactions can also be performed in the presence of a catalytic amount of a suitable acid. Suitable acids include both Bronsted-Lowry and Lewis acids. Furthermore, these reactions are generally performed in a solvent or mixture of solvents that will not interfere with desired chemical reaction. Furthermore, appropriate solvents include those that are known to those of skill in the art to be compatible with the reaction conditions and include alkyl esters and aryl esters, alkyl, heterocyclic, and aryl ethers, hydrocarbons, alkyl and aryl alcohols, alkyl and aryl halogenated compounds, alkyl or aryl nitriles, alkyl and aryl ketones, and non-protic heterocyclic solvents. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent if it will not interfere with the desired transformation. Finally, such reactions can be performed at a temperature in the range of from about 0° C. to about 100° C., or in the range of from about 25° C. to about 100° C., or in the range of from about 35° C. to about 75° C., or in the range of from about 45° C. to about 55° C., or at about 50° C. The choice of a particular reducing agent, solvent, and temperature will depend on several factors including, but not limited to, the identity of the particular reactants and the functional groups present in such reactants. Such choices are within the knowledge of one of ordinary skill in the art and can be made without undue experimentation.

Such reactions may be performed using a suitable base in a suitable solvent. Suitable bases include, but are not limited to, potassium carbonate, sodium carbonate, potassium bicarbonate, sodium bicarbonate, potassium hydroxide, and sodium hydroxide. Solvents that may be used include, but are not limited to, methyl alcohol, ethyl alcohol, iso-propyl alcohol, n-propyl alcohol, acetonitrile, and DMF, or a mixture of them. Additionally, water may be used as a cosolvent if necessary. These reactions may be performed at a temperature of from about 0° C. to about 150° C. The particular choice of a base or combination of bases, solvent or combination of solvents, and reaction temperature will depend on the particular starting material being used and such choices are within the knowledge of one of ordinary skill in the art and can be made without undue experimentation.

These reactions are generally performed in the presence of a reducing agent, such as a borane source or hydrogen in the presence of suitable catalyst. Suitable borane sources include, but are not limited to, borane-trimethylamine complex, borane-dimethylamine complex, borane t-butyl amine complex, and borane-pyridine complex. Suitable catalysts for use in the presence of a reducing agent such as hydrogen include, but are not limited to, nickel, palladium, rhodium and ruthenium. Furthermore, such reactions are performed in a solvent or mixture of solvents that will not interfere with desired chemical reaction. Furthermore, appropriate solvents include those that are known to those of skill in the art to be compatible with the reaction conditions and include alkyl esters and aryl esters, alkyl, heterocyclic, and aryl ethers, hydrocarbons, alkyl and aryl alcohols, alkyl and aryl halogenated compounds, alkyl or aryl nitriles, alkyl and aryl ketones, and non-protic heterocyclic solvents. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, benzene, toluene, anisole, xylenes, and pyridine, or any mixture of the above solvents. Additionally, water may be used as a co-solvent if it will not interfere with the desired transformation. Finally, such reactions can be performed at a temperature in the range of from about 0° C. to about 75° C., preferably in the range of from about 0° C. to about 32° C., most preferably at room or ambient temperature. The choice of a particular reducing agent, solvent, and temperature will depend on several factors including, but not limited to, the identity of the particular reactants and the functional groups present in such reactants. Such choices are within the knowledge of one of ordinary skill in the art and can be made without undue experimentation.

The following Examples are meant to illustrate particular embodiments of the present invention only and are not intended to limit its scope in any manner.

Unless otherwise indicated, all numbers expressing quantities of ingredients, properties such as molecular weight, reaction conditions, and so forth used in the specification and claims are to be understood as being modified in all instances by the term “about”. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

### EXAMPLES

In the examples described below, unless otherwise indicated, all temperatures in the following description are in degrees Celsius (° C.) and all parts and percentages are by weight, unless indicated otherwise.

Various starting materials and other reagents were purchased from commercial suppliers, such as Aldrich Chemical Company or Lancaster Synthesis Ltd., and used without further purification, unless otherwise indicated.

The reactions set forth below were performed under a positive pressure of nitrogen, argon or with a drying tube, at ambient temperature (unless otherwise stated), in anhydrous solvents. Analytical thin-layer chromatography was performed on glass-backed silica gel 60° F. 254 plates (Analtech (0.25 mm)) and eluted with the appropriate solvent ratios (v/v). The reactions were assayed by high-pressure liquid chromatography (HPLC) or thin-layer chromatography (TLC) and terminated as judged by the consumption of starting material. The TLC plates were visualized by UV, phosphomolybdic acid stain, or iodine stain.

¹H-NMR spectra were recorded on a Bruker instrument operating at 300 MHz or 400 MHz and ¹³C-NMR spectra were recorded at 75 MHz. NMR spectra are obtained as DMSO-d₆ or CDCl₃ solutions (reported in ppm), using chloroform as the reference standard (7.25 ppm and 77.00 ppm) or DMSO-d₆ (2.50 ppm and 39.52 ppm). Other NMR solvents were used as needed. When peak multiplicities are reported, the following abbreviations are used: 5=singlet, d=doublet, t=triplet, m=multiplet, br=broadened, dd=doublet of doublets, dt=doublet of triplets. Coupling constants, when given, are reported in Hertz.

Infrared spectra were recorded on a Perkin-Elmer FT-IR Spectrometer as neat oils, as KBr pellets, or as CDCl₃ solutions, and when reported are in wave numbers (cm⁻¹). The mass spectra were obtained using LC/MS or APCI. All melting points are uncorrected.

All final products had greater than 95% purity (by HPLC at wavelengths of 220 nm and 254 nm).

In the following examples and preparations, “Et” means ethyl, “Ac” means acetyl, “Me” means methyl, “Ph” means phenyl, “(PhO)₂POCl” means chlorodiphenylphosphate, “HCl” means hydrochloric acid, “EtOAc” means ethyl acetate, “Na₂CO₃” means sodium carbonate, “NaOH” means sodium hydroxide, “NaCl” means sodium chloride, “NEt₃” means triethylamine, “THF” means tetrahydrofuran, “DIC” means diisopropylcarbodiimide, “HOBt” means hydroxy benzotriazole, “H₂O” means water, “NaHCO₃” means sodium hydrogen carbonate, “K₂CO₃” means potassium carbonate, “MeOH” means methanol, “i-PrOAc” means isopropyl acetate, “MgSO₄” means magnesium sulfate, “DMSO” means dimethylsulfoxide, “AcCl” means acetyl chloride, “CH₂Cl₂” means methylene chloride, “MTBE” means methyl t-butyl ether, “DMF” means dimethyl formamide, “SOCl₂” means thionyl chloride, “H₃PO₄” means phosphoric acid, “CH₃SO₃H” means methanesulfonic acid, “Ac₂O” means acetic anhydride, “CH₃CN” means acetonitrile, “KOH” means potassium hydroxide, “CDI” means carbonyl diimidazole, “DABCO” means 1,4-diazabicyclo[2.2.2]octane, “IPE” means isopropyl ether, “MTBE” means methyl tert-butyl ether, “Et₂O” means diethylether, “Na₂SO₄” means sodium sulfate, “NBS” means N-bromosuccinimide, “TEA” means triethylamine, “DCM” means dichloromethane, “TBAB” means tetrabutylammonium bromide, “HMPA” means hexamethylphosphoramide, “NMP” means 1-methyl-2-pyrrolidinone, “DMAC” means N,N-dimethylacetamide, “h” means hours, “min” means minutes, “mol” means moles, and “rt” means room temperature.

### Example 1

### 3-Thienylamine oxylate

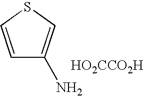

Methyl 3-aminothiophene-2-carboxylate (10.0 g, 64 mmol, 1.0 equiv) was refluxed in 1N sodium hydroxide (NaOH) (318 mL, 320 mmol, 5.0 equiv) for 2 h. The reaction mixture was cooled to 0° C. and acidified to pH 5 using 12.4 N hydrochloric acid (HCl). The crude beige acid was filtered, and the solids were taken-up in 1-propanol (100 mL), treated with oxalic acid (11.58 g, 128 mmol, 2.0 equiv) and heated at 38° C. for 1 h. The off-white product was filtered and the solids were taken on without further purification (5.55 g, 46% yield): ¹H NMR (400 MHz, DMSO-d6) δ 7.24 (dd, J=5.0, 3.0 Hz, 1H), 6.64 (dd, J=5.0, 1.3 Hz, 1H), 6.17 (dd, J=3.0, 1.5 Hz, 1H); LCMS (ESI+) for C₄H₅NS*C₂H₃O₄ m/z 191 (M+H)⁺.

### Example 2

### 5-Pyridin-2-ylthieno[3,2-b]pyridin-7-ol

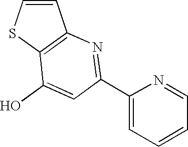

3-Thienylamine oxylate (5.55 g, 30 mmol, 1.0 equiv) and methyl 3-oxo-3-pyridin-2-ylpropanoate (5.67 g, 30 mmol, 1.0 equiv) were combined in a round bottom flask equipped with a Dean Stark reflux condenser and taken up in anhydrous toluene (100 mL). 4N HCl in 1,4-dioxane (0.733 mL, 3 mmol, 0.10 equiv) was added and the reaction mixture was refluxed for 12 h. The crude product was filtered and the black solids were taken on without further purification (6.02 g, 90% yield): ¹H NMR (400 MHz, DMSO-d6) δ 8.79 (d, J=4.5 Hz, 1H), 8.26 (d, J=8.1 Hz, 1H), 8.01-7.97 (m, 1H), 7.92 (d, J=5.3 Hz, 1H), 7.53 (dd J=7.2, 4.9 Hz, 1H), 7.07 (s, 1H), 7.05 (d, J=5.3 Hz, 1H); LCMS (ESI+) for C₁₂H₈N₂OS m/z 229 (M+H)⁺.

### Example 3

### 7-Chloro-5-pyridin-2-ylthieno[3,2-b]pyridine

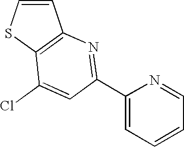

5-Pyridin-2-ylthieno[3,2-b]pyridin-7-ol (3.0 g, 13 mmol, 1.0 equiv) was taken up in phosphorous oxychloride (POCl₃) (100 mL) and refluxed for 6 h. The reaction mixture was concentrated in vacuo, and washed slowly with 1N NaOH. The organic layer was extracted with ethyl acetate, washed with saturated sodium chloride, dried over magnesium sulfate and concentrated in vacuo which gave a brown solid that was taken on without further purification (2.00 g, 62% yield): ¹H NMR (400 MHz, DMSO-d6) δ 8.87 (d, J=4.04 Hz, 1H), 8.50 (d, J=8.1 Hz, 1H), 8.31 (d, J=5.6 Hz, 1H), 8.22 (s, 1H), 8.07 (dt, J=7.8, 1.8 Hz, 1H), 7.61-7.56 (m, 2H); LCMS (ESI+) for C₁₂H₇ClN₂S m/z 247 (M+H)⁺.

### Example 4

### (4R)-1-(Tert-butoxycarbonyl)-4-[(5-pyridin-2-ylthieno[3,2-b]pyridin-7-yl)oxy]-L-proline

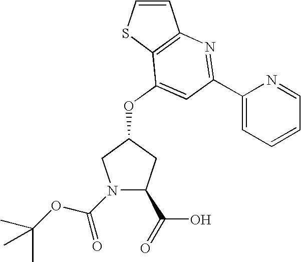

7-Chloro-5-pyridin-2-ylthieno[3,2-b]pyridine (1.97 g, 8 mmol, 1.0 equiv) and (4R)-1-(tert-butoxycarbonyl)-4-hydroxy-L-proline (1.85 g, 8 mmol, 1.0 equiv) were taken up in anhydrous DMSO (32 mL) and treated with potassium tert-butoxide (1.88 g, 17 mmol, 2.1 equiv). The reaction mixture was stirred at ambient temperature. Additional (4R)-1-(tert-butoxycarbonyl)-4-hydroxy-L-proline (1.85 g, 8 mmol, 1.0 equiv) and potassium tert-butoxide (1.88 g, 17 mmol, 2.1 equiv) were added after 6 h and 22 h. The resulting reaction mixture was stirred for 48 h. The reaction mixture was diluted with 0.5 M sodium citrate buffer (pH=4.5), and the organic layer was extracted with ethyl acetate, washed with saturated sodium chloride, dried over magnesium sulfate, filtered and concentrated in vacuo which gave a brown oil (3.56 g, 100% yield): ¹H NMR (400 MHz, DMSO-d6) δ 12.62 (s, 1H), 8.83 (d, J=4.0 Hz, 1H), 8.42 (d, J=8.1 Hz, 1H), 8.13-8.09 (m, 1H), 8.01 (td, J=7.8, 1.8 Hz, 1H), 7.62-7.59 (m, 1H), 7.53 (dd, J=7.1, 5.0 Hz, 1H), 7.47-7.43 (m, 1H), 5.68-5.62 (m, 1H), 4.30-4.23 (m, 1H), 3.80 (ddd, J=16.3, 11.8, 4.8 Hz, 1H), 3.60 (t, J=11.6 Hz, 1H), 2.60-2.52 (m, 1H), 2.37-2.28 (m, 1H), 1.41-1.31 (m, 9H); LCMS (ESI+) for C₂₂H₂₃N₃O₅S m/z 442 (M+H)⁺.

### Example 5

### Tert-butyl (2S,4R)-2-({[(1R,2S)-1-(methoxycarbonyl)-2-vinylcyclopropyl]amino}carbonyl)-4-[(S-pyridin-2-ylthieno[3,2-b]pyridin-7-yl)oxy]pyrrolidine-1-carboxylate

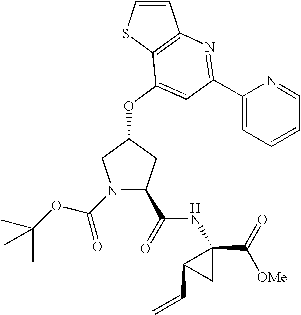

(4R)-1-(Tert-butoxycarbonyl)-4-[(5-pyridin-2-ylthieno[3,2-b]pyridin-7-yl)oxy]-L-proline (1.41 g, 8 mmol, 1.0 equiv) and methyl (1R,2S)-1-amino-2-vinylcyclopropanecarboxylate hydrochloride (1.41 g, 8 mmol, 1.0 equiv) were taken up in anhydrous dichloromethane (264 mL) and sequentially treated with diisopropylethylamine (DIPEA) (6.9 mL, 40 mmol, 5.0 equiv) and O-(7-azabenzotriazol-1-yl)-N,N,N′,N′-tetramethyluronium hexafluorophosphate (HATU) (3.01 g, 8 mmol, 1.0 equiv). The reaction mixture was stirred at 40° C. for 47 minutes. The reaction mixture was concentrated in vacuo, diluted with ethyl acetate, washed with saturated sodium bicarbonate, 0.5 M sodium citrate buffer and saturated sodium chloride, dried over magnesium sulfate, filtered and concentrated in vacuo which gave a yellow foam. The crude product was purified over silica (Biotage Horizon silica gel 40M column) and eluted with 2.5% methanol in dichloromethane (0.1% ammonium hydroxide) which provided an off-white solid (3.58 g, 80% yield): ¹H NMR (400 MHz, DMSO-d6) δ 8.84 (d, J=4.0 Hz, 1H), 8.76 (s, 1H), 8.43 (d, J=8.1 Hz, 1H), 8.12 (d, J=5.6 Hz, 1H), 8.01 (dt, J=7.8, 1.5 Hz, 1H), 7.61 (s, 1H), 7.53 (dd, J=7.1, 4.8 Hz, 1H), 7.46 (d, J=5.6 Hz, 1H), 5.73-5.54 (m, 2H), 5.26 (dd, J=16.9, 1.3 Hz, 1H), 5.10 (dd, J=10.2, 1.6 Hz, 1H), 4.27-4.18 (m, 1H), 3.90-3.78 (m, 1H), 3.60 (s, 3H), 2.34-2.06 (m, 2H), 1.72-1.63 (m, 1H), 1.38-1.33 (m, 10H), 1.32-1.18 (m, 2H); LCMS (ESI+) for C₂₉H₃₂N₄O₆S m/z 565 (M+H)⁺.

### Example 6

### Methyl (1R,2S)-1-({(4R)-4-[(5-pyridin-2-ylthieno[3,2-b]pyridin-7-yl)oxy]-L-prolyl}amino)-2-vinylcyclopropanecarboxylate

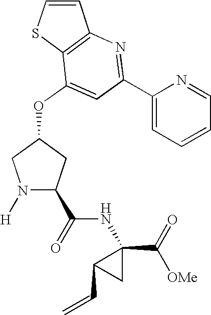

Tert-butyl (2S,4R)-2-({[(1R,2S)-1-(methoxycarbonyl)-2-vinylcyclopropyl]amino}carbonyl)-4-[(5-pyridin-2-ylthieno[3,2-b]pyridin-7-yl)oxy]pyrrolidine-1-carboxylate (3.58 g, 6 mmol, 1.0 equiv) was taken up in 1,4-dioxane (16 mL) and treated with 4N HCl in dioxane (16 mL, 64 mmol, 10.7 equiv). The solution was stirred at ambient temperature for 24 h. The reaction mixture was concentrated in vacuo, hexanes added, and the crude product was collected as a solid (3.58 g, 100% yield): ¹H NMR (400 MHz, DMSO-d6) δ 9.51 (s, 1H), 8.97 (s, 1H), 8.85 (d, J=4.0 Hz, 1H), 8.37 (t, J=8.5 Hz, 1H), 8.19-8.12 (m, 1H), 8.04 (td, J=7.8, 1.8 Hz, 1H), 7.59-7.53 (m, 2H), 7.49-7.44 (m, 1H), 5.73 (s, 1H), 5.65 (dt, J=17.2, 9.7 Hz, 1H), 5.28 (dd, J=17.2, 1.5 Hz, 2H), 4.41 (dd, J=10.0, 6.7 Hz, 1H), 3.84-3.74 (m, 1H), 3.62 (s, 3H), 3.52-3.41 (m, 1H), 2.75-2.66 (m, 1H), 2.34-2.19 (m, 2H), 1.67 (dd, J=8.1, 5.3 Hz, 1H), 1.43 (dd, J=9.5, 5.2 Hz, 1H); LCMS (ESI+) for C₂₄H₂₄N₄O₄S m/z 465 (M+H)⁺.

### Example 7

### Methyl (1R,2S)-1-({(4R)-1-{2-[(tert-butoxycarbonyl)amino]non-8-enoyl}-4-[(5-pyridin-2-ylthieno[3,2-b]pyridin-7-yl)oxy]-L-prolyl}amino)-2-vinylcyclopropanecarboxylate

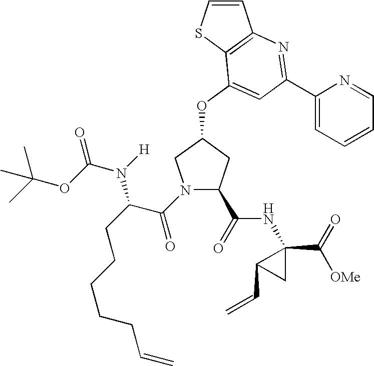

Methyl (1R,2S)-1-({(4R)-4-[(5-pyridin-2-ylthieno[3,2-b]pyridin-7-yl)oxy]-L-prolyl}amino)-2-vinylcyclopropanecarboxylate (1.7 g, 3.6 mmol, 1.0 equiv) and (2S)-2-[(tert-butoxycarbonyl)amino]non-8-enoic acid (0.98 g, 3.6 mmol, 1.0 equiv) was taken up in anhydrous DMA (37 mL) to which triethylamine (1.0 mL, 7.2 mmol, 2.0 equiv) was added followed by HATU (1.37 g, 3.6 mmol, 1.0 equiv). The reaction mixture was stirred at 50° C. for 2 h and poured into 50% saturated sodium bicarbonate. The organic layer was extracted with tert-Butyl methyl ether (MTBE), washed with saturated sodium chloride, dried over magnesium sulfate and concentrated in vacuo. The crude product was taken on without any further purification (2.0 g, 98% yield): ¹H NMR (400 MHz, DMSO-d6) δ 8.87-8.83 (m, 1H), 8.60 (s, 1H), 8.43-8.34 (m, 1H), 8.13 (m, 1H), 8.01-7.98 (m, 1H), 7.58 (s, 1H), 7.54-7.51 (m, 1H), 7.49-7.44 (m, 1H), 5.78 (s, 1H), 5.72-5.61 (m, 1H), 5.23 (dd, J=17.1, 1.6 Hz, 1H), 5.10 (dd, J=10.1, 1.8 Hz, 1H), 5.00-4.89 (m, 3H), 4.41 (t, J=8.18 Hz, 1H), 4.08-4.01 (m, 2H), 3.59 (s, 3H), 2.32-2.23 (m, 1H), 1.99-1.94 (m, 0.3H), 1.64-1.58 (m, 2H), 1.36-1.27 (m, 11H), 1.18 (s, 9H); LCMS (ESI+) for C₃₈H₄₇N₅O₇S m/z 718 (M+H)⁺.

### Example 8

### Methyl (2R,12Z,13aS,14aR,16aS)-6-[(tert-butoxycarbonyl)amino]-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate

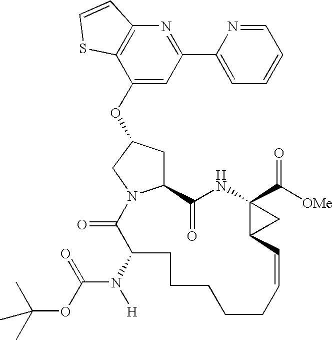

Methyl (1R,2S)-1-({(4R)-1-{2-[(tert-butoxycarbonyl)amino]non-8-enoyl}-4-[(5-pyridin-2-ylthieno[3,2-b]pyridin-7-yl)oxy]-L-prolyl}amino)-2-vinylcyclopropanecarboxylate (1.2 g, 14.2 mmol, 1.0 equiv) was taken up in anhydrous dichloromethane (705 mL). The reaction vessel was evacuated and purged with nitrogen gas. 1,3-Bis-(2,4,6-trimethylphenyl)-2-imidazolidinylidene)dichloro(phenylmethylene)-(tricyclohexylphospine)ruthenium (Grubbs Catalyst 2^{nd} Generation) was added (0.301 g, 0.355 mmol, 0.25 equiv) and the resultant mixture was stirred at 40° C. for 4 h. The reaction mixture was concentrated in vacuo, and the crude product was purified over silica gel (Biotage Horizon silica gel 40M column), which was eluted with 1-2.5% methanol in dichloromethane (0.1% ammonium hydroxide). The solids were triturated with MTBE/hexanes and provided a beige solid (0.221 g, 24% yield): ¹H NMR (400 MHz, DMSO-d6) δ 8.83 (d, J=4.3 Hz, 1H), 8.73 (s, 1H), 8.38 (d, J=8.1 Hz, 1H), 8.12-8.09 (m, 1H), 8.01-7.97 (m, 1H), 7.54-7.51 (m, 2H), 7.47 (d, J=5.6 Hz, 1H), 6.89 (d, J=7.1 Hz, 1H), 5.81 (br. s, 1H), 5.56-5.49 (m, 1H), 5.27 (t, J=9.6 Hz, 1H), 4.51 (t, J=7.8 Hz, 1H), 4.34-4.31 (m, 1H), 4.06-4.01 (m, 2H), 3.93-3.89 (m, 1H), 3.57 (s, 3H), 2.39-2.37 (m, 2H), 2.31-2.21 (m, 2H), 1.38-1.23 (m, 10H), 1.10 (d, J=5.8 Hz, 9H); LCMS (ESI+) for C₃₆H₄₃N₅O₇S m/z 690 (M+H)⁺.

### Example 9

### Methyl (2R,12Z,13aS,14aR,16aS)-6-amino-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate

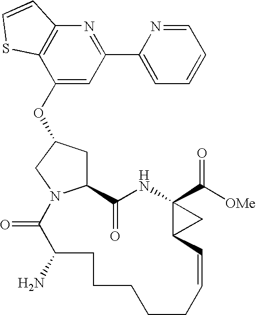

Methyl (2R,12Z,13aS,14aR,16aS)-6-[(tert-butoxycarbonyl)amino]-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.452 g, 0.7 mmol, 1.0 equiv) was taken up in anhydrous dichloromethane (5 mL) to which trifluoroacetic acid (5 mL) added. The resultant mixture was stirred at ambient temperature for 0.5 h. The reaction mixture was diluted with dichloromethane, quenched with saturated sodium bicarbonate, washed with saturated sodium chloride, dried over magnesium sulfate, filtered and concentrated in vacuo which provided a brown foam (0.151 g, >95% yield): ¹H NMR (400 MHz, DMSO-d6) δ 8.83 (d, J=4.0 Hz, 2H), 8.71 (s, 1H), 8.42 (d, J=8.1 Hz, 1H), 8.12 (d, J=5.6 Hz, 1H), 8.00 (td, J=7.8, 1.6 Hz, 1H), 7.60-7.56 (m, 1H), 7.53 (dd, J=7.2, 5.2 Hz, 1H), 7.46 (t, J=5.6 Hz, 1H), 5.84-5.79 (m, 1H), 5.30 (t, J=9.9 Hz, 1H), 4.49 (t, J=7.8 Hz, 1H), 3.95 (s, 2H), 3.66-3.58 (m, 2H), 3.58-3.54 (m, 3H), 2.54-2.51 (m, 1H), 2.44-2.40 (m, 1H), 2.37 (s, 2H), 2.31 (dd, J=3.8, 1.8 Hz, 2H), 1.56-1.46 (m, 4H), 1.24 (s, 6H); LCMS (ESI+) for C₃₁H₃₅N₅O₅S m/z 590 (M+H)⁺.

### Example 10

### Methyl (2R,12Z,13aS,14aR,16aS)-6-{[(cyclopentyloxy)carbonyl]amino}-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate

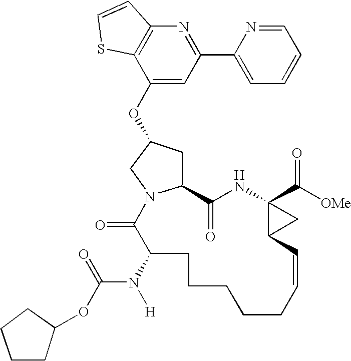

Methyl (2R,12Z,13aS,14aR,16aS)-6-amino-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (275 mg, 0.47 mmol, 1.0 equiv) and triethylamine (0.078 mL, 0.56 mmol, 1.2 equiv) were taken up in anhydrous DMA (1.3 mL). Cyclopentyl 4-nitrophenyl carbonate (0.117 g, 0.47 mmol, 1.0 equiv) was added and the reaction mixture was heated at 80° C. for 15.5 h. The reaction mixture was diluted with ethyl acetate, poured into saturated sodium bicarbonate, washed with saturated sodium chloride, dried over magnesium sulfate, filtered and concentrated in vacuo which provided a brown oil. The crude product was purified over silica gel (Biotage Horizon silica gel 40M column), and eluted with 1-2.5% methanol in dichloromethane (0.1% ammonium hydroxide) which gave the product as a brown residue (0.068 g, 94% yield): ¹H NMR (400 MHz, DMSO-d6) δ 8.83 (d, J=4.3 Hz, 1H), 8.70 (s, 1H), 8.42 (d, J=8.1 Hz, 1H), 8.10 (m, 2H), 8.00 (s, 1H), 7.57-7.50 (m, 2H), 7.50-7.44 (m, 1H), 6.93-6.87 (m, 1H), 5.83 (br. s, 1H), 5.56-5.49 (m, 1H), 5.27 (t, J=9.7 Hz, 1H), 4.49 (t, J=8.0 Hz, 1H), 3.96-3.92 (m, 1H), 3.57 (m, 3H), 2.44 (m, 1H), 2.41-2.31 (m, 2H), 2.26-2.19 (m, 1H), 1.93-1.48 (m, 12H), 1.30 (s, 9H); LCMS (ESI+) for C₃₇H₄₃N₅O₇S m/z 702 (M+H)⁺.

### Example 11

### (2R,12Z,13aS,14aR,16aS)-6-{[(cyclopentyloxy)carbonyl]amino}-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

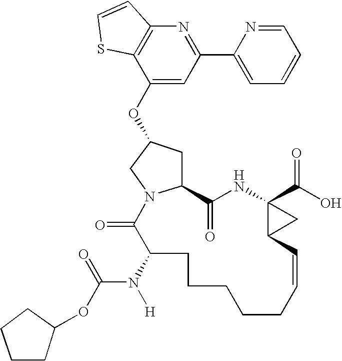

Methyl (2R,12Z,13aS,14aR,16aS)-6-{[(cyclopentyloxy)carbonyl]amino}-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.307 g, 0.44 mmol, 1.0 equiv) was taken up in 1:1 anhydrous tetrahydrofuran/anhydrous methanol and treated with aqueous lithium hydroxide (4.6 mL of 40 mg/mL LiOH—H₂O, 10.0 equiv). The resultant solution was stirred at ambient temperature for 2 h. The reaction mixture was concentrated in vacuo, diluted with ethyl acetate, poured into 0.05M sodium citrate buffer (pH 4.5), washed with saturated sodium chloride, dried over magnesium sulfate, filtered and concentrated in vacuo. The crude product was purified by reverse phase chromatography (C18) and eluted with 30-75% acetonitrile in water (0.1% acetic acid) which provided the product as a white solid (0.053 g, 18% yield): ¹H NMR (400 MHz, DMSO-d6) δ 12.22 (br. s, 1H), 8.84 (s, 1H), 8.64 (s, 1H), 8.42 (s, 1H), 8.11 (d, J=5.6 Hz, 1H), 8.05-7.96 (m, 1H), 7.59-7.50 (m, 2H), 7.47 (d, J=5.6 Hz, 1H), 7.29-7.09 (m, 2H), 5.82 (s, 1H), 5.56-5.49 (m, 1H), 5.29 (t, J=9.5 Hz, 1H), 4.59-4.52 (m, 1H), 4.52-4.42 (m, 1H), 4.29-4.19 (m, 1H), 4.17-4.05 (m, 1H), 3.99-3.89 (m, 1H), 1.56-1.14 (m, 22H); LCMS (ESI+) for C₃₆H₄₁N₅O₇S m/z 688 (M+H)⁺; Anal. calcd. for C₃₆H₄₁N₅O₇S•0.37 MTBE, •1.08 acetic acid •1.26H₂O: C, 59.48; H, 6.52; N, 8.67. Found: C, 59.74; H, 6.13; N, 8.27.

### Example 12

### Methyl (2R,6S,12Z,13aS,14aR,16aS)-6-{[(cyclobutyloxy)carbonyl]amino}-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate

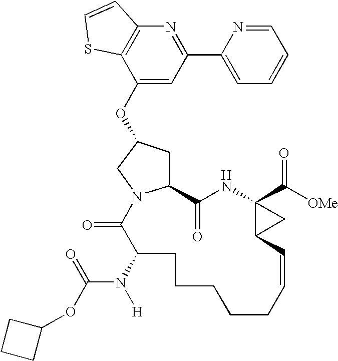

Methyl (2R,12Z,13aS,14aR,16aS)-6-amino-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (75 mg, 0.127 mmol, 1.0 equiv) and triethylamine (0.021 mL, 0.153 mmol, 1.2 equiv) were taken up in anhydrous DMA (1.3 mL). Cyclobutyl 4-nitrophenyl carbonate (0.030 g, 0.127 mmol, 1.0 equiv) was added and the reaction mixture was heated at 80° C. for 15.5 h. The reaction mixture was diluted with ethyl acetate, poured into saturated sodium bicarbonate, washed with saturated sodium chloride, dried over magnesium sulfate, filtered and concentrated in vacuo which provided a brown oil. The crude product was purified over silica gel (Biotage Horizon silica gel 12S column), which was eluted with 1-2.5% methanol in dichloromethane (0.1% ammonium hydroxide) and gave the product as a brown residue (62 mg, 71% yield): ¹H NMR (400 MHz, DMSO-d6) δ 8.83 (d, J=4.6 Hz, 2H), 8.71 (s, 1H), 8.44 (d, J=8.1 Hz, 1H), 8.31 (s, 1H), 8.02-7.99 (m, 1H), 7.47-7.46 (d, J=5.6 Hz, 1H), 7.27-7.25 (d, J=7.6 Hz, 2H), 5.83 (s, 1H), 5.54-5.51 (m, 1H), 5.27 (t, J=9.6 Hz, 1H), 4.49 (t, J=8.3 Hz, 1H), 4.41 (t, J=7.3 Hz, 1H), 4.26-4.23 (m, 1H), 3.94-3.89 (m, 1H), 3.66-3.58 (m, 2H), 3.57 (s, 3H), 2.54 (m, 1H), 2.44-2.38 (m, 4H), 2.26-2.19 (m, 1H), 1.91-1.82 (m, 4H), 1.75-1.63 (m, 4H), 1.59-1.42 (m, 6H); LCMS (ESI+) for C₃₆H₄, N₅O₇S m/z 688 (M+H)⁺.

### Example 13

### (2R,6S,12Z,13aS,14aR,16aS)-6-{[(Cyclobutyloxy)carbonyl]amino}-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

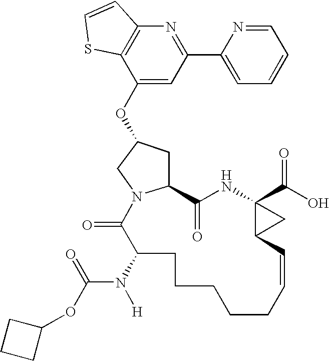

Methyl (2R,6S,12Z,13aS,14aR,16aS)-6-{[(cyclobutyloxy)carbonyl]amino}-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.060 g, 0.087 mmol, 1.0 equiv) was taken up in 1:1 anhydrous tetrahydrofuran/anhydrous methanol (2 mL) and treated with aqueous lithium hydroxide (0.92 mL of 40 mg/mL LiOH—H₂O, 10.0 equiv). The resultant mixture was stirred at ambient temperature for 17 h. The reaction mixture was concentrated in vacuo, diluted with ethyl acetate, poured into 0.5 M sodium citrate buffer (pH 4.5), washed with saturated sodium chloride, dried over magnesium sulfate, filtered and concentrated in vacuo. The crude product was purified by reversed phase chromatography (C18) and eluted with 30-75% acetonitrile in water (0.1% acetic acid) which provided a white solid (0.024 g, 41% yield): ¹H NMR (400 MHz, DMSO-d6) δ 12.25 (s, 1H), 8.83 (d, J=4.0 Hz, 1H), 8.63 (s, 1H), 8.44 (d, J=8.1 Hz, 1H), 8.10 (d, J=5.6 Hz, 1H), 8.03-7.98 (m, 1H), 7.56-7.52 (m, 2H), 7.47 (d, J=5.8 Hz, 1H), 7.24 (d, J=7.3 Hz, 1H), 5.82 (s, 1H), 5.49 (m, 1H), 5.29 (t, J=9.7 Hz, 1H), 5.17 (br. s, 1H), 4.49-4.39 (m, 2H), 4.26-4.22 (m, 1H), 4.09-4.07 (m, 1H), 3.94-3.91 (m, 1H), 2.44-2.32 (m, 3H), 2.18 (q, J=8.8 Hz, 1H), 1.90-1.81 (m, 3H), 1.74-1.65 (m, 3H), 1.54-1.42 (m, 4H), 1.40-1.22 (m, 6H); LCMS (ESI+) for C₃₅H₃₉N₅O₇S m/z 674 (M+H)⁺; Anal. calcd. for C₃₅H₃₉N₅O₇S•0.96 ethyl acetate•1.41 TFA•1.0H₂O: C, 53.39; H, 5.39; N, 7.47. Found: C, 53.74; H, 5.61; N, 7.17.

### Example 14

### (2R,6S,12Z,13aS,14aR,16aS)-6-[(Tert-butoxycarbonyl)amino]-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

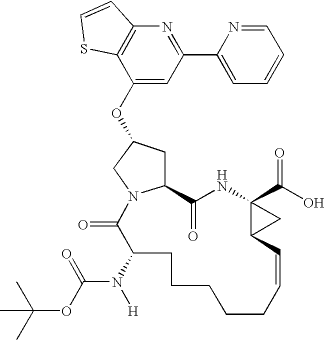

Methyl (2R,12Z,13aS,14aR,16aS)-6-[(tert-butoxycarbonyl)amino]-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.070 g, 0.102 mmol, 1.0 equiv) was taken up in 1:1 anhydrous tetrahydrofuran and anhydrous methanol (2 mL) and treated with aqueous lithium hydroxide (1.07 mL of 40 mg/mL LiOH—H₂O, 1.02 mmol, 10.0 equiv). The resultant mixture was stirred at ambient temperature for 17 h. The reaction mixture was concentrated in vacuo, diluted with ethyl acetate, poured into 0.5 M sodium citrate buffer (pH 4.5), washed with saturated sodium chloride, dried over magnesium sulfate, filtered and concentrated in vacuo. The crude product was purified by reversed phase chromatography (C18) and eluted with 30-75% acetonitrile in water (0.1% acetic acid) which provided a white solid (0.021 g, 30% yield): ¹H NMR (400 MHz, DMSO-d6) δ 12.26 (s, 1H), 8.83 (d, J=4.3 Hz, 1H), 8.66 (s, 1H), 8.38 (d, J=8.1 Hz, 1H), 8.10 (d, J=5.6 Hz, 1H), 8.04-7.96 (m, 1H), 7.57-7.50 (m, 2H), 7.47 (d, J=5.8 Hz, 1H), 6.88 (d, J=7.1 Hz, 1H), 5.81 (s, 1H), 5.58-5.43 (m, 1H), 5.29 (t, J=9.6 Hz, 1H), 4.50 (t, J=8.0 Hz, 1H), 4.31 (d, J=11.1 Hz, 1H), 4.04 (m, 1H), 3.92 (dd, J=11.2, 3.9 Hz, 1H), 2.42-2.29 (m, 3H), 2.18 (q, J=8.9 Hz, 1H), 1.85-1.65 (m, 3H), 1.55-1.41 (m, 3H), 1.40-1.27 (m, 5H), 1.04 (s, 9H); LCMS (ESI+) for C₃₅H₄₁N₅O₇S m/z 676 (M+H)⁺; Anal. calcd. for C₃₅H₄₁N₅O₇S•0.90 ethyl acetate•0.89 TFA•0.99H₂O: C, 55.47; H, 5.89; N, 8.01. Found: C, 55.77; H, 5.95; N, 7.88.

### Example 15

### Methyl (2R,6S,12Z,13aS,14aR,16aS)-6-[(cyclopropylacetyl)amino]-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate

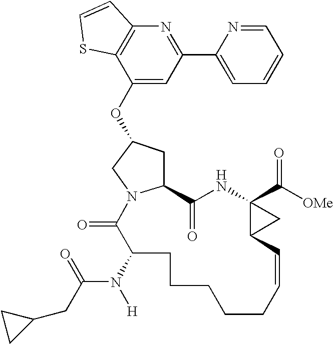

Methyl (2R,12Z,13aS,14aR,16aS)-6-amino-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (249 mg, 0.423 mmol, 1.0 equiv) and cyclopropylacetic acid (0.042 g, 0.423 mmol, 1.0 equiv) were taken up in anhydrous dichloromethane (14 mL, 0.03 M). Diisopropylethylamine (0.496 mL, 2.1 mmol, 5.0 equiv) was added followed by HATU (0.161 g, 0.423 mmol, 1.0 equiv) and the resultant solution was stirred for 15.5 h. The reaction mixture was diluted with ethyl acetate, poured into saturated sodium bicarbonate, washed with 0.5 M sodium citrate buffer (pH=4.5), saturated sodium chloride, dried over magnesium sulfate and concentrated in vacuo which provided a brown oil (0.278 g, 99% yield): ¹H NMR (400 MHz, DMSO-d6) δ 8.86-8.83 (m, 2H), 8.73-8.70 (m, 1H), 8.42 (m, 1H), 8.12 (d, J=5.6 Hz, 1H), 8.02-7.98 (m, 1H), 7.93 (d, J=7.6 Hz, 1H), 7.53 (m, 1H), 7.47 (d, J=5.6 Hz, 1H), 5.85 (s, 1H), 5.56-5.49 (m, 1H), 5.28 (t, J=9.8 Hz, 1H), 4.49-4.44 (m, 2H), 4.15 (d, J=11.6 Hz, 1H), 4.02 (dd, J=11.5, 4.4 Hz, 1H), 3.64-3.61 (m, 1H), 3.57 (s, 3H), 1.91-1.89 (m, 2H), 1.54-1.49 (m, 4H), 1.49-1.11 (m, 15H); LCMS (ESI+) for C₃₆H₄₁N₅O₆S m/z 672 (M+H)⁺.

### Example 16

### (2R,6S,12Z,13aS,14aR,16aS)-6-[(Cyclopropylacetyl)amino]-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

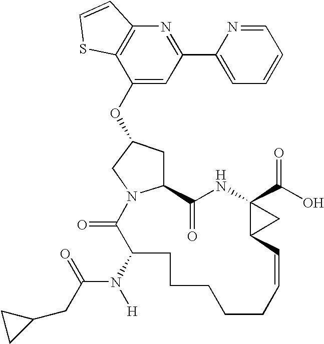

Methyl (2R,6S,12Z,13aS,14aR,16aS)-6-[(cyclopropylacetyl)amino]-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.261 g, 0.39 mmol, 1.0 equiv) was taken up in 1:1 anhydrous tetrahydrofuran and anhydrous methanol (2 mL) and treated with aqueous lithium hydroxide (4.1 mL of 40 mg/mL LiOH—H₂O, 9 mmol, 10.0 equiv). The resultant mixture was stirred at ambient temperature for 1 h. The reaction mixture was concentrated in vacuo, diluted with ethyl acetate, poured into 0.5 M sodium citrate buffer (pH 4.5), washed with saturated sodium chloride, dried over magnesium sulfate, filtered and concentrated in vacuo. The crude product was purified by reversed phase chromatography (C18) and eluted with 30-75% acetonitrile in water (0.1% acetic acid) which provided a white solid (0.065 g, 25% yield): ¹H NMR (400 MHz, DMSO-d6) δ 12.28 (br. s, 1H), 8.83 (m, 1H), 8.65 (s, 1H), 8.42 (d, J=8.1 Hz, 1H), 8.12 (d, J=5.6 Hz, 1H), 8.02-7.98 (m, 1H), 7.92 (d, J=7.3 Hz, 1H), 7.57 (s, 1H), 7.55-7.51 (m, 1H), 7.48 (d, J=7.3 Hz, 1H), 5.86 (s, 1H), 5.54-5.47 (m, 1H), 5.31 (t, J=9.8 Hz, 1H), 4.48-4.41 (m, 2H), 4.15 (d, J=12.1 Hz, 4H), 2.44-2.31 (m, 4H), 2.23-2.16 (m, 1H), 1.91-1.89 (m, 3H), 1.73 (br. s, 1H), 1.47-1.35 (m, 8H), 1.28-1.21 (m, 4H), 0.27-0.24 (m, 2H); LCMS (ESI+) for C₃₅H₃₉N₅O₆S m/z 658 (M+H)⁺; Anal. calcd. for C₃₅H₃₉N₅O₆S•0.75 ethyl acetate•0.35 acetic acid•0.62H₂O: C, 61.48; H, 6.35; N, 9.26. Found: C, 61.67; H, 6.02; N, 8.94.

### Example 17

### 2-Pyridin-2-ylthieno[2,3-d]pyrimidin-4-ol

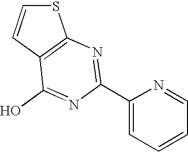

Methyl 2-aminothiophene-3-carboxylate (5.0 g, 31.8 mmol, 1.0 equiv) and pyridine-2-carbonitrile (3.1 mL, 31.8 mmol, 1.0 equiv) were taken up in anhydrous tetrahydrofuran (125 mL). The resultant beige mixture was cooled to 0° C., to which potassium tert-butoxide (5.3 g, 48.0 mmol, 1.5 equiv) was added. The reaction mixture was stirred for 1 h, concentrated in vacuo, diluted with dichloromethane and poured into 50% saturated ammonium chloride. The organic layer washed with water, saturated sodium chloride, dried over magnesium sulfate, filtered and concentrated in vacuo. The solids were triturated with MTBE and provided a tan solid (1.4 g, 20% yield): ¹H NMR (400 MHz, DMSO-d6) δ 11.98 (s, 1H), 8.75 (d, J=4.3 Hz, 1H), 8.37 (d, J=7.8 Hz, 1H), 8.05 (td, J=7.8, 1.6 Hz, 1H), 7.68-7.62 (m, 2H), 7.47 (d, J=5.8 Hz, 1H); LCMS (ESI+) for C₁₁H₇N₃OS m/z 230 (M+H)⁺.

### Example 18

### 1-tert-butyl 2-methyl (2S,4R)-4-[(2-pyridin-2-ylthieno[2,3-d]pyrimidin-4-yl)oxy]pyrrolidine-1,2-dicarboxylate

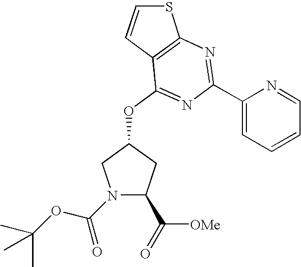

2-Pyridin-2-ylthieno[2,3-d]pyrimidin-4-ol (1.6 g, 7.0 mmol, 1.0 equiv), 1-tert-butyl 2-methyl (2S,4S)-4-hydroxypyrrolidine-1,2-dicarboxylate (1.7 g, 7.0 mmol, 1.0 equiv) and triphenylphosphine (3.7 g, 14 mmol, 2.0 equiv) were taken up in anhydrous tetrahydrofuran (140 mL). The resultant white slurry was cooled to 0° C., to which diisopropyl azodicarboxylate (DIAD) (2.7 mL, 14 mmol, 2.0 equiv) was added. The amber solution was warmed to room temperature and allowed to stir for 17 h. The reaction mixture was concentrated in vacuo, poured into 5% saturated bicarbonate and extracted with ethyl acetate. The organic layer washed with saturated sodium chloride, dried over magnesium sulfate and concentrated in vacuo. The crude product was triturated with MTBE and provided the product as a tan solid (2.2 g, 69% yield): ¹H NMR (400 MHz, DMSO-d6) δ 8.76 (d, J=4.0 Hz, 1H), 8.44 (d, J=8.1 Hz, 1H), 8.00-7.96 (s, 1H), 7.89 (d, J=6.1 Hz, 1H), 7.54-7.48 (m, 2H), 5.94 (s, 1H), 4.48-4.43 (m, 1H), 3.92-3.85 (m, 1H), 3.76-3.65 (m, 4H), 2.73-2.64 (m, 1H), 2.47-2.40 (m, 1H), 1.35 (s, 9H); LCMS (ESI+) for C₂₂H₂₄N₄O₅S m/z 457 (M+H)⁺.

### Example 19

### (4R)-1-(Tert-butoxycarbonyl)-4-[(2-pyridin-2-ylthieno[2,3-d]pyrimidin-4-yl)oxy]-L-proline

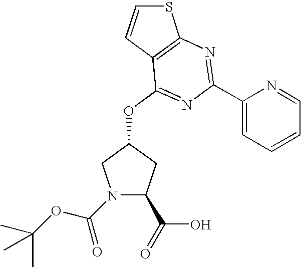

1-Tert-butyl 2-methyl (2S,4R)-4-[(2-pyridin-2-ylthieno[2,3-d]pyrimidin-4-yl)oxy]pyrrolidine-1, 2-dicarboxylate (2.2 g, 4.8 mmol, 1.0 equiv) was taken up in a 1:1 solution of anhydrous tetrahydrofuran and anhydrous methanol (50 mL). A solution of aqueous lithium hydroxide (7.5 mL of 40 mg/mL LiOH—H₂O, 1.5 equiv) was added and the reaction mixture was stirred at room temperature for 35 minutes. The reaction mixture was concentrated in vacuo, poured into 0.5 M sodium citrate buffer (pH 4.5), extracted with ethyl acetate, washed with saturated sodium chloride, dried over magnesium sulfate, filtered and concentrated in vacuo which provided a beige solid (2.10 g, >95% yield): ¹H NMR (400 MHz, DMSO-d6) δ 12.77 (s, 1H), 8.77 (d, J=3.8 Hz, 1H), 8.45 (t, J=7.4 Hz, 1H), 7.99 (td, J=7.8, 1.5 Hz, 1H), 7.89 (d, J=5.8 Hz, 1H), 7.57-7.48 (m, 2H), 5.92 (d, J=2.0 Hz, 1H), 4.38-4.31 (m, 1H), 3.88 (td, J=12.1, 4.7 Hz, 1H), 3.76-3.68 (m, 1H), 2.72-2.61 (m, 1H), 2.43 (td, J=13.2, 7.2 Hz, 1H), 1.38-1.34 (m, 9H); LCMS (ESI+) for C₂₁H₂₂N₄O₅S m/z 443 (M+H)⁺.

### Example 20

### Tert-butyl (2S,4R)-2-({[(1R,2S)-1-(methoxycarbonyl)-2-vinylcyclopropyl]amino}carbonyl)-4-[(2-pyridin-2-ylthieno[2,3-d]pyrimidin-4-yl)oxy]pyrrolidine-1-carboxylate

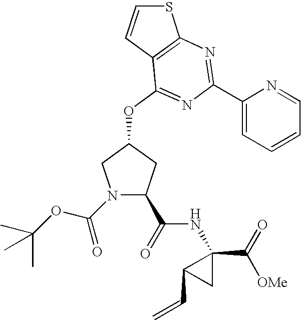

(4R)-1-(Tert-butoxycarbonyl)-4-[(2-pyridin-2-ylthieno[2,3-d]pyrimidin-4-yl)oxy]-L-proline (2.1 g, 4.7 mmol, 1.0 equiv) and methyl (1R,2S)-1-amino-2-vinylcyclopropanecarboxylate hydrochloride (0.83 g, 4.7 mmol, 1.0 equiv) were taken up in anhydrous DMA (50 mL) to which triethylamine (2.0 mL, 14.1 mmol, 3.0 equiv) followed by HATU (1.8 g, 4.7 mmol, 1.0 equiv) was added. The reaction mixture was stirred at 50° C. for 18 h. The reaction mixture was poured into saturated sodium bicarbonate and the crude product was collected as a solid. The crude product was purified over silica gel (Biotage Horizon silica gel 40M column) and eluted with 0.5-5% methanol in chloroform (0.1% ammonium hydroxide). The semi-pure solid was triturated with MTBE/chloroform/hexanes and provided a light beige solid (1.9 g, 73% yield): ¹H NMR (400 MHz, DMSO-d6) δ 7.96 (m, 2H), 7.63 (d, J=7.8 Hz, 1H), 7.20-7.14 (m, 1H), 7.08 (d, J=6.1 Hz, 1H), 6.72 (m, 2H), 6.65 (d, J=5.8 Hz, 1H), 5.08 (m, 1H), 4.87-4.78 (m, 1H), 4.49-4.42 (m, 1H), 4.31 (m, 1H), 3.47 (m, 1H), 3.10 (m, 1H), 2.90 (m, 1H), 2.78 (s, 3H), 1.58 (m, 2H), 1.34 (m, 1H), 0.87 (m, 1H), 0.54 (s, 9H); LCMS (ESI+) for C₂₈H₃₁N₅O₆S m/z 566 (M+H)⁺.

### Example 21

### Methyl (1R,2S)-1-({(4R)-4-[(2-pyridin-2-ylthieno[2,3-d]pyrimidin-4-yl)oxy]-L-prolyl}amino)-2-vinylcyclopropanecarboxylate

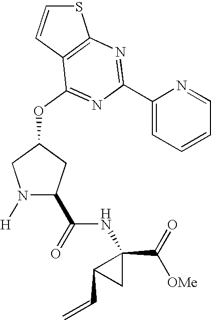

Tert-butyl (2S,4R)-2-({[(1R,2S)-1-(methoxycarbonyl)-2-vinylcyclopropyl]amino}carbonyl)-4-[(2-pyridin-2-ylthieno[2,3-d]pyrimidin-4-yl)oxy]pyrrolidine-1-carboxylate (1.9 g, 3.4 mmol, 1.0 equiv) taken up in anhydrous dichloromethane (20 mL), treated with trifluoroacetic acid (10 mL), and stirred at ambient temperature for 10 h. The reaction mixture was poured into saturated bicarbonate. The organic layer was extracted with dichloromethane, washed with saturated sodium chloride, dried over magnesium sulfate, filtered and concentrated in vacuo. The crude product was triturated with MTBE/dichloromethane/hexanes and provided a beige solid (1.5 g, 94% yield): ¹H NMR (400 MHz, DMSO-d6) δ 8.86 (s, 1H), 8.76 (d, J=4.0 Hz, 1H), 8.46 (d, J=7.8 Hz, 1H), 7.99 (td, J=7.8, 1.6 Hz, 1H), 7.93-7.88 (m, 1H), 7.54 (dd, J=7.2, 5.2 Hz, 1H), 7.49 (d, J=5.8 Hz, 1H), 5.89 (s, 1H), 5.69-5.58 (m, 1H), 5.28 (dd, J=17.2, 1.8 Hz, 1H), 5.10 (dd, J=10.4, 1.8 Hz, 1H), 4.05 (m, 1H), 3.61 (s, 3H), 3.47 (m, 1H), 3.38 (m, 1H), 2.32-2.22 (m, 2H), 1.67 (dd, J=7.8, 5.3 Hz, 1H), 1.37 (dd, J=9.4, 5.0 Hz, 1H), 1.23 (s, 1H), 0.87-0.81 (m, 1H); LCMS (ESI+) for C₂₃H₂₃N₅O₄S m/z 466 (M+H)⁺.

### Example 22

### Methyl (1R,2S)-1-({(4R)-1-{(2S)-2-[(tert-butoxycarbonyl)amino]non-8-enoyl}-4-[(2-pyridin-2-ylthieno[2,3-d]pyrimidin-4-yl)oxy]-L-prolyl}amino)-2-vinylcyclopropanecarboxylate

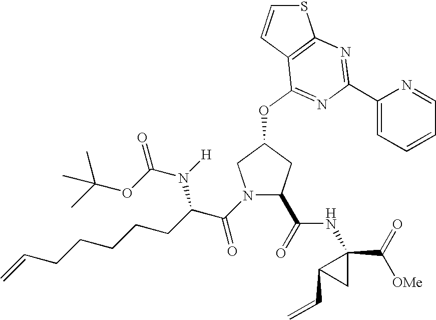

Methyl (1R,2S)-1-({(4R)-4-[(2-pyridin-2-ylthieno[2,3-d]pyrimidin-4-yl)oxy]-L-prolyl}amino)-2-vinylcyclopropanecarboxylate (1.5 g, 3.2 mmol, 1.0 equiv) and (2S)-2-[(tert-butoxycarbonyl)amino]non-8-enoic acid (0.87 g, 3.2 mmol, 1.0 equiv) was taken up in anhydrous DMA (30 mL) to which triethylamine (0.53 mL, 3.8 mmol, 1.2 equiv) followed by HATU (1.2 g, 3.2 mmol, 1.0 equiv) was added. The reaction mixture was stirred at 50° C. for 3 h, and then poured into 5% saturated sodium bicarbonate. The organic layer was extracted with MTBE, washed with saturated sodium chloride, dried over magnesium sulfate, filtered and concentrated in vacuo. The crude product was purified over silica gel (Biotage Horizon silica gel 40M column) and eluted with 0.5-5% methanol in chloroform (0.1% ammonium hydroxide) which provided a brown oil (2.1 g, 91% yield): ¹H NMR (400 MHz, DMSO-d6) δ 8.76 (d, J=8.8 Hz, 1H), 8.47 (d, J=7.8 Hz, 1H), 8.01-7.97 (m, 1H), 7.86 (d, J=6.1 HZ, 1H), 7.55-7.52 (m, 1H), 7.46 (d, J=5.8 Hz, 1H), 7.05 (d, J=7.3 Hz, 1H), 6.01 (s, 1H), 5.79-5.74 (m, 1H), 5.65-5.59 (m, 1H), 5.23 (dd, J=17.1, 1.8 Hz, 1H), 5.09 (dd, J=10.4, 1.8 Hz, 1H), 5.07-4.89 (m, 2H), 4.48 (t, J=8.1 Hz, 1H), 4.25 (d, J=12.1 Hz, 1H), 4.11-4.02 (m, 2H), 3.58 (s, 3H), 2.60-2.54 (m, 1H), 2.40-2.32 (m, 1H), 2.10-2.06 (m, 1H), 1.99-1.94 (m, 2H), 1.65-1.58 (m, 2H), 1.49-1.23 (m, 9H), 1.17 (s, 9H); LCMS (ESI+) for C₃₇H₄₆N₆O₇S m/z 719 (M+H)⁺.

### Example 23

### Methyl (2R,6S,12Z,13aS,14aR,16aS)-6-[(tert-butoxycarbonyl)amino]-5,16-dioxo-2-[(2-pyridin-2-ylthieno[2,3-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate

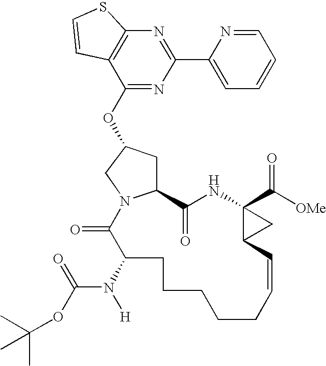 Methyl (1R,2S)-1-({(4R)-1-{(2S)-2-[(tert-butoxycarbonyl)amino]non-8-enoyl}-4-[(2-pyridin-2-ylthieno[2,3-d]pyrimidin-4-yl)oxy]-L-prolyl}amino)-2-vinylcyclopropanecarboxylate (2.1 g, 3 mmol, 1.0 equiv) was taken up in anhydrous dichloromethane (600 mL, 0.005 M). The reaction vessel was evacuated and purged with nitrogen gas. The Grubbs Catalyst 2^{nd} Generation was added (0.363 g, 0.43 mmol, 0.15 equiv) and the reaction mixture was stirred at 40° C. for 2 h. The crude reaction mixture was concentrated in vacuo and the crude product was purified over silica gel (Biotage Horizon silica gel 40M column) and eluted with 1-2.5% methanol in chloroform (0.1% ammonium hydroxide). The semi-pure product was triturated with MTBE/hexanes and provided a white solid (0.788 g, 38% yield): ¹H NMR (400 MHz, DMSO-d6) δ 8.79-8.72 (m, 2H), 8.48 (d, J=7.8 Hz, 1H), 7.99 (td, J=7.7, 1.8 Hz, 1H), 7.88-7.82 (m, 1H), 7.56-7.52 (m, 1H), 7.40 (d, J=6.1 Hz, 1H), 6.98 (d, J=6.3 Hz, 1H), 6.01 (s, 1H), 5.58-5.49 (m, 1H), 5.25 (t, J=9.6 Hz, 1H), 4.61-4.52 (m, 2H), 3.96 (d, J=8.3 Hz, 2H), 3.51 (s, 3H), 2.42 (m, 1H), 2.21 (q, J=8.8 Hz, 1H), 1.75-1.63 (m, 2H), 1.58-1.47 (m, 2H), 1.36-1.12 (m, 9H), 1.06 (s, 9H); LCMS (ESI+) for C₃₅H₄₂N₆O₇S m/z 691 (M+H)⁺.

### Example 24

### Methyl (2R,6S,12Z,13aS,14aR,16aS)-6-amino-5,16-dioxo-2-[(2-pyridin-2-ylthieno[2,3-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate

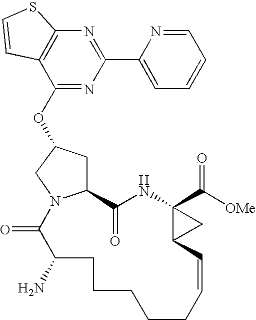

Methyl (2R,6S,12Z,13aS,14aR,16aS)-6-[(tert-butoxycarbonyl)amino]-5,16-dioxo-2-[(2-pyridin-2-ylthieno[2,3-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.700 g, 1.0 mmol, 1.0 equiv) was taken up in anhydrous dichloromethane (7.5 mL) to which trifluoroacetic acid (2.5 mL) was added. The reaction mixture was stirred at ambient temperature for 2 h. The reaction mixture was quenched with saturated sodium bicarbonate and the organic layer washed with saturated sodium chloride, dried over magnesium sulfate, filtered and concentrated in vacuo. The crude product was triturated with MTBE and provided a tan solid (0.590 g, >95% yield): ¹H NMR (400 MHz, DMSO-d6) δ 8.77 (d, J=3.8 Hz, 1H), 8.69 (s, 1H), 8.48 (d, J=7.8 Hz, 1H), 8.01-7.97 (m, 1H), 7.90 (d, J=5.8 Hz, 1H), 7.55-7.52 (m, 1H), 7.46 (d, J=5.8 Hz, 1H), 6.08 (s, 1H), 5.51 (q, J=8.9 Hz, 1H), 5.29 (t, J=9.8 Hz, 1H), 4.55 (t, J=7.5 Hz, 1H), 4.13-4.02 (m, 2H), 3.61-3.54 (m, 4H), 2.40-2.29 (m, 1H), 2.21 (q, J=8.8 Hz, 1H), 1.99-1.92 (m, 2H), 1.57-1.13 (m, 4H), 1.28 (s, 7H); LCMS (ESI+) for C₃₀H₃₄N₆O₅S m/z 591 (M+H)⁺.

### Example 25

### Methyl (2R,6S,12Z,13aS,14aR,16aS)-6-{[(cyclopentyloxy)carbonyl]amino}-5,16-dioxo-2-[(2-pyridin-2-ylthieno[2,3-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate

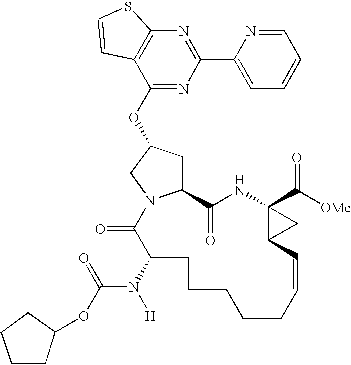

Methyl (2R,6S,12Z,13aS,14aR,16aS)-6-amino-5,16-dioxo-2-[(2-pyridin-2-ylthieno[2,3-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.200 g, 0.34 mmol, 1.0 equiv) and triethylamine (0.056 mL, 0.34 mmol, 1.2 equiv) were taken up in anhydrous DMA (3.4 mL). Cyclopentyl 4-nitrophenyl carbonate (0.086 g, 0.34 mmol, 1.0 equiv) was added and the reaction mixture was heated at 80° C. for 16 h. The reaction mixture was poured into 50% saturated sodium bicarbonate and a light yellow solid was collected (0.210 g, 88% yield): LCMS (ESI+) for C₃₆H₄₂N₆O₇S m/z 703 (M+H)⁺.

### Example 26

### (2R,6S,12Z,13aS,14aR,16aS)-6-{[(Cyclopentyloxy)carbonyl]amino}-5,16-dioxo-2-[(2-pyridin-2-ylthieno[2,3-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

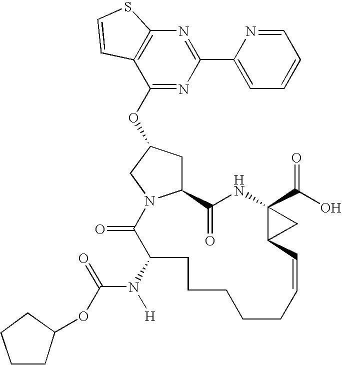

Methyl (2R,6S,12Z,13aS,14aR,16aS)-6-{[(cyclopentyloxy)carbonyl]amino}-5,16-dioxo-2-[(2-pyridin-2-ylthieno[2,3-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.205 g, 0.29 mmol, 1.0 equiv) was taken up in 1:1 anhydrous tetrahydrofuran and anhydrous methanol (2 mL) to which aqueous lithium hydroxide (1.2 mL of 40 mg/mL LiOH—H₂O, 1.16 mmol, 4.0 equiv) was added. The reaction mixture was stirred at ambient temperature for 2 h. The reaction mixture concentrated in vacuo, and then poured into 0.5 M sodium citrate buffer (pH 4.5). The aqueous layer was extracted with dichloromethane, washed with saturated sodium chloride, dried over magnesium sulfate, filtered and concentrated in vacuo. The crude product was purified by reverse phase chromatography (C18) and eluted with 30-75% acetonitrile in water (0.1% acetic acid) which provided a white solid (0.063 g, 31% yield): ¹H NMR (400 MHz, DMSO-d6) δ 12.23 (s, 1H), 8.77 (d, J=3.8 Hz, 1H), 8.63 (s, 1H), 8.49 (d, J=7.8 Hz, 1H), 8.00 (td, J=7.7, 1.5 Hz, 1H), 7.87 (d, J=5.8 Hz, 1H), 7.54 (dd, J=6.8, 5.0 Hz, 1H), 7.42 (d, J=6.1 Hz, 1H), 7.24-7.13 (m, 2H), 6.04 (s, 1H), 5.56-5.45 (m, 1H), 5.27 (t, J=9.5 Hz, 1H), 4.55-4.44 (m, 3H), 4.07-3.98 (m, 2H), 3.16 (d, J=5.3 Hz, 1H), 2.16 (q, J=8.6 Hz, 1H), 1.77-1.66 (m, 2H), 1.59-1.14 (m, 18H); LCMS (ESI+) for C₃₅H₄₀N₆O₇S m/z 689 (M+H)⁺; Anal. calcd. for C₃₅H₄₀N₆O₇S•0.20 MTBE•0.65H₂O: C, 60.21; H, 6.13; N, 11.70. Found: C, 60.22; H, 6.10; N, 11.30.

### Example 27

### Methyl (2R,6S,12Z,13aS,14aR,16aS)-6-{[(cyclobutyloxy)carbonyl]amino}-5,16-dioxo-2-[(2-pyridin-2-ylthieno[2,3-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate

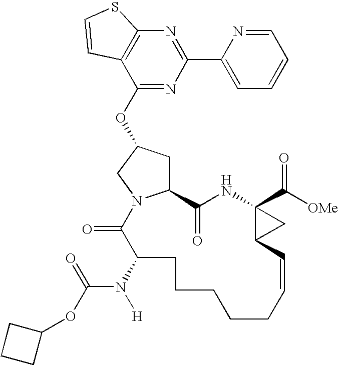

Methyl (2R,6S,12Z,13aS,14aR,16aS)-6-amino-5,16-dioxo-2-[(2-pyridin-2-ylthieno[2,3-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.183 g, 0.31 mmol, 1.0 equiv) and triethylamine (0.051 mL, 0.372 mmol, 1.2 equiv) were taken up in anhydrous DMA (3.0 mL). Cyclobutyl 4-nitrophenyl carbonate (0.074 g, 0.31 mmol, 1.0 equiv) was added and the reaction mixture was heated at 80° C. for 16 h. The reaction mixture was poured into 50% saturated sodium bicarbonate. The organic layer was extracted with ethyl acetate, washed with saturated sodium chloride, dried over magnesium sulfate, filtered and concentrated in vacuo which provided a brown oil (0.283 g, >95% yield): ¹H NMR (400 MHz, DMSO-d6) δ 11.04 (s, 1H), 8.77 (d, J=3.8 Hz, 1H), 8.71 (s, 1H), 8.49 (d, J=5.8 Hz, 1H), 8.13-8.09 (m, 2H), 8.00 (m, 1H), 7.89 (d, J=5.8 Hz, 1H), 7.56-7.54 (m, 2H), 7.42 (d, J=6.1 Hz, 1H), 7.31 (d, J=6.6 Hz, 1H), 6.04 (s, 1H), 5.5-5.51 (m, 1H), 5.56-5.45 (m, 1H), 5.27 (t, J=9.5 Hz, 1H), 4.55-4.44 (m, 1H), 4.35-4.27 (m, 1H), 3.56 (s, 3H), 2.56-2.51 (m, 1H), 2.44-2.35 (m, 1H), 2.21 (m, 1H), 1.81-1.66 (m, 4H), 1.62-1.48 (m, 3H), 1.36-1.20 (m, 19H); LCMS (ESI+) for C₃₅H₄₀N₆O₇S m/z 689 (M+H)⁺.

### Example 28

### (2R,6S,12Z,13aS,14aR,16aS)-6-{[(cyclobutyloxy)carbonyl]amino}-5,16-dioxo-2-[(2-pyridin-2-ylthieno[2,3-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

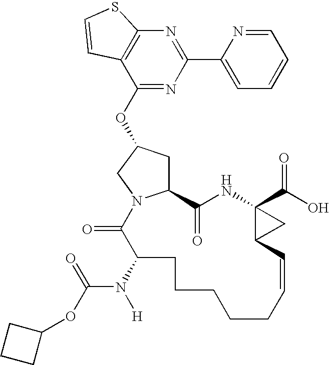

Methyl (2R,6S,12Z,13aS,14aR,16aS)-6-{[(cyclobutyloxy)carbonyl]amino}-5,16-dioxo-2-[(2-pyridin-2-ylthieno[2,3-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.263 g, 0.38 mmol, 1.0 equiv) was taken up in 1:1 anhydrous tetrahydrofuran and anhydrous methanol (4 mL) to which aqueous lithium hydroxide (1.6 mL of 40 mg/mL LiOH—H₂O, 1.53 mmol, 4.0 equiv) was added. The reaction mixture was stirred at ambient temperature for 2 h. The reaction mixture was concentrated in vacuo, poured into 0.5 M sodium citrate buffer (pH 4.5). The aqueous layer was extracted with dichloromethane, washed with saturated sodium chloride, dried over magnesium sulfate, filtered and concentrated in vacuo. The crude product was purified by reverse phase chromatography (C18) and eluted with 30-75% acetonitrile in water (0.1% acetic acid) which provided a white solid (0.047 g, 18% yield): ¹H NMR (400 MHz, DMSO-d6) δ 11.93 (s, 1H), 8.77 (s, 1H), 8.63 (s, 1H), 8.49 (m, 1H), 8.00 (m, 1H), 7.89 (d, J=5.6 Hz, 1H), 7.54 (s, 1H), 7.44 (d, J=5.6 Hz, 1H), 7.29 (d, 1H), 6.04 (s, 1H), 5.56-5.45 (m, 1H), 5.27 (t, J=9.6 Hz, 1H), 4.51 (t, J=7.7 Hz, 1H), 4.48 (d, J=11.4 Hz, 1H), 4.34-4.30 (m, 1H), 4.02-3.97 (m, 2H), 2.45-2.42 (m, 2H), 2.16 (q, J=8.6 Hz, 1H), 1.85-1.63 (m, 6H), 1.52-1.29 (m, 9H), 1.23 (br. s, 3H); LCMS (ESI+) for C₃₄H₃₈N₆O₇S m/z 675 (M+H)⁺; Anal. calcd. for C₃₄H₃₈N₆O₇S•0.3 MTBE•1.0H₂O•0.3 acetic acid: C, 57.56; H, 6.18; N, 10.74. Found: C, 57.17; H, 5.96; N, 10.46.

### Example 29

### (2R,6S,12Z,13aS,14aR,16aS)-6-[(tert-butoxycarbonyl)amino]-5,16-dioxo-2-[(2-pyridin-2-ylthieno[2,3-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

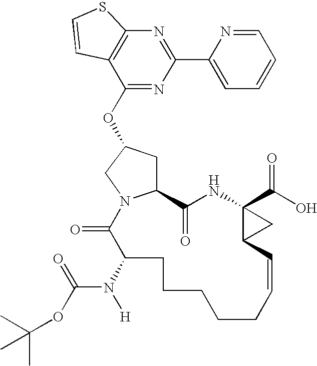

Methyl (2R,6S,12Z,13aS,14aR,16aS)-6-[(tert-butoxycarbonyl)amino]-5,16-dioxo-2-[(2-pyridin-2-ylthieno[2,3-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.100 g, 0.15 mmol, 1.0 equiv) was taken up in 1:1 anhydrous tetrahydrofuran and anhydrous methanol (1.2 mL) to which aqueous lithium hydroxide (0.61 mL of 40 mg/mL LiOH—H₂O, 0.58 mmol, 4.0 equiv) was added. The reaction mixture was stirred at ambient temperature for 2.5 h. The reaction mixture was concentrated in vacuo, and poured into 0.5 M sodium citrate buffer (pH 4.5). A white solid was collected and purified by reversed phase chromatography (C18) and eluted with 30-75% acetonitrile in water (0.1% acetic acid) which provided a white solid (0.008 g, 8% yield): ¹H NMR (400 MHz, DMSO-d6) δ 8.88 (m, 1H), 8.75-8.68 (m, 2H), 8.39-8.37 (m, 1H), 8.00-7.95 (m, 1H), 7.86 (s, 1H), 7.50-7.45 (m, 1H), 7.00-6.97 (m, 1H), 6.12 (s, 1H), 5.52-5.48 (m, 1H), 5.27 (t, J=9.4 Hz, 1H), 4.62-4.52 (m, 3H), 4.03-3.83 (m, 5H), 2.58-2.51 (m, 1H), 2.40-2.38 (m, 1H), 2.16 (q, J=9.4 Hz, 1H), 1.74-1.60 (m, 3H), 1.54-1.41 (m, 3H), 1.39-1.27 (m, 6H), 1.22 (br. s, 3H), 1.16-1.10 (m, 3H); LCMS (ESI+) for C₃₄H₄₀N₆O₇S m/z 677 (M+H)⁺.

### Example 30

### Methyl (2R,6S,12Z,13aS,14aR,16aS)-6-[(cyclopropylacetyl)amino]-5,16-dioxo-2-[(2-pyridin-2-ylthieno[2,3-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate

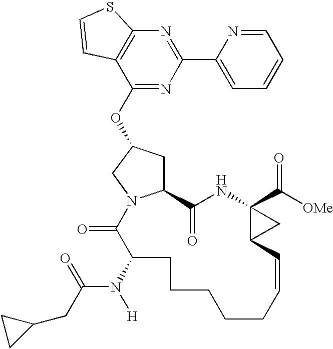

Methyl (2R,6S,12Z,13aS,14aR,16aS)-6-amino-5,16-dioxo-2-[(2-pyridin-2-ylthieno[2,3-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.187 g, 0.32 mmol, 1.0 equiv) and cyclopropylacetic acid (0.032 g, 0.32 mmol, 1.0 equiv) were taken up in anhydrous dichloromethane (10.6 mL, 0.03 M). Diisopropylethylamine (0.275 mL, 1.6 mmol, 5.0 equiv) was added followed by HATU (0.120 g, 0.32 mmol, 1.0 equiv). The reaction mixture was stirred for 15 h, then diluted with ethyl acetate and poured into saturated sodium bicarbonate. The organic layer was then washed with 0.5 M sodium citrate buffer (pH=4.5), saturated sodium chloride, dried over magnesium sulfate, filtered and concentrated in vacuo which provided a white solid (0.231 g, >95% yield): ¹H NMR (400 MHz, DMSO-d6) δ 8.77 (m, 1H), 8.72 (s, 1H), 8.49 (d, J=8.1 Hz, 1H), 8.05 (d, J=6.8 Hz, 1H), 8.02-7.98 (m, 1H), 7.88 (d, J=5.8 Hz, 1H), 7.56-7.52 (m, 2H), 7.43 (d, J=6.1 Hz, 1H), 6.04 (s, 1H), 5.56-5.50 (m, 1H), 5.27 (t, J=9.8 Hz, 1H), 4.53-4.49 (m, 1H), 4.40-4.33 (m, 2H), 4.12-4.09 (m, 1H), 3.56 (s, 3H), 2.67-2.64 (m, 2H), 2.59-2.53 (m, 1H), 2.50-2.42 (m, 1H), 2.28-2.21 (m, 1H), 1.91-1.68 (m, 2H), 1.55-1.50 (m, 2H), 1.43-1.29 (m, 5H), 1.29-1.22 (m, 2H), 0.73-0.69 (m, 1H), 0.27-0.24 (m, 2H), −0.01-(−0.02) (m, 2H); LCMS (ESI+) for C₃₅H₄₀N₆O₆S m/z 673 (M+H)⁺.

### Example 31

### (2R,6S,12Z,13aS,14aR,16aS)-6-[(Cyclopropylacetyl)amino]-5,16-dioxo-2-[(2-pyridin-2-ylthieno[2,3-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

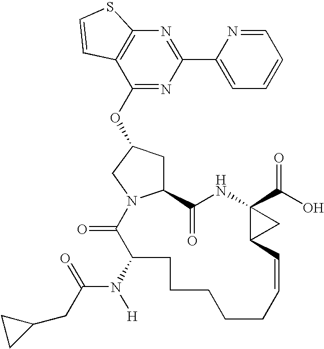

Methyl (2R,6S,12Z,13aS,14aR,16aS)-6-[(cyclopropylacetyl)amino]-5,16-dioxo-2-[(2-pyridin-2-ylthieno[2,3-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.211 g, 0.314 mmol, 1.0 equiv) was taken up in 1:1 anhydrous tetrahydrofuran and anhydrous methanol (2 mL) to which aqueous lithium hydroxide (1.3 mL of 40 mg/mL LiOH—H₂O, 1.25 mmol, 4.0 equiv) was added. The reaction mixture was stirred at ambient temperature for 2 h, and poured into 0.5 M sodium citrate buffer (pH 4.5). The resultant solid was purified by reverse phase chromatography (C18) and eluted with 30-75% acetonitrile in water (0.1% acetic acid) which provided a white solid (0.045 g, 22% yield): ¹H NMR (400 MHz, DMSO-d6) δ 12.20 (s, 1H), 8.77 (d, J=4.0 Hz, 1H), 8.64 (s, 1H), 8.49 (d, J=7.6 Hz, 1H), 8.02-7.87 (m, 3H), 7.55-7.53 (m, 1H), 7.43 (d, J=5.8 Hz, 1H), 7.25-7.15 (m, 1H), 6.06 (s, 1H), 5.51-5.45 (m, 1H), 5.29 (t, J=9.6 Hz, 1H), 4.49 (t, J=7.7 Hz, 1H), 4.37 (m, 2H), 4.12-4.08 (m, 1H), 3.15 (d, J=4.8 Hz, 2H), 2.23-2.16 (m, 1H), 1.92-1.69 (m, 2H), 1.49-1.23 (m, 9H), 0.90-0.67 (m, 3H), 0.28-0.26 (m, 2H), −0.02-(−0.03) (m, 2H); LCMS (ESI+) for C₃₄H₃₆N₆O₆S m/z 659 (M+H)⁺; Anal. calcd. for C₃₄H₃₈N₆O₆S•0.29 MTBE•0.64 acetic acid•0.96H₂O: C, 59.61; H, 6.26; N, 11.36. Found: C, 59.46; H, 5.86; N, 10.98.

### Example 32

### 2-Pyridin-2-ylthieno[3,2-d]pyrimidin-4-ol

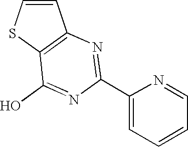

Methyl 3-aminothiophene-2-carboxylate (4.0 g, 25.4 mmol, 1.0 equiv) and pyridine-2-carbonitrile (2.4 mL, 25.4 mmol, 1.0 equiv) were taken up in anhydrous tetrahydrofuran (100 mL). The resultant, beige mixture was cooled to 0° C., to which potassium tert-butoxide (4.3 g, 38.1 mmol, 1.5 equiv) was added. Reaction mixture stirred for 12 h, concentrated in vacuo and poured into 50% saturated ammonium chloride. The crude, beige colored solids were collected by filtration. (3.9 g, 67% yield): ¹H NMR (400 MHz, DMSO-d6) δ 12.01 (s, 1H), 8.75 (d, J=4.8 Hz, 1H), 8.39 (d, J=7.3 Hz, 1H), 8.25 (d, J=5.3 Hz, 1H), 8.06 (t, J=7.8 Hz, 1H), 7.64 (dd, J=7.3, 4.8 Hz, 1H), 7.51 (d, J=5.3 Hz, 1H); LCMS (ESI+) for C₁₁H₇N₃OS m/z 230 (M+H)⁺.

### Example 33

### 1-Tert-butyl 2-methyl (2S,4R)-4-[(2-pyridin-2-ylthieno[3,2-d]pyrimidin-4-yl)oxy]pyrrolidine-1,2-dicarboxylate

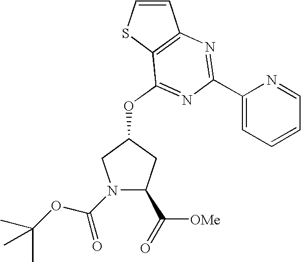

2-Pyridin-2-ylthieno[3,2-d]pyrimidin-4-ol (1.6 g, 7.0 mmol, 1.0 equiv) and 1-tert-butyl 2-methyl (2S,4S)-4-hydroxypyrrolidine-1,2-dicarboxylate (1.7 g, 7.0 mmol, 1.0 equiv) and triphenylphosphine (3.7 g, 14 mmol, 2.0 equiv) were taken up in anhydrous tetrahydrofuran (140 mL). The resultant white slurry was cooled to 0° C., followed by the addition of DIAD (2.7 mL, 14 mmol, 2.0 equiv). The amber solution was warmed to ambient temperature and stirred for 16 h. The reaction mixture was concentrated in vacuo, poured into 5% saturated bicarbonate and the organic layer extracted with ethyl acetate. The organic layer was extracted into 1N HCl. The aqueous layer washed with ethyl, acetate, and then basified to pH 9 using sodium bicarbonate. The basic aqueous layer was extracted into ethyl acetate. The organic layer washed with saturated sodium chloride, dried over magnesium sulfate, filtered and concentrated in vacuo. The resultant solid was triturated with MTBE/hexanes and provided a white foam (2.2 g, 69% yield): ¹H NMR (400 MHz, DMSO-d6) δ 8.76 (d, J=4.3 Hz, 1H), 8.42 (t, J=6.9 Hz, 2H), 7.98 (t, J=7.7 Hz, 1H), 7.69 (d, J=5.3 Hz, 1H), 7.54-7.51 (m, 1H), 5.97 (s, 1H), 4.42-4.38 (m, 1H), 3.89-3.84 (m, 1H), 3.76-3.65 (m, 4H), 2.72-2.68 (m, 1H), 2.47-2.41 (m, 1H), 1.35 (s, 9H); LCMS (ESI+) for C₂₂H₂₄N₄O₅S m/z 457 (M+H)⁺.

### Example 34

### (4R)-1-(tert-butoxycarbonyl)-4-[(2-pyridin-2-ylthieno[3,2-d]pyrimidin-4-yl)oxy]-L-proline

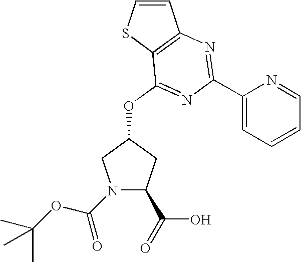

1-Tert-butyl 2-methyl (2S,4R)-4-[(2-pyridin-2-ylthieno[3,2-d]pyrimidin-4-yl)oxy]pyrrolidine-1, 2-dicarboxylate (2.2 g, 4.8 mmol, 1.0 equiv) was taken up in a 1:1 solution of anhydrous tetrahydrofuran and anhydrous methanol (2 mL). A solution of aqueous lithium hydroxide (20 mL of 40 mg/mL LiOH—H₂O, 2 equiv) was added and the reaction mixture was stirred at ambient for 25 minutes. The reaction mixture was concentrated in vacuo, poured into 0.5 M sodium citrate buffer (pH 4.5), extracted with ethyl acetate. The combined organic extracts were washed with saturated sodium chloride, dried over magnesium sulfate, filtered and concentrated in vacuo which provided a white solid (2.10 g, >95% yield): ¹H NMR (400 MHz, DMSO-d6) δ 12.81 (s, 1H), 8.77 (d, J=3.8 Hz, 1H), 8.48-8.38 (m, 2H), 8.04-7.95 (m, 1H), 7.69 (d, J=5.3 Hz 1H), 7.57-7.48 (m, 1H), 5.96 (d, J=2.0 Hz, 1H), 4.31 (q, J=8.3 Hz, 1H), 3.91-3.81 (m, 1H), 3.71 (d, J=11.1 Hz, 1H), 2.72-2.61 (m, 1H), 2.46-2.38 (m, 1H), 1.6 (s, 9H); LCMS (ESI+) for C₂₁H₂₂N₄O₅S m/z 443 (M+H)⁺.

### Example 35

### tert-Butyl (2S,4R)-2-({[(1R,2S)-1-(methoxycarbonyl)-2-vinylcyclopropyl]amino}carbonyl)-4-[(2-pyridin-2-ylthieno[3,2-d]pyrimidin-4-yl)oxy]pyrrolidine-1-carboxylate

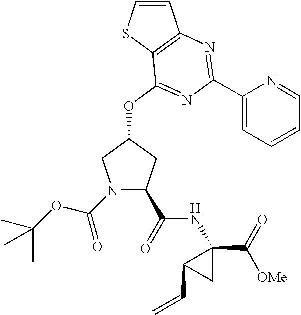

(4R)-1-(Tert-butoxycarbonyl)-4-[(2-pyridin-2-ylthieno[3,2-d]pyrimidin-4-yl)oxy]-L-proline (2.1 g, 4.7 mmol, 1.0 equiv) and methyl (1R,2S)-1-amino-2-vinylcyclopropanecarboxylate hydrochloride (0.84 g, 4.7 mmol, 1.0 equiv) were taken up in anhydrous DMA (47 mL) to which triethylamine (2.0 mL, 14.1 mmol, 3.0 equiv) was added followed by HATU (1.8 g, 4.7 mmol, 3.0 equiv). The reaction mixture was stirred at 50° C. for 1 h and poured into saturated sodium bicarbonate. A solid was collected and purified over silica gel (Biotage Horizon silica gel 40M column) which was eluted with 0-5% methanol in chloroform (0.1% ammonium hydroxide). The semi-pure product was triturated with MTBE/hexanes which provided a white foam (2.4 g, 90% yield): ¹H NMR (400 MHz, DMSO-d6) δ 8.77 (d, J=9.8 Hz, 2H) 8.47-8.40 (m, 2H) 8.02-7.96 (m, 1H) 7.70 (d, J=5.3 Hz, 1H) 7.53 (dd, J=7.2, 4.7 Hz, 1H) 5.93 (s, 1H) 5.64 (d, J=9.8 Hz, 1H) 5.29 (s, 1H) 5.12 (s, 1H), 4.33-4.23 (m, 1H) 3.93-3.86 (m, 1H), 3.73 (s, 1H) 3.63-3.56 (m, 3H) 2.35 (t, J=13.0 Hz, 1H), 2.16 (d, J=8.8 Hz, 1H) 1.67 (d, J=7.6 Hz, 1H) 1.39-1.29 (m, 11H); LCMS (ESI+) for C₂₈H₃₁N₅O₆S m/z 566 (M+H)⁺.

### Example 36

### Methyl (1R,2S)-1-({(4R)-4-[(2-pyridin-2-ylthieno[3,2-d]pyrimidin-4-yl)oxy]-L-prolyl}amino)-2-vinylcyclopropanecarboxylate

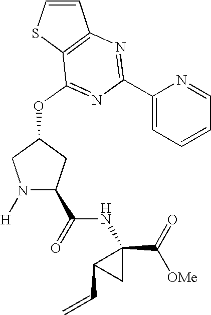

Tert-butyl (2S,4R)-2-({[(1R,2S)-1-(methoxycarbonyl)-2-vinylcyclopropyl]amino}carbonyl)-4-[(2-pyridin-2-ylthieno[3,2-d]pyrimidin-4-yl)oxy]pyrrolidine-1-carboxylate (2.4 g, 4.2 mmol, 1.0 equiv) was taken up in anhydrous dichloromethane (30 mL) to which trifluoroacetic acid (10 mL) was added. The reaction mixture was stirred at ambient temperature for 1 h and poured into saturated bicarbonate. The aqueous layer was extracted with dichloromethane, washed with saturated sodium chloride, dried over magnesium sulfate, filtered and concentrated in vacuo. The semi-pure product was triturated with MTBE which provided a beige solid (0.46 g, 75% yield): ¹H NMR (400 MHz, DMSO-d6) δ 8.76 (d, J=3.8 Hz, 1H), 8.70 (s, 1H), 8.45 (d, J=7.8 Hz, 1H), 8.40 (d, J=5.6 Hz, 1H), 7.98 (td, J=7.7, 1.8 Hz, 1H), 7.68 (d, J=5.3 Hz, 1H), 7.54-7.49 (m, 1H), 5.87 (t, J=4.9 Hz, 1H), 5.67-5.57 (m, 1H), 5.28 (dd, J=17.2, 2.0 Hz, 1H), 5.09 (dd, J=10.2, 1.9 Hz, 1H), 3.82 (t, J=7.8 Hz, 1H), 3.60 (s, 3H), 3.35 (dd, J=12.6, 4.8 Hz, 1H), 3.17 (d, J=12.6 Hz, 1H), 2.32-2.27 (m, 1H), 2.25-2.20 (m, 2H), 1.65 (dd, J=8.0, 5.2 Hz, 1H), 1.33 (dd, J=9.2, 5.2 Hz, 1H); LCMS (ESI+) for C₂₃H₂₃N₅O₄S m/z 466 (M+H)⁺.

### Example 37

### Methyl (1R,2S)-1-({(4R)-1-{(2S)-2-[(tert-butoxycarbonyl)amino]non-8-enoyl}-4-[(2-pyridin-2-ylthieno[3,2-d]pyrimidin-4-yl)oxy]-L-prolyl}amino)-2-vinylcyclopropanecarboxylate

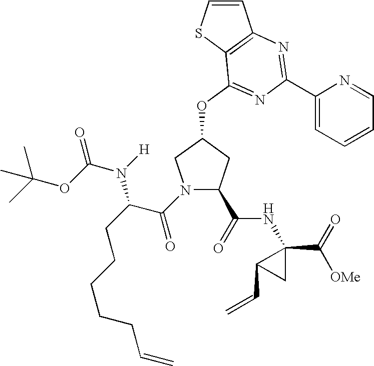

Methyl (1R,2S)-1-({(4R)-4-[(2-pyridin-2-ylthieno[3,2-d]pyrimidin-4-yl)oxy]-L-prolyl}amino)-2-vinylcyclopropanecarboxylate (1.0 g, 2.15 mmol, 1.0 equiv) and (2S)-2-[(tert-butoxycarbonyl)amino]non-8-enoic acid (0.58 g, 2.15 mmol, 1.0 equiv) was taken up in anhydrous DMA (22 mL) to which triethylamine (0.356 mL, 2.6 mmol, 1.2 equiv) was added followed by HATU (0.817 g, 2.15 mmol, 1.0 equiv). The reaction mixture was stirred at 50° C. for 1 h and poured into 50% saturated sodium bicarbonate which provided an beige solid (1.26 g, 84% yield): ¹H NMR (400 MHz, DMSO-d6) δ 8.76 (d, J=4.0 Hz, 1H), 8.64 (s, 1H), 8.48-8.38 (m, 2H), 8.02-7.96 (m, 1H), 7.74-7.66 (m, 1H), 7.58-7.50 (m, 1H), 7.01 (s, 1H), 6.05 (s, 1H), 5.81-5.59 (m, 2H), 5.09 (d, J=11.6 Hz, 1H), 4.98-4.89 (m, 3H), 4.48-4.44 (m, 1H), 4.27-4.23 (m, 1H), 4.11-4.00 (m, 2H), 3.80 (s, 3H), 2.42-2.31 (m, 1H), 2.12-2.03 (m, 1H), 2.02-1.92 (m, 2H), 1.65-1.53 (m, 2H), 1.32-1.19 (m, 9H), 1.50 (s, 9H); LCMS (ESI+) for C₃₇H₄₆N₆O₇S m/z 719 (M+H)⁺.

### Example 38

### Methyl (2R,6S,12Z,13aS,14aR,16aS)-6-[(tert-butoxycarbonyl)amino]-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate

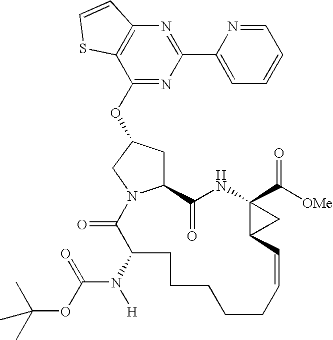

Methyl (1R,2S)-1-({(4R)-1-{(2S)-2-[(tert-butoxycarbonyl)amino]non-8-enoyl}-4-[(2-pyridin-2-ylthieno[3,2-d]pyrimidin-4-yl)oxy]-L-prolyl }amino)-2-vinylcyclopropanecarboxylate (1.2 g, 1.7 mmol, 1.0 equiv) was taken up in anhydrous dichloromethane (340 mL, 0.005 M). The reaction vessel was evacuated and purged with nitrogen gas. The Grubbs Catalyst 2^{nd} Generation was added (0.216 g, 0.26 mmol, 0.15 equiv) and the reaction mixture was stirred at 40° C. for 2 h. The reaction mixture was concentrated in vacuo and the product was purified over silica gel (Biotage Horizon silica gel 40M column) which was eluted with 1-2.5% methanol in dichloromethane (0.1% ammonium hydroxide). The semi-pure product was triturated with MTBE/hexanes and provided a white solid (0.275 g, 25% yield): ¹H NMR (400 MHz, DMSO-d6) δ 8.76 (s, 2H), 8.46 (d, J=7.8 Hz, 1H), 8.35 (d, J=5.1 Hz, 1H), 7.99 (td, J=7.7, 1.5 Hz, 1H), 7.66 (d, J=5.6 Hz, 1H), 7.53 (dd, J=7.3, 4.8 Hz, 1H), 6.97 (d, J=6.6 Hz, 1H), 6.06 (s, 1H), 5.58-5.48 (m, 1H), 5.25 (t, J=9.6 Hz, 1H), 4.54 (t, J=8.1 Hz, 1H), 4.03-3.92 (m, 2H), 3.61-3.54 (m, 3H), 2.41 (ddd, J=13.1, 8.9, 4.0 Hz, 2H), 2.24 (q, J=8.8 Hz, 1H), 1.77-1.65 (m, 3H), 1.58-1.47 (m, 2H), 1.36 (s, 1H), 1.31 (s, 4H), 1.20-1.12 (m, 3H), 1.08-0.99 (m, 9H); LCMS (ESI+) for C₃₅H₄₂N₆O₇S m/z 691 (M+H)⁺.

### Example 39

### Methyl (2R,6S,12Z,13aS,14aR,16aS)-6-amino-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate

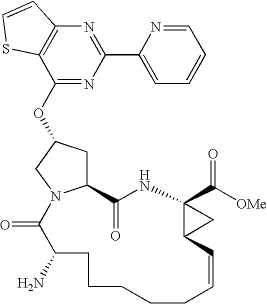 Methyl (2R,6S,12Z,13aS,14aR,16aS)-6-[(tert-butoxycarbonyl)amino]-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.100 g, 0.14 mmol, 1.0 equiv) was taken up in anhydrous dichloromethane (1 mL) to which trifluoroacetic acid added (0.5 mL). The reaction mixture was stirred at ambient temperature for 1 h and quenched with saturated sodium bicarbonate. The organic layer washed with saturated sodium chloride, dried over magnesium sulfate, filtered and concentrated in vacuo. The semi-pure product was triturated with dichloromethane/hexanes which provided a white solid (0.088 g, >95% yield): ¹H NMR (400 MHz, DMSO-d6) δ 8.79-8.74 (m, 1H), 8.68 (s, 1H), 8.47 (d, J=7.8 Hz, 1H), 8.41 (d, J=5.3 Hz, 1H), 7.99 (td, J=7.7, 1.8 Hz, 1H), 7.70 (d, J=5.6 Hz, 1H), 7.56-7.51 (m, 1H), 6.12 (d, J=2.5 Hz, 1H), 5.55-5.45 (m, 1H), 5.28 (t, J=9.8 Hz, 1H), 4.54 (t, J=7.4 Hz, 1H), 4.13-4.01 (m, 2H), 3.59-3.55 (m, 3H), 3.50 (dd, J=8.2, 2.6 Hz, 1H), 2.33-2.22 (m, 2H), 1.97-1.85 (m, 1H), 1.81-1.67 (m, 2H), 1.58-1.46 (m, 4H), 1.26-1.13 (m, 8H); LCMS (ESI+) for C₃₀H₃₄N₆O₅S m/z 591 (M+H)⁺.

### Example 40

### Methyl (2R,6S,12Z,13aS,14aR,16aS)-6-{[(cyclopentyloxy)carbonyl]amino}-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate

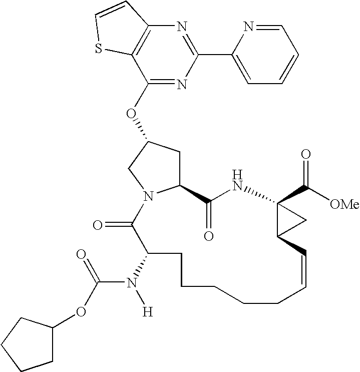 Methyl (2R,6S,12Z,13aS,14aR,16aS)-6-amino-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.086 g, 0.14 mmol, 1.0 equiv) and triethylamine (0.023 mL, 0.18 mmol, 1.2 equiv) were taken up in anhydrous DMA (1.5 mL). Cyclopentyl 4-nitrophenyl carbonate (0.037 g, 0.14 mmol, 1.0 equiv) was added and the reaction mixture was heated at 80° C. for 16 h. The reaction mixture was poured into saturated sodium bicarbonate, and the aqueous layer was extracted with ethyl acetate. The combined organic layers were washed with saturated sodium chloride, dried over magnesium sulfate, filtered and concentrated in vacuo which provided a brown oil. The crude product was purified over silica gel (Biotage Horizon silica gel 40M column), which was eluted with 1-5% methanol in chloroform (0.1% ammonium hydroxide) and provided a white solid (0.084 g, 86% yield): ¹H NMR (400 MHz, DMSO-d6) δ 8.76 (d, J=4.8 Hz, 1H), 8.73 (s, 1H), 8.48 (d, J=8.1 Hz, 1H), 8.38 (d, J=5.3 Hz, 1H), 8.01 (m, 1H), 7.67 (d, J=5.3 Hz, 1H), 7.55-7.52 (m, 1H), 7.17 (d, J=6.8 Hz, 1H), 6.11 (s, 1H), 5.53 (q, J=8.7 Hz, 1H), 5.26 (t, J=9.7 Hz, 1H), 4.55-4.48 (m, 2H), 4.37 (s, 1H), 4.03-3.99 (m, 2H), 3.56 (s, 3H), 2.47-2.41 (m, 1H), 2.26-2.22 (m, 1H), 1.80-1.60 (m, 3H), 1.57-1.20 (m, 18H); LCMS (ESI+) for C₃₆H₄₂N₆O₇S m/z 703 (M+H)⁺.

### Example 41

### (2R,6S,12Z,13aS,14aR,16aS)-6-{[(cyclopentyloxy)carbonyl]amino}-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

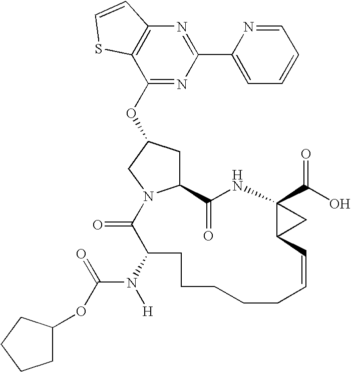

Methyl (2R,6S,12Z,13aS,14aR,16aS)-6-{[(cyclopentyloxy)carbonyl]amino}-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.082 g, 0.12 mmol, 1.0 equiv) was taken up in 1:1 anhydrous tetrahydrofuran and anhydrous methanol (1 mL) to which aqueous lithium hydroxide (0.50 mL of 40 mg/mL LiOH—H₂O, 0.48 mmol, 4 equiv) was added. The reaction mixture was stirred at ambient temperature for 3 h, concentrated in vacuo, and treated with 0.5 M sodium citrate buffer (pH 4.5). The aqueous layer was extracted with ethyl acetate, and the combined organic layers were washed with saturated sodium chloride, dried over magnesium sulfate, filtered and concentrated in vacuo. The semi-pure product was triturated with MTBE/hexanes and provided a white solid (0.036 g, 43% yield): ¹H NMR (400 MHz, DMSO-d6) δ 12.19 (br. s, 1H), 8.77 (d, J=4.3 Hz, 1H), 8.66 (s, 1H), 8.48 (d, J=7.6 Hz, 1H), 8.39 (d, J=5.3 Hz, 1H), 8.00 (t, J=7.7 Hz, 1H), 7.68 (d, J=5.3 Hz, 1H), 7.55-7.52 (m, 1H), 7.16 (d, J=6.8 Hz, 1H), 6.10 (s, 1H), 5.53 (q, J=8.8 Hz, 1H), 5.27 (t, J=9.6 Hz, 1H), 4.52 (q, J=9.9 Hz, 2H), 4.39 (s, 1H), 4.04-3.99 (m, 2H), 2.44-2.41 (m, 1H), 2.22-2.12 (m, 1H), 1.80-1.60 (m, 3H), 1.51-1.20 (m, 18H); LCMS (ESI+) for C₃₅H₄₀N₆O₇S m/z 689 (M+H)⁺; Anal. calcd. for C₃₅H₄₀N₆O₇S•0.70 MTBE, •0.32 TFA•1.3H₂O: C, 58.38; H, 5.96; N, 10.32. Found: C, 58.32; H, 6.21; N, 10.01.

### Example 42

### Methyl (2R,6S,12Z,13aS,14aR,16aS)-6-{[(cyclobutyloxy)carbonyl]amino}-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate

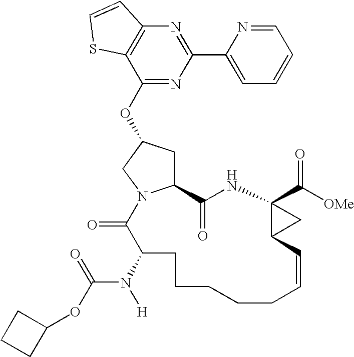

Methyl (2R,6S,12Z,13aS,14aR,16aS)-6-amino-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.063 g, 0.107 mmol, 1.0 equiv) and triethylamine (0.018 mL, 0.13 mmol, 1.2 equiv) were taken up in anhydrous DMA (1.5 mL). Cyclopentyl 4-nitrophenyl carbonate (0.025 g, 0.107 mmol, 1.0 equiv) was added and the reaction mixture was heated at 80° C. for 16 h. The reaction mixture was poured into saturated sodium bicarbonate, and the aqueous layer was extracted with ethyl acetate. The combined organic extracts were washed with saturated sodium chloride, dried over magnesium sulfate, filtered and concentrated in vacuo, which provided a yellow solid (0.092 g, >95% yield): ¹H NMR (400 MHz, DMSO-d6) δ 11.04 (s, 1H), 8.81-8.72 (m, 2H), 8.50-8.39 (m, 2H), 8.13-8.08 (m, 2H), 7.99 (td, J=7.7, 1.5 Hz, 1H), 7.70 (d, J=5.3 Hz, 1H), 7.53 (dd, J=7.2, 4.6 Hz, 1H), 7.29 (d, J=7.1 Hz, 1H), 6.97-6.89 (m, 2H), 5.56-5.49 (m, 1H), (d, J=10.11 Hz, 1H), 5.28 (t, J=9.7 Hz, 1H), 4.55-4.46 (m, 2H), 4.24-4.14 (m, 1H), 3.99-3.94 (m, 1H), 3.56 (s, 3H), 2.44-2.20 (m, 4H), 1.91-1.85 (m, 4H), 1.64-1.46 (m, 4H), 1.42-1.26 (m, 5H); LCMS (ESI+) for C₃₅H₄₀N₆O₇S m/z 689 (M+H)⁺.

### Example 43

### (2R,6S,12Z,13aS,14aR,16aS)-6-{[(cyclobutyloxy)carbonyl]amino}-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

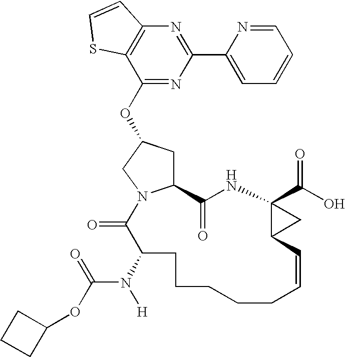

Methyl (2R,6S,12Z,13aS,14aR,16aS)-6-{[(cyclobutyloxy)carbonyl]amino}-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.091 g, 0.132 mmol, 1.0 equiv) was taken up in 1:1 anhydrous tetrahydrofuran and anhydrous methanol (1 mL) to which aqueous lithium hydroxide (1.4 mL of 40 mg/mL LiOH—H₂O, 1.32 mmol, 10 equiv) was added. The reaction mixture was stirred at ambient temperature for 5 h, concentrated in vacuo, and poured into 0.5 M sodium citrate buffer (pH 4.5). Aqueous layer was extracted with ethyl acetate, and the combined organic layers were washed with saturated sodium chloride, dried over magnesium sulfate, filtered and concentrated in vacuo. The crude product was purified by reverse phase chromatography (C18) and eluted with 30-75% acetonitrile in water (0.1% acetic acid) which provided a white solid (0.026 g, 39% yield): ¹H NMR (400 MHz, DMSO-d6) δ 11.98 (s, 1H), 8.78 (d, J=4.3 Hz, 1H), 8.66 (s, 1H), 8.48 (d, J=7.8 Hz, 1H), 8.40 (d, J=5.3 Hz, 1H), 8.01-7.90 (m, 1H), 7.69 (d, J=6.8 Hz, 1H), 7.53 (m, 1H), 7.28 (d, J=6.8 Hz, 1H), 6.10 (s, 1H), 5.50-5.45 (m, 1H), 5.27 (t, J=9.4 Hz, 1H), 4.53-4.45 (m, 2H), 4.25-4.16 (m, 1H), 4.04-3.98 (m, 3H), 2.21-2.16 (m, 1H), 1.76-1.66 (m, 5H), 1.62-1.42 (m, 5H), 1.32 (br. s, 9H); LCMS (ESI+) for C₃₄H₃BN₆O₇S m/z 675 (M+H)⁺.

### Example 44

### (2R,6S,12Z,13aS,14aR,16aS)-6-[(Tert-butoxycarbonyl)amino]-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

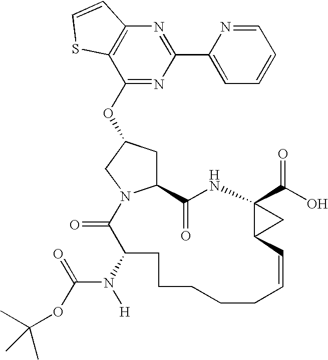

Methyl (2R,6S,12Z,13aS,14aR,16aS)-6-[(tert-butoxycarbonyl)amino]-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.065 g, 0.09 mmol, 1.0 equiv) was taken up in 1:1 anhydrous tetrahydrofuran and anhydrous methanol (0.8 mL) to which aqueous lithium hydroxide (0.40 mL of 40 mg/mL LiOH—H₂O, 0.38 mmol, 4 equiv) was added. The reaction mixture was stirred at ambient temperature for 2 h, concentrated in vacuo, and poured into 0.5 M sodium citrate buffer (pH 4.5). The aqueous layer was extracted with ethyl acetate, and the combined organic layers were washed with saturated sodium chloride, dried over magnesium sulfate, filtered and concentrated in vacuo. The crude product was purified by reverse phase chromatography (C18) and eluted with 30-75% acetonitrile in water (0.1% acetic acid) which provided a white solid (0.044 g, 72% yield): ¹H NMR (400 MHz, DMSO-d6) δ 12.22 (s, 1H), 8.78 (d, J=4.5 Hz, 1H), 8.69 (s, 1H), 8.48 (d, J=8.1 Hz, 1H), 8.36 (d, J=5.3 Hz, 1H), 8.02 (t, J=7.6 Hz, 1H), 7.66 (d, J=5.3 Hz, 1H), 7.56-7.52 (m, 1H), 6.95 (d, J=6.8 Hz, 1H), 6.07 (s, 1H), 5.54-5.47 (m, 1H), 5.26 (t, J=9.4 Hz, 1H), 4.60 (d, J=11.9 Hz, 1H), 4.52 (t, J=7.8 Hz, 1H), 4.03-3.93 (m, 2H), 2.42-2.34 (m, 2H), 2.44-2.38 (m, 1H), 2.23-2.16 (m, 1H), 1.74-1.67 (m, 2H), 1.53-1.43 (m, 3H), 1.41-1.32 (m, 6H), 1.21-0.98 (s, 9H); LCMS (ESI+) for C₃₄H₄₀N₆O₇S m/z 677 (M+H)⁺; Anal. calcd. for C₃₄H₄₀N₆O₇S•1.68 TFA•1.0H₂O: C, 50.63; H, 4.97; N, 9.48. Found: C, 50.27; H, 5.49; N, 9.13.

### Example 45

### Methyl (2R,6S,12Z,13aS,14aR,16aS)-6-[(cyclopropylacetyl)amino]-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate

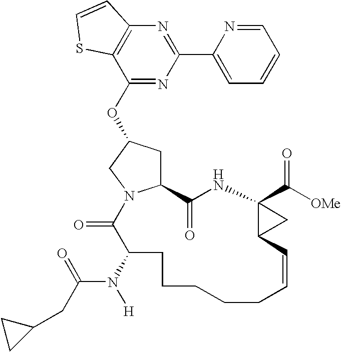

Methyl (2R,6S,12Z,13aS,14aR,16aS)-6-amino-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.140 g, 0.24 mmol, 1.0 equiv) and cyclopropylacetic acid (0.024 g, 0.24 mmol, 1.0 equiv) were taken up in anhydrous DMA (2.5 mL, 0.03 M). Triethylamine (0.070 mL, 0.48 mmol, 2.0 equiv) was added followed by HATU (0.091 g, 0.24 mmol, 1.0 equiv). The reaction mixture was stirred for 1.5 h at 50° C., diluted with ethyl acetate, and poured into saturated sodium bicarbonate. The organic layer washed with 0.5 M sodium citrate buffer (pH=4.5), saturated sodium chloride, dried over magnesium sulfate, filtered and concentrated in vacuo. The crude product which was taken on without further purification (0.140 g, 89% yield): ¹H NMR (400 MHz, DMSO-d6) δ 8.77-8.76 (m, 1H), 8.74 (s, 1H), 8.48 (d, J=7.8 Hz, 1H), 8.39 (d, J=5.3 Hz, 1H), 8.01-7.97 (m, 1H), 7.93 (d, J=7.6 Hz, 1H), 7.68 (d, J=5.3 Hz, 1H), 7.55-7.52 (m, 1H), 6.13 (s, 1H), 5.56-5.49 (m, 1H), 5.27 (t, J=9.6 Hz, 1H), 4.51 (t, J=7.8 Hz, 1H), 4.38-4.34 (m, 2H), 4.12 (dd, J=11.6, 4.3 Hz, 1H), 3.56 (s, 3H), 2.42-2.38 (m, 2H), 2.32-2.24 (m, 1H), 1.88-1.81 (m, 4H), 1.56-1.48 (m, 2H), 1.41-1.14 (m, 8H), 0.69-0.64 (m, 1H), 0.25-0.21 (m, 2H), −0.04-(−0.07) (m, 2H); LCMS (ESI+) for C₃₅H₄₀N₆O₆S m/z 673 (M+H)⁺.

### Example 46

### (2R,6S,12Z,13aS,14aR,16aS)-6-[(Cyclopropylacetyl)amino]-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

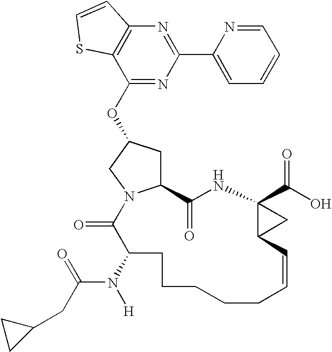

Methyl (2R,6S,12Z,13aS,14aR,16aS)-6-[(cyclopropylacetyl)amino]-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.135 g, 0.20 mmol, 1.0 equiv) was taken up in 1:1 anhydrous tetrahydrofuran and anhydrous methanol (1.6 mL) to which aqueous lithium hydroxide (0.84 mL of 40 mg/mL LiOH—H₂O, 0.8 mmol, 4 equiv) was added. The reaction mixture was stirred at ambient temperature for 2.5 h, concentrated in vacuo, and poured into 0.5 M sodium citrate buffer (pH 4.5). The aqueous layer was extracted with ethyl acetate, and the combined organic layers were washed with saturated sodium chloride, dried over magnesium sulfate, filtered and concentrated in vacuo. The crude product was purified by reverse phase chromatography (C18) and eluted with 30-75% acetonitrile in water (0.1% acetic acid) which provided a white solid (0.031 g, 23% yield): ¹H NMR (400 MHz, DMSO-d6) δ 12.23 (s, 1H), 8.77 (d, J=4.0 Hz, 1H), 8.66 (s, 1H), 8.48 (d, J=7.8 Hz, 1H), 8.39 (d, J=5.3 Hz, 1H), 8.02-7.98 (m, 1H), 7.92 (d, J=7.3 Hz, 1H), 7.68 (d, J=5.3 Hz, 1H), 7.54 (m, 1H), 6.13 (s, 1H), 5.51 (q, J=8.9 Hz, 1H), 5.29 (t, J=9.7 Hz, 1H), 4.50 (t, J=7.7 Hz, 1H), 4.44-4.27 (m, 2H), 4.12 (dd, J=11.6, 4.3 Hz, 1H), 2.47-2.41 (m, 1H), 2.22 (q, J=8.9 Hz, 1H), 1.85-1.76 (m, 3H), 1.50-1.14 (m, 12H), 0.76-0.65 (m, 1H), 0.25-0.22 (m, 2H), −0.03-(−0.07) (m, 2H); LCMS (ESI+) for C₃₄H₃₈N₆O₆S m/z 659 (M+H)⁺; Anal. calcd. for C₃₄H₃₈N₆O₆S•1.2 acetic acid: C, 59.80; H, 5.90; N, 11.50. Found: C, 59.79; H, 6.13; N, 11.18.

### Example 47

### 1,3-Dimethyl-1H-pyrazole-5-carboxamide

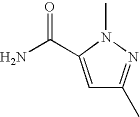

2,5-Dimethyl-2H-pyrazole-3-carboxylic acid ethyl ester (9.5 g, 56.5 mmol, 1.0 equiv) was taken up in ammonium hydroxide and stirred at ambient temperature for 16.5 h. The organic layer was extracted with 10% isopropanol/chloroform, washed with saturated sodium chloride, dried over magnesium sulfate and concentrated in vacuo which provided a white solid (6.0 g, 77% yield): ¹H NMR (400 MHz, DMSO-d6) δ 7.78 (s, 1H), 7.38 (s, 1H), 6.59 (s, 1H), 3.94 (s, 3H), 2.12 (s, 3H), LCMS (ESI+) for C₆H₉N₃O m/z 140 (M+H)⁺.

### Example 48

### 1,3-Dimethyl-1H-pyrazole-5-carbonitrile

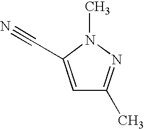

1,3-dimethyl-1H-pyrazole-5-carboxamide (6.0 g, 43.2 mmol, 1.0 equiv) was taken up in anhydrous pyridine (90 mL) and cooled to −5° C. Phosphorous oxychloride (5.8 mL, 60.4 mmol, 1.4 equiv) was added to the white slurry and the resulting beige reaction mixture was stirred at ambient temperature for 2.5 h. The reaction mixture was poured into ice water (300 mL) and the pH was adjusted to 3.1 with 6N HCl followed by 1N hydrochloric acid. The organic layer was extracted with 10% isopropanol/chloroform, washed with 5% sodium bicarbonate and saturated sodium chloride, dried over magnesium sulfate, filtered and concentrated in vacuo which provided a clear oil. The crude product was purified over silica gel (Biotage Horizon silica gel 40M column) and eluted with 5-7% ethyl acetate in dichloromethane/hexanes (1:1), which provided a clear oil (4.5 g, 86% yield): ¹H NMR (400 MHz, DMSO-d6) δ 6.88 (s, 1H), 3.91 (s, 3H), 2.19 (s, 3H); LCMS (ESI+) for C₆H₇N₃ m/z 122 (M+H)⁺.

### Example 49

### 2-(1,3-Dimethyl-1H-pyrazol-5-yl)thieno[3,2-d]pyrimidin-4-ol

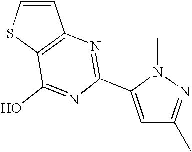

3-Amino-thiophene-2-carboxylic acid methyl ester (2.8 g, 18.2 mmol, 1.0 equiv) and 1,3-dimethyl-1H-pyrazole-5-carbonitrile (2.2 g, 18.2 mmol, 1.0 equiv) were taken up in anhydrous tetrahydrofuran (100 mL). The resultant beige mixture was cooled to 0° C., to which potassium tert-butoxide was added (3.0 g, 27.3 mmol, 1.5 equiv). The reaction mixture was stirred for 14.5 h, concentrated in vacuo, and poured into 50% saturated ammonium chloride, which provided a white solid (1.6 g, 35% yield): ¹H NMR (DMSO-d₆), δ 12.56 (s, 1H), 8.22 (d, J=5.3 Hz, 1H), 7.44 (d, J=5.0 Hz, 1H), 6.93 (s, 1H), 4.11 (s, 3H), 2.19 (s, 3H); LCMS (ESI+) for C₁₁H₁₀N₄OS m/z 247 (M+H)⁺.

### Example 50

### 1-Tert-butyl 2-methyl (2S,4R)-4-{[2-(1,3-dimethyl-1H-pyrazol-5-yl)thieno[3,2-d]pyrimidin-4-yl]oxy}pyrrolidine-1,2-dicarboxylate

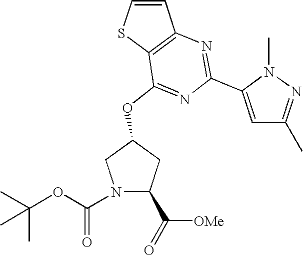

2-(1,3-Dimethyl-1H-pyrazol-5-yl)thieno[3,2-d]pyrimidin-4-ol (1.6 g, 6.5 mmol, 1.0 equiv) cis 4-hydroxy-pyrrolidine-1,2-dicarboxylic acid 1-tert-butyl ester (1.6 g, 6.5 mmol, 1.0 equiv) and triphenylphosphine (3.4 g, 13 mmol, 2.0 equiv) were taken up in anhydrous tetrahydrofuran (130 mL). The resultant white slurry was cooled to 0° C., followed by the addition of DIAD (2.5 mL, 13 mmol, 2.0 equiv). The amber mixture was warmed to ambient temperature and stirred for 17.5 h. The reaction mixture was concentrated in vacuo, poured into 5% saturated bicarbonate and the aqueous layer was extracted with ethyl acetate. The combined organic layers were washed with saturated sodium chloride, dried over magnesium sulfate and concentrated in vacuo. The crude product was purified over silica gel (Biotage Horizon silica gel 40M column) and eluted with 0-5% methanol in chloroform (0.1% ammonium hydroxide), which provided a clear thick oil (2.95 g, 95% yield): ¹H NMR (400 MHz, DMSO-d6) δ 8.39 (d, J=5.3 Hz, 1H), 8.31 (s, 1H), 6.81 (s, 1H), 5.87 (d, J=2.3 Hz, 1H), 4.42-4.36 (m, 1H), 4.21 (s, 3H), 3.85-3.76 (m, 2H), 3.70 (s, 3H), 2.73-2.68 (m, 1H), 2.45-2.35 (m, 1H), 2.20 (s, 3H), 1.34 (s, 9H); LCMS (ESI+) for C₂₂H₂₇N₅O₅S m/z 474 (M+H)⁺.

### Example 51

### (4R)-1-(Tert-butoxycarbonyl)-4-{[2-(1,3-dimethyl-1H-pyrazol-5-yl)thieno[3,2-d]pyrimidin-4-yl]oxy}-L-proline

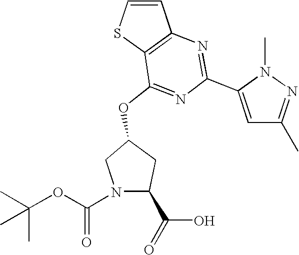

1-Tert-butyl 2-methyl (2S,4R)-4-{[2-(1,3-dimethyl-1H-pyrazol-5-yl)thieno[3,2-d]pyrimidin-4-yl]oxy}pyrrolidine-1,2-dicarboxylate (2.92 g, 6.2 mmol, 1.0 equiv) was taken up in a 1:1 solution of anhydrous tetrahydrofuran and anhydrous methanol (60 mL). A solution of aqueous lithium hydroxide (12.9 mL of 40 mg/mL LiOH—H₂O, 12.4 mmol, 2 equiv) was added and the reaction mixture was stirred at ambient temperature for 10 minutes. The reaction mixture was concentrated in vacuo, and poured into 0.5 M sodium citrate buffer (pH 4.5). The aqueous layer was extracted with ethyl acetate, washed with saturated sodium chloride, dried over magnesium sulfate, filtered and concentrated in vacuo which provided a white solid (2.00 g, 71% yield): ¹H NMR (400 MHz, DMSO-d6) δ 12.81 (s, 1H), 8.39 (d, J=5.4 Hz, 1H), 7.59 (s, 1H), 6.81 (s, 1H), 5.86 (s, 1H), 4.32-4.24 (m, 1H), 4.21 (s, 3H), 3.84-3.77 (m, 1H), 3.72 (d, J=12.1 Hz, 1H), 2.70-2.64 (m, 1H), 2.44-2.37 (m, 1H), 2.20 (s, 3H), 1.36 (s, 9H); LCMS (ESI+) for C₂₁H₂₅N₅O₅S m/z 460 (M+H)⁺.

### Example 52

### Tert-butyl (2S,4R)-4-{[2-(1,3-dimethyl-1H-pyrazol-5-yl)thieno[3,2-d]pyrimidin-4-yl]oxy}-2-({[(1R,2S)-1-(methoxycarbonyl)-2-vinylcyclopropyl]amino}carbonyl)pyrrolidine-1-carboxylate

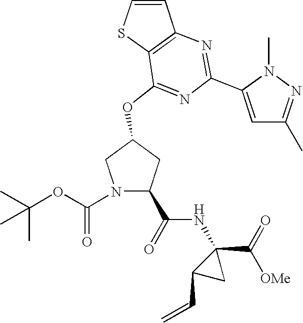

(4R)-1-(Tert-butoxycarbonyl)-4-{[2-(1,3-dimethyl-1H-pyrazol-5-yl)thieno[3,2-d]pyrimidin-4-yl]oxy}-L-proline (1.97 g, 4 mmol, 1.0 equiv) and methyl (1R,2S)-1-amino-2-vinylcyclopropanecarboxylate hydrochloride (0.759 g, 4 mmol, 1.0 equiv) were taken up in anhydrous DMA (30 mL) to which triethylamine (1.8 mL, 13 mmol, 3.0 equiv) followed by HATU (1.63 g, 4 mmol, 1.0 equiv) were added. The reaction mixture was stirred at 40° C. for 1 h, concentrated in vacuo, and diluted with ethyl acetate. The organic layer washed with saturated sodium bicarbonate, 0.5 M sodium citrate buffer (pH 4.5), saturated sodium chloride, dried over magnesium sulfate, filtered and concentrated in vacuo which provided a brown thick oil (3.07 g, >95% yield). ¹H NMR (400 MHz, DMSO-d6) δ 8.39 (d, J=5.3 Hz, 1H), 7.59 (s, 1H), 6.81 (s, 1H), 5.84 (s, 1H), 5.69-5.60 (m, 1H), 5.31-5.21 (m, 1H), 5.12-5.09 (m, 1H), 4.28-4.24 (m, 1H), 4.22 (s, 3H), 3.83 (dd, J=12.2, 4.4 Hz, 1H), 3.77-3.67 (m, 1H), 3.59 (s, 3H), 2.60-2.52 (m, 1H), 2.38-2.28 (m, 1H), 2.17-2.10 (m, 1H), 2.20 (s, 3H), 1.70-1.60 (m, 1H), 1.36 (m, 9H), 1.31-1.29 (m, 1H); LCMS (ESI+) for C₂₈H₃₄N₆O₆S m/z 583 (M+H)⁺.

### Example 53

### Methyl (1R,2S)-1-[((4R)-4-{[2-(1,3-dimethyl-1H-pyrazol-5-yl)thieno[3,2-d]pyrimidin-4-yl]oxy}-L-prolyl)amino]-2-vinylcyclopropanecarboxylate

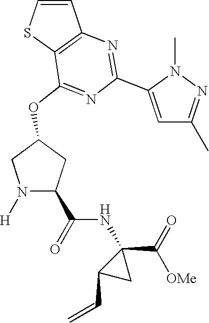

Tert-butyl (2S,4R)-4-{[2-(1,3-dimethyl-1H-pyrazol-5-yl)thieno[3,2-d]pyrimidin-4-yl]oxy}-2-({[(1R,2S)-1-(methoxycarbonyl)-2-vinylcyclopropyl]amino}carbonyl)pyrrolidine-1-carboxylate (3.05 g, 5.24 mmol, 1.0 equiv) was taken up in anhydrous dichloromethane (18 mL) to which trifluoroacetic acid (6 mL) was added and stirred at ambient temperature for 10 h. The reaction mixture was poured into saturated bicarbonate, extracted with dichloromethane, and the combined organic layers were washed with saturated sodium chloride, dried over magnesium sulfate, filtered and concentrated in vacuo which provided the crude product as a thick, brown oil (2.19 g, 87% yield): ¹H NMR (400 MHz, DMSO-d6) δ 8.84 (s, 1H), 8.38 (d, J=5.3 Hz, 1H), 6.82 (s, 1H), 5.81 (s, 1H), 5.63 (dt, J=17.2, 9.6 Hz, 1H), 5.31-5.26 (m, 1H), 5.10 (dd, J=10.2, 1.6 Hz, 1H), 4.22 (s, 3H), 3.98-3.94 (m, 1H), 3.59 (s, 3H), 3.46-3.42 (m, 1H), 2.42-2.38 (m, 1H), 2.26-2.22 (m, 1H), 2.22 (s, 3H), 1.67 (dd, J=7.8, 5.0 Hz, 1H), 1.38-1.34 (m, 2H), 1.19-1.10 (m, 2H); LCMS (ESI+) for C₂₃H₂₆N₆O₄S m/z 483 (M+H)⁺.

### Example 54

### 1-({1-(2-Tert-butoxycarbonylamino-non-8-enoyl)-4-[2-(2,5-dimethyl-2H-pyrazol-3-yl)thieno[3,2-d]pyrimidin-4-yloxy]-pyrrolidine-2-carbonyl}-amino)-2-vinyl-cyclopropanecarboxylic acid methyl ester

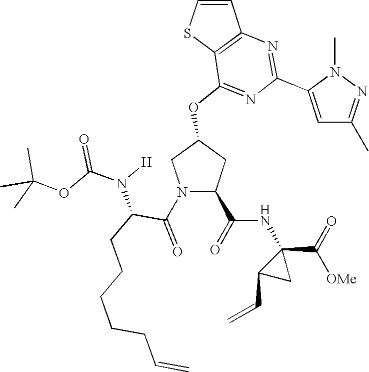

Methyl (1R,2S)-1-[((4R)-4-{[2-(1,3-dimethyl-1H-pyrazol-5-yl)thieno[3,2-d]pyrimidin-4-yl]oxy}-L-prolyl)amino]-2-vinylcyclopropanecarboxylate (2.17 g, 4.5 mmol, 1.0 equiv) and (2S)-2-[(tert-butoxycarbonyl)amino]non-8-enoic acid (1.22 g, 9 mmol, 1.0 equiv) were taken up in anhydrous DMA (30 mL) to which triethylamine (1.29 mL, 0.009 mmol, 2.0 equiv) followed by HATU (1.71 g, 4.5 mmol, 1.0 equiv) were added. The reaction mixture was stirred at 50° C. for 1.25 h, poured into 50% saturated sodium bicarbonate, and extracted with ethyl acetate. The combined organic extracts were washed with saturated sodium chloride, dried over magnesium sulfate, filtered and concentrated in vacuo. The crude product was purified over silica gel (Biotage Horizon silica gel 40M column) and eluted with 1-2.5% methanol in dichloromethane (0.1% ammonium hydroxide), which provided a brown oil (2.12 g, 64% yield): ¹H NMR (400 MHz, DMSO-d6) δ 8.36 (s, 1H), 8.12-8.08 (m, 1H), 7.27 (m, 1H), 6.76 (d, J=7.6 Hz, 1H), 6.54 (s, 1H), 5.66 (s, 1H), 5.51-5.31 (m, 2H), 5.47 (s, 3H), 4.96-4.92 (m, 1H), 4.82-4.79 (m, 1H), 4.72-4.70 (m, 1H), 4.68-4.60 (m, 2H), 4.18-4.14 (m, 1H), 4.03 (d, J=11.9 Hz, 1H), 3.95 (s, 3H), 3.76-3.71 (m, 2H), 2.21 (s, 6H), 2.06-1.98 (m, 1H), 1.82-1.75 (m, 1H), 1.36-1.28 (m, 2H), 1.02-0.94 (m, 7H), 0.84 (s, 9H); LCMS (ESI+) for C₃₇H₄₉N₇O₇S m/z 736 (M+H)⁺.

### Example 55

### Methyl (2R,6S,12Z,13aS,14aR,16aS)-6-[(tert-butoxycarbonyl)amino]-2-{[2-(1,3-dimethyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

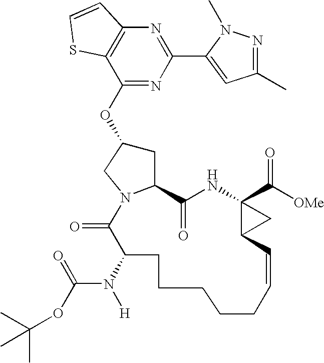

1-({1-(2-Tert-butoxycarbonylamino-non-8-enoyl)-4-[2-(2,5-dimethyl-2H-pyrazol-3-yl)-thieno[3,2-d]pyrimidin-4-yloxy]-pyrrolidine-2-carbonyl}-amino)-2-vinyl-cyclopropanecarboxylic acid methyl ester (2.1 g, 3 mmol, 1.0 equiv) was taken up in anhydrous dichloromethane (570 mL, 0.005M). The reaction vessel was evacuated and purged with nitrogen gas. The Grubbs' second generation ruthenium catalyst was added (0.363 g, 0.43 mmol, 0.15 equiv) and the reaction mixture was stirred at 40° C. for 2 h. The reaction mixture was concentrated in vacuo. The crude product was purified over silica gel (Biotage Horizon silica gel 40M column) and eluted with 1-2.5% methanol in dichloromethane (0.1% ammonium hydroxide). The semi-pure product was triturated with MTBE/hexanes and provided a white solid (0.788 g, 38% yield): ¹H NMR (400 MHz, DMSO-d6) δ 8.77 (s, 1H), 8.34 (d, J=5.3 Hz, 1H), 7.58 (d, J=5.3 Hz, 1H), 7.00 (d, J=6.6 Hz, 1H), 6.82 (s, 1H), 5.94 (s, 1H), 5.56-5.49 (m, 1H), 5.27 (t, J=9.6 Hz, 1H), 4.67 (d, J=11.4 Hz, 1H), 4.54-4.50 (m, 1H), 4.23 (s, 3H), 3.94-3.91 (m, 2H), 3.56 (s, 3H), 2.60-2.52 (m, 1H), 2.41-2.38 (m, 1H), 2.20 (s, 3H), 1.71-1.65 (m, 2H), 1.57-1.48 (m, 2H), 1.36-1.23 (m, 9H), 0.98 (s, 9H); LCMS (ESI+) for C₃₅H₄₅N₇O₇S m/z 708 (M+H)⁺.

### Example 56

### Methyl (2R,6S,12Z,13aS,14aR,16aS)-6-amino-2-{[2-(1,3-dimethyl-1H-pyrazol-5-yl)thieno[3,2-d]pyrimidin-4-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylate

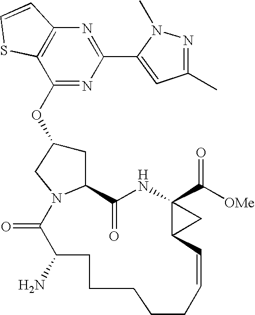

Methyl (2R,6S,12Z,13aS,14aR,16aS)-6-[(tert-butoxycarbonyl)amino]-2-{[2-(1,3-dimethyl-1H-pyrazol-5-yl)thieno[3,2-d]pyrimidin-4-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylate (0.623 g, 0.96 mmol, 1.0 equiv) was taken up in dichloromethane (9 mL) to which trifluoroacetic acid was added (3 mL). The reaction mixture was stirred at ambient temperature for 1 h. The reaction mixture was quenched with saturated sodium bicarbonate, and the combined organic layers were washed with saturated sodium chloride, dried over magnesium sulfate, filtered and concentrated in vacuo, which provided a white solid (0.554 g, >95% yield): ¹H NMR (400 MHz, DMSO-d6) δ 8.71 (s, 1H), 8.39 (d, J=5.3 Hz, 1H), 7.63 (d, J=5.3 Hz, 1H), 6.84 (s, 1H), 6.03 (s, 1H), 5.55-5.47 (m, 1H), 5.28 (t, J=9.7 Hz, 1H), 4.52 (t, J=7.6 Hz, 1H), 4.23 (s, 3H), 4.08-3.99 (m, 2H), 3.59-3.51 (m, 4H), 2.48-2.41 (m, 4H), 2.39-2.28 (m, 2H), 2.25-2.15 (m, 4H), 1.98-1.87 (m, 1H), 1.56-1.47 (m, 3H), 1.38 (s, 1H), 1.25 (s, 3H), 1.21-1.11 (m, 2H); LCMS (ESI+) for C₃₀H₃₇N₇O₅S m/z 608 (M+H)⁺.

### Example 57

### Methyl (2R,6S,12Z,13aS,14aR,16aS)-6-{[(cyclopentyloxy)carbonyl]amino}-2-{[2-(1,3-dimethyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

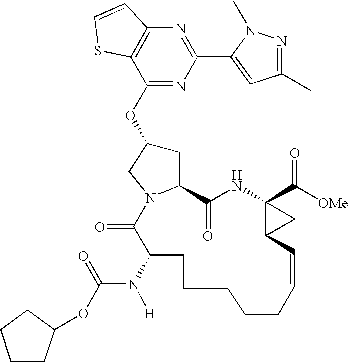

Methyl (2R,6S,12Z,13aS,14aR,16aS)-6-amino-2-{[2-(1,3-dimethyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.151 g, 0.22 mmol, 1.0 equiv) and triethylamine (0.036 mL, 0.26 mmol, 1.2 equiv) were taken up in anhydrous DMA (2.2 mL). Cyclopentyl 4-nitrophenyl carbonate (0.054 g, 0.22 mmol, 1.0 equiv) was added and the reaction mixture was heated at 80° C. for 21 h. The reaction mixture was poured into saturated sodium bicarbonate and extracted with ethyl acetate. The combined organic extracts were washed with saturated sodium chloride, dried over magnesium sulfate, filtered and concentrated in vacuo, which provide a white solid (0.185 g, 100% yield): ¹H NMR (400 MHz, DMSO-d6) δ 8.74 (s, 1H), 8.40-8.29 (m, 1H), 7.56-7.54 (m, 1H), 7.21-7.19 (m, 1H), 6.83 (s, 1H), 5.99 (s, 1H), 5.56-5.49 (m, 1H), 5.25 (t, J=9.6 Hz, 1H), 4.56-4.48 (m, 2H), 4.32 (m, 1H), 4.24 (s, 3H), 3.98-3.94 (m, 1H), 3.56 (s, 3H), 2.20 (s, 4H), 1.79-1.20 (m, 22H); LCMS (ESI+) for C₃₆H₄₅N₇O₇S m/z 720 (M+H)⁺.

### Example 58

### (2R,6S,12Z,13aS,14aR,16aS)-6-{[(Cyclopentyloxy)carbonyl]amino}-2-{[2-(1,3-dimethyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

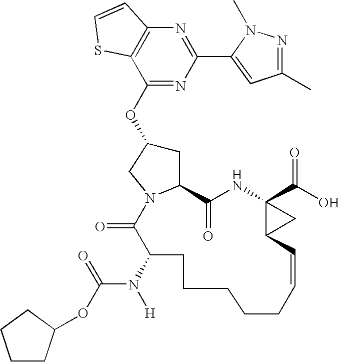

Methyl (2R,12Z,13aS,14aR,16aS)-6-{[(cyclopentyloxy)carbonyl]amino}-2-{[2-(1,3-dimethyl-1H -pyrazol-5-yl)thieno[3,2-d]pyrimidin-4-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylate (0.182 g, 0.25 mmol, 1.0 equiv) was taken up in 1:1 anhydrous tetrahydrofuran and anhydrous methanol (12 mL) to which an aqueous solution of lithium hydroxide (1.06 mL of 40 mg/mL LiOH—H₂O, 1.0 mmol, 4 equiv) was added. The reaction mixture was stirred at ambient temperature for 3 h. The reaction mixture was concentrated in vacuo, poured into 0.5 M sodium citrate buffer (pH=4.5) and extracted with ethyl acetate. The combined organic extracts were washed with saturated sodium chloride, dried over magnesium sulfate, filtered and concentrated in vacuo. The crude product was purified by reverse phase chromatography (C18) and eluted with 30-75% acetonitrile in water (0.1% acetic acid) which provided a white solid (0.059 g, 33% yield): ¹H NMR (400 MHz, DMSO-d6) δ 12.23 (s, 1H), 8.65 (s, 1H), 8.37 (s, 1H), 7.60 (s, 1H), 7.18 (s, 1H), 6.83 (s, 1H), 5.99 (s, 1H), 5.49 (m, 1H), 5.27 (t, J=9.8 Hz, 1H), 4.52-4.50 (m, 2H), 4.34 (s, 1H), 4.23 (s, 3H), 4.02-3.97 (m, 2H), 2.75-2.58 (m, 1H), 2.43-2.32 (m, 1H), 2.20 (s, 4H), 1.71 (br. s, 2H), 1.54-1.10 (m, 19H); LCMS (ESI+) for C₃₅H₄₃N₇O₇S m/z 706 (M+H)⁺.

### Example 59

### Methyl (2R,6S,12Z,13aS,14aR,16aS)-6-{[(cyclobutyloxy)carbonyl]amino}-2-{[2-(1,3-dimethyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

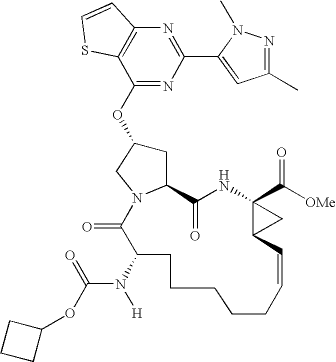

Methyl (2R,6S,12Z,13aS,14aR,16aS)-6-amino-2-{[2-(1,3-dimethyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.131 g, 0.188 mmol, 1.0 equiv) and triethylamine (0.031 mL, 0.23 mmol, 1.2 equiv) were taken up in anhydrous DMA (2 mL). Cyclobutyl 4-nitrophenyl carbonate (0.045 g, 0.188 mmol, 1.0 equiv) was added and the reaction mixture was heated at 80° C. for 22 h. The reaction mixture was poured into saturated sodium bicarbonate and extracted with ethyl acetate. The combined organic extracts were washed with saturated sodium chloride, dried over magnesium sulfate, filtered and concentrated in vacuo, which provided a white solid (0.152 g, >95% yield): LCMS (ESI+) for C₃₅H₄₃N₇O₇S m/z 706 (M+H)⁺.

### Example 60

### (2R,6S,12Z,13aS,14aR,16aS)-6-{[(Cyclobutyloxy)carbonyl]amino}-2-{[2-(1,3-dimethyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

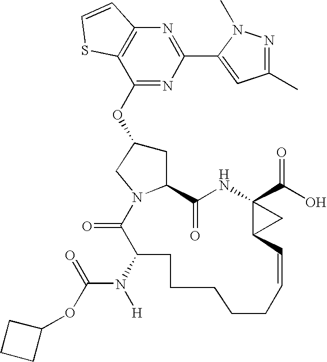

Methyl (2R,6S,12Z,13aS,14aR,16aS)-6-{[(cyclobutyloxy)carbonyl]amino}-2-{[2-(1,3-dimethyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.148 g, 0.21 mmol, 1.0 equiv) was taken up in 1:1 anhydrous tetrahydrofuran and anhydrous methanol (2 mL) to which an aqueous solution of lithium hydroxide (0.879 mL of 40 mg/mL LiOH—H₂O, 0.84 mmol, 4 equiv) was added. The reaction mixture was stirred at ambient temperature for 3 h. The reaction mixture was concentrated in vacuo, and poured into 0.5 M sodium citrate buffer (pH=4.5). The aqueous layer was extracted with ethyl acetate, and the combined organic extracts were washed with saturated sodium chloride, dried over magnesium sulfate, filtered and concentrated in vacuo. The crude product was purified by reverse phase chromatography (C18) and eluted with 30-75% acetonitrile in water (0.1% acetic acid) which provided a white solid (0.026 g, 18% yield): ¹H NMR (400 MHz, DMSO-d6) δ 12.25 (s, 1H), 8.65 (s, 1H), 8.39 (d, J=5.3 Hz, 1H), 7.62 (d, J=5.3 Hz, 1H), 7.29 (d, J=5.3 Hz, 1H), 6.83 (s, 1H), 5.99 (s, 1H), 5.49 (m, 1H), 5.29 (t, J=9.6 Hz, 1H), 5.17 (br. s, 1H), 4.52-4.47 (m, 2H), 4.23 (s, 3H), 4.19-4.14 (m, 1H), 4.01-3.94 (m, 2H), 2.42-2.32 (m, 1H), 2.20 (s, 3H), 2.19-2.14 (m, 1H), 1.92-1.81 (m, 2H), 1.79-1.62 (m, 4H), 1.60-1.41 (m, 4H), 1.40-1.27 (m, 6H), 1.25-1.12 (m, 2H); LCMS (ESI+) for C₃₄H₄₁N₇O₇S m/z 692 (M+H)⁺.

### Example 61

### (2R,6S,12Z,13aS,14aR,16aS)-6-[(Tert-butoxycarbonyl)amino]-2-{[2-(1,3-dimethyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

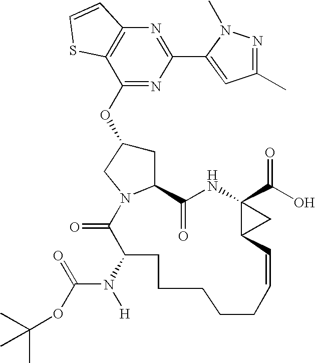

Methyl (2R,6S,12Z,13aS,14aR,16aS)-6-[(tert-butoxycarbonyl)amino]-2-{[2-(1,3-dimethyl-1H-pyrazol-5-yl)thieno[3,2-d]pyrimidin-4-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylate (0.075 g, 0.106 mmol, 1.0 equiv) was taken up in 1:1 anhydrous tetrahydrofuran and anhydrous methanol (2 mL) to which an aqueous solution of lithium hydroxide (0.445 mL of 40 mg/mL LiOH—H₂O, 0.424 mmol, 4 equiv) was added. The reaction mixture was stirred at ambient temperature for 3.5 h. The reaction mixture was concentrated in vacuo, and poured into 0.5 M sodium citrate buffer (pH=4.5). The aqueous layer was extracted with ethyl acetate, and the combined organic extracts were washed with saturated sodium chloride, dried over magnesium sulfate, filtered and concentrated in vacuo. The crude product was purified by reverse phase chromatography (C18) and eluted with 30-75% acetonitrile in water (0.1% acetic acid) which provided a white solid (0.010 g, 13% yield): ¹H NMR (400 MHz, DMSO-d6) δ 11.66 (br. s, 1H), 8.63 (br. s, 1H), 8.30 (d, J=5.3 Hz, 1H), 7.58 (d, J=5.3 Hz, 1H), 6.97 (d, J=6.6 Hz, 1H), 6.80 (s, 1H), 5.94 (s, 1H), 5.46 (br. s, 1H), 5.31 (br. s, 1H), 4.60 (d, J=11.4 Hz, 1H), 4.50 (t, J=8.2 Hz, 1H), 4.22 (s, 3H), 4.01-3.89 (m, 2H), 2.43-2.28 (m, 1H), 2.20 (s, 3H), 2.17-2.06 (m, 1H), 1.89 (s, 1H), 1.79-1.59 (m, 2H), 1.58-1.16 (m, 10H), 0.99 (s, 9H); LCMS (ESI+) for C₃₄H₄₃N₇O₇S m/z 694 (M+H)⁺.

### Example 62

### Methyl (2R,6S,12Z,13aS,14aR,16aS)-6-[(cyclopropylacetyl)amino]-2-{[2-(1,3-dimethyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

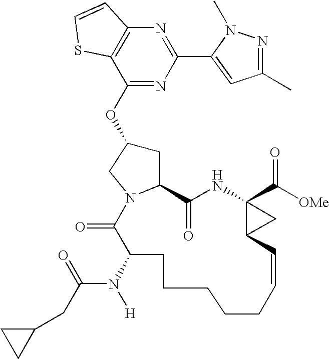

Methyl (2R,6S, 12Z,13aS,14aR,16aS)-6-amino-2-{[2-(1,3-dimethyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.142 g, 0.23 mmol, 1.0 equiv) and cyclopropylacetic acid (0.020 g, 0.23 mmol, 1.0 equiv) were taken up in anhydrous dichloromethane (6.8 mL, 0.03 M). Diisopropylethylamine (0.179 mL, 1.03 mmol, 5.0 equiv) was added followed by HATU (0.078 g, 0.20 mmol, 1.0 equiv). The reaction mixture was stirred for 22 h at ambient temperature. The reaction mixture was diluted with ethyl acetate and poured into saturated sodium bicarbonate. The organic layer washed with 0.5 M sodium citrate buffer (pH=4.5), saturated sodium chloride, dried over magnesium sulfate, filtered and concentrated in vacuo which provided the crude product (0.126 g, 77% yield): ¹H NMR (400 MHz, DMSO-d6) δ 8.77-8.70 (m, 1H), 8.42-8.35 (m, 1H), 8.00-7.92 (m, 1H), 7.63-7.55 (m, 1H), 6.85-6.80 (m, 1H), 6.03 (s, 1H), 5.57-5.49 (m, 1H), 5.27 (t, J=10.1 Hz, 1H); 4.50 (t, J=7.8 Hz, 1H), 4.42-4.31 (m, 2H), 4.26-4.21 (m, 3H), 4.08-4.04 (m, 1H), 3.56 (s, 3H), 2.46-2.36 (m, 2H), 2.33-2.23 (m, 1H), 2.21-2.16 (m, 3H), 1.84-1.76 (m, 4H), 1.52 (ddd, J=20.0, 8.8, 4.8 Hz, 2H), 1.38-1.20 (m, 8H), 0.69-0.58 (m, 1H), 0.25-0.17 (m, 2H), −0.04-(−0.11) (m, 2H); LCMS (ESI+) for C₃₅H₄₃N₇O₆S m/z 690 (M+H)⁺.

### Example 63

### (2R,6S,12Z,13aS,14aR,16aS)-6-[(cyclopropylacetyl)amino]-2-{[2-(1,3-dimethyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

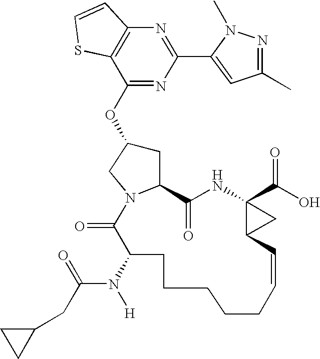

Methyl (2R,6S,12Z,13aS,14aR,16aS)-6-[(cyclopropylacetyl)amino]-2-{[2-(1,3-dimethyl-1H-pyrazol-5-yl)thieno[3,2-d]pyrimidin-4-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylate (0.119 g, 0.172 mmol, 1.0 equiv) was taken up in 1:1 anhydrous tetrahydrofuran and anhydrous methanol (2 mL) to which aqueous lithium hydroxide (0.723 mL of 40 mg/mL LiOH—H₂O, 0.689 mmol, 4 equiv) was added. The reaction mixture was stirred at ambient temperature for 3 h, poured into 0.5 M sodium citrate buffer (pH=4.5), and the crude product was filtered and collected as a white solid. The crude product was purified by reverse phase chromatography (C18) and eluted with 30-75% acetonitrile in water (0.1% acetic acid), which provided a white solid (0.052 g, 44% yield): ¹H NMR (400 MHz, DMSO-d6) δ 12.22 (s, 1H), 8.66 (s, 1H), 8.37 (d, J=5.3 Hz, 1H), 7.94 (d, J=5.3 Hz, 1H), 7.60 (d, J=5.3 Hz, 1H), 6.83 (s, 1H), 6.03 (s, 1H), 5.54-5.47 (m, 1H), 5.29 (t, J=10.1 Hz, 1H), 4.48 (t, J=7.8 Hz, 1H), 4.42-4.32 (m, 2H), 4.24 (s, 3H), 4.08-4.00 (m, 1H), 2.72-2.57 (m, 2H), 2.45-2.31 (m, 1H), 2.23-2.19 (m, 4H), 1.90-1.73 (m, 4H), 1.50-1.44 (m, 2H), 1.34 (br. s, 5H), 1.27-1.13 (m, 2H), 0.69-0.60 (m, 1H), 0.24-0.19 (m, 2H), −0.05-(−0.09) (m, 2H); LCMS (ESI+) for C₃₄H₄₁N₇O₆S m/z 676 (M+H)⁺; Anal. calcd. for C₃₄H₄₁N₇O₆S•1.2H₂O•1.4 acetic acid: C, 56.53; H, 6.32; N, 12.52. Found: C, 56.31; H, 5.88; N, 12.25.

### Example 64

### Methyl (2R,6S,12Z,13aS,14aR,16aS)-2-{[2-(1,3-dimethyl-1H-pyrazol-5-yl)thieno[3,2-d]pyrimidin-4-yl]oxy}-5,16-dioxo-6-({[(3S)-tetrahydrofuran-3-yloxy]carbonyl}amino)-1,2,3,6,7,8,9,10,11,13a,14,15,16,16a-tetradecahydrocyclopropa[e]pyrrolo[1,2-a][1,4]diazacyclopentadecine-14a(5H)-carboxylate

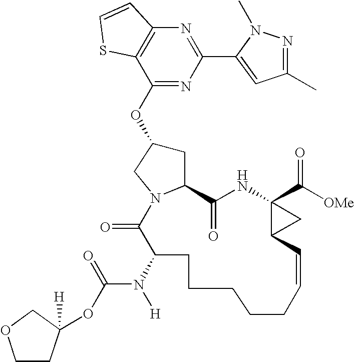

Methyl (2R,6S,12Z,13aS,14aR,16aS)-6-amino-2-{[2-(1,3-dimethyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.109 g, 0.18 mmol, 1.0 equiv) and triethylamine (0.075 mL, 0.54 mmol, 3.0 equiv) were taken up in anhydrous dichloromethane (4.0 mL). (3S)-Tetrahydrofuran-3-yl chloridocarbonate (0.032 g, 0.21 mmol, 1.2 equiv) was added and the reaction mixture was stirred at ambient temperature for 0.2 h. The reaction mixture was concentrated in vacuo and diluted with ethyl acetate. The organics were washed with water and saturated sodium chloride, dried over magnesium sulfate, filtered and concentrated in vacuo, which provided a white solid (0.093 g, 72% yield): ¹H NMR (400 MHz, DMSO-d6) δ 8.74 (s, 1H), 8.40 (m, 1H), 7.60 (m, 2H) 7.40 (d, J=6.8 Hz, 1H), 6.83 (s, 1H), 6.00 (s, 1H), 5.56-5.49 (m, 1H), 5.25 (t, J=9.6 Hz, 1H), 4.54-4.47 (m, 3H), 4.24 (m, 3H), 3.98-3.94 (m, 1H), 3.60-3.54 (m, 5H), 3.47-3.44 (m, 1H), 3.39-3.37 (m, 1H), 2.47-2.39 (m, 3H), 2.20 (s, 3H), 1.76-1.17 (m, 14H); LCMS (ESI+) for C₃₅H₄₃N₇O₈S m/z 722 (M+H)⁺.

### Example 65

### (2R,6S,12Z,13aS,14aR,16aS)-2-{[2-(1,3-Dimethyl-1H-pyrazol-5-yl)thieno[3,2-d]pyrimidin-4-yl]oxy}-5,16-dioxo-6-({[(3S)-tetrahydrofuran-3-yloxy]carbonyl}amino)-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

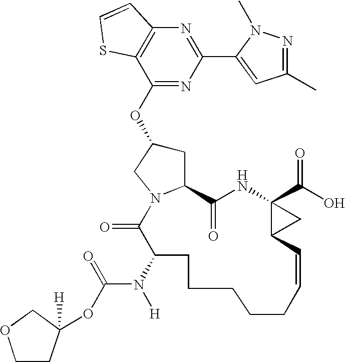

Using the procedure described for Example 58 using methyl (2R,6S,12Z,13aS,14aR,16aS)-2-{[2-(1,3-dimethyl-1H-pyrazol-5-yl)thieno[3,2-d]pyrimidin-4-yl]oxy}-5,16-dioxo-6-({[(3S)-tetrahydrofuran-3-yloxy]carbonyl}amino)-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,6S,12Z,13aS,14aR,16aS)-6-{[(cyclopentyloxy)carbonyl]amino}-2-{[2-(1,3-dimethyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate yielded the title compound of Example 65 as a white solid (0.010 g, 13% yield): ¹H NMR (400 MHz, DMSO-d6) δ 12.09 (br. s, 1H), 8.65 (s, 1H), 8.37 (d, J=5.3 Hz, 1H), 7.60 (d, J=5.3 Hz, 1H), 7.40 (d, J=6.8 Hz, 1H), 6.80 (s, 1H), 6.00 (s, 1H), 5.54-5.47 (m, 1H), 5.27 (t J=9.6 Hz, 1H), 4.53-4.45 (m, 3H), 4.23 (s, 3H), 4.06-3.95 (m, 2H), 3.60-3.55 (m, 2H), 3.47-3.45 (m, 1H), 3.40-3.37 (m, 1H), 2.42-2.36 (m, 2H), 2.20 (s, 4H), 1.90-1.70 (m, 3H), 1.53-1.44 (m, 3H), 1.31 (m, 7H); LCMS (ESI+) for C₃₄H₄₁N₇O₈S m/z 708 (M+H)⁺.

### Example 66

### Tert-butyl (6-bromopyridin-2-yl)carbamate

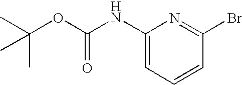

Diphenylphosphoryl azide (10.7 mL, 50 mmol, 1.0 equiv) was added to a solution of 6-bromopyridine-2-carboxylic acid (10.0 g, 50 mmol, 1.0 equiv) and triethylamine (6.8 mL, 50 mmol, 1.0 equiv) in anhydrous tert-butyl alcohol (250 mL). The reaction mixture was refluxed for 2 hours, concentrated in vacuo and diluted with ethyl acetate. The organic layers were washed with 0.5 M sodium citrate buffer (pH=4.5), saturated sodium bicarbonate and saturated sodium chloride, dried over magnesium sulfate, filtered and concentrated in vacuo. The crude product was purified over silica (Biotage Horizon silica gel 40M column) and eluted with 8% ethyl acetate in hexanes which provided a light yellow solid (8.9 g, 66% yield): ¹H NMR (400 MHz, DMSO-d6) δ 10.13 (s, 1H), 7.78 (d, J=8.1 Hz, 1H), 7.65 (t, J=8.0 Hz, 1H), 7.20 (d, J=7.6 Hz, 1H), 1.45 (s, 9H); LCMS (ESI+) for C₁₀H₁₃BrN₂O₂ m/z 295/297 (M+Na)⁺.

### Example 67

### Tert-butyl (6-bromopyridin-2-yl)isopropylcarbamate

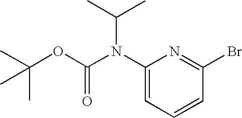

Sodium hydride (2.2 g, 65 mmol, 2.0 equiv) was slowly added to a solution of tert-butyl (6-bromopyridin-2-yl)carbamate (8.9 g, 32 mmol, 1.0 equiv) in DMF (120 mL) at 0° C. and stirred for 0.25 h. 2-iodopropane (6.5 mL, 65 mmol, 2.0 equiv) added and the reaction mixture was warmed to ambient temperature and stirred for 23 h. Additional 2-iodopropane (6.5 mL, 65 mmol, 2.0 equiv) was added after 23.5 h, 25 h and 25.5 h. The reaction was quenched with 0.5 M sodium citrate buffer (pH=4.5) and the aqueous layer was extracted with MTBE. The organics were washed with saturated sodium chloride, dried over magnesium sulfate, filtered and concentrated in vacuo to give a light yellow solid (11.69 g, >100% yield) which was taken on without further purification: ¹H NMR (400 MHz, DMSO-d6) δ 7.74 (t, J=7.8 Hz, 1H), 7.50 (t, J=7.8 Hz, 1H), 7.30 (d, J=7.8 Hz, 1H), 4.43-4.33 (m, 1H), 1.37 (s, 9H), 1.17 (d, J=6.8 Hz, 6H); LCMS (ESI+) for C₁₃H₁₉BrN₂O₂ m/z 337/339 (M+Na)⁺.

### Example 68

### Tert-butyl (6-cyanopyridin-2-yl)isopropylcarbamate

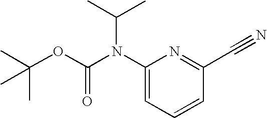

A mixture of tert-butyl (6-bromopyridin-2-yl)isopropylcarbamate (11.69 g, 37 mmol, 1.0 equiv), triphenylphosphine (2.14 g, 1.8 mmol, 0.05 equiv), zinc cyamide (4.34 g, 37 mmol, 1.0 equiv) and tetrakis(triphenylphosphine)palladium(0) (0.971 g, 37 mmol, 1.0 equiv) was suspended in anhydrous DMF (200 mL). The reaction vessel was degassed, purged with nitrogen gas and heated at 125° C. for 2.25 h. The cooled reaction mixture was poured into water (1 L) and the aqueous layer was extracted with MTBE. The organic layer was washed with saturated sodium chloride, dried over magnesium sulfate, filtered and concentrated in vacuo. The crude product was purified over silica (Biotage Horizon silica gel 65M column) and eluted with 6% ethyl acetate in hexanes which provided a clear oil (5.5 g, 57% yield): ¹H NMR (400 MHz, DMSO-d6) δ 8.0 (m, 1H), 7.89-7.87 (m, 1H), 7.64-7.62 (m, 1H), 4.49-4.39 (m, 1H), 1.38 (s, 9H), 1.21 (d, J=6.8 Hz, 6H); LCMS (ESI+) for C₁₄H₁₉N₃O₂ m/z 284 (M+Na)⁺.

### Example 69

### Tert-butyl [6-(4-hydroxythieno[3,2-d]pyrimidin-2-yl)pyridin-2-yl]isopropylcarbamate

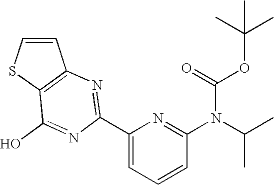

Using the procedure described for Example 32 and using tert-butyl (6-cyanopyridin-2-yl)isopropylcarbamate instead of pyridine-2-carbonitrile yielded the title compound of Example 69 as a white solid (2.4 g, 38% yield): ¹H NMR (400 MHz, DMSO-d6) δ 11.66 (s, 1H), 8.25 (d, J=5.1 Hz, 1H), 8.19 (d, J=7.1 Hz, 1H), 8.04-8.00 (m, 1H), 7.53-7.50 (m, 2H), 4.66-4.60 (m, 1H), 1.40 (s, 9H), 1.30 (d, J=6.6 Hz, 6H); LCMS (ESI+) for C₁₉H₂₂N₄O₃S m/z 387 (M+H)⁺.

### Example 70

### 1-Tert-butyl 2-methyl (2S,4R)-4-[(2-{6-[(tert-butoxycarbonyl)(isopropyl)amino]pyridin-2-yl}thieno[3,2-d]pyrimidin-4-yl)oxy]pyrrolidine-1,2-dicarboxylate

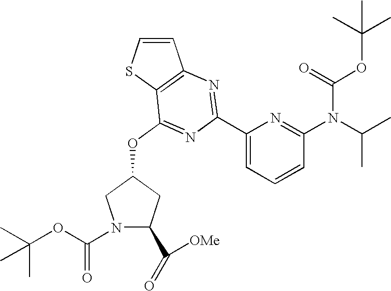

Using the procedure described for Example 33 and using tert-butyl [6-(4-hydroxythieno[3,2-d]pyrimidin-2-yl)pyridin-2-yl]isopropylcarbamate instead of 2-(2,5-dimethyl-2H-pyrazol-3-yl)-thieno[3,2-d]pyrimidin-4-ol yielded the title compound of Example 70 as an off-white foam (5.78 g, >100% yield): ¹H NMR (400 MHz, DMSO-d6) δ 8.41 (d, J=5.3 Hz, 1H), 8.27 (d, J=7.6 Hz, 1H), 7.94 (t, J=7.8 Hz, 1H), 7.69 (d, J=5.3 Hz, 1H), 7.38 (d, J=7.8 Hz, 1H), 5.89 (br. s, 1H), 4.57-4.53 (m, 1H), 4.45-4.38 (m, 1H), 3.97-3.88 (m, 1H), 3.73-3.67 (m, 4H), 2.77-2.66 (m, 1H), 2.44-2.39 (m, 1H), 1.40 (s, 9H), 1.35-1.34 (m, 9H), 1.18-1.16 (m, 6H); LCMS (ESI+) for C₃₀H₃₉N₅O₇S m/z 614 (M+H)⁺.

### Example 71

### (4R)-1-(Tert-butoxycarbonyl)-4-[(2-{6-[(tert-butoxycarbonyl)(isopropyl)amino]pyridin-2-yl}thieno[3,2-d]pyrimidin-4-yl)oxy]-L-proline

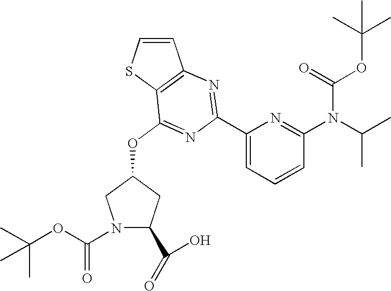

Using the procedure described for Example and using 1-tert-butyl 2-methyl (2S,4R)-4-[(2-{6-[(tert-butoxycarbonyl)(isopropyl)amino]pyridin-2-yl}thieno[3,2-d]pyrimidin-4-yl)oxy]pyrrolidine-1,2-dicarboxylate instead of 1-tert-butyl 2-methyl (2S,4R)-4-[(2-pyridin-2-ylthieno[3,2-d]pyrimidin-4-yl)oxy]pyrrolidine-1,2-dicarboxylate yielded the title compound of Example 71 as a white foam (5.46 g, 97% yield): ¹H NMR (400 MHz, DMSO-d6) δ 12.82 (br. s, 1H), 8.42 (d, J=5.6 Hz, 1H), 8.28 (d, J=7.3 Hz, 1H), 7.95 (d, J=7.8 Hz, 1H), 7.69 (d, J=5.3 Hz, 1H), 7.39 (d, J=8.1 Hz, 1H), 5.91-5.89 (m, 1H), 4.60-4.51 (m, 1H), 4.34-4.28 (m, 1H), 3.96-3.85 (m, 1H), 3.72-3.70 (m, 1H), 2.74-2.59 (m, 1H), 2.46-2.38 (m, 1H), 1.41 (s, 9H), 1.36 (d, J=2.8 Hz, 9H), 1.18-1.17 (m, 6H); LCMS (ESI+) for C₂₉H₃₇N₅O₇S m/z 600 (M+H)⁺.

### Example 72

### Tert-butyl (2S,4R)₄-[(2-{6-[(tert-butoxycarbonyl)(isopropyl)amino]pyridin-2-yl}thieno[3,2-d]pyrimidin-4-yl)oxy]-2-({[(1R,2S)-1-(methoxycarbonyl)-2-vinylcyclopropyl]amino}carbonyl)pyrrolidine-1-carboxylate

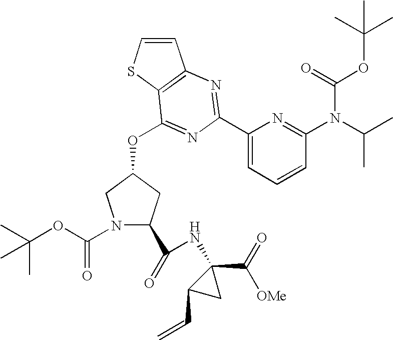

Using the procedure described for Example 35 and using (4R)-1-(tert-butoxycarbonyl)-4-[(2-{6-[(tert-butoxycarbonyl)(isopropyl)amino]pyridin-2-yl}thieno[3,2-d]pyrimidin-4-yl)oxy]-L-proline instead of (4R)-1-(tert-butoxycarbonyl)-4-[(2-pyridin-2-ylthieno[3,2-d]pyrimidin-4-yl)oxy]-L-proline yielded the title compound of Example 72 as an amber oil (8.87 g, >100% yield): ¹H NMR (400 MHz, DMSO-d6) δ 8.70 (d, J=13.6 Hz, 1H), 8.41 (d, J=5.6 Hz, 1H), 8.28 (d, J=7.6 Hz, 1H), 7.95 (d, J=7.8 Hz, 1H), 7.70-7.68 (m, 1H), 7.39 (d, J=8.1 Hz, 1H), 5.88 (s, 1H), 5.68-5.59 (m, 1H), 5.29-5.23 (m, 1H), 5.12-5.08 (m, 1H), 4.58-4.50 (m, 1H), 4.31-4.22 (m, 1H), 3.63-3.56 (m, 4H), 2.68 (m, 4H), 2.32-2.24 (m, 1H), 2.18-2.08 (m, 1H), 1.40 (s, 9H), 1.35-1.34 (m, 9H), 1.18-1.16 (m, 6H); LCMS (ESI+) for C₃₆H₄₆N₆O₈S m/z 723 (M+H)⁺.

### Example 73

### Methyl (1R,2S)-1-{[(4R)-4-({2-[6-(isopropylamino)pyridin-2-yl]thieno[3,2-d]pyrimidin-4-yl}oxy)-L-prolyl]amino}-2-vinylcyclopropanecarboxylate

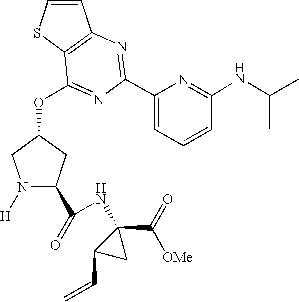

Using the procedure described for Example 36 and using tert-butyl (2S,4R)-4-[(2-{6-[(tert-butoxycarbonyl)(isopropyl)amino]pyridin-2-yl}-thieno[3,2-d]pyrimidin-4-yl)oxy]-2-({[(1R,2S)-1-(methoxycarbonyl)-2-vinylcyclopropyl]amino}carbonyl)pyrrolidine-1-carboxylate instead of tert-butyl (2S,4R)-2-({[(1R,2S)-1-(methoxycarbonyl)-2-vinylcyclopropyl]amino}carbonyl)-4-[(2-pyridin-2-ylthieno[3,2-d]pyrimidin-4-yl)oxy]pyrrolidine-1-carboxylate yielded the title compound of Example 73 as a white solid (2.1 g, 33% yield): ¹H NMR (400 MHz, DMSO-d6) δ 8.74 (s, 1H), 8.34 (d, J=5.6 Hz, 1H), 7.63 (d, J=5.3 Hz, 1H), 7.56-7.49 (m, 2H), 6.56-6.54 (m, 1H), 6.46-6.44 (m, 1H), 5.79 (br. s, 1H), 5.67-5.58 (m, 1H), 5.30-5.25 (m, 1H), 5.10-5.07 (m, 1H), 4.15-4.07 (m, 1H), 3.89-3.85 (m, 1H), 3.58 (s, 3H), 3.41-3.38 (m, 1H), 3.28-3.23 (m, 2H), 2.43-2.32 (m, 1H), 2.26-2.18 (m, 2H), 1.68-1.64 (m, 1H), 1.35-1.31 (m, 1H), 1.19 (d, J=6.3 Hz, 6H); LCMS (ESI+) for C₂₆H₃₀N₆O₄S m/z 523 (M+H)⁺.

### Example 74

### Methyl (1R,2S)-1-{[(4R)-1-{(2S)-2-[(tert-butoxycarbonyl)amino]non-8-enoyl}-4-({2-[6-(isopropylamino)pyridin-2-yl]thieno[3,2-d]pyrimidin-4-yl}oxy)-L-prolyl]amino}-2-vinylcyclopropanecarboxylate

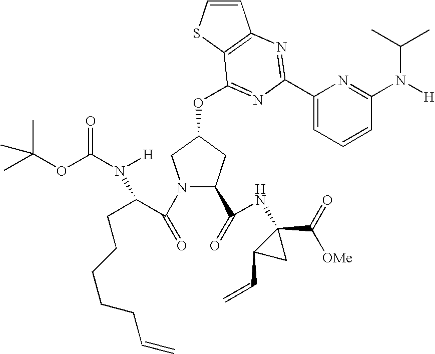

Using the procedure described for Example 37 and using methyl (1R,2S)-1-{[(4R)-4-({2-[6-(isopropylamino)pyridin-2-yl]thieno[3,2-d]pyrimidin-4-yl}oxy)-L-prolyl]amino}-2-vinylcyclopropanecarboxylate instead of methyl (1R,2S)-1-({(4R)-4-[(2-pyridin-2-ylthieno[3,2-d]pyrimidin-4-yl)oxy]-L-prolyl}amino)-2-vinylcyclopropanecarboxylate yielded the title compound of Example 74 as a beige foam (2.8 g, 90% yield): ¹H NMR (400 MHz, DMSO-d6) δ 8.83 (s, 0.2H), 8.63 (s, 0.8H), 8.37-8.33 (m, 1H), 7.66-7.62 (m, 1H), 7.56-7.49 (m, 2H), 6.99 (d, J=7.6 Hz, 0.8H), 6.69 (d, J=7.6 Hz, 0.2H), 6.55 (d, J=8.1 Hz, 1H), 6.46 (d, J=7.3 Hz, 1H), 5.97 (s, 0.8H), 5.85 (s, 0.2H), 5.79-5.71 (m, 1H), 5.69-5.59 (m, 1H), 5.28-5.20 (m, 1H), 5.09 (dd, J=10.4, 1.8 Hz, 1H), 4.99-4.88 (m, 2H), 4.51 (t, J=7.3 Hz, 0.2H), 4.44 (t, J=8.2 Hz, 0.8H), 4.30 (d, J=11.9 Hz, 0.8H), 4.15-4.10 (m, 1.2H), 4.06-4.00 (m, 2H), 3.57 (s, 3H), 2.34-2.30 (m, 1H), 2.08-1.95 (m, 3H), 1.64-1.50 (m, 2H), 1.37-1.16 (m, 24H); LCMS (ESI+) for C₄₀H₅₃N₇₆O₇S m/z 776 (M+H)⁺.

### Example 75

### Methyl (2R,6S,12Z,13aS,14aR,16aS)-6-[(tert-butoxycarbonyl)amino]-2-({2-[6-(isopropylamino)pyridin-2-yl]thieno[3,2-d]pyrimidin-4-yl}oxy)-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

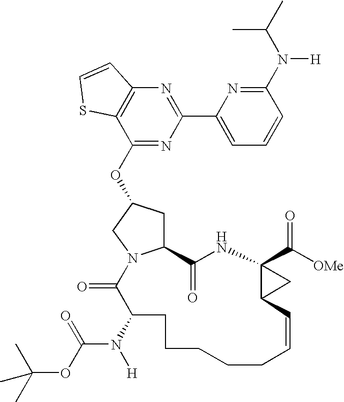

Using the procedure described for Example 38 and using methyl (1R,2S)-1-{[(4R)-1-{(2S)-2-[(tert-butoxycarbonyl)amino]non-8-enoyl}-4-({2-[6-(isopropylamino)pyridin-2-yl]thieno[3,2-d]pyrimidin-4-yl}oxy)-L-prolyl]amino}-2-vinylcyclopropanecarboxylate instead of methyl (1R,2S)-1-({(4R)-1-{(2S)-2-[(tert-butoxycarbonyl)amino]non-8-enoyl}-4-[(2-pyridin-2-ylthieno[3,2-d]pyrimidin-4-yl)oxy]-L-prolyl}amino)-2-vinylcyclopropanecarboxylate yielded the title compound of Example 75 as a grey solid (1.05 g, 39% yield): ¹H NMR (400 MHz, DMSO-d6) δ 8.76 (s, 1H), 8.30 (d, J=5.3 Hz, 1H), 7.61 (d, J=5.6 Hz, 1H), 7.56-7.49 (m, 2H), 6.98 (d, J=6.6 Hz, 1H), 6.56 (dd, J=8.0, 0.9 Hz, 1H), 6.47 (d, J=7.6 Hz, 1H), 5.97 (br. s, 1H), 5.56-5.49 (m, 1H), 5.25 (t, J=9.6 Hz, 1H), 4.68 (d, J=11.6 Hz, 1H), 4.52 (t, J=8.2 Hz, 1H), 4.16-4.11 (m, 1H), 3.93-3.89 (m, 2H), 3.55 (s, 3H), 2.60-2.50 (m, 2H), 2.40-2.32 (m, 1H), 2.25-2.20 (m, 1H), 1.80-1.70 (m, 2H), 1.56-1.53 (m, 1H), 1.50-1.47 (m, 1H), 1.37-1.25 (m, 5H), 1.21 (d, J=6.3 Hz, 6H), 1.16-1.08 (m, 2H), 1.03 (s, 9H); LCMS (ESI+) for C₃₈H₄₉N₇₆O₇S m/z 748 (M+H)⁺.

### Example 76

### Methyl (2R,6S,12Z,13aS,14aR,16aS)-6-amino-2-({2-[6-(isopropylamino)pyridin-2-yl]thieno[3,2-d]pyrimidin-4-yl}oxy)-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

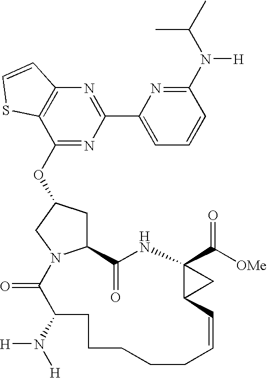

Using the procedure described for Example 39 and using methyl (2R,6S,12Z,13aS,14aR,16aS)-6-[(tert-butoxycarbonyl)amino]-2-({2-[6-(isopropylamino)pyridin-2-yl]thieno[3,2-d]pyrimidin-4-yl}oxy)-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,6S,12Z,13aS,14aR,16aS)-6-[(tert-butoxycarbonyl)amino]-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate yielded the title compound of Example 76 as a grey solid (0.903 g, >100% yield): ¹H NMR (400 MHz, DMSO-d6) δ 8.70 (s, 1H), 8.37 (d, J=5.6 Hz, 1H), 7.66 (d, J=5.3 Hz, 1H), 7.58-7.50 (m, 2H), 6.57 (dd, J=8.01, 0.9 Hz, 1H), 6.49 (d, J=7.6 Hz, 1H), 6.02 (s, 1H), 5.55-5.48 (m, 1H), 5.32-5.27 (t, J=9.8 Hz, 1H), 4.52 (t, J=7.7 Hz, 1H), 4.18-4.12 (m, 2H), 4.05-4.0 (m, 1H), 3.56 (s, 3H), 2.47-2.42 (m, 1H), 2.36-2.28 (m, 1H), 1.99 (s, 3H), 1.96-1.90 (m, 1H), 1.57-1.48 (m, 6H), 1.32-1.21 (m, 12H); LCMS (ESI+) for C₃₃H₄₁N₇O₅S m/z 648 (M+H)⁺.

### Example 77

### Methyl (2R,6S,12Z,13aS,14aR,16aS)-6-{[(cyclopentyloxy)carbonyl]amino}-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate

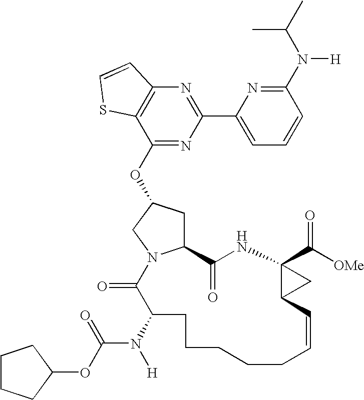

Using the procedure described for Example 40 and using methyl (2R,6S,12Z,13aS,14aR,16aS)-6-amino-2-({2-[6-(isopropylamino)pyridin-2-yl]thieno[3,2-d]pyrimidin-4-yl}oxy)-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,6S,12Z,13aS,14aR,16aS)-6-amino-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate yielded the title compound of Example 77 as a green foam (1.12 g, >100% yield): ¹H NMR (400 MHz, DMSO-d6) δ 8.72 (s, 1H), 8.33 (d, J=5.6 Hz, 1H), 7.64-7.62 (m, 1H), 7.60-7.49 (m, 2H), 7.17 (d, J=7.1 Hz, 1H), 6.55 (d, J=7.3 Hz, 1H), 6.46 (d, J=7.3 Hz, 1H), 6.01 (br. s, 1H), 5.55-5.49 (m, 1H), 5.25 (t, J=9.6 Hz, 1H), 4.56-4.49 (m, 2H), 4.44-4.38 (m, 1H), 4.19-4.08 (m, 1H), 4.04-3.92 (m, 2H), 3.55 (s, 3H), 2.42-2.37 (m, 1H), 2.27-2.17 (m, 1H), 1.94 (s, 6H), 1.67-1.47 (m, 8H), 1.40-1.26 (m, 7H), 1.21 (d, J=6.3 Hz, 6H); LCMS (ESI+) for C₃₉H₄₉N₇O₇S m/z 760 (M+H)⁺.

### Example 78

### (2R,6S,12Z,13aS,14aR,16aS)-6-{[(Cyclopentyloxy)carbonyl]amino}-2-({2-[6-(isopropylamino)pyridin-2-yl]thieno[2,3-d]pyrimidin-4-yl}oxy)-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

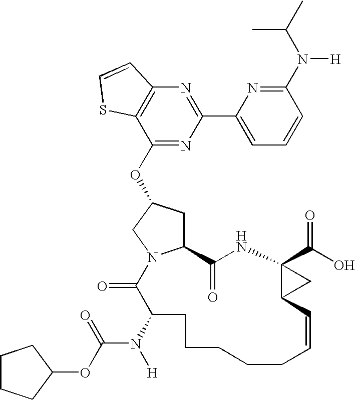

Using the procedure described for Example 11 and using methyl (2R,6S,12Z,13aS,14aR,16aS)-6-{[(cyclopentyloxy)carbonyl]amino}-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-{[(cyclopentyloxy)carbonyl]amino}-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate yielded the title compound of Example 78 as a white solid (115 mg, 11% yield): ¹H NMR (400 MHz, DMSO-d6) δ 11.79 (br. s, 1H), 8.65 (s, 1H), 8.34 (d, J=5.3 Hz, 1H), 7.62 (d, J=5.3 Hz, 1H), 7.60-7.48 (m, 2H), 7.16 (d, J=6.8 Hz, 1H), 6.61-6.43 (m, 2H), 6.01 (s, 1H), 5.54-5.47 (m, 1H), 5.26 (t, J=9.7 Hz, 1H), 4.56-4.48 (m, 2H), 4.41 (br. s, 1H), 4.15-4.10 (m, 1H), 4.03-3.93 (m, 2H), 2.53 (s, 3H), 2.44-2.35 (m, 2H), 2.22-2.15 (m, 1H), 1.82-1.62 (m, 2H), 1.55-1.44 (m, 7H), 1.42-1.27 (m, 8H), 1.21 (d, J=6.3 Hz, 6H); LCMS (ESI+) for C₃₈H₄₇N₇O₇S m/z 746 (M+H)⁺; Anal. calcd. for C₃₈H₄₇N₇O₇S•0.09 ethyl acetate•0.34 acetic acid•2.91H₂O: C, 56.72; H, 6.69; N, 11.86. Found: C, 56.48; H, 6.33; N, 11.50.

### Example 79

### (2R,6S,12Z,13aS,14aR,16aS)-6-[(Tert-butoxycarbonyl)amino]-2-({2-[6-(isopropylamino)pyridin-2-yl]thieno[3,2-d]pyrimidin-4-yl}oxy)-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylic acid

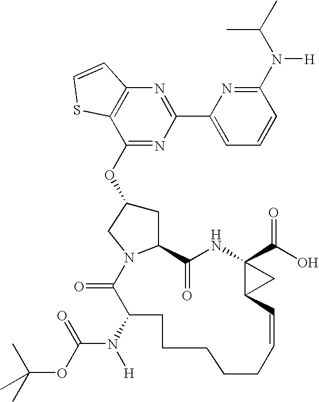

Using the procedure described for Example 11 and using methyl (2R,6S,12Z,13aS,14aR,16aS)-6-[(tert-butoxycarbonyl)amino]-2-({2-[6-(isopropylamino)pyridin-2-yl]thieno[3,2-d]pyrimidin-4-yl}oxy)-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-{[(cyclopentyloxy)carbonyl]amino}-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate yielded the title compound of Example 79 as a white solid (13 mg, 8.5% yield): ¹H NMR (400 MHz, DMSO-d6) δ 13.14 (br. s, 1H), 8.71 (s, 1H), 8.50-8.48 (m, 1H), 7.88 (br. s, 1H), 7.70-7.68 (m, 1H), 7.00 (d, J=6.6 Hz, 1H), 6.15 (br. s, 1H), 5.54-5.47 (m, 1H), 5.27 (t, J=9.5 Hz, 1H), 4.66 (d, J=11.6 Hz, 1H), 4.52 (t, J=8.2 Hz, 1H), 4.19-4.00 (m, 2H), 3.95-3.89 (m, 3H), 2.42-2.29 (m, 3H), 2.20-2.16 (m, 1H), 1.77-1.63 (m, 2H), 1.53-1.44 (m, 3H), 1.27 (d, J=6.3 Hz, 1H), 1.17-1.11 (m, 3H), 0.99 (s, 9H), 0.88-0.83 (m, 1H); LCMS (ESI+) for C₃₇H₄₇N₇O₇S m/z 734 (M+H)⁺.

### Example 80

### 7-Chloro-2-(1-methyl-1H-imidazol-2-yl)-thieno[3,2-b]pyridine

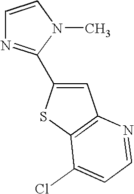

7-Chloro-2-(1-methyl-1H-imidazol-2-yl)-thieno[3,2-b]pyridine was prepared according to the procedure in WO 9924440 and U.S. Pat. No. 6,492,383.

### Example 81

### 4-[2-(1-Methyl-1H-imidazol-2-yl)-thieno[3,2-b]pyridin-7-yloxy]-pyrrolidine-1,2-dicarboxylic acid 1-tert-butyl ester

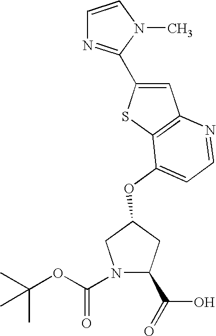

Using the procedure described for Example 4 and using 7-chloro-2-(1-methyl-1H-imidazol-2-yl)-thieno[3,2-b]pyridine instead of 7-chloro-5-pyridin-2-ylthieno[3,2-b]pyridine yielded the title compound of Example 81 as a tan solid (1.9 g, 76% yield): ¹H NMR (400 MHz, DMSO-d6) δ 12.78 (br. s, 1H), 8.53 (dd, J=5.3, 1.5 Hz, 1H), 7.81 (d, J=1.5 Hz, 1H), 7.38 (s, 1H), 7.05 (dd, J=5.4, 1.1 Hz, 1H), 7.03 (s, 1H), 5.36 (br. s, 1H), 4.31-4.24 (m, 1H), 3.96 (s, 3H), 3.73-3.65 (m, 2H), 2.60-2.57 (m, 1H), 2.35-2.29 (m, 1H), 1.35 (s, 9H); MS (ESI+) for C₂₁H₂₄N₄O₅S m/z 445 (M+H)⁺.

### Example 82

### 2-(1-Methoxycarbonyl-2-vinyl-cyclopropylcarbamoyl)-4-[2-(1-methyl-1H-imidazol-2-yl)-thieno[3,2-b]pyridin-7-yloxy]-pyrrolidine-1-carboxylic acid tert-butyl ester

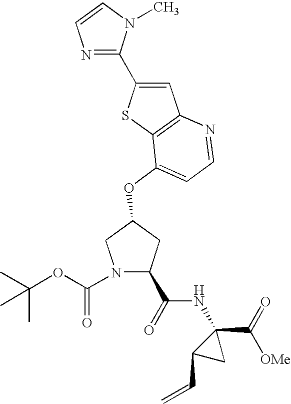

Using the procedure described for Example 5, using 4-[2-(1-methyl-1H-imidazol-2-yl)-thieno[3,2-b]pyridin-7-yloxy]-pyrrolidine-1,2-dicarboxylic acid 1-tert-butyl ester 5165-171-3 instead of (4R)-1-(tert-butoxycarbonyl)-4-[(5-pyridin-2-ylthieno[3,2-b]pyridin-7-yl)oxy]-L-proline, yielded the title compound of Example 82 as a beige foam (1.9 g, 83% yield): ¹H NMR (400 MHz, DMSO-d6) δ 8.77 (s, 0.7H), 8.73 (s, 0.3H), 8.54 (d, J=5.3 Hz, 1H), 8.30 (s, 1H), 7.81 (s, 1H), 7.38 (s, 1H), 7.06 (d, J=5.6 Hz, 1H), 7.02 (d, J=1.0 Hz, 1H), 5.68-5.59 (m, 1H), 5.35 (br. s, 1H), 5.29-5.24 (m, 1H), 5.11-5.07 (m, 1H), 4.26-4.20 (m, 1H), 3.96 (s, 3H), 3.72 (s, 2H), 3.60-3.58 (m, 3H), 2.25-2.11 (m, 2H), 1.68-1.66 (m, 1H), 1.34 (s, 9H), 1.32-1.27 (m, 1H); MS (ESI+) for C₂₈H₃₃N₅O₆S m/z 568 (M+H)⁺.

### Example 83

### 1-({4-[2-(1-Methyl-1H-imidazol-2-yl)-thieno[3,2-b]pyridin-7-yloxy]-pyrrolidine-2-carbonyl}-amino)-2-vinyl-cyclopropanecarboxylic acid methyl ester

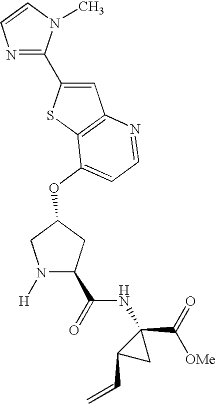

2-(1-Methoxycarbonyl-2-vinyl-cyclopropylcarbamoyl)-4-[2-(1-methyl-1H-imidazol-2-yl)-thieno[3,2-b]pyridin-7-yloxy]-pyrrolidine-1-carboxylic acid tert-butyl ester (5165-173-2) was dissolved in dichloromethane (15 mL) and treated with TFA (15 mL). The red solution was stirred for one hour, concentrated under reduced pressure and re-concentrated from chloroform and toluene which provided an amber oil (1.6 g; 100% yield); MS (ESI+) for C₂₃H₂₅N₅O₄S m/z 468 (M+H)⁺ and 490 (M+Na)⁺.

### Example 84

### 1-({1-(2-tert-Butoxycarbonylamino-non-8-enoyl)-4-[2-(1-methyl-1H-imidazol-2-yl)-thieno[3,2-b]pyridin-7-yloxy]-pyrrolidine-2-carbonyl}-amino)-2-vinyl-cyclopropanecarboxylic acid methyl ester

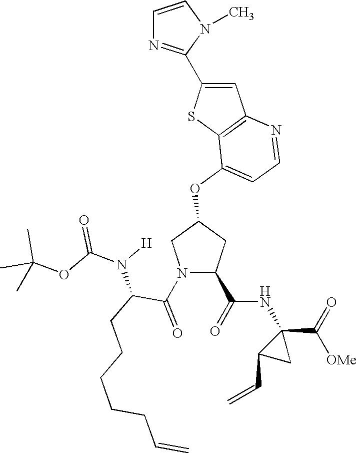

Using the procedure described for Example 7, using 1-({4-[2-(1-methyl-1H-imidazol-2-yl)-thieno[3,2-b]pyridin-7-yloxy]-pyrrolidine-2-carbonyl}-amino)-2-vinyl-cyclopropanecarboxylic acid methyl ester instead of methyl (1R,2S)-1-({(4R)-4-[(5-pyridin-2-ylthieno[3,2-b]pyridin-7-yl)oxy]-L-prolyl}amino)-2-vinylcyclopropanecarboxylate, yielded the title compound of Example 84 as a beige solid (1.4 g, 58% yield): ¹H NMR (400 MHz, DMSO-d6) δ 8.88 (s, 0.3H), 8.58-8.53 (m, 1.7H), 7.80-7.79 (m, 1H), 7.37 (s, 1H), 7.10-6.99 (m, 2.7H), 6.64 (d, J=8.1 Hz, 0.3H), 5.81-5.72 (m, 1H), 5.70-5.58 (m, 1H), 5.48 (br. s, 0.7H), 5.42 (br. s, 0.3H), 5.27-5.19 (m, 1H), 5.10-5.06 (m, 1H), 4.99-4.79 (m, 2H), 4.44 (t, J=8.0 Hz, 0.3H), 4.38 (t, J=8.3 Hz, 0.7H), 4.18 (d, J=12.4 Hz, 0.5H), 4.06-4.00 (m, 1.5H), 3.95 (s, 3H), 3.60-3.58 (m, 3H), 2.63-2.53 (m, 0.4H), 2.45-2.43 (m, 1.6H), 2.26-2.17 (m, 1H), 2.11-2.05 (m, 1H), 2.00-1.85 (m, 2H), 1.64-1.50 (m, 2H), 1.31-1.14 (m, 17H); MS (ESI+) for C₃₇H₄₈N₆O₇S m/z 721 (M+H)⁺.

### Example 85

### Methyl (2R,6S,12Z,13aS,14aR,16aS)-6-[(tert-butoxycarbonyl)amino]-2-{[2-(1-methyl-1H-imidazol-2-yl)thieno[3,2-b]pyridin-7-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

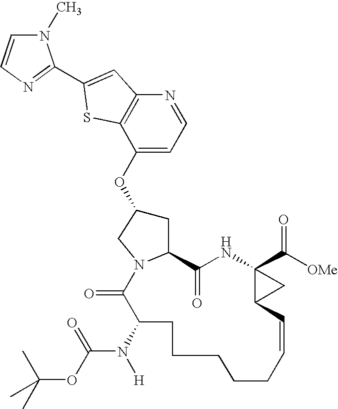

Using the procedure described for Example 8, using 1-({1-(2-tert-Butoxycarbonylamino-non-8-enoyl)-4-[2-(1-methyl-1H-imidazol-2-yl)-thieno[3,2-b]pyridin-7-yloxy]-pyrrolidine-2-carbonyl}-amino)-2-vinyl-cyclopropanecarboxylic acid methyl ester instead of methyl (1R,2S)-1-({(4R)-1-{2-[(tert-butoxycarbonyl)amino]non-8-enoyl }-4-[(5-pyridin-2-ylthieno[3,2-b]pyridin-7-yl)oxy]-L-prolyl}amino)-2-vinylcyclopropanecarboxylate, yielded the title compound of Example 85 as a beige solid (940 mg, 72% yield): ¹H NMR (400 MHz, DMSO-d6) δ 8.76 (s, 1H), 8.52 (d, J=5.6 Hz, 1H), 7.77 (s, 1H), 7.36 (s, 1H), 7.04 (d, J=5.6 Hz, 1H), 7.00 (s, 1H), 6.97 (d, J=6.8 Hz, 1H), 5.56-5.49 (m, 2H), 5.25 (t, J=9.6 Hz, 1H), 5.53-4.46 (m, 2H), 4.01-3.99 (m, 1H), 3.94 (s, 3H), 3.89-3.84 (m, 1H), 3.55 (s, 3H), 2.44-2.22 (m, 4H), 1.72-1.70 (m, 2H), 1.57-1.53 (m, 1H), 1.50-1.46 (m, 1H), 1.36-1.25 (m, 4H), 1.19-1.14 (m, 3H), 1.04 (s, 9H); MS (ESI+) for C₃₅H₄₄N₆O₇S m/z 693 (M+H)⁺.

### Example 86

### (2R,6S,12Z,13aS,14aR,16aS)-6-[(tert-butoxycarbonyl)amino]-2-{[2-(1-methyl-1H-imidazol-2-yl)thieno[3,2-b]pyridin-7-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylic acid

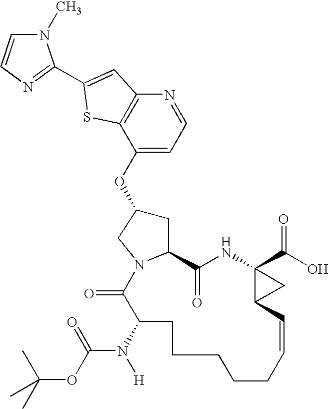

Using the procedure described for Example 14, using methyl (2R,6S,12Z,13aS,14aR,16aS)-6-[(tert-butoxycarbonyl)amino]-2-{[2-(1-methyl-1H-imidazol-2-yl)thieno[3,2-b]pyridin-7-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-[(tert-butoxycarbonyl)amino]-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 86 as a white solid (21 mg, 28% yield): ¹H NMR (400 MHz, DMSO-d6) δ 11.96 (br. s, 1H), 8.67 (s, 1H), 8.52 (d, J=5.6 Hz, 1H), 7.77 (s, 1H), 7.36 (s, 1H), 7.03 (d, J=5.6 Hz, 1H), 7.00 (s, 1H), 6.95 (d, J=7.1 Hz, 1H), 5.53-5.47 (m, 2H), 5.26 (t, J=9.6 Hz, 1H), 4.51-4.44 (m, 2H), 4.03-3.99 (m, 1H), 3.94 (s, 3H), 3.90-3.85 (m, 1H), 2.44-2.29 (m, 2H), 2.24-2.17 (m, 1H), 1.77-1.66 (m, 2H), 1.49-1.42 (m, 2H), 1.36-1.28 (m, 5H), 1.20-1.10 (m, 3H), 1.05 (s, 9H); MS (ESI+) for C₃₄H₄₂N₆O₇S m/z 679 (M+H)⁺.

### Example 87

### Methyl (2R,6S,12Z,13aS,14aR,16aS)-6-amino-2-{[2-(1-methyl-1H-imidazol-2-yl)thieno[3,2-b]pyridin-7-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylate

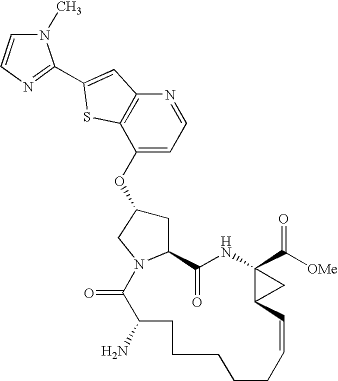

Using the procedure described for Example 9, using methyl (2R,6S,12Z,13aS,14aR,16aS)-6-[(tert-butoxycarbonyl)amino]-2-{[2-(1-methyl-1H-imidazol-2-yl)thieno[3,2-b]pyridin-7-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-[(tert-butoxycarbonyl)amino]-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 87 as a beige solid (0.58 g, 81% yield): ¹H NMR (400 MHz, DMSO-d6) δ 8.71 (s, 1H), 8.55 (d, J=5.3 Hz, 1H), 7.81 (s, 1H), 7.38 (d, J=1.0 Hz, 1H), 7.07 (d, J=5.6 Hz, 1H), 7.02 (d, J=1.0 Hz, 1H), 5.54-5.47 (m, 2H), 5.29 (t, J=10.0 Hz, 1H), 4.48 (t, J=8.0 Hz, 1H), 4.04-3.89 (m, 5H), 3.61-3.54 (m, 4H), 2.45-2.21 (m, 4H), 1.90-1.80 (m, 3H), 1.56-1.53 (m, 2H), 1.49-1.42 (m, 2H), 1.30-1.20 (m, 6H); MS (ESI+) for C₃₀H₃₆N₆O₅S m/z 593 (M+H)⁺.

### Example 88

### Methyl (2R,6S,12Z,13aS,14aR,16aS)-6-{[(cyclopentyloxy)carbonyl]amino}-2-{[2-(1-methyl-1H-imidazol-2-yl)thieno[3,2-b]pyridin-7-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

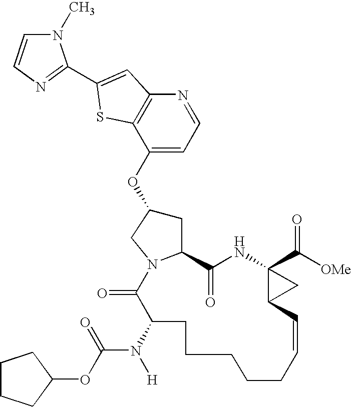

Using the procedure described for Example 10, using methyl (2R,6S,12Z,13aS,14aR,16aS)-6-amino-2-{[2-(1-methyl-1H-imidazol-2-yl)thieno[3,2-b]pyridin-7-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-amino-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 88 as a beige solid (130 mg, 74% yield): ¹H NMR (400 MHz, DMSO-d6) δ 8.73 (s, 1H), 8.53 (d, J=5.3 Hz, 1H), 7.78 (s, 1H), 7.36 (s, 1H), 7.20 (d, J=7.1 Hz, 1H), 7.06 (d, J=5.6 Hz, 1H), 6.99 (s, 1H), 5.56-5.49 (m, 2H), 5.25 (t, J=9.6 Hz, 1H), 4.57 (br. s, 1H), 4.48-4.41 (m, 2H), 4.06-4.03 (m, 1H), 3.95 (s, 3H), 3.88-3.85 (m, 1H), 3.55 (s, 3H), 2.34-2.24 (m, 4H), 1.75-1.70 (m, 2H), 1.57-1.16 (m, 17H); MS (ESI+) for C₃₆H₄₄N₆O₇S m/z 705 (M+H)⁺.

### Example 89

### (2R,6S,12Z,13aS,14aR,16aS)-6-{[(Cyclopentyloxy)carbonyl]amino}-2-{[2-(1-methyl-1H-imidazol-2-yl)thieno[3,2-b]pyridin-7-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

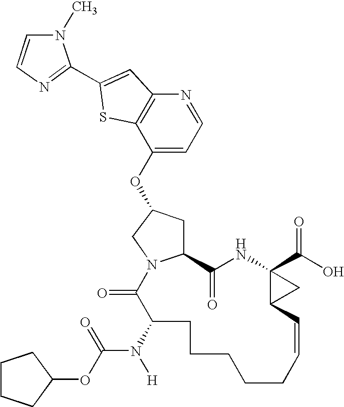

Using the procedure described for Example 14, using methyl (2R,6S,12Z,13aS,14aR,16aS)-6-{[(cyclopentyloxy)carbonyl]amino}-2-{[2-(1-methyl-1H-imidazol-2-yl)thieno[3,2-b]pyridin-7-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-[(tert-butoxycarbonyl)amino]-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 89 as a beige solid (47 mg, 40% yield): ¹H NMR (400 MHz, DMSO-d6) δ 12.22 (br. s, 1H), 8.65 (s, 1H), 8.57 (d, J=5.3 Hz, 1H), 7.81 (s, 1H), 7.40 (s, 1H), 7.26-7.04 (m, 4H), 5.54-5.47 (m, 2H), 5.26 (t, J=9.6 Hz, 1H), 4.55 (br. s, 1H), 4.47-4.41 (m, 2H), 4.07-4.03 (m, 1H), 3.96 (s, 3H), 3.91-3.86 (m, 1H), 2.44-2.41 (m, 1H), 2.33-2.29 (m, 2H), 2.23-2.17 (m, 1H), 1.80-1.70 (m, 2H), 1.58-1.17 (m, 16H); MS (ESI+) for C₃₅H₄₂N₆O₇S m/z 691 (M+H)⁺; Anal. calcd. for C₃₅H₄₂N₆O₇S•1.41 acetic acid: C, 58.58; H, 6.19; N, 10.84. Found: C, 58.57; H, 6.15; N, 10.59.

### Example 90

### Methyl (2R,6S,12Z,13aS,14aR,16aS)-6-[(cyclopropylacetyl)amino]-2-{[2-(1-methyl-1H-imidazol-2-yl)thieno[3,2-b]pyridin-7-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

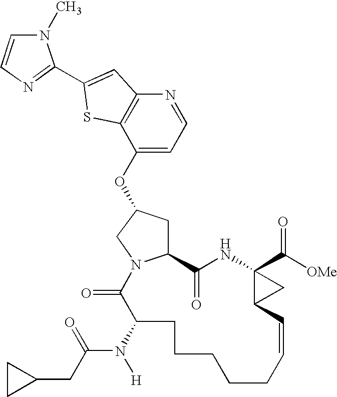

Using the procedure described for Example 15, using methyl (2R,6S,12Z,13aS,14aR,16aS)-6-amino-2-{[2-(1-methyl-1H-imidazol-2-yl)thieno[3,2-b]pyridin-7-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-amino-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 90 as a beige solid (160 mg, 95% yield): ¹H NMR (400 MHz, DMSO-d6) δ 8.73 (s, 1H), 8.54 (d, J=5.6 Hz, 1H), 7.94 (d, J=7.8 Hz, 1H), 7.79 (s, 1H), 7.36 (d, J=1.0 Hz, 1H), 7.07 (d, J=5.8 Hz, 1H), 7.00 (d, J=1.3 Hz, 1H), 5.55-5.50 (m, 2H), 5.27 (t, J=9.7 Hz, 1H), 4.47-4.40 (m, 2H), 4.32 (d, J=11.6 Hz, 1H), 3.97-3.93 (m, 4H), 3.55 (s, 3H), 2.45-2.28 (m, 3H), 1.88-1.75 (m, 4H), 1.56-1.52 (m, 1H), 1.50-1.47 (m, 1H), 1.40-1.15 (m, 8H), 0.74-0.68 (m, 1H), 0.25-0.21 (m, 2H), 0.04-(−0.07) (m, 2H); MS (ESI+) for C₃₅H₄₂N₆O₆S m/z 675 (M+H)⁺.

### Example 91

### (2R,6S,12Z,13aS,14aR,16aS)-6-[(Cyclopropylacetyl)amino]-2-{[2-(1-methyl-1H-imidazol-2-yl)thieno[3,2-b]pyridin-7-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylic acid

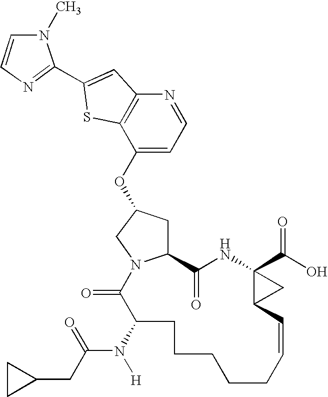

Using the procedure described for Example 14 and using methyl (2R,6S,12Z,13aS,14aR,16aS)-6-[(cyclopropylacetyl)amino]-2-{[2-(1-methyl-1H-imidazol-2-yl)thieno[3,2-b]pyridin-7-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-[(tert-butoxycarbonyl)amino]-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate yielded the title compound of Example 91 as an off-white solid (59 mg, 42% yield): ¹H NMR (400 MHz, DMSO-d6) δ 12.24 (br. s, 1H), 8.65 (s, 1H), 8.56 (d, J=5.6 Hz, 1H), 7.93 (d, J=7.6 Hz, 1H), 7.80 (s, 1H), 7.38 (s, 1H), 7.10 (d, J=5.6 Hz, 1H), 7.03 (s, 1H), 5.52-5.47 (m, 2H), 5.29 (t, J=9.7 Hz, 1H), 4.46-4.40 (m, 2H), 4.30 (d, J=11.6 Hz, 1H), 3.98-3.97 (m, 1H), 3.95 (s, 3H), 2.41-2.23 (m, 4H), 1.90-1.70 (m, 4H), 1.50-1.34 (m, 9H), 0.80-0.70 (m, 1H), 0.25-0.22 (m, 2H), −0.04-(−0.07) (m, 2H); MS (ESI+) C₃₄H₄₀N₆O₆S m/z 661 (M+H)⁺; Anal. calcd. for C₃₄H₄₀N₆O₆S•1.1 acetic acid•2.0H₂O: C, 56.99; H, 6.39; N, 11.02. Found: C, 56.57; H, 5.91; N, 10.81.

### Example 92

### 2-Methyl-2H-pyrazole-3-carboxylic acid methyl ester

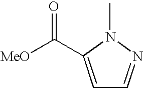

2-Methyl-2H-pyrazole-3-carboxylic acid (3.0 g, 24 mmol, 1.0 equiv) was dissolved in methanol (100 mL) and toluene (100 mL). The clear solution was slowly treated with (trimethylsilyl)diazomethane (24 mL of 2.0M in ether, 48 mmol, 2.0 equiv), stirred for 1 hour, concentrated in vacuo, and gave a clear oil (3.1 g, 94% yield): ¹H NMR (400 MHz, DMSO-d6) δ 7.52 (d, J=2.0 Hz, 1H), 6.86 (d, J=2.0 Hz, 1H), 4.07 (s, 3H), 3.82 (s, 3H).

### Example 93

### 2-Methyl-2H-pyrazole-3-carboxylic acid amide

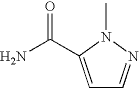

Concentrated ammonium hydroxide (30 mL) was added to 2-methyl-2H-pyrazole-3-carboxylic acid methyl ester (3.1 g, 22 mmol). The biphasic mixture was stirred for 16 hours and extracted with 10% IPA in chloroform, which gave a white solid (2.3 g, 82% yield): ¹H NMR (400 MHz, DMSO-d6) δ 7.88 (br. s, 1H), 7.44 (br. s, 1H), 7.41 (d, J=2.0 Hz, 1H), 6.83 (d, J=2.0 Hz, 1H), 4.03 (s, 3H).

### Example 94

### 2-Methyl-2H-pyrazole-3-carbonitrile

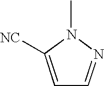

2-Methyl-2H-pyrazole-3-carboxylic acid amide (2.3 g, 18 mmol. 1.0 equiv) was dissolved in pyridine (36 mL) and treated with POCl₃ (2.5 mL, 26 mmol, 1.4 equiv). The resultant amber solution was stirred for 3 hours at room temperature. The reaction mixture was diluted with ice and the aqueous layer, which was adjusted to pH 3 with 6M HCl, was extracted with MTBE. The MTBE extract washed with water, brine, dried over magnesium sulfate, filtered, concentrated in vacuo and gave the product as a clear oil (1.8 g, 90% yield): ¹H NMR (400 MHz, DMSO-d6) δ 7.68 (d, J=2.0 Hz, 1H), 7.13 (d, J=2.0 Hz, 1H), 4.00 (s, 3H).

### Example 95

### 2-(2-Methyl-2H-pyrazol-3-yl)-thieno[3,2-d]pyrimidin-4-ol

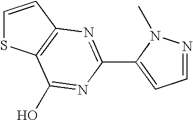

Methyl 3-aminothiophene-2-carboxylate (2.6 g, 17 mmol, 1.0 equiv) and 2-methyl-2H-pyrazole-3-carbonitrile (1.8 g, 17 mmol, 1.0 equiv) were taken up in anhydrous tetrahydrofuran (70 mL). The resultant solution was cooled to 0° C. and treated with potassium tert-butoxide (3.2 g, 29 mmol, 2.0 equiv). The resultant orange slurry was stirred for 18 h, concentrated in vacuo and poured into saturated ammonium chloride (100 mL). An off-white solid was collected by filtration (1.4 g, 36% yield): ¹H NMR (400 MHz, DMSO-d6) δ 12.68 (s, 1H), 8.23 (d, J=5.3 Hz, 1H), 7.56 (d, J=2.3 Hz, 1H), 7.46 (d, J=5.0 Hz, 1H), 7.17 (d, J=2.0 Hz, 1H), 4.19 (s, 3H); LCMS (ESI+) C₁₀H₈N₄OS m/z 233 (M+H)⁺.

### Example 96

### 4-[2-(2-Methyl-2H-pyrazol-3-yl)-thieno[3,2-d]pyrimidin-4-yloxy]-pyrrolidine-2-carboxylic acid methyl ester

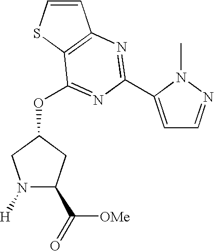

2-(2-Methyl-2H-pyrazol-3-yl)-thieno[3,2-d]pyrimidin-4-ol (1.4 g, 6.0 mmol, 1.0 equiv), c is 4-hydroxy-pyrrolidine-1,2-dicarboxylic acid 1-tert-butyl ester (1.5 g, 6.0 mmol, 1.0 equiv) and triphenylphosphine (3.1 g, 12 mmol, 2.0 equiv) were taken up in anhydrous tetrahydrofuran (120 mL). The resultant white slurry was treated with DIAD (2.3 mL, 12 mmol, 2.0 equiv). The light orange solution was stirred at ambient temperature for 15 h. The reaction mixture was analyzed by LCMS (ESI+) and gave the product [C₂₁H₂₅N₅O₅S m/z 460 (M+H)⁺] and no starting materials. The solvent was concentrated in vacuo, and the resultant amber oil was dissolved in ethyl acetate and washed with water and brine. The organic layer was dried over magnesium sulfate and concentrated in vacuo and gave an amber oil (10.3 g). The crude product was dissolved in dichloromethane (12 mL) and treated with trifluoroacetic acid (12 mL). The amber solution was stirred for two hours at ambient temperature. The solvents were removed in vacuo and the resultant amber oil was dissolved in dichloromethane. The organic layer washed with 50% saturated sodium bicarbonate, brine and extracted with 1.2M HCl. The acidic extract washed with dichloromethane. The combined organic extracts were discarded. The acidic aqueous layer was saturated with sodium bicarbonate and extracted with dichloromethane. The dichloromethane layer washed with brine, dried over magnesium sulfate, filtered, concentrated in vacuo and gave the product as a white solid (1.5 g, 2-step 71% yield): ¹H NMR (400 MHz, DMSO-d6) δ 8.36 (d, J=5.3 Hz, 1H), 7.61 (d, J=5.3 Hz, 1H), 7.51 (d, J=1.8 Hz, 1H), 7.02 (d, J=1.8 Hz, 1H), 5.79 (br. s, 1H), 4.30 (s, 3H), 3.96 (t, J=7.6 Hz, 1H), 3.65 (s, 3H), 3.37 (dd, J=12.4, 5.0 Hz, 1H), 3.12 (dd, J=12.4, 1.3 Hz, 1H), 3.04 (br. s, 1H), 2.34-2.31 (m, 2H); LCMS (ESI+) C₁₆H₁₇N₅O₃S m/z 360 (M+H)⁺.

### Example 97

### 1-(2-Tert-butoxycarbonylamino-non-8-enoyl)-4-[2-(2-methyl-2H-pyrazol-3-yl)-thieno[3,2-d]pyrimidin-4-yloxy]-pyrrolidine-2-carboxylic acid methyl ester

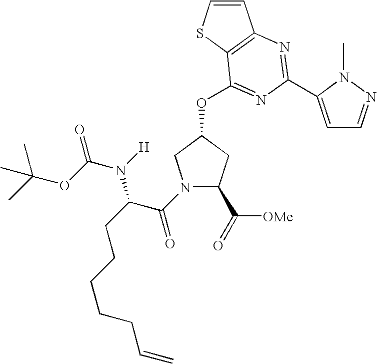

Using the procedure described for Example 7, using 4-[2-(2-methyl-2H-pyrazol-3-yl)-thieno[3,2-d]pyrimidin-4-yloxy]-pyrrolidine-2-carboxylic acid methyl ester instead of methyl (1R,2S)-1-({(4R)-4-[(5-pyridin-2-ylthieno[3,2-b]pyridin-7-yl)oxy]-L-prolyl}amino)-2-vinylcyclopropanecarboxylate, yielded the title compound of Example 97 as a beige foam (2.4 g, 100% yield): ¹H NMR (400 MHz, DMSO-d6) δ 8.40-8.37 (m, 1H), 7.63-7.61 (m, 1H), 7.52 (d, J=1.8 Hz, 1H), 7.03 (s, 2H), 5.95 (br. s, 1H), 5.81-5.73 (m, 1H), 5.01-4.91 (m, 2H), 4.52 (t, J=8.7 Hz, 1H), 4.45 (d, J=11.9 Hz, 1H), 4.30 (s, 3H), 4.12-4.05 (m, 1H), 4.00 (dd, J=11.9, 4.0 Hz, 1H), 3.64 (s, 3H), 2.70-2.60 (m, 1H), 2.39-2.32 (m, 1H), 2.02-1.97 (m, 2H), 1.59-1.54 (m, 1H), 1.45-1.40 (m, 1H), 1.36-1.21 (m, 6H), 1.18-1.09 (m, 9H); LCMS (ESI+) C₃₀H₄₀N₆O₆S m/z 612 (M+H)⁺.

### Example 98

### 1-(2-Tert-butoxycarbonylamino-non-8-enoyl)-4-[2-(2-methyl-2H-pyrazol-3-yl)-thieno[3,2-d]pyrimidin-4-yloxy]-pyrrolidine-2-carboxylic acid potassium salt

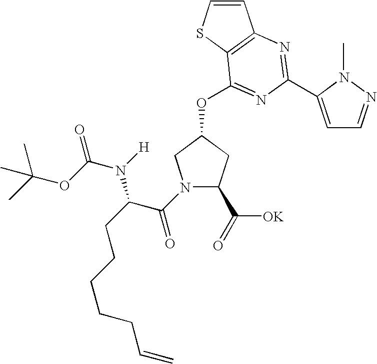

1-(2-tert-butoxycarbonylamino-non-8-enoyl)-4-[2-(2-methyl-2H-pyrazol-3-yl)-thieno[3,2-d]pyrimidin-4-yloxy]-pyrrolidine-2-carboxylic acid methyl ester (2.4 g, 3.9 mmol, 1.0 equiv) was dissolved in ether (25 mL) and treated with potassium trimethylsilanolate (0.67 g, 4.7 mmol, 1.2 equiv). The amber solution was stirred for 15 minutes and then concentrated under reduced pressure which gave the product as a white solid (2.3 g, 92%): LCMS (ESI+) C₂₉H₃₈N₆O₆S m/z 599 (M+H)⁺.

### Example 99

### 1-({1-(2-Tert-butoxycarbonylamino-non-8-enoyl)-4-[2-(2-methyl-2H-pyrazol-3-yl)-thieno[3,2-d]pyrimidin-4-yloxy]-pyrrolidine-2-carbonyl}-amino)-2-vinyl-cyclopropanecarboxylic acid methyl ester

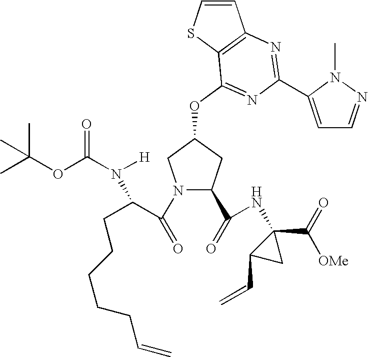

Using the procedure described for methyl Example 7, using 1-(2-tert-butoxycarbonylamino-non-8-enoyl)-4-[2-(2-methyl-2H-pyrazol-3-yl)-thieno[3,2-d]pyrimidin-4-yloxy]-pyrrolidine-2-carboxylic acid potassium salt instead of methyl (1R,2S)-1-({(4R)-4-[(5-pyridin-2-ylthieno[3,2-b]pyridin-7-yl)oxy]-L-prolyl}amino)-2-vinylcyclopropanecarboxylate, yielded the title compound of Example 99 as a white foam (1.8 g, 50% yield): ¹H NMR (400 MHz, DMSO-d6) δ 8.88 (s, 0.2H), 8.63 (s, 0.8H), 8.41-8.38 (m, 1H), 7.65-7.61 (m, 2H), 7.53-7.52 (m, 1H), 7.06-7.03 (m, 1.8H), 6.70 (d, J=7.8 Hz, 0.2H), 5.95 (br. s, 0.8H), 5.92 (br. s, 0.2H), 5.80-5.74 (m, 1H), 5.73-5.59 (m, 1H), 5.28-5.20 (m, 1H), 5.09 (dd, J=10.2, 1.6 Hz, 1H), 5.00-4.98 (m, 2H), 4.49-4.42 (m, 1H), 4.32 (s, 2.5H), 4.31 (s, 0.5H), 4.06-3.99 (m, 2H), 3.57 (s, 3H), 2.54-2.53 (m, 1H), 2.32-2.30 (m, 1H), 2.09-2.00 (m, 1H), 1.98-1.95 (m, 2H), 1.64-1.58 (m, 2H), 1.39-1.27 (m, 9H), 1.12 (m, 9H); LCMS (ESI+) C₃₆H₄₇N₇O₇S m/z 722 (M+H)⁺.

### Example 100

### Methyl (2R,6S,12Z,13aS,14aR,16aS)-6-[(tert-butoxycarbonyl)amino]-2-{[2-(1-methyl-1H-pyrazol-5-yl)thieno[3,2-d]pyrimidin-4-yl]oxy}-5,16-dioxo-,2,3,6,7,8,9,10,11,13a,14,15,16,16a-tetradecahydrocyclopropa[e]pyrrolo[1,2-a][1,4]diazacyclopentadecine-14a(5H)-carboxylate

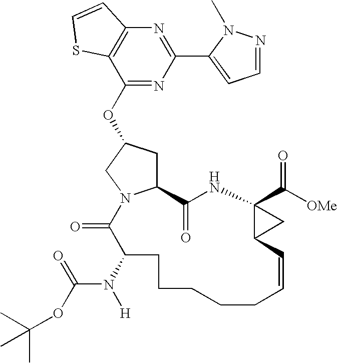

Using the procedure described for methyl Example 8, using 1-({1-(2-tert-butoxycarbonylamino-non-8-enoyl)-4-[2-(2-methyl-2H-pyrazol-3-yl)-thieno[3,2-d]pyrimidin-4-yloxy]-pyrrolidine-2-carbonyl}-amino)-2-vinyl-cyclopropanecarboxylic acid methyl ester instead of methyl (1R,2S)-1-({(4R)-1-{2-[(tert-butoxycarbonyl)amino]non-8-enoyl}-4-[(5-pyridin-2-ylthieno[3,2-b]pyridin-7-yl)oxy]-L-prolyl }amino)-2-vinylcyclopropanecarboxylate, yielded the title compound of Example 100 as a tan solid (1.0 g, 59% yield): ¹H NMR (400 MHz, DMSO-d6) δ 8.76 (s, 1H), 8.34 (d, J=5.3 Hz, 1H), 7.60 (d, J=5.3 Hz, 1H), 7.52 (d, J=2.0 Hz, 1H), 7.04 (d, J=1.8 Hz, 1H), 6.98 (d, J=6.6 Hz, 1H), 5.96 (br. s, 1H), 5.56-5.49 (m, 1H), 5.25 (t, J=9.6 Hz, 1H), 4.68 (d, J=11.9 Hz, 1H), 4.53 (t, J=8.2 Hz, 1H), 4.31 (s, 3H), 3.95-3.92 (m, 2H), 3.56 (s, 3H), 2.60-2.52 (m, 1H), 2.42-2.36 (m, 1H), 2.23 (q, J=8.6 Hz, 1H), 1.72-1.67 (m, 2H), 1.58-1.55 (m, 1H), 1.51-1.46 (m, 1H), 1.38-1.27 (m, 5H), 1.15-1.11 (m, 3H), 0.98 (s, 9H); LCMS (ESI+) C₃₄H₄₃N₇O₇S m/z 694 (M+H)⁺.

### Example 101

### (2R,6S,12Z,13aS,14aR,16aS)-6-[(Tert-butoxycarbonyl)amino]-2-{[2-(1-methyl-1H-pyrazol-5-yl)thieno[3,2-d]pyrimidin-4-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylic acid

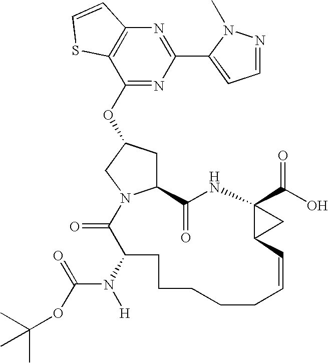

A mixture of methyl (2R,6S,12Z,13aS,14aR,16aS)-6-[(tert-butoxycarbonyl)amino]-2-{[2-(1-methyl-1H-pyrazol-5-yl)thieno[3,2-d]pyrimidin-4-yl]oxy}-5,16-dioxo-,2,3, 6,7,8,9,10,11,13a,14,15,16,16a-tetradecahydrocyclopropa[e]pyrrolo[1,2-a][1,4]diazacyclopentadecine-14a(5H)-carboxylate (45 mg, 0.065 mmol, 1.0 equiv) and potassium trimethylsilanolate (18 mg, 0.143 mmol, 2.2 equiv) in ether (2 mL) was stirred for 2 hours. A white solid was collected (44 mg, 94% yield): ¹H NMR (400 MHz, DMSO-d6) δ 8.36 (d, J=5.3 Hz, 1H), 7.72 (s, 1H), 7.60 (d, J=5.3 Hz, 1H), 7.51 (d, J=2.0 Hz, 1H), 7.06 (d, J=1.8 Hz, 1H), 6.85 (d, J=6.8 Hz, 1H), 5.96 (br. s, 1H), 5.41 (t, J=10.0 Hz, 1H), 5.12-5.05 (m, 1H), 4.59 (d, J=11.6 Hz, 1H), 4.36-4.33 (m, 1H), 4.32 (s, 3H), 4.15-4.11 (m, 2H), 2.59-2.54 (m, 1H), 2.44-2.32 (m, 1H), 2.12-1.95 (m, 3H), 1.73-1.64 (m, 2H), 1.39-1.14 (m, 7H), 1.05 (s, 9H); LCMS (ESI+) C₃₃H₄₁N₇O₇S m/z 680 (M+H)⁺. The white solid was dissolved in dichloromethane and the organic layer washed with 0.5M sodium citrate (pH 4.5), brine, dried over magnesium sulfate, filtered and concentrated in vacuo, which gave a white solid from MTBE-hexanes (15 mg): Anal. calcd. for C₃₃H₄₁N₇O₇S•1.0 H₂O•0.18 CH₂Cl₂: C, 55.89; H, 6.13; N, 13.75. Found: C, 56.39; H, 6.16; N, 13.34.

### Example 102

### Methyl (2R,6S,12Z,13aS,14aR,16aS)-6-amino-2-{[2-(1-methyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

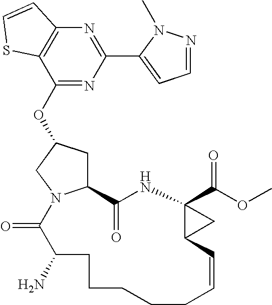

Using the procedure described for Example 9 using methyl (2R,6S,12Z,13aS,14aR,16aS)-6-[(tert-butoxycarbonyl)amino]-2-{[2-(1-methyl-1H-pyrazol-5-yl)thieno[3,2-d]pyrimidin-4-yl]oxy}-5,16-dioxo-,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-[(tert-butoxycarbonyl)amino]-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate yielded the title compound of Example 102 as a white solid (0.72 g, 94% yield): ¹H NMR (400 MHz, DMSO-d6) δ 8.70 (s, 1H), 8.40 (d, J=5.3 Hz, 1H), 7.64 (d, J=5.6 Hz, 1H), 7.53 (d, J=2.0 Hz, 1H), 7.05 (d, J=2.0 Hz, 1H), 6.05 (br. s, 1H), 5.54-5.48 (m, 1H), 5.29 (t, J=9.9 Hz, 1H), 4.52 (t, J=7.6 Hz, 1H), 4.32 (s, 3H), 4.09-3.99 (m, 2H), 3.56 (s, 3H), 3.54-3.52 (m, 1H), 2.47-2.44 (m, 1H), 2.37-2.29 (m, 1H), 2.25-2.00 (m, 3H), 1.96-1.85 (m, 1H), 1.57-1.33 (m, 5H), 1.24-1.23 (m, 6H); LCMS (ESI+) C₂₉H₃₅N₇O₅S m/z 594 (M+H)⁺.

### Example 103

### Methyl (2R,6S,12Z,13aS,14aR,16aS)-6-[(cyclopropylacetyl)amino]-2-{[2-(1-methyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

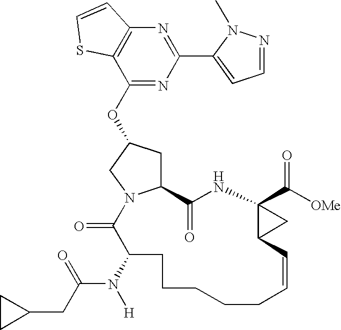

Using the procedure described for Example 15 using methyl (2R,6S,12Z,13aS,14aR,16aS)-6-amino-2-{[2-(1-methyl-1H-pyrazol-5-yl)thieno[3,2-d]pyrimidin-4-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-amino-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate yielded the title compound of Example 103 as a white solid (141 mg, 83% yield): ¹H NMR (400 MHz, DMSO-d6) δ 8.73 (s, 1H), 8.38 (d, J=5.3 Hz, 1H), 7.94 (d, J=7.6 Hz, 1H), 7.62 (d, J=5.6 Hz, 1H), 7.53 (d, J=1.8 Hz, 1H), 7.05 (d, J=2.0 Hz, 1H), 6.05 (br. s, 1H), 5.56-5.50 (m, 1H), 5.27 (t, J=9.7 Hz, 1H), 4.50 (t, J=8.0 Hz, 1H), 4.42 (d, J=11.9 Hz, 1H), 4.36-4.34 (m, 1H), 4.33 (s, 3H), 4.05 (dd, J=11.6, 4.3 Hz, 1H), 3.56 (s, 3H), 2.46-2.39 (m, 1H), 2.27 (q, J=8.8 Hz, 1H), 1.84-1.77 (m, 4H), 1.56-1.53 (m, 1H), 1.52-1.48 (m, 1H), 1.33-1.23 (m, 9H), 0.67-0.60 (m, 1H), 0.24-0.19 (m, 2H), −0.05-(−0.09) (m, 2H); LCMS (ESI+) C₃₄H₄₁N₇O₆S m/z 676 (M+H)⁺.

### Example 104

### (2R,6S,12Z,13aS,14aR,16aS)-6-[(Cyclopropylacetyl)amino]-2-{[2-(1-methyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

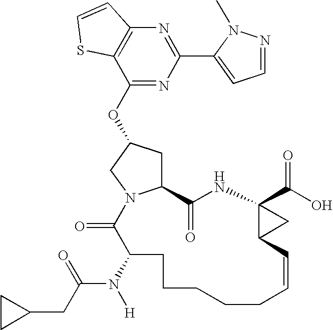

A mixture of methyl (2R,6S,12Z,13aS,14aR,16aS)-6-[(cyclopropylacetyl)amino]-2-{[2-(1-methyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate (120 mg, 0.18 mmol, 1.0 equiv) and potassium trimethylsilanolate (100 mg, 0.78 mmol, 4.4 equiv) in ether (6 mL) was stirred for 41 hours. A white solid was collected by filtration, which was dissolved in dichloromethane. The organic layer washed with 0.5M sodium citrate (pH 4.5), brine, dried over magnesium sulfate, filtered and concentrated in vacuo, which gave a white solid from MTBE-hexanes (64 mg, 54% yield): ¹H NMR (400 MHz, DMSO-d6) δ 12.22 (br. s, 1H), 8.62 (s, 1H), 8.38 (d, J=5.6 Hz, 1H), 7.92 (d, J=7.3 Hz, 1H), 7.62 (d, J=5.3 Hz, 1H), 7.52 (d, J=1.8 Hz, 1H), 7.05 (d, J=2.0 Hz, 1H), 6.04 (br. s, 1H), 5.53-5.46 (m, 1H), 5.30 (t, J=9.7 Hz, 1H), 4.49 (t, J=7.8 Hz, 1H), 4.42-4.33 (m, 2H), 4.32 (s, 3H), 4.06 (dd, J=11.6, 4.0 Hz, 1H), 2.46-2.43 (m, 2H), 2.31-2.17 (m, 1H), 1.88-1.78 (m, 4H), 1.50-1.45 (m, 2H), 1.34-1.08 (m, 8H), 0.68-0.61 (m, 1H), 0.25-0.19 (m, 2H), −0.03-(−0.09) (m, 2H); LCMS (ESI+) C₃₃H₃₉N₇O₆S m/z 661 (M+H)⁺; Anal. calcd. for C₃₃H₃₉N₇O₆S•1.0H₂O: C, 58.31; H, 6.08; N, 14.42. Found: C, 58.32; H, 5.85; N, 14.17.

### Example 105

### Methyl (2R,6S,12Z,13aS,14aR,16aS)-6-[(cyclopentylacetyl)amino]-2-{[2-(1-methyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

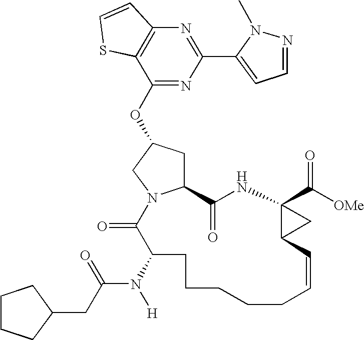

Using the procedure described for Example 15, using methyl (2R,6S,12Z,13aS,14aR,16aS)-6-amino-2-{[2-(1-methyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-amino-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate and cyclopentylacetic acid instead of cyclopropylacetic acid, yielded the title compound of Example 105 as a white solid (135 mg, 91% yield): ¹H NMR (400 MHz, DMSO-d6) δ 8.73 (s, 1H), 8.38 (d, J=5.3 Hz, 1H), 8.00 (d, J=7.6 Hz, 1H), 7.62 (d, J=5.3 Hz, 1H), 7.53 (d, J=1.8 Hz, 1H), 7.04 (d, J=2.0 Hz, 1H), 6.02 (br. s, 1H), 5.56-5.49 (m, 1H), 5.26 (t, J=9.6 Hz, 1H), 4.51-4.47 (m, 2H), 4.32 (s, 3H), 4.02 (dd, J=11.6, 4.0 Hz, 1H), 3.56 (s, 3H), 2.54-2.51 (m, 1H), 2.40-2.34 (m, 1H), 2.29 (q, J=8.8 Hz, 1H), 1.88-1.85 (m, 2H), 1.81-1.67 (m, 3H), 1.57-1.52 (m, 1H), 1.51-1.48 (m, 1H), 1.44-1.23 (m, 14H), 0.89-0.82 (m, 3H); LCMS (ESI+) C₃₆H₄₅N₇O₆S m/z 704 (M+H)⁺.

### Example 106

### (2R,6S,12Z,13aS,14aR,16aS)-6-[(Cyclopentylacetyl)amino]-2-{[2-(1-methyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

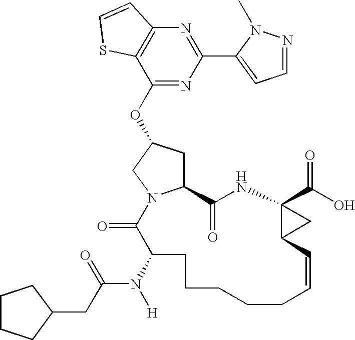

Using the procedure described for Example 104, using methyl (2R,6S,12Z,13aS,14aR,16aS)-6-[(cyclopentylacetyl)amino]-2-{[2-(1-methyl-1H-pyrazol-5-yl)thieno[3,2-d]pyrimidin-4-yl]oxy }-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,6S,12Z,13aS,14aR,16aS)-6-[(cyclopropylacetyl)amino]-2-{[2-(1-methyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 106 as a white solid (44 mg, 38% yield): ¹H NMR (400 MHz, DMSO-d6) δ 12.23 (s, 1H), 8.64 (s, 1H), 8.38 (d, J=5.6 Hz, 1H), 7.99 (d, J=6.3 Hz, 1H), 7.62 (d, J=5.3 Hz, 1H), 7.52 (d, J=2.0 Hz, 1H), 7.04 (d, J=1.8 Hz, 1H), 6.02 (br. s, 1H), 5.53-5.47 (m, 1H), 5.28 (t, J=9.6 Hz, 1H), 4.49-4.46 (m, 2H), 4.32 (s, 3H), 4.05-4.02 (m, 1H), 2.59-2.53 (m, 1H), 2.42-2.40 (m, 2H), 2.26-2.21 (m, 1H), 1.88-1.86 (m, 2H), 1.81-1.67 (m, 3H), 1.49-1.15 (m, 16H), 0.93-0.82 (m, 2H); LCMS (ESI+) C₃₅H₄₃N₇O₆S m/z 690 (M+H)⁺; Anal. calcd. for C₃₅H₄₃N₇O₆S•0.6H₂O•0.3 MTBE: C, 60.30; H, 6.63; N, 13.48. Found: C, 59.97; H, 6.43; N, 13.22.

### Example 107

### Methyl (2R,6S,12Z,13aS,14aR,16aS)-6-{[(2S)-2-hydroxy-3-methylbutanoyl]-amino}-2-{[2-(1-methyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

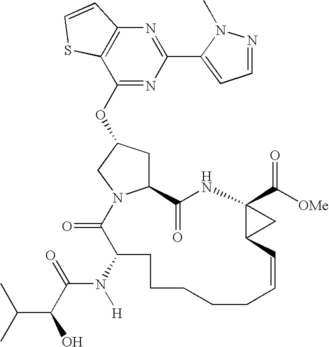

Using the procedure described for Example 15, using methyl (2R,6S,12Z,13aS,14aR,16aS)-6-amino-2-{[2-(1-methyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-amino-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate and (2S)-2-hydroxy-3-methylbutanoic acid instead of cyclopropylacetic acid, yielded the title compound of Example 107 as a white solid (36 mg, 52% yield): ¹H NMR (400 MHz, DMSO-d6) δ 8.79 (s, 1H), 8.39 (d, J=5.3 Hz, 1H), 7.68 (d, J=7.3 Hz, 1H), 7.64 (d, J=5.3 Hz, 1H), 7.53 (s, 1H), 7.05 (s, 1H), 6.06 (br. s, 1H), 5.56-5.49 (m, 1H), 5.30 (t, J=9.8 Hz, 1H), 5.19 (d, J=6.1 Hz, 1H), 5.53 (t, J=7.6 Hz, 2H), 4.33-4.28 (m, 4H), 4.08 (dd, J=11.8, 4.2 Hz, 1H), 3.57 (s, 3H), 3.56-3.52 (m, 1H), 2.47-2.37 (m, 2H), 2.20 (q, J=8.8 Hz, 1H), 1.95-1.87 (m, 1H), 1.78-1.75 (m, 2H), 1.57-1.13 (m, 10H), 0.76 (d, J=6.8 Hz, 3H), 0.64 (d, J=6.8 Hz, 3H); LCMS (ESI+) C₃₄H₄₃N₇O₇S m/z 694 (M+H)⁺.

### Example 108

### (2R,6S,12Z,13aS,14aR,16aS)-6-{[(2S)-2-Hydroxy-3-methylbutanoyl]amino}-2-{[2-(1-methyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

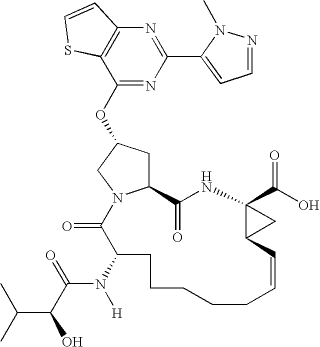

A mixture of methyl (2R,6S,12Z,13aS,14aR,16aS)-6-{[(2S)-2-hydroxy-3-methylbutanoyl]amino}-2-{[2-(1-methyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate (35 mg, 0.05 mmol, 1.0 equiv) and potassium trimethylsilanolate (19 mg, 0.15 mmol, 3.0 equiv) in ether (2 mL) and THF (1 mL) was stirred for 6 hours. A white solid was collected by filtration, which was dissolved in dichloromethane. The organic layer washed with 0.5M sodium citrate (pH 4.5), brine, dried over magnesium sulfate, filtered and concentrated in vacuo, which gave a white solid from MTBE-hexanes (18 mg, 53% yield): ¹H NMR (400 MHz, DMSO-d6) δ12.03 (br. s, 1H), 8.69 (s, 1H), 8.39 (d, J=5.3 Hz, 1H), 7.68 (d, J=7.6 Hz, 1H), 7.63 (d, J=5.6 Hz, 1H), 7.53 (s, 1H), 7.05 (s, 1H), 6.05 (br. s, 1H), 5.53-5.47 (m, 1H), 5.33 (t, J=10.0 Hz, 1H), 5.22 (d, J=6.1 Hz, 1H), 4.54-4.50 (m, 2H), 4.32 (s, 3H), 4.28 (d, J=11.9 Hz, 1H), 4.08 (dd, J=11.5, 3.9 Hz, 1H), 3.58-3.53 (m, 1H), 2.44-2.32 (m, 1H), 2.15 (q, J=8.6 Hz, 1H), 1.95-1.90 (m, 1H), 1.81-1.73 (m, 2H), 1.49-1.14 (m, 11H), 0.76 (d, J=6.8 Hz, 3H), 0.63 (d, J=6.6 Hz, 3H); LCMS (ESI+) C₃₃H₄₁N₇O₇S m/z 680 (M+H)⁺.

### Example 109

### Methyl (2R,6S,12Z,13aS,14aR,16aS)-6-{[(cyclopentyloxy)carbonyl]amino}-2-{[2-(1-methyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

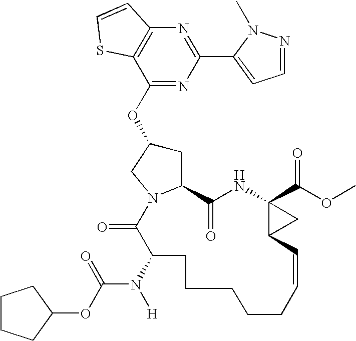

Using the procedure described for Example 40, using methyl (2R,12Z,13aS,14aR,16aS)-6-amino-2-{[2-(1-methyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,6S,12Z,13aS,14aR,16aS)-6-amino-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 109 as a white solid (0.136 g, 76% yield): ¹H NMR (400 MHz, DMSO-d6) δ 8.74 (s, 1H), 8.37 (d, J=5.3 Hz, 1H), 7.62 (d, J=5.3 Hz, 1H), 7.53 (d, J=2 Hz, 1H), 7.20 (d, J=7.1 Hz, 1H), 7.05 (d, J=1.8 Hz, 1H), 6.01 (s, 1H), 5.56-5.49 (m, 1H), 5.25 (t, J=9.6 Hz, 1H), 4.56-4.50 (m, 2H), 4.32 (s, 4H), 4.04-3.92 (m, 2H), 3.56 (s, 3H), 2.44-2.20 (m, 3H), 1.87-1.19 (m, 20H); LCMS (ESI+) for C₃₅H₄₃N₇O₇S m/z 706 (M+H)⁺.

### Example 110

### (2R,6S,12Z,13aS,14aR,16aS)-6{[(cyclopentyloxy)carbonyl]amino}-2-{[2-(1-methyl-1H-pyrazol-5-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

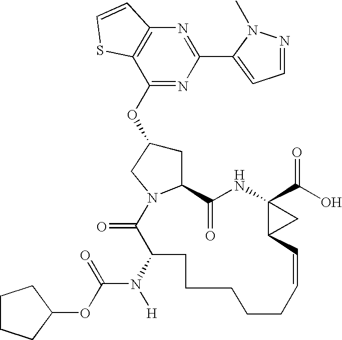

Using the procedure described for Example 108, using methyl (2R,6S,12Z,13aS,14aR,16aS)-6-{[(cyclopentyloxy)carbonyl]amino}-2-{[2-(1-methyl-1H-pyrazol-5-yl)thieno[3,2-d]pyrimidin-4-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,6S,12Z,13aS,14aR,16aS)-6-{[(2S)-2-hydroxy-3-methylbutanoyl]amino}-2-{[2-(1-methyl-1H-pyrazol-5-yl)thieno[3,2-d]pyrimidin-4-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylate, gave a crude residue which washed with 0.5 M sodium citrate buffer (pH=4.5), brine, dried over magnesium sulfate, filtered and concentrated in vacuo to yielded the title compound of Example 110 as a white solid (0.103 g, 84% yield): ¹H NMR (400 MHz, DMSO-d6) δ 12.19 (br. s, 1H), 8.63 (s, 1H), 8.38 (d, J=5.3 Hz, 1H), 7.62 (d, J=5.3 Hz, 1H), 7.52 (d, J=1.8 Hz, 1H), 7.18 (d, J=7.1 Hz, 1H), 7.05 (d, J=1.8 Hz, 1H), 6.01 (s, 1H), 5.52-5.46 (m, 1H), 5.27 (t, J=9.6 Hz, 1H), 4.55-4.49 (m, 2H), 4.32 (s, 4H), 4.04-3.95 (m, 2H), 2.33-2.45 (m, 2H), 2.07-2.25 (m, 1H), 1.83-1.60 (m, 2H), 1.57-1.20 (m, 18H); LCMS (ESI+) for C₃₄H₄₁N₇O₇S m/z 692 (M+H)⁺. Anal. calcd. for C₃₄H₄₁N₇O₇S•0.11 MTBE•1.17H₂O•0.48 EtOAc: C, 57.27; H, 6.39; N, 12.82. Found: C, 57.26; H, 6.06; N, 12.82.

### Example 111

### 1,3-oxazole-2-carboxamide

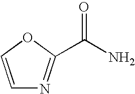

Concentrated ammonium hydroxide (30 mL) was added to ethyl 1,3-oxazole-2-carboxylate (J & W Pharmlab; 1.5 g, 10.6 mmol). The resultant cloudy suspension was stirred overnight and gave a white slurry. A white solid was collected (0.98 g, 82% yield): ¹H NMR (400 MHz, DMSO-d6) δ 8.28 (s, 1H), 8.21 (br. s, 1H), 7.87 (br. s, 1H), 7.42 (s, 1H).

### Example 112

### 1,3-Oxazole-2-carbonitrile

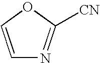

1,3-Oxazole-2-carboxamide (0.98 g, 8.8 mmol. 1.0 equiv) was dissolved in pyridine (17 mL) and treated with POCl₃ (1.2 mL, 12.2 mmol, 1.4 equiv). The resultant beige slurry, which gave a brown solution, was stirred for 5 hours at room temperature. The reaction mixture was diluted with ice and the aqueous layer, which was adjusted to pH 3 with 6M HCl, was extracted with ether. The ether extract washed with water, brine, dried over magnesium sulfate, filtered, concentrated in vacuo and gave the product as an amber oil (0.61 g, 74% yield): ¹H NMR (400 MHz, DMSO-d6) δ 8.608 (s, 1H), 7.67 (s, 1H).

### Example 113

### 2-(1,3-Oxazol-2-yl)thieno[3,2-d]pyrimidin-4-ol

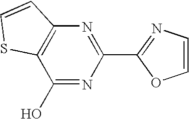

Methyl 3-aminothiophene-2-carboxylate (0.95 g, 6.1 mmol, 1.0 equiv) and 1,3-oxazole-2-carbonitrile (0.57 g, 6.1 mmol, 1.0 equiv) were taken up in anhydrous tetrahydrofuran (24 mL). The resultant solution was cooled to 0° C. and treated with potassium tert-butoxide (1.0 g, 9.2 mmol, 1.5 equiv). The resultant yellow slurry was stirred for 2 h, concentrated in vacuo and poured into saturated ammonium chloride (100 mL). An off-white solid was collected by filtration, which was triturated with MTBE, and gave an off-white (0.56 g, 43% yield) product: ¹H NMR (400 MHz, DMSO-d6) δ 8.24 (s, 1H), 7.94 (d, J=5.3 Hz, 1H), 7.41 (s, 1H), 7.70 (br. s, 1H), 7.32 (d, J=5.3 Hz, 1H); LCMS (ESI+) C₉H₅N₃O₂S m/z 220 (M+H)⁺.

### Example 114

### Methyl (2R,6S,12Z,13aS,14aR,16aS)-6-amino-2-{[2-(1,3-oxazol-2-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

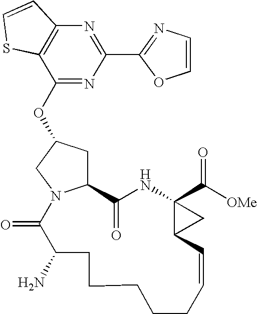

A mixture of methyl (2S,6S,12Z,13aS,14aR,16aS)-6-[(tert-butoxycarbonyl)amino]-2-hydroxy-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.87 g, 1.8 mmol, 1.0 equiv), 2-(1,3-oxazol-2-yl)thieno[3,2-d]pyrimidin-4-ol (0.39 g, 1.8 mmol, 1.0 equiv) and triphenylphosphine (1.4 g, 5.4 mmol, 3.0 equiv) in tetrahydrofuran (36 mL) was treated with diisopropylazodicarboxylate (1.0 mL, 5.4 mmol, 3.0 equiv). The reaction mixture, which became homogeneous within several minutes, was stirred for 24 hours and concentrated in vacuo. Analysis of the crude product by LCMS (ESI+) gave mostly product (681; M+H)⁺. The crude product (an amber oil) was dissolved in dichloromethane (9 mL) and treated with TFA (9 mL). The reaction mixture was concentrated in vacuo after 1.0 hour and the resultant oil was dissolved in ethyl acetate. The organic layer was extracted with 1.2 M HCl. The aqueous extract washed with ethyl acetate and the combined ethyl acetate extracts were discarded. The aqueous layer was saturated with sodium bicarbonate and extracted with dichloromethane. The dichloromethane layer washed with 5% NaHCO₃, brine, dried over MgSO₄ and filtered. The solvent was removed in vacuo and a white solid was collected from CH₂Cl₂-MTBE-hexanes (0.43 g, 43% yield): ¹H NMR (400 MHz, DMSO-d6) δ 8.69 (s, 1H), 8.46 (d, J=5.3 Hz, 1H), 8.37 (s, 1H), 7.72 (d, J=5.3 Hz, 1H), 7.53 (s, 1H), 6.04 (br. s, 1H), 5.54-5.47 (m, 1H), 5.28 (t, J=9.8 Hz, 1H), 4.52 (t, J=7.7 Hz, 1H), 4.07-4.01 (m, 1H), 3.58-3.54 (m, 3H), 3.48 (dd, J=8.2, 2.2 Hz, 1H), 3.32 (s, 3H), 2.46-2.42 (m, 2H), 2.40-2.28 (m, 2H), 1.98-1.85 (m, 2H), 1.56-1.47 (m, 2H), 1.25-1.21 (m, 7H); LCMS (ESI+) C₂₈H₃₂N₆O₆S m/z 581 (M+H)⁺.

### Example 115

### Methyl (2R,6S,12Z,13aS,14aR,16aS)-6-{[(cyclopentyloxy)carbonyl]amino}-2-{[2-(1,3-oxazol-2-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

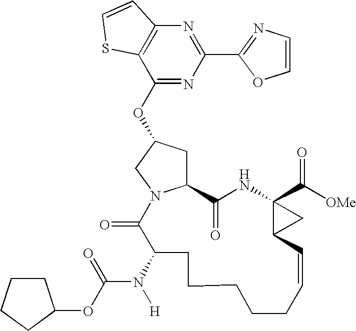

Using the procedure described for Example 10, using methyl (2R,6S,12Z,13aS,14aR,16aS)-6-amino-2-{[2-(1,3-oxazol-2-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-amino-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 115 as a white solid (85 mg, 56% yield): ¹H NMR (400 MHz, DMSO-d6) δ 8.69 (s, 1H), 8.46 (d, J=5.3 Hz, 1H), 8.37 (s, 1H), 7.72 (d, J=5.3 Hz, 1H), 7.53 (s, 1H), 6.04 (br. s, 1H), 5.54-5.47 (m, 1H), 5.28 (t, J=9.8 Hz, 1H), 4.52 (t, J=7.7 Hz, 1H), 4.07-4.01 (m, 1H), 3.58-3.54 (m, 3H), 3.48 (dd, J=8.2, 2.2 Hz, 1H), 3.32 (s, 3H), 2.46-2.42 (m, 2H), 2.40-2.28 (m, 2H), 1.98-1.85 (m, 2H), 1.56-1.47 (m, 2H), 1.25-1.21 (m, 7H); LCMS (ESI+) C₃₄H₄₀N₆O₈S m/z 693 (M+H)⁺.

### Example 116

### (2R,6S,12Z,13aS,14aR,16aS)-6-{[(Cyclopentyloxy)carbonyl]amino}-2-{[2-(1,3-oxazol-2-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

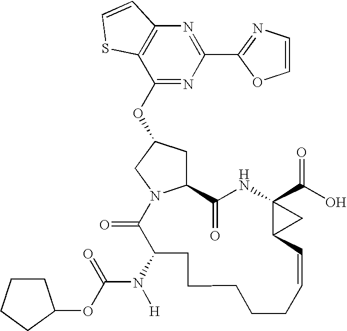

Using the procedure described for Example 108, using methyl (2R,6S,12Z,13aS,14aR,16aS)-6-{[(cyclopentyloxy)carbonyl]amino}-2-{[2-(1,3-oxazol-2-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of (2R,6S,12Z,13aS,14aR,16aS)-6-{[(2S)-2-hydroxy-3-methylbutanoyl]amino}-2-{[2-(1-methyl-1H-pyrazol-5-yl)thieno[3,2-d]pyrimidin-4-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 116 as a white solid (8 mg, 5% yield): ¹H NMR (400 MHz, DMSO-d6) δ 8.62 (br. s, 1H), 8.42 (d, J=5.3 Hz, 1H), 8.37 (s, 1H), 7.69 (d, J=5.6 Hz, 1H), 7.52 (s, 1H), 7.15 (d, J=6.6 Hz, 1H), 6.01 (br. s, 1H), 5.47 (br. s, 1H), 5.30 (br. s, 1H), 4.55-4.48 (m, 2H), 4.27 (br. s, 1H), 4.00-3.92 (m, 2H), 2.44-2.32 (m, 2H), 2.15 (br. s, 1H), 1.73-1.68 (m, 2H), 1.49-1.17 (m, 18H); MS (ESI+) C₃₃H₃₈N₆O₈S m/z 679 (M+H)⁺.

### Example 117

### Methyl (2R,6S,12Z,13aS,14aR,16aS)-6-[(cyclopropylacetyl)amino]-2-{[2-(1,3-oxazol-2-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

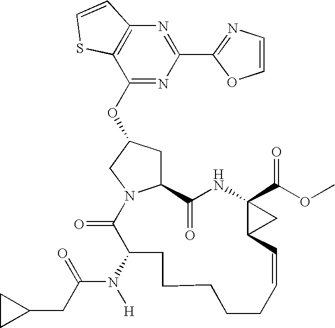

Using the procedure described for Example 15, using methyl (2R,6S,12Z,13aS,14aR,16aS)-6-amino-2-{[2-(1,3-oxazol-2-yl)thieno[3,2-d]pyrimidin-4-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-amino-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 117 as a white residue (0.090 g, 84% yield): ¹H NMR (400 MHz, DMSO-d6) δ 8.73 (s, 1H), 8.43 (d, J=5.3 Hz, 1H), 8.37 (s, 1H), 7.92 (d, J=7.3 Hz, 1H), 7.70 (d, J=5.3 Hz, 1H), 7.52 (s, 1H), 6.05 (br. s, 1H), 5.56-5.49 (m, 1H), 5.27 (t, J=9.7 Hz, 1H), 4.50 (t, J=8.0 Hz, 1H), 4.40 (d, J=11.3 Hz, 1H), 4.33-4.29 (m, 1H), 4.04 (dd, J=11.5, 3.9 Hz, 1H), 3.57 (s, 3H), 2.33-2.23 (m, 1H), 1.85-1.73 (m, 4H), 1.57-1.47 (m, 2H), 1.44-1.11 (m, 10H), 0.68-0.58 (m, 1H), 0.24-0.20 (m, 2H), −0.06-(−0.09) (m, 2H); LCMS (ESI+) for C₃₃H₃₈N₆O₇S m/z 663 (M+H)⁺.

### Example 118

### (2R,6S,12Z,13aS,14aR,16aS)-6-[(cyclopropylacetyl)amino]-2-{[2-(1,3-oxazol-2-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

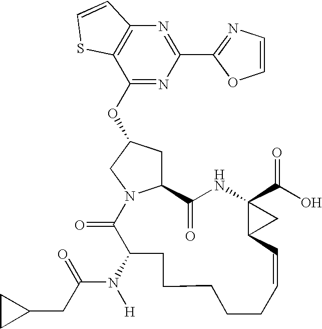

Using the procedure described for Example 108, using methyl (2R,6S,12Z,13aS,14aR,16aS)-6-[(cyclopropylacetyl)amino]-2-{[2-(1,3-oxazol-2-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of (2R,6S,12Z,13aS,14aR,16aS)-6-{[(2S)-2-hydroxy-3-methylbutanoyl]amino}-2-{[2-(1-methyl-1H-pyrazol-5-yl)thieno[3,2-d]pyrimidin-4-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylate, yielded an off-white solid. The crude product was purified by reversed phase chromatography (C18) and eluted with 5-95% acetonitrile in water (50 mM NH₄₀ Ac/acetonitrile) which yielded the title compound of Example 118 as a white solid (0.046 g, 56% yield): ¹H NMR (400 MHz, DMSO-d6) δ 12.14 (br. s, 1H), 8.67 (s, 1H), 8.437 (d, J=5.3 Hz, 1H), 8.37 (s, 1H), 7.93 (d, J=7.3 Hz, 1H), 7.70 (d, J=5.3 Hz, 1H), 7.53 (s, 1H), 6.05 (s, 1H), 5.54-5.47 (m, 1H), 5.29 (t, J=9.7 Hz, 1H), 4.50-4.43 (m, 2H), 4.31 (t, J=8.6 Hz, 1H), 4.05-4.01 (m, 1H), 3.90-3.88 (m, 1H), 3.57-2.54 (m, 1H), 2.20 (q, J=8.7 Hz, 1H), 1.86-1.74 (m, 4H), 1.55-1.26 (m, 10H), 0.66-0.61 (m, 1H), 0.22 (d, J=7.3 Hz, 2H), −0.07 (d, J=4.0 Hz, 2H); LCMS (ESI+) for C₃₂H₃₆N₆O₇ m/z 649 (M+H)⁺.

### Example 119

### 5-Methyl-isoxazole-3-carboxylic acid amide

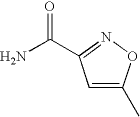

Using the procedure described for Example 47, using 5-methyl-isoxazole-3-carboxylic acid methyl ester (Avocado) instead of 2,5-dimethyl-2H-pyrazole-3-carboxylic acid ethyl ester, yielded the title compound of Example 119 as a white solid (5.9 g, 66% yield): ¹H NMR (400 MHz, DMSO-d6) δ 8.01 (br. s, 1H), 7.73 (br. s, 1H), 6.48 (s, 1H), 2.44 (s, 3H); LCMS (ESI+) C₅H₆N₂O₂ m/z 127 (M+H)⁺.

### Example 120

### 5-Methyl-isoxazole-3-carbonitrile

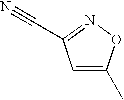

Using the procedure described for Example 48, using 5-methyl-isoxazole-3-carboxylic acid amide instead of 1,3-dimethyl-1H-pyrazole-5-carboxamide, yielded the title compound of Example 120 as a light amber oil (4.8 g, 96% yield): ¹H NMR (400 MHz, DMSO-d6) δ 6.98 (s, 1H), 2.53 (s, 3H).

### Example 121

### 2-(5-methylisoxazol-3-yl)thieno[3,2-d]pyrimidin-4-ol

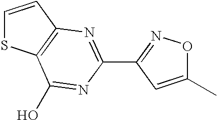

Methyl 3-aminothiophene-2-carboxylate (6.8 g, 44 mmol, 1.0 equiv) and 5-methyl-isoxazole-3-carbonitrile (4.7 g, 44 mmol, 1.0 equiv) were taken up in anhydrous tetrahydrofuran (170 mL). The resultant solution was cooled to 0° C. and treated with potassium tert-butoxide (7.3 g, 65 mmol, 1.5 equiv). The reaction mixture, which was a thick slurry after 30 minutes, was diluted with 100 mL of THF. The resultant slurry was stirred for 2 h, concentrated in vacuo and poured into 50% saturated ammonium chloride (100 mL) and extracted with MTBE. The organic layer washed with brine, dried over magnesium sulfate, filtered and concentrated in vacuo. The resultant solid was triturated with MTBE and gave an off-white (6.1 g, 60% yield) product: ¹H NMR (400 MHz, DMSO-d6) δ 12.90 (br. s, 1H), 8.22 (d, J=5.3 Hz, 1H), 7.48 (d, J=5.3 Hz, 1H), 6.82 (d, J=1.0 Hz, 1H), 2.51 (d, J=0.8 Hz, 3H); LCMS (ESI+) C₁₀H₇N₃O₂S m/z 234 (M+H)⁺.

### Example 122

### Methyl (4R)-4-{[2-(5-methylisoxazol-3-yl)thieno[3,2-d]pyrimidin-4-yl]oxy}-L-prolinate

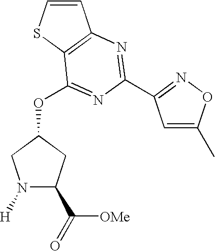

2-(5-Methylisoxazol-3-yl)thieno[3,2-d]pyrimidin-4-ol (3.0 g, 13 mmol, 1.0 equiv), c is 4-hydroxy-pyrrolidine-1,2-dicarboxylic acid 1-tert-butyl ester (3.2 g, 13 mmol, 1.0 equiv) and triphenylphosphine (6.8 g, 26 mmol, 2.0 equiv) were taken up in anhydrous tetrahydrofuran (250 mL). The amber solution was cooled to 0° C. and treated with DIAD (5.0 mL, 26 mmol, 2.0 equiv). The light orange solution was gradually warmed to ambient temperature and stirred for 15 h. The reaction mixture was analyzed by LCMS (ESI+) and gave the product [C₂₁H₂₄N₄O₆S m/z 461 (M+H)⁺] and no starting materials. The solvent was concentrated in vacuo, and the resultant amber oil was dissolved in MTBE and washed with water and brine. The organic layer was dried over magnesium sulfate, filtered, concentrated in vacuo and gave an amber oil (20 g). The crude product was purified over silica (500 g), eluted with 0-5% methanol-dichloromethane and gave 10 g of an impure product as an amber oil. The crude product was dissolved in dichloromethane (25 mL) and treated with trifluoroacetic acid (25 mL). The amber solution was stirred for two hours at ambient temperature. The solvent was removed in vacuo and the resultant amber oil was dissolved in dichloromethane. The organic layer washed with 50% saturated sodium bicarbonate, brine and extracted with 1.2M HCl. The acidic extract washed with dichloromethane. The combined organic extracts were discarded. The acidic aqueous layer was saturated with sodium bicarbonate and extracted with dichloromethane. The dichloromethane layer washed with brine, dried over magnesium sulfate, filtered, concentrated in vacuo and yielded the title compound of Example 122 as a white solid from MTBE (2.6 g, 2-step 56% yield): ¹H NMR (400 MHz, DMSO-d6) δ 8.42 (d, J=5.3 Hz, 1H), 7.67 (d, J=5.3 Hz, 1H), 6.84 (d, J=1.0 Hz, 1H), 5.81-5.80 (m, 1H), 3.96 (t, J=7.6 Hz, 1H), 3.65 (s, 3H), 3.37 (dd, J=12.4, 5.0 Hz, 1H), 3.08 (dd, J=12.4, 2.0 Hz, 1H), 2.50 (d, J=1.0 Hz, 3H), 2.34-2.31 (m, 2H); LCMS (ESI+) C₁₆H₁₆N₄O₄S m/z 361 (M+H)⁺.

### Example 123

### Methyl (4R)-1-{(2S)-2-[(tert-butoxycarbonyl)amino]non-8-enoyl})₄-{[2-(5-methylisoxazol-3-yl)thieno[3,2-d]pyrimidin-4-yl]oxy}-L-prolinate

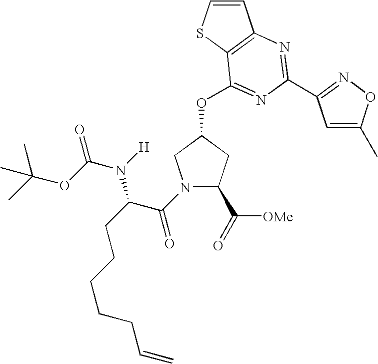

Using the procedure described for methyl Example 7, using methyl (4R)-4-{[2-(5-methylisoxazol-3-yl)thieno[3,2-d]pyrimidin-4-yl]oxy}-L-prolinate instead of methyl (1R,2S)-1-({(4R)-4-[(5-pyridin-2-ylthieno[3,2-b]pyridin-7-yl)oxy]-L-prolyl }amino)-2-vinylcyclopropanecarboxylate, yielded the title compound of Example 123 as a white foam from MTBE-hexanes (3.5 g, 80% yield): ¹H NMR (400 MHz, DMSO-d6) δ 8.44-8.41 (m, 1H), 7.69-7.65 (m, 1H), 7.00 (d, J=7.6 Hz, 1H), 6.84 (s, 1H), 5.94 (br. s, 1H), 5.83-5.73 (m, 1H), 5.01-4.91 (m, 2H), 4.53 (t, J=8.3 Hz, 1H), 4.39 (d, J=12.1 Hz, 1H), 4.10-3.99 (m, 2H), 3.65 (s, 3H), 2.70-2.64 (m, 1H), 2.50 (d, J=0.8 Hz, 3H), 2.39-2.32 (m, 2H), 2.02-1.97 (m, 2H), 1.59-1.53 (m, 1H), 1.46-1.41 (m, 1H), 1.34-1.29 (m, 5H), 1.14-1.10 (m, 9H); LCMS (ESI+) C₃₀H₃₉N₅O₇S m/z 614 (M+H)⁺.

### Example 124

### Potassium (2S,4R)-1-{(2S)-2-[(tert-butoxycarbonyl)amino]non-8-enoyl}-4-{([2-(5-methylisoxazol-3-yl)thieno[3,2-d]pyrimidin-4-yl]oxy}pyrrolidine-2-carboxylate

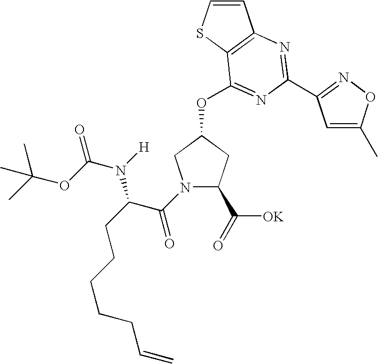

Using the procedure described for Example 98, using methyl (4R)-1-{(2S)-2-[(tert-butoxycarbonyl)amino]non-8-enoyl}-4-{[2-(5-methylisoxazol-3-yl)thieno[3,2-d]pyrimidin-4-yl]oxy}-L-prolinate instead of 1-(2-tert-butoxycarbonylamino-non-8-enoyl)-4-[2-(2-methyl-2H-pyrazol-3-yl)-thieno[3,2-d]pyrimidin-4-yloxy]-pyrrolidine-2-carboxylic acid methyl ester, yielded the title compound of Example 124 as a white solid (3.3 g, 100% yield): LCMS (ESI+) C₂₉H₃₇KN₅O₇S m/z 600 (M+H)⁺.

### Example 125

### Methyl (1R,2S)-1-[((4R)-1-{(2S)-2-[(tert-butoxycarbonyl)amino]non-8-enoyl}-4-{([2-(5-methylisoxazol-3-yl)thieno[3,2-d]pyrimidin-4-yl]oxy}-L-prolyl)amino]-2-vinylcyclopropanecarboxylate

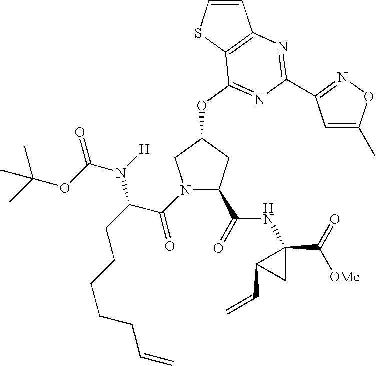

Using the procedure described for Example 20, using potassium (2S,4R)-1-{(2S)-2-[(tert-butoxycarbonyl)amino]non-8-enoyl }-4-{[2-(5-methylisoxazol-3-yl)thieno[3,2-d]pyrimidin-4-yl]oxy}pyrrolidine-2-carboxylate instead of (4R)-1-(Tert-butoxycarbonyl)-4-[(2-pyridin-2-ylthieno[3,2-d]pyrimidin-4-yl)oxy]-L-proline, yielded the title compound of Example 125 as a beige solid (2.5 g, 69% yield): ¹H NMR (400 MHz, DMSO-d6) δ 8.89 (s, 0.25H), 8.65 (s, 0.75H), 8.45-8.41 (m, 1H), 7.70-7.66 (m, 1H), 7.03 (d, J=7.3 Hz, 0.75H), 6.86-6.85 (m, 1H), 6.69 (d, J=8.0 Hz, 0.25H), 6.02-5.81 (m, 1H), 5.80-5.71 (m, 1H), 5.68-5.59 (m, 1H), 5.29-5.10 (m, 1H), 5.09 (dd, J=10.2, 1.9 Hz, 1H), 5.00-4.88 (m, 2H), 4.50 (t, J=7.3 Hz, 0.25H), 4.44 (t, J=8.0 Hz, 0.75H), 4.26 (d, J=11.6 Hz, 1H), 4.06-3.99 (m, 2H), 3.60 (s, 0.75H), 3.58 (s, 2.25H), 2.51 (s, 3H), 2.37-2.28 (m, 1H), 2.11-2.05 (m, 1H), 1.64-1.40 (m, 3H), 1.36-1.18 (m, 10H), 1.13-1.09 (m, 9H); LCMS (ESI+) C₃₆H₄₆N₆O₈S m/z 723 (M+H)⁺.

### Example 126

### Methyl (2R,6S,12Z,13aS,14aR,16aS)-6-[(tert-butoxycarbonyl)amino]-2-{[2-(5-methylisoxazol-3-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

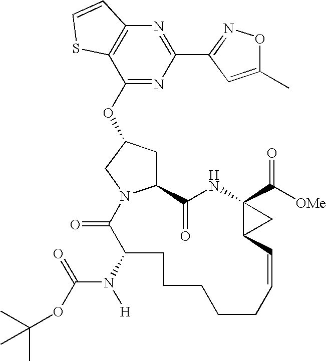

Using the procedure described for methyl Example 8, using methyl (1R,2S)-1-[((4R)-1-{(2S)-2-[(tert-butoxycarbonyl)amino]non-8-enoyl}-4-{[2-(5-methylisoxazol-3-yl)thieno[3,2-d]pyrimidin-4-yl]oxy}-L-prolyl)amino]-2-vinylcyclopropanecarboxylate instead of methyl (1R,2S)-1-({(4R)-1-{2-[(ter-butoxycarbonyl)amino]non-8-enoyl}-4-[(5-pyridin-2-ylthieno[3,2-b]pyridin-7-yl)oxy]-L-prolyl}amino)-2-vinylcyclopropanecarboxylate yielded the title compound of Example 126 as an off-white solid (193 mg, 9% yield): ¹H NMR (400 MHz, DMSO-d6) δ 8.76 (s, 1H), 8.38 (d, J=5.0 Hz, 1H), 7.65 (d, J=5.0 Hz, 1H), 6.97 (d, J=6.6 Hz, 1H), 6.86 (s, 1H), 5.96 (br. s, 1H), 5.56-5.49 (m, 1H), 5.25 (t, J=9.4 Hz, 1H), 4.65-4.62 (m, 1H), 4.52 (t, J=8.1 Hz, 1H), 3.94-3.89 (m, 2H), 3.56 (s, 3H), 2.51 (s, 3H), 2.41-2.39 (m, 1H), 2.24-2.20 (m, 1H), 1.74-1.67 (m, 2H), 1.57-1.54 (m, 1H), 1.51-1.47 (m, 1H), 1.36-1.27 (m, 6H), 1.13-1.09 (m, 4H), 0.98 (s, 8H); LCMS (ESI+) C₃₄H₄₂N₆O₈S m/z 695 (M+H)⁺.

### Example 127

### Methyl (2R,6S,12Z,13aS,14aR,16aS)-6-amino-2-{[2-(5-methylisoxazol-3-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

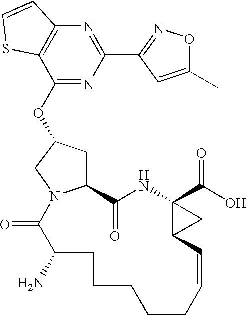

Using the procedure described for Example 9, using methyl (2R,6S,12Z,13aS,14aR,16aS)-6-[(tert-butoxycarbonyl)amino]-2-{[2-(5-methylisoxazol-3-yl)thieno[3,2-d]pyrimidin-4-yl]oxy }-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-[(tert-butoxycarbonyl)amino]-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 127 as a white solid (124 mg, 78% yield): ¹H NMR (400 MHz, DMSO-d6) δ 8.22 (d, J=5.3 Hz, 1H), 7.57 (d, J=5.3 Hz, 1H), 6.84 (d, J=1.0 Hz, 1H), 6.15 (br. s, 1H), 5.63-5.56 (m, 1H), 5.37 (t, J=9.8 Hz, 1H), 4.71 (t, J=7.8 Hz, 1H), 4.22-4.15 (m, 2H), 3.88-3.85 (m, 1H), 3.67 (s, 3H), 2.67-2.61 (m, 2H), 2.54 (s, 3H), 2.36-2.30 (m, 2H), 2.16-2.09 (m, 1H), 1.77-1.60 (m, 4H), 1.55-1.22 (m, 9H).

### Example 128

### Methyl (2R,6S,12Z,13aS,14aR,16aS)-6-{[(cyclopentyloxy)carbonyl]amino}-2-{([2-(5-methylisoxazol-3-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

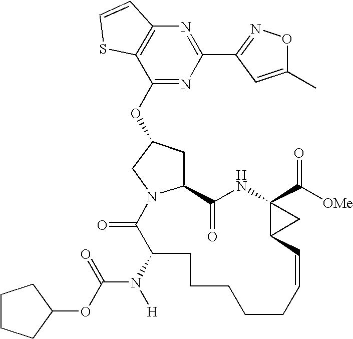

Using the procedure described for Example 10, using methyl (2R,6S,12Z,13aS,14aR,16aS)-6-amino-2-{[2-(5-methylisoxazol-3-yl)thieno[3,2-d]pyrimidin-4-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-amino-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 128 as a white solid (42 mg, 58% yield): ¹H NMR (400 MHz, CD₃OD) δ 8.20 (d, J=5.3 Hz, 1H), 7.56 (d, J=5.3 Hz, 1H), 6.87 (s, 1H), 6.13 (br. s, 1H), 5.63-5.56 (m, 1H), 5.33 (t, J=9.6 Hz, 1H), 4.74-4.67 (m, 2H), 4.39 (br. s, 1H), 4.19 (dd, J=10.4, 2.8 Hz, 1H), 4.08 (dd, J=11.9, 3.8 Hz, 1H), 3.67 (s, 3H), 2.68-2.63 (m, 1H), 2.59-2.55 (m, 4H), 2.50-2.44 (m, 1H), 1.96-1.91 (m, 1H), 1.82-1.71 (m, 1H), 1.67-1.21 (m, 18H); LCMS (ESI+) C₃₅H₄₂N₆O₈S m/z 707 (M+H)⁺.

### Example 129

### (2R,6S,12Z,13aS,14aR,16aS)-6-{[(Cyclopentyloxy)carbonyl]amino}-2-{[2-(5-methylisoxazol-3-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

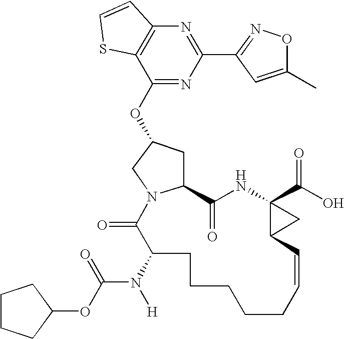

Using the procedure described for Example 14, using methyl (2R,6S,12Z,13aS,14aR,16aS)-6-{[(cyclopentyloxy)carbonyl]amino }-2-{[2-(5-methylisoxazol-3-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-[(tert-butoxycarbonyl)amino]-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 129 as a white solid (9 mg, 21% yield): ¹H NMR (400 MHz, CD₃OD) δ 8.74 (s, 1H), 8.20 (d, J=5.6 Hz, 1H), 7.56 (d, J=5.6 Hz, 1H), 6.87 (s, 1H), 6.13 (br. s, 1H), 5.61-5.56 (m, 1H), 5.35 (t, J=9.7 Hz, 1H), 4.75-4.67 (m, 2H), 4.36 (s, 1H), 4.18-4.15 (m, 1H), 2.66-2.63 (m, 1H), 2.59-2.55 (m, 5H), 2.36-2.30 (m, 1H), 1.98-1.95 (m, 1H), 1.84-1.75 (m, 1H), 1.66-1.38 (m, 18H); LCMS (ESI+) C₃₄H₄₀N₆O₈S m/z 693 (M+H)⁺.

### Example 130

### Methyl (2R,6S,12Z,13aS,14aR,16aS)-6-[(cyclopropylacetyl)amino]-2-{[2-(5-methylisoxazol-3-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

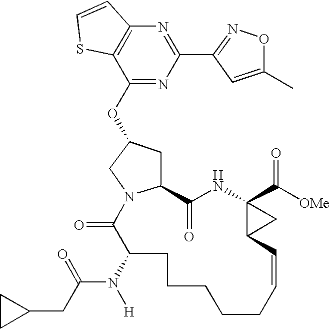

Using the procedure described for Example 15, using methyl (2R,6S,12Z,13aS,14aR,16aS)-6-amino-2-{[2-(5-methylisoxazol-3-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-amino-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 130 as a white solid from MTBE-hexanes (42 mg, 62% yield): ¹H NMR (400 MHz, DMSO-d6) δ 8.73 (s, 1H), 8.42 (d, J=5.3 Hz, 1H), 7.93 (d, J=7.3 Hz, 1H), 7.64 (d, J=5.3 Hz, 1H), 6.87 (d, J=0.8 Hz, 1H), 6.04 (br. s, 1H), 5.56-5.46 (m, 1H), 5.27 (t, J=9.8 Hz, 1H), 4.50 (t, J=7.8 Hz, 1H), 4.40-4.31 (m, 2H), 4.06-4.01 (m, 1H), 3.57 (s, 3H), 2.52 (s, 3H), 2.45-2.38 (m, 2H), 2.32-2.23 (m, 1H), 1.84-1.74 (m, 4H), 1.56-1.53 (m, 1H), 1.51-1.48 (m, 1H), 1.40-1.10 (m, 8H), 0.68-0.62 (m, 1H), 0.25-0.20 (m, 2H), −0.05-(−0.08) (m, 2H); LCMS (ESI+) C₃₄H₄₀N₆O₇S m/z 677 (M+H)⁺.

### Example 131

### (2R,6S,12Z,13aS,14aR,16aS)-6-[(Cyclopropylacetyl)amino]-2-{[2-(5-methylisoxazol-3-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

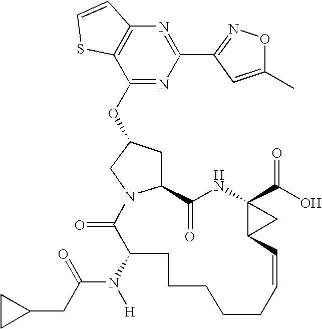

Using the procedure described for Example 108, using methyl (2R,6S,12Z,13aS,14aR,16aS)-6-[(cyclopropylacetyl)amino]-2-{[2-(5-methylisoxazol-3-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of (2R,6S,12Z,13aS,14aR,16aS)-6-{[(2S)-2-hydroxy-3-methylbutanoyl]amino}-2-{[2-(1-methyl-1H-pyrazol-5-yl)thieno[3,2-d]pyrimidin-4-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 131 as a white solid from MTBE (16 mg, 40% yield): ¹H NMR (400 MHz, DMSO-d6) δ 12.23 (s, 1H), 8.65 (s, 1H), 8.42 (d, J=5.3 Hz, 1H), 7.92 (d, J=7.3 Hz, 1H), 7.67 (d, J=5.3 Hz, 1H), 6.87 (s, 1H), 6.04 (br. s, 1H), 5.54-5.47 (m, 1H), 5.30 (t, J=9.6 Hz, 1H), 4.48 (t, J=7.8 Hz, 1H), 4.40-4.31 (m, 2H), 4.04 (dd, J=11.6, 4.0 Hz, 1H), 2.50 (s, 3H), 2.45-2.41 (m, 1H), 2.25-2.19 (m, 1H), 1.84-1.74 (m, 4H), 1.50-1.45 (m, 2H), 1.35-1.15 (m, 9H), 0.25-0.20 (m, 2H), 0.67-0.61 (m, 1H), −0.05-(−0.08) (m, 2H); LCMS (ESI+) C₃₃H₃₈N₆O₇S m/z 663 (M+H)⁺.

### Example 132

### 1-Methyl-1H-imidazole-2-carboxamide

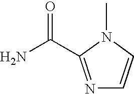

Using the procedure described for Example 47, using ethyl 1-methyl-1H-imidazole-2-carboxylate (Toronto Research Chemicals) instead of 2,5-dimethyl-2H-pyrazole-3-carboxylic acid ethyl ester, yielded the title compound of Example 132 as a white solid (2.16 g, 53% yield): ¹H NMR (400 MHz, DMSO-d6) δ 7.69 (br. s, 1H), 7.37 (br. s, 1H), 7.30 (s, 1H), 6.94 (s, 1H), 3.91 (s, 3H).

### Example 133

### 1-Methyl-1H-imidazole-2-carbonitrile

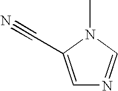

Using the procedure described for Example 48, using 1-methyl-1H-imidazole-2-carboxamide instead of 1,3-dimethyl-1H-pyrazole-5-carboxamide, yielded the title compound of Example 133 as a brown oil (1.29 g, 71% yield): ¹H NMR (400 MHz, DMSO-d6) δ 7.58 (s, 1H), 7.17 (s, 1H), 3.83 (s, 3H).

### Example 134

### 2-(1-Methyl-1H-imidazol-2-yl)thieno[3,2-d]pyrimidin-4-ol

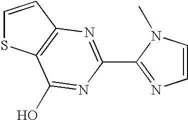

Using the procedure described for Example 32, using 1-methyl-1H-imidazole-2-carbonitrile instead of pyridine-2-carbonitrile, yielded the title compound of Example 134 as a light beige solid (0.527 g, 20% yield): ¹H NMR (400 MHz, DMSO-d6) δ 8.2 (d, J=5.3 Hz, 1H), 7.51 (s, 1H), 7.44 (d, J=5.3, 1H), 7.15 (s, 1H), 4.09 (s, 3H); LCMS (ESI+) for C₁₀H₈N₄OS m/z 233 (M+H)⁺.

### Example 135

### Methyl (2R,12Z,13aS,14aR,16aS)-6-amino-2-{[2-(1-methyl-1H-imidazol-2-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

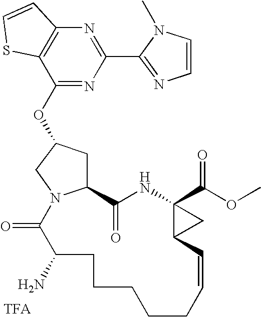

Diisopropylazodicarboxylate (DIAD) (0.511 mL, 2.63 mmol, 2.0 equiv) was added dropwise to a suspension of methyl (2S,12Z,13aS,14aR,16aS)-6-[(tert-butoxycarbonyl)amino]-2-hydroxy-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.630 g, 1.31 mmol, 1.0 equiv), 2-(1-methyl-1H-imidazol-2-yl)thieno[2,3-d]pyrimidin-4-ol (0.306 g, 1.31 mmol, 1.0 equiv) and triphenylphosphine (0.689 g, 2.63 mmol, 2.0 equiv) in anhydrous THF (22 mL, 0.06M) at 0° C. The reaction was warmed to ambient temperature, stirred for 16 h and concentrated in vacuo. The crude residue was taken up in dichloromethane (10 mL) and treated with trifluoroacetic acid (10 mL). The reaction mixture was stirred for 1 h and then concentrated in vacuo. Trituration from MTBE/hexanes afforded the title compound of Example 135 as a tan solid (0.576 g, 74%): ¹H NMR (400 MHz, DMSO-d6) δ 8.86 (s, 1H), 8.39 (d, J=5.3 Hz, 1H), 7.63 (d, J=5.3 Hz, 1H), 7.39 (s, 1H), 7.07 (d, J=1.0 Hz, 1H), 6.04 (br. s, 1H), 5.54-5.47 (m, 1H), 5.28 (t, J=9.6 Hz, 1H), 4.79-4.73 (m, 1H), 4.5 (t, J=7.7 Hz, 1H), 4.08 (s, 3H), 4.06-3.98 (m, 2H), 3.56 (s, 3H), 3.54-3.47 (m, 2H), 2.46-2.40 (m, 2H), 2.38-2.27 (m, 2H), 2.22 (q, J=9.1 Hz, 1H), 1.97-1.85 (m, 1H), 1.56-1.47 (m, 4H), 1.29-1.20 (m, 4H); LCMS (ESI+) for C₂₉H₃₅N₇O₅S m/z 594 (M+H)⁺.

### Example 136

### Methyl (2R,6S,12Z,13aS,14aR,16aS)-6-{[(cyclopentyloxy)carbonyl]amino}-2-{[2-(1-methyl-1H-imidazol-2-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

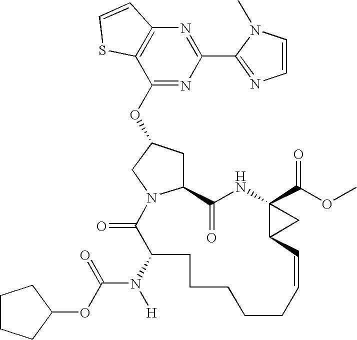

Using the procedure described for Example 40, using methyl (2R,12Z,13aS,14aR,16aS)-6-amino-2-{[2-(1-methyl-1H-imidazol-2-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,6S,12Z,13aS,14aR,16aS)-6-amino-5,16-dioxo-2-[(2-pyridin-2-ylthieno[3,2-d]pyrimidin-4-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 136 as an off-white solid (0.033 g, 28% yield): ¹H NMR (400 MHz, DMSO-d6) δ 8.73 (s, 1H), 8.36 (d, J=5.3 Hz, 1H), 7.61 (d, J=5.3 Hz, 1H), 7.38 (s, 1H), 7.19 (d, J=7.1 Hz, 1H), 7.07 (d, J=1.0 Hz, 1H), 6.00 (br. s, 1H), 5.56-5.49 (m, 1H), 5.25 (t, J=9.6 Hz, 1H), 4.61-4.44 (m, 2H), 4.33 (d, J=3.5 Hz, 1H), 4.08 (s, 3H), 3.98-3.85 (m, 1H), 3.56 (s, 3H), 2.42-2.22 (m, 2H), 1.71-1.20 (m, 21H), 1.14-1.07 (m, 1H); LCMS (ESI+) for C₃₅H₄₃N₇O₇S m/z 706 (M+H)⁺.

### Example 137

### Methyl (2R,6S,12Z,13aS,14aR,16aS)-6-[(cyclopentylacetyl)amino]-2-{[2-(1-methyl-1H-imidazol-2-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

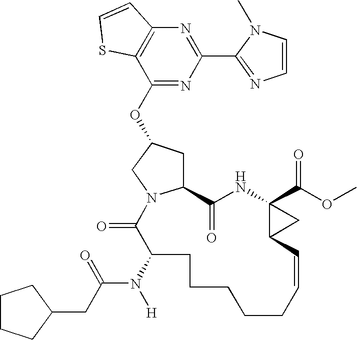

Using the procedure described for Example 15, using methyl (2R,12Z,13aS,14aR,16aS)-6-amino-2-{[2-(1-methyl-1H-imidazol-2-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-amino-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate and cyclopentylacetic acid instead of cyclopropylacetic acid, yielded the title compound of Example 137 as a tan solid (0.032 g, 27% yield): ¹H NMR (400 MHz, DMSO-d6) δ 8.73 (s, 1H), 8.38 (d, J=5.3 Hz, 1H), 8.01 (d, J=7.8 Hz, 1H), 7.62 (d, J=5.3 Hz, 1H), 6.41 (s, 1H), 7.11 (s, 1H), 6.02 (br. s, 1H), 5.56-5.49 (m, 1H), 5.28-5.26 (m, 1H), 4.51-4.47 (m, 2H), 4.32 (t, J=9.1 Hz, 1H), 4.10 (s, 3H), 4.01 (dd, J=11.6, 3.8 Hz, 1H), 3.56 (s, 1H), 2.41-2.26 (m, 2H), 1.91-1.68 (m, 5H), 1.53-1.15 (m, 19H), 0.91-0.84 (m, 2H); LCMS (ESI+) for C₃₆H₄₅N₇O₆S m/z 704 (M+H)⁺.

### Example 138

### Methyl (2R,6S,12Z,13aS,14aR,16aS)-6-[(cyclopropylacetyl)amino]-2-{[2-(1-methyl-1H-imidazol-2-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

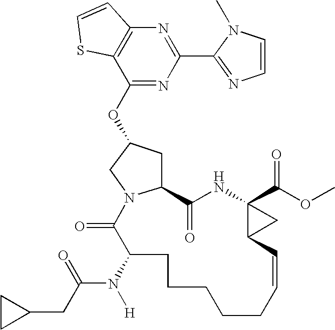

Using the procedure described for Example 15, using methyl (2R,12Z,13aS,14aR,16aS)-6-amino-2-{[2-(1-methyl-1H-imidazol-2-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of methyl (2R,12Z,13aS,14aR,16aS)-6-amino-5,16-dioxo-2-[(5-pyridin-2-ylthieno[3,2-b]pyridin-7-yl)oxy]-1,2,3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 138 as an off-white solid (0.062 g, 61% yield): ¹H NMR (400 MHz, DMSO-d6) δ 8.73 (s, 1H), 8.36 (d, J=5.6 Hz, 1H), 7.94 (d, J=7.3 Hz, 1H), 7.61 (d, J=5.6 Hz, 1H), 7.39 (s, 1H), 7.07 (s, 1H), 6.04 (br. s, 1H), 5.56-5.49 (m, 1H), 5.27 (t, J=9.6 Hz, 1H), 4.50 (t, J=8.0 Hz, 1H), 4.42-4.32 (m, 2H), 4.09 (s, 3H), 4.03 (dd, J=11.7, 4.2 Hz, 1H), 3.56 (s, 3H), 2.31-2.24 (m, 1H), 1.85-1.78 (m, 4H), 1.58-1.23 (m, 12H), 0.67-0.63 (m, 1H), 0.25-0.18 (m, 2H), −0.05-(−0.09) (m, 2H); LCMS (ESI+) for C₃₄H₄₁N₇O₆S m/z 676 (M+H)⁺.

### Example 139

### (2R,12Z,13aS,14aR,16aS)-6-{[(cyclopentyloxy)carbonyl]amino}-2-{[2-(1-methyl-1H-imidazol-2-yl)thieno[3,2-d]pyrimidin-4-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylic acid

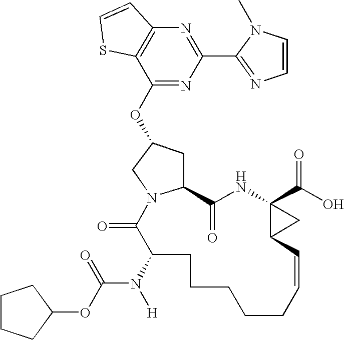

Using the procedure described for Example 108, using methyl (2R,6S,12Z,13aS,14aR,16aS)-6-{[(cyclopentyloxy)carbonyl]amino}-2-{[2-(1-methyl-1H-imidazol-2-yl)thieno[3,2-d]pyrimidin-4-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylate instead of (2R,6S,12Z,13aS,14aR,16aS)-6-{[(2S)-2-hydroxy-3-methylbutanoyl]amino}-2-{[2-(1-methyl-1H-pyrazol-5-yl)thieno[3,2-d]pyrimidin-4-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 139 as an off-white solid (0.01 g, 34% yield): ¹H NMR (400 MHz, DMSO-d6) δ 8.58 (s, 1H), 8.36 (d, J=5.3 Hz, 1H), 7.62 (d, J=5.7 Hz, 1H), 7.39 (s, 1H), 7.14-7.09 (m, 3H), 6.02 (s, 1H), 5.46-5.45 (m, 1H), 5.28-5.29 (m, 1H), 4.53-4.39 (m, 3H), 4.09-3.96 (m, 4H), 2.21-1.23 (m, 24H); LCMS (ESI+) for C₃₄H₄₁N₇O₇S m/z 692 (M+H)⁺.

### Example 140

### (2R,6S,12Z,13aS,14aR,16aS)-6-[(cyclopentylacetyl)amino]-2-{[2-(1-methyl-1H-imidazol-2-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

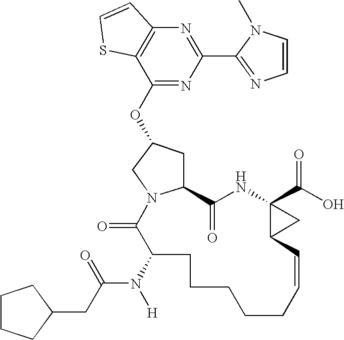

Using the procedure described for Example 108, using methyl (2R,6S,12Z,13aS,14aR,16aS)-6-[(cyclopentylacetyl)amino]-2-{[2-(1-methyl-1H-imidazol-2-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of (2R,6S,12Z,13aS,14aR,16aS)-6-{[(2S)-2-hydroxy-3-methylbutanoyl]amino}-2-{[2-(1-methyl-1H-pyrazol-5-yl)thieno[3,2-d]pyrimidin-4-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 140 as an off-white solid (0.030 g, 61% yield): ¹H NMR (400 MHz, DMSO-d6) δ 12.57 (br. s, 1H), 8.63 (s, 1H), 8.37 (d, J=5.3 Hz, 1H), 8.00 (d, J=7.8 Hz, 1H), 7.62 (d, J=5.3 Hz, 1H), 7.39 (s, 1H), 7.09 (s, 1H), 6.04 (s, 1H), 5.53-5.47 (m, 1H), 5.28 (t, J=10.1 Hz, 1H), 4.48 (t, J=7.8 Hz, 1H), 4.42-4.35 (m, 2H), 4.09 (s, 3H), 2.38-2.18 (m, 2H), 2.10-2.00 (m, 1H), 1.93-1.71 (m, 6H), 1.56-1.29 (m, 18H); LCMS (ESI+) for C₃₅H₄₃N₇O₆S m/z 690 (M+H)⁺. Anal. calcd. for C₃₅H₄₃N₇O₆S•0.36 DCM•1.08H₂O•0.88 Hexanes: C, 59.84; H, 7.19; N, 12.02. Found: C, 59.86; H, 6.84; N, 11.62

### Example 141

### (2R,6S,12Z,13aS,14aR,16aS)-6-[(cyclopropylacetyl)amino]-2-{[2-(1-methyl-1H-imidazol-2-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylic acid

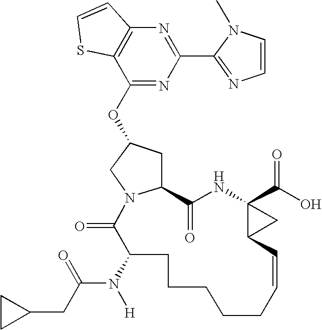

Using the procedure described for Example 108, using methyl (2R,6S,12Z,13aS,14aR,16aS)-6-[(cyclopropylacetyl)amino]-2-{[2-(1-methyl-1H-imidazol-2-yl)thieno[3,2-d]pyrimidin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate instead of (2R,6S,12Z,13aS,14aR,16aS)-6-{[(2S)-2-hydroxy-3-methylbutanoyl]amino}-2-{[2-(1-methyl-1H-pyrazol-5-yl)thieno[3,2-d]pyrimidin-4-yl]oxy}-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylate, yielded the title compound of Example 141 as an off-white solid. (0.017 g, 31% yield): ¹H NMR (400 MHz, DMSO-d6) δ 12.57 (br. s, 1H), 8.62 (s, 1H), 8.37 (d, J=5.3 Hz, 1H), 7.92 (d, J=7.3 Hz, 1H), 7.62 (d, J=5.3 Hz, 1H), 7.39 (s, 1H), 7.10 (s, 1H), 6.07 (s, 1H), 5.52-5.46 (m, 1H), 5.30 (t, J=9.7 Hz, 1H), 4.50 (t, J=7.4 Hz, 1H), 4.39-4.31 (m, 2H), 4.10 (s, 3H), 2.20 (d, J=8.9 Hz, 1H), 1.94-1.66 (m, 4H), 1.53-1.19 (m, 13H), 0.73-0.62 (m, 1H), 0.23 (d, J=8.1 Hz, 2H), −0.05 (d, J=4.5 Hz, 2H); LCMS (ESI+) for C₃₃H₃₉N₇O₆S m/z 662 (M+H)⁺.

### Example 142

### (4S)-1-(Tert-butoxycarbonyl)-4-hydroxy-L-proline tert-butyl dimethyl silyl ether

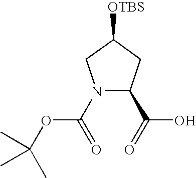

(4S)-1-(Tert-butoxycarbonyl)-4-hydroxy-L-proline (14.4 g, 62 mmol, 1.0 equiv) and imidazole (21.1 g, 310 mmol, 5 equiv) were dissolved in dichloromethane (100 mL) and N,N-dimethylformamide (DMF) (20 mL). Tert-butyl chlorodimethyl silane (20.6 g, 137 mmol, 2.2 equiv) was added and the reaction mixture was stirred for 2 h at room temperature. The reaction mixture was poured into water (600 mL), the dichloromethane layer was withdrawn, concentrated in vacuo and then taken up in 20% ether/hexanes. The organic layer was washed with brine and concentrated in vacuo. The crude product was dissolved in methanol (80 mL) and a solution of lithium hydroxide monohydrate (4.4 g, 105 mmol, 1.7 equiv) in water (100 mL) was added. The homogeneous mixture was stirred at ambient temperature for 2 h. The reaction mixture was poured into water (600 mL) and acidified to pH 3.0 using 1N hydrochloric acid (HCl). The aqueous layer was extracted three times with 10% ether/hexanes. The organic layer washed with brine and concentrated in vacuo for 18 h which gave the title compound of Example 142 as a solid. (22.0 g, 100% yield): LCMS (ESI+) for C₁₆H₃₁NO₅Si m/z 346 (M+H)⁺.

### Example 143

### Tert-butyl (2S,4S)-4-hydroxy-2-({[(1R,2S)-1-(methoxycarbonyl)-2-vinylcyclopropyl]amino}carbonyl)pyrrolidine-1-carboxylate tert-butyl dimethyl ether

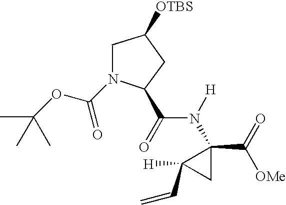

Using the procedure described for Example 5 and the compound of Example 142 instead of the compound of Example 4 yielded the title compound of Example 143 as an off-white foam (13.3 g, 100% yield): ¹H NMR (400 MHz, DMSO-d6) δ 8.51 (s, 1H), 5.69-5.49 (m, 1H), 5.29-5.19 (m, 1H), 5.11-5.05 (m, 1H), 4.38-4.30 (m, 1H), 3.97 (t, J=8.0 Hz, 1H), 3.61-3.57 (m, 4H), 3.17-3.02 (m, 1H), 3.14-3.04 (m, 1H), 2.43-2.29 (m, 1H), 2.17-2.10 (m, 1H), 1.72-1.64 (m, 2H), 1.37 (s, 2H), 1.31 (s, 7H), 0.84 (s, 9H), 0.04 (s, 6H); LCMS (ESI+) for C₂₃H₄₀N₂O₆Si m/z 469 (M+H)⁺.

### Example 144

### Methyl (1R,2S)-1-{[(4S)-4-hydroxy-L-prolyl]amino}-2-vinylcyclopropanecarboxylate hydrochloride

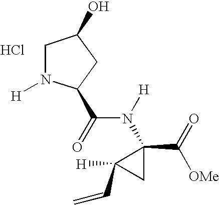

4N HCl in dioxane (15 mL) was added to the compound of Example 143 in dioxane (15 mL) and stirred at ambient temperature for 2 h. The reaction mixture was concentrated in vacuo and gave a white solid (3.44 g, 100% yield): ¹H NMR (400 MHz, DMSO-d6) δ 9.23 (s, 1H), 5.70-5.55 (m, 1H), 5.30 (d, J=17.2 Hz, 1H), 5.24 (s, 1H), 5.12 (d, J=10.1 Hz, 1H), 4.33 (s, 1H), 4.15 (s, 1H), 3.61 (s, 3H), 3.24-3.16 (m, 1H), 3.14-3.04 (m, 1H), 2.22 (q, J=8.8 Hz, 1H), 1.95-1.82 (m, 1H), 1.69 (t, J=6.8 Hz, 1H), 1.33 (dd, J=9.6, 5.1 Hz, 1H); LCMS (ESI+) for C₁₂H₁₈N₂O₄ m/z 255 (M+H)⁺.

### Example 145

### Methyl (1R,2S)-1-[((4S)-1-{(2S)-2-[(3,3-dimethylbutanoyl)amino]non-8-enoyl}-4-hydroxy-L-prolyl)amino]-2-vinylcyclopropanecarboxylate

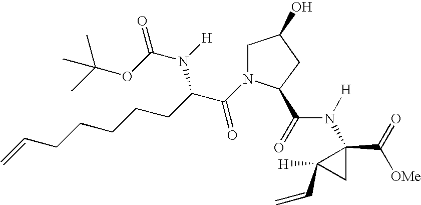

Using the procedure described for the compound of Example 7 and using the compound of Example 144 instead of the compound of Example 6 yielded the title compound of Example 145 as an amber oil (13.4 g, 88% yield): ¹H NMR (400 MHz, DMSO-d6) δ 8.67-8.56 (m, 1H), 6.95 (d, J=7.3 Hz, 0.75H), 6.68 (d, J=7.1 Hz, 0.15H), 6.53 (br., s, 0.1H), 5.83-5.73 (m, 1H), 5.65-5.56 (m, 1H), 5.30-4.91 (m, 5H), 4.22-3.81 (m, 4H), 3.56 (s, 3H), 2.32-2.27 (m, 1H), 2.08-1.98 (m, 3H), 1.73-1.66 (m, 1H), 1.61-1.58 (m, 2H), 1.51-1.16 (m, 18H); LCMS (ESI+) for C₂₆H₄₁N₃O₇ m/z 508 (M+H)⁺.

### Example 146

### Methyl (2S,6S,12Z,13aS,14aR,16aS)-6-[(tert-butoxycarbonyl)amino]-2-hydroxy-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

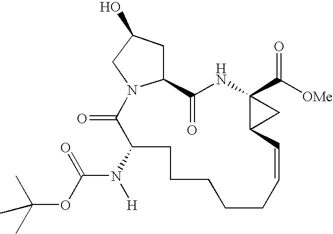

Using the procedure described for the compound of Example 8 and using the compound of Example 145 instead of the compound of Example 7 and the Hoveyda-Grubbs Catalyst 2^{nd} Generation (CAS# 301224-40-8) instead of the Grubbs Catalyst 2^{nd} Generation gave the title compound of Example 146 as an off-white solid (4.8 g, 43% yield): ¹H NMR (400 MHz, DMSO-d6) δ 8.81 (s, 1H), 6.92 (d, J=6.6 Hz, 1H), 5.56-5.49 (m, 2H), 5.25 (t, J=9.7 Hz, 1H), 4.26-4.20 (m, 2H), 4.13-4.08 (m, 1H), 3.95-3.91 (m, 1H), 3.35 (s, 3H), 3.39-3.36 (m, 1H), 2.44-2.40 (m, 1H), 2.32-2.26 (m, 1H), 2.11-2.07 (m, 1H), 1.79-1.74 (m, 2H), 1.64-1.60 (m, 1H), 1.57-1.52 (m, 1H), 1.50-1.47 (m, 1H), 1.33-1.15 (m, 16H); LCMS (ESI+) for C₂₄H₃₇N₃O₇ m/z 479 (M+H)⁺.

### Example 147

### 7-Methyl-2-pyridin-2-ylthieno[3,2-d]pyrimidin-4-ol

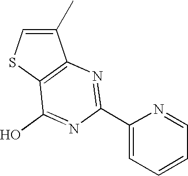

Methyl 3-amino-4-methylthiophene-2-carboxylate (5 g, 29 mmol 1 eq) and 2-cyanopyridine (3 g, 29 mmol, 1 eq) in 30 mL HCl-dioxane (4M, 120 mmol 4 eq) was warmed to 85° C. for 18 hours. The reaction mixture was poured into ice and made basic with ammonium hydroxide. The resultant solid was collected by filtration and purified over silica gel (2-10% methanol-dichloromethane), which provided the product as a beige solid (4 g, 57% yield): ¹H NMR (400 MHz, DMSO-d₆) δ 11.54 (br., 1H), 8.72-8.45 (m, 1H), 8.15-8.11 (m, 1H), 7.99-7.87 (m, 1H), 7.85-7.68 (m, 1H), 7.60-7.43 (m, 1H), 2.47 (s, 3H); LCMS (ESI+) for C₁₂H₉N₃OS m/z 244(M+H)⁺.

### Example 148

### Methyl (2R,6S,12Z,13aS,14aR,16aS)-6-amino-2-[(7-methyl-2-pyridin-2-ylthieno[3,2-d]pyrimidin-4-yl)oxy]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

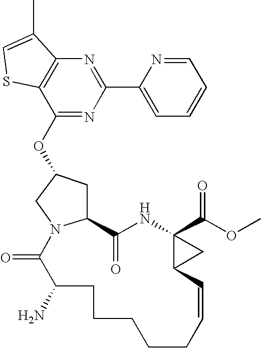

Diisopropylazodicarboxylate (DIAD) (0.17 mL, 0.87 mmol, 2.0 equiv) was added dropwise to a solution of methyl (2S,12Z,13aS,14aR,16aS)-6-[(tert-butoxycarbonyl)amino]-2-hydroxy-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate (0.21 g, 0.43 mmol, 1.0 equiv), 7-methyl-2-pyridin-2-ylthieno[3,2-d]pyrimidin-4-ol (0.105 g, 0.43 mmol, 1.0 equiv) and triphenylphosphine (0.23 g, 0.87 mmol, 2.0 equiv) in anhydrous THF (10 mL). The reaction was stirred for 18 h and concentrated in vacuo. The crude residue was dissolved in dichloromethane (1 mL) and trifluoroacetic acid (1 mL). The reaction mixture was stirred for 1.5 h, concentrated in vacuo and dissolved in ethyl acetate. The organic layer was extracted with 1.2 M HCl. The aqueous extract washed with ethyl acetate and the combined ethyl acetate extracts were discarded. The aqueous layer was saturated with solid sodium bicarbonate and extracted with dichloromethane. The dichloromethane layer washed with 5% NaHCO₃, brine, dried over MgSO₄, filtered and concentrated in vacuo, and gave the product as a white solid (0.116 g, 45% yield): ¹H NMR (400 MHz, DMSO-d6) δ 8.82-8.80 (m, 1H), 8.70 (s, 1H), 8.53-8.51 (d, J=7.8 Hz, 1H), 8.06-8.00 (m, 2H), 7.57-7.54 (m, 1H), 6.13 (br. s, 1H), 5.51-5.49 (m, 1H), 5.29 (t, J=9.6 Hz, 11H), 4.60-4.56 (m, 1H), 4.54-4.51 (m, 1H), 4.13-4.05 (m, 2H), 3.58 (s, 3H), 2.51 (s, 3H), 2.44-2.36 (m, 3H), 1.97-1.11 (m, 13H); LCMS (APCI+) for C₃₀H₃₄N₆O₅S m/z 591 (M+H)⁺.

### Example 149

### Methyl (2R,6S,12Z,13aS,14aR,16aS)-6-{[(cyclopentyloxy)carbonyl]amino }-2-[(7-methyl-2-pyridin-2-ylthieno[3,2-d]pyrimidin-4-yl)oxy]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxylate

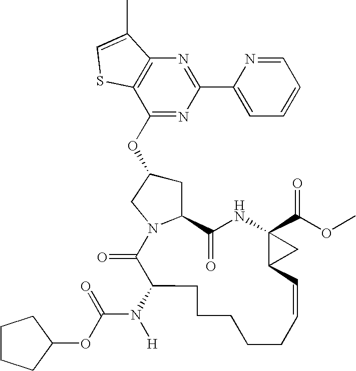

Using the procedure described for Example 40 and using the compound of Example 148 instead of the compound of Example 39 yielded the title compound of Example 149 as an off-white solid (0.107 g, 91% yield): ¹H NMR (400 MHz, DMSO-d6) δ 8.80-8.75 (m, 2H), 8.53-8.51 (d, J=7.8 Hz, 1H), 8.13-8.10 (m, 1H), 8.03-7.99 (m, 1H), 7.57-7.54 (m, 1H), 7.22-7.15 (m, 1H), 6.94-6.90 (m, 1H), 6.11 (br. s, 1H), 5.56-5.50 (m, 1H), 5.27 (t, J=9.8 Hz, 1H), 4.56-4.48 (m, 2H), 4.36 (br. s, 1H), 4.08-397 (m, 1H), 3.58 (s, 3H), 2.49 (s, 3H), 2.42-2.20 (m, 2H), 1.80-1.11 (m, 21H); LCMS (APCI+) for C₃₇H₄₄N₆O₇S m/z 717 (M+H)⁺.

### Example 150

### (2R,6S,12Z,13aS,14aR,16aS)-6-{[(cyclopentyloxy)carbonyl]amino}-2-[(7-methyl-2-pyridin-2-ylthieno[3,2-d]pyrimidin-4-yl)oxy]-5,16-dioxo-1,2, 3,6,7,8,9,10,11,13a,14,15,16,16a-tetradecahydrocyclopropa[e]pyrrolo[1,2-a][1,4]diazacyclopentadecine-14a(5H)-carboxylic acid

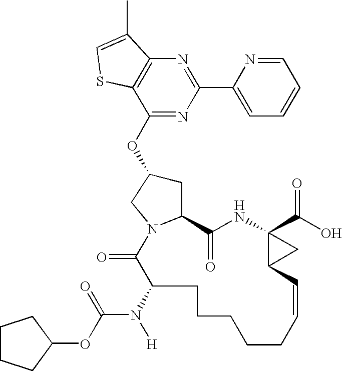

Using the procedure described for Example 108 and using the compound of Example 149 instead of the compound of Example 107 yielded the title compound of Example 150 as a white solid (0.028 g, 35% yield): ¹H NMR (400 MHz, DMSO-d6) δ 12.25 (br. s, 1H), 8.80 (d, J=4.3 Hz, 1H), 8.68 (s, 1H), 8.52 (d, J=8.08 Hz, 1H), 8.04-8.00 (m, 2H), 7.57-7.54 (m, 1H), 7.19 (d, J=7.1 Hz, 1H), 6.11 (br. s, 1H), 5.55-5.48 (m, 1H), 5.28 (t, J=9.8 Hz, 1H), 4.54-4.48 (m, 2H), 4.40-4.33 (m, 1H), 4.06-3.96 (m, 2H), 2.48 (s, 3H), 2.45-2.35 (m, 1H), 2.33-2.20 (m, 1H), 1.83-1.08 (m, 21H); LCMS (APCI) for C₃₆H₄₂N₆O₇S m/z 703 (M+H); Anal. calcd. for C₃₆H₄₂N₆O₇•1.05H₂O: C, 56.75; H, 6.42; N, 11.03. Found: C, 56.87; H, 6.52; N, 10.89.

HCV Protease Assay

HCV protease activity and compound inhibition was monitored using a continuous, fluorescence resonance energy transfer (FRET) assay. Test compounds at various concentrations were added to assay buffer (50 mM MOPS pH 7.5, 50 mM NaCl, 20% glycerol, 0.025% Triton-X 100, 1 mM tris(2-carboxyethyl)phosphine) containing 3 uM depsipeptide FRET substrate S1 (Anaspec) (see Taliani et al. Analytical Biochemistry, 240, 60-67 (1996)) in a white, non-binding 96-well plate (Corning). The reaction was started by the addition of 3 nM full-length NS3-NS4A enzyme. The increase in fluorescence intensity following peptide cleavage was monitored using a Safire fluorescence plate reader (Tecan). The excitation and emission wavelengths were 340 nm and 500 nm, respectively. Inhibition constants (K_{I}) were calculated by non-linear regression analysis using an equation derived for competitive inhibition.

Antiviral Activity

The compounds described herein were tested for antiviral activity utilizing a dual-reporter replicon assay as described in U.S. patent application Ser. No. 10/818,075 (the '075 application), filed Apr. 5, 2004. The disclosure of the '075 application is incorporated herein.

Cell-based antiviral activity was evaluated in the previously described HCV dual reporter subgenomic replicon system (the '075 application). With this system, the effects of a compound on both HCV replication and cell viability can be simultaneously determined by measuring the intracellular levels of two separate reporter proteins. The dicistronic selectable replicon contains the Renilla luciferase gene such that Renilla luciferase activity within stably-transfected cells serves as a marker of HCV replication. The firefly luciferase gene is stably integrated into and expressed by the Huh-7 host cells. Since firefly luciferase activity is dependent upon cellular transcription and translation, firefly luciferase activity serves as an indicator of cell viability.

The dual reporter selectable replicon cell line (B6b) was maintained in DMEM supplemented with 10% FBS, L-glutamine, non-essential amino acids, penicillin, streptomycin, and selection agents (200 ug/ml G418 and 6 ug/ml blastocidin). On the first day of the experiment, cells were trypsinized, washed, and diluted in medium lacking the selection agents. Cells were transferred to 96-well, black-walled, clear bottom plates at a density of 2×10⁴ cells in 150 ul of medium per well. Cells were allowed to settle for 60 to 90 minutes while compounds were being prepared. Each compound was initially diluted to a 4× working stock (2.56% DMSO in tissue culture medium) that was then serially diluted nine times in half log increments in medium with 2.56% DMSO. Fifty microliters of each dilution were then added in triplicate to the plated cells in order to obtain final IX compound concentrations with 0.64% DMSO. Typical testing concentrations ranged from 320 uM to 10 nM. Control wells containing 0.64% DMSO without compound were also included on each plate.

After 3 days of incubation under humidity at 37° C. with 5% CO₂, plates were microscopically examined for compound precipitation and cell death. Tissue culture medium was then aspirated and cells were lysed with 20 ul of 1× Passive Lysis Buffer (Promega, Madison, Wis.). Reporter activity was measured using a MicroBeta Jet 1450 (Perkin Elmer, Boston, Mass.) following sequential 50 ul additions of firefly and Renilla luciferase substrates as described in the manufacturers protocol (Promega). Luciferase activities were expressed as a percentage of the signals observed in the compound-free control wells. The amount of compound required to reduce Renilla luciferase expression by 50% is defined as the 50% effective concentration, or EC₅₀. The amount of compound that reduces firefly luciferase expression by 50% is defined as the 50% cytotoxic concentration, or CC₅₀. A therapeutic index (TI) is calculated by dividing the CC₅₀ by the EC₅₀.

Similarly, an EC₉₀ value is the concentration of the inhibitor at which 50% inhibition of viral replication is achieved, and an analysis of the antiviral component of a data set allows for a calculation of the ninety-percent effective concentration (EC₉₀).

CC₅₀ and EC₅₀ data as determined for exemplary compounds of the invention are presented in Table 1 below.

TABLE 1

| | | HCV | | | |
|---|---|---|---|---|---|
| | | protease | | | |
| | | Full | Replicon | Replicon | |
| | Mol. | Length Ki | EC50 | CC50 | Replicon |
| Compound | Weight | (nM) | (μM) | (μM) | TI |
| Example 79 | 733.9 | 11.3 | 0.18 | 212 +/− 33 | 1200 |
| Example 91 | 660.8 | 91; 70 | 0.29 | >320 | >1100 |
| Example 89 | 690.8 | 14; 13 | 0.50 | >320 | >640 |
| Example 65 | 707.8 | 10.3; 7.9 | 2.6 | >320 | >120 |
| Example 86 | 678.8 | 69 | 15 | >320 | >21 |
| Example 61 | 693.8 | 11.3 | 0.87 | 244 +/− 28 | 280 |
| Example 63 | 675.8 | 8.1 | 0.011 | >320 | >29000 |
| Example 60 | 691.8 | 6.7 | 0.70 | 222 +/− 25 | 320 |
| Example 58 | 705.8 | 2.3 | 0.062 | 216 +/− 16 | 3500 |
| Example 31 | 658.8 | 39 | 0.25 | 168 +/− 6 | 670 |
| Example 28 | 674.8 | 21 | 0.42 | 59 +/− 3 | 140 |
| Example 26 | 688.8 | 8.5 | 0.10 | 117 +/− 23 | 1200 |
| Example 29 | 676.8 | 42 | 0.42 | 13 | 31 |
| Example 46 | 658.8 | 15 | 0.092 | >320 | >3500 |
| Example 11 | 687.8 | 6.6 | 0.11 | 86 +/− 25 | 780 |
| Example 16 | 657.8 | 11 | 0.14 | 251 | 1800 |
| Example 14 | 675.8 | 8.9 | 0.61 | 92 +/− 15 | 150 |
| Example 13 | 673.8 | 9 | 0.31 | 161 +/− 3 | 520 |
| Example 43 | 674.8 | 20 | 0.19 | >320 | >1700 |
| Example 41 | 688.8 | 5.2 | 0.034 | 87 +/− 1 | 2600 |
| Example 44 | 676.8 | 20 | 0.15 | 159 +/− 55 | 1100 |

We claim:

1. A compound of Formula IV: 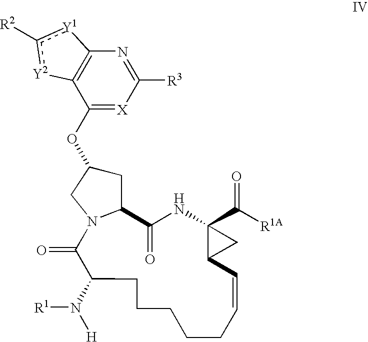
wherein:
R¹ is selected from C₁-C₁₀ alkyl, C₂-C₆ alkenyl, C₂-C₆ alkynyl, —OR⁵, —C(O)R⁵, —C(O)OR⁵, —C(O)NR⁵R⁶, —SO₂NR⁵R⁶, heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;
R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, heteroaryl, —(CR⁷R⁸)ₜ(C6C₁₀ aryl), —(CR⁷R⁸)ₜ(4-10 membered heterocyclic), C₃-C₁₀ cycloalkoxy, and C₃-C₁₀ cycloalkyl, wherein each of said heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;
R² is selected from H, halo, cyano, nitro, azido, C₁-C₆ alkyl, C₂-C₆ alkenyl, C₂-C₅ alkynyl, —C(O)NR⁵R⁶, —OR⁵, —C(O)R⁵, —C(O)OR⁵, —OC(O)R⁵, —NR⁵C(O)R⁶, —NR⁵C(O)NR⁶—NR⁵R⁶, —NR⁵OR⁶, —SO₂NR⁵R⁶, —NR⁵SO₂R⁶—(CR⁷R⁸)ₜ(C₆-C₁₀ aryl), —(CR⁷R⁸)ₜ(4-10 membered heterocyclic), heteroaryl, and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R² groups are optionally substituted with at least one R⁴ group;
R³ is selected from H, halo, cyano, nitro, azido, C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, heteroaryl, —(CR⁷R⁸)ₜ(C₆-C₁₀ aryl), —(CR⁷R⁸)ₜ(4-10 membered heterocyclic), and C₃-C₁₀ cycloalkyl, wherein each of said heteroaryl, —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;
each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;
each R⁵ and R⁶, which may be the same or different, is independently selected from H, C₁-C₆ alkyl, C₃-C₁₀ cycloalkyl, C₃-C₁₀ cycloalkoxy, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);
each R⁷ and R⁸, which may be the same or different, is independently selected from H and C₁-C₆ alkyl;
each t is independently selected from 0, 1, 2, 3, 4, and 5;
X is CH or N; and
Y¹ and Y² are each independently selected from CH, CR¹, O, S, and NR²;
or pharmaceutically acceptable salts or solvates thereof.

2. A compound of Formula IV: 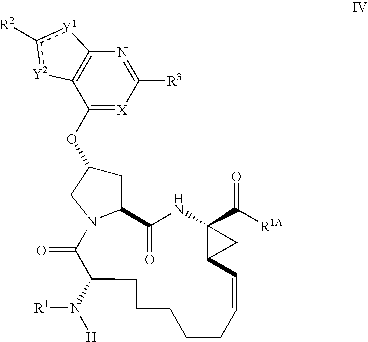
wherein:
R¹ is selected from C₁-C₁₀ alkyl, —OR⁵, —C(O)R⁵, —C(O)OR⁵, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said C₆-C₁₀ aryl, 4-10 membered heterocyclic, C₃-C₁₀ cycloalkoxy, and C₃-C₁₀ cycloalkyl moieties of said R¹ groups are optionally substituted with at least one R⁴ group;
R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;
R² is selected from H, halo, cyano, nitro, azido, C₁-C₆ alkyl, C₂-C₆ alkenyl, C₂-C₆ alkynyl, —C(O)NR⁵R⁶, —OR⁵, —C(O)R⁵, —C(O)OR⁵, —OC(O)R⁵, —NR⁵C(O)R⁶, —NR⁵C(O)NR⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), heteroaryl, and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R² groups are optionally substituted with at least one R⁴ group;
R³ is selected from H, halo, cyano, nitro, azido, C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;
each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;
each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);
each R⁷ and R⁸, which may be the same or different, is independently selected from H and C₁-C₆ alkyl;
each t is independently selected from 0, 1, 2, 3, 4, and 5;
X is CH or N;
Y¹ and Y² are each independently selected from CH, CR¹, O, S, and NR²;
or pharmaceutically acceptable salts or solvates thereof.

3. A compound of Formula IV: 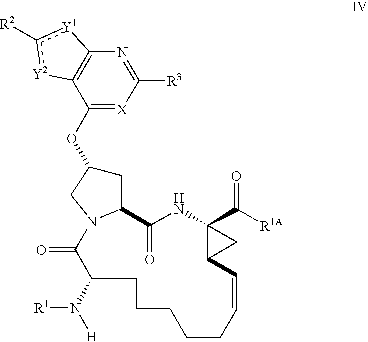
wherein:
R¹ is selected from C₁-C₁₀ alkyl, —OR⁵, C₃-C₁₀ cycloalkoxy, and C₃-C₁₀ cycloalkyl, wherein each of said C₁-C₁₀ alkyl, C₃-C₁₀ cycloalkoxy, and C₃-C₁₀ cycloalkyl moieties of said R¹ groups are optionally substituted with at least one R⁴ group, and wherein at least one carbon in each of said C₁-C₁₀ alkyl, C₃-C₁₀ cycloalkoxy, and C₃-C₁₀ cycloalkyl moieties of said R¹ groups is optionally replaced by —NH—, O or S, with the proviso that said C₁-C₁₀ alkyl, C₃-C₁₀ cycloalkoxy, and C₃-C₁₀ cycloalkyl moieties do not have O—O or S—S bonds;
R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;
R² is selected from H, halo, cyano, nitro, azido, C₁-C₆ alkyl, C₂-C₆ alkenyl, C₂-C₆ alkynyl, —C(O)NR⁵R⁶, —OR⁵, —C(O)R⁵, —C(O)OR⁵, —OC(O)R⁵, —NR⁵C(O)R⁶, —NR⁵C(O)NR⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁵R⁶)ₜ(4-10 membered heterocyclic), heteroaryl, and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R² groups are optionally substituted with at least one R⁴ group;
R³ is selected from H, halo, cyano, nitro, azido, C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶—(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;
each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;
each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);
each R⁷ and R⁸, which may be the same or different, is independently selected from H and C₁-C₆ alkyl;
each t is independently selected from 0, 1, 2, 3, 4, and 5;
X is CH or N;
Y¹ and Y² are each independently selected from CH, CR¹, O, S, and NR²;
or pharmaceutically acceptable salts or solvates thereof.

4. A compound of Formula IV: 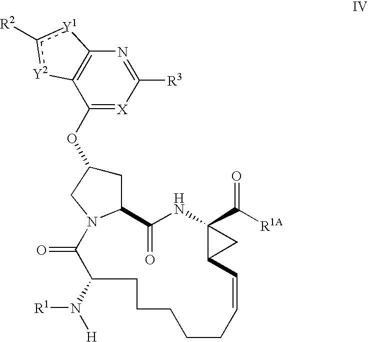
wherein: 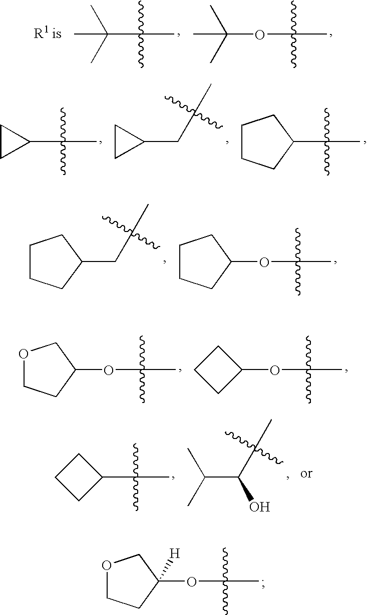
R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;
R² is selected from H, halo, cyano, nitro, azido, C₁-C₆ alkyl, C₂-C₆ alkenyl, C₂-C₆ alkynyl, —C(O)NR⁵R⁶, —OR⁵, —C(O)R⁵, —C(O)OR⁵, —OC(O)R⁵, —NR⁵C(O)R⁶, —NR⁵C(O)NR⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), heteroaryl, and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R² groups are optionally substituted with at least one R⁴ group;
R³ is selected from H, halo, cyano, nitro, azido, C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;
each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;
each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);
each R⁷ and R⁸, which may be the same or different, is independently selected from H and C₁-C₆ alkyl;
each t is independently selected from 0, 1, 2, 3, 4, and 5;
X is CH or N;
Y¹ and Y² are each independently selected from CH, CR¹, O, S, and NR²;
or pharmaceutically acceptable salts or solvates thereof.

5. A compound of Formula I 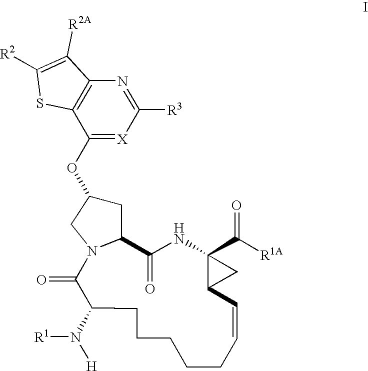
wherein:
R¹ is selected from C₁-C₁₀ alkyl, C₂-C₆ alkenyl, C₂-C₆ alkynyl, —OR⁵, —C(O)R⁵, —C(O)OR⁵, —C(O)NR⁵R⁶, —SO₂NR⁵R⁶, heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;
R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;
R² and R^{2A}, which may be the same or different, are each independently selected from H, halo, cyano, nitro, azido, C₁-C₆ alkyl, C₂-C₆ alkenyl, C₂-C₆ alkynyl, —C(O)NR⁵R⁶, —OR⁵, —C(O)R⁵, —C(O)OR⁵, —OC(O)R⁵, —NR⁵C(O)R⁶, —NR⁵C(O)NR⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), heteroaryl, and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R² groups are optionally substituted with at least one R⁴ group;
R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, heteroaryl, —(CR⁷R⁸)ₜ(C₆-C₁₀ aryl), —(CR⁷R⁸)ₜ(4-10 membered heterocyclic), and C₃-C₁₀ cycloalkyl, wherein each of said heteroaryl, —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;
each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶—NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;
each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);
each R⁷ and R⁸, which may be the same or different, is independently selected from H and C₁-C₆ alkyl;
each t is independently selected from 0, 1, 2, 3, 4, and 5; and
X is CH or N;
or pharmaceutically acceptable salts or solvates thereof.

6. A compound of Formula I 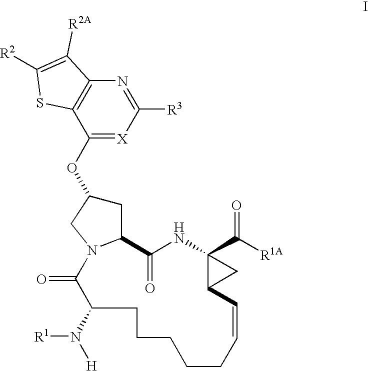
wherein:
R¹ is selected from C₁-C₁₀ alkyl, —OR⁵, —C(O)R⁵, —C(O)OR⁵, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;
R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶—SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;
R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;
R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;
each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶—NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;
each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);
each R⁷ and R⁸, which may be the same or different, is independently selected from H and C₁-C₆ alkyl;
each t is independently selected from 0, 1, 2, 3, 4, and 5; and
X is CH or N;
or pharmaceutically acceptable salts or solvates thereof.

7. A compound of Formula I 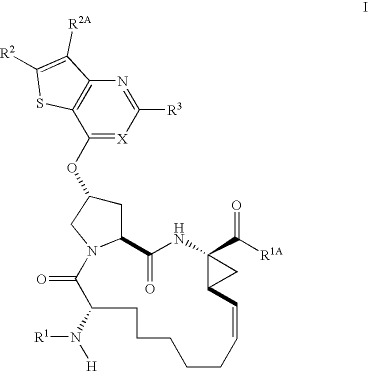
wherein:
R¹ is selected from C₁-C₁₀ alkyl, —OR⁵, C₃-C₁₀ cycloalkoxy, and C₃-C₁₀ cycloalkyl, wherein each of said C₁-C₁₀ alkyl, C₃-C₁₀ cycloalkoxy, and C₃-C₁₀ cycloalkyl moieties of said R¹ groups are optionally substituted with at least one R⁴ group, and wherein at least one carbon in each of said C₁-C₁₀ alkyl, C₃-C₁₀ cycloalkoxy, and C₃-C₁₀ cycloalkyl moieties of said R¹ groups is optionally replaced by —NH—, O or S, with the proviso that said C₁-C₁₀ alkyl, C₃-C₁₀ cycloalkoxy, and C₃-C₁₀ cycloalkyl moieties do not have O—O or S—S bonds;
R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;
R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;
R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;
each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;
each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);
each R⁷ and R⁸, which may be the same or different, is independently selected from H and C₁-C₆ alkyl;
each t is independently selected from 0, 1, 2, 3, 4, and 5; and
X is CH or N;
or pharmaceutically acceptable salts or solvates thereof.

8. A compound of Formula I 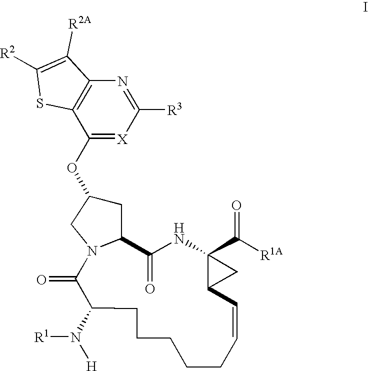
wherein: 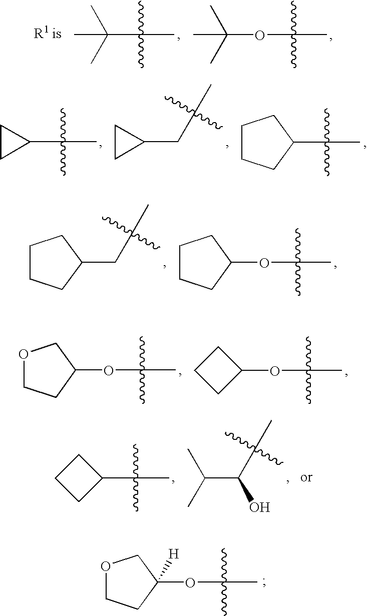
R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;
R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;
R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₅-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;
each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;
each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R³)ₜ(4-10 membered heterocyclic);
each R⁷ and R⁸, which may be the same or different, is independently selected from H and C₁-C₆ alkyl;
each t is independently selected from 0, 1, 2, 3, 4, and 5; and
X is CH or N;
or pharmaceutically acceptable salts or solvates thereof.

9. A compound of Formula II 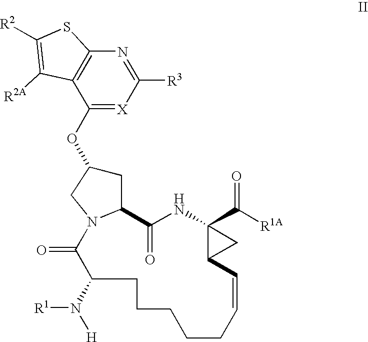
wherein:
R¹ is selected from C₁-C₁₀ alkyl, C₂-C₆ alkenyl, C₂-C₆ alkynyl, —OR⁵, —C(O)R⁵, —C(O)OR⁵, —C(O)NR⁵R⁶, —SO₂NR⁵R⁶, heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;
R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said heteroaryl, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;
R² and R^{2A}, which may be the same or different, are each independently selected from H, halo, cyano, nitro, azido, C₁-C₆ alkyl, C₂-C₆ alkenyl, C₂-C₆ alkynyl, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶—(CR⁷R⁸)ₜ(C₆-C₁₀ aryl), and heteroaryl, wherein each of said —(CR⁷R⁸)ₜ(C₆-C₁₀ aryl) and heteroaryl moieties of said R² and R^{2A} groups are optionally substituted with at least one R⁴ group;
R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(C R⁷R⁸)ₜ(4-10 membered heterocyclic), and C₃-C₁₀ cycloalkyl moieties of said R³ groups are optionally substituted with at least one R⁴ group;
each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;
each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);
each R⁷ and R⁸, which may be the same or different, is independently selected from H and C₁-C₆ alkyl;
each t is independently selected from 0, 1, 2, 3, 4, and 5; and
X is CH or N;
or pharmaceutically acceptable salts or solvates thereof.

10. A compound of Formula II 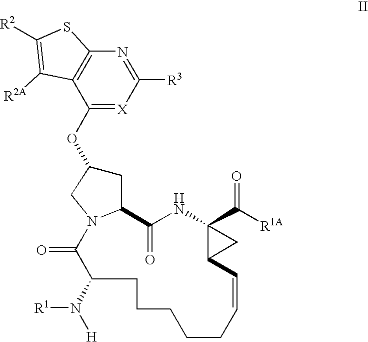
wherein:
R¹ is selected from C₁-C₁₀ alkyl, —OR⁵, —C(O)R⁵, —C(O)OR⁵, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R¹ groups are optionally substituted with at least one R⁴ group;
R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;
R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;
R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;
each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;
each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);
each R⁷ and R⁸, which may be the same or different, is independently selected from H and C₁-C₆ alkyl;
each t is independently selected from 0, 1, 2, 3, 4, and 5; and
X is CH or N;
or pharmaceutically acceptable salts or solvates thereof.

11. A compound of Formula II 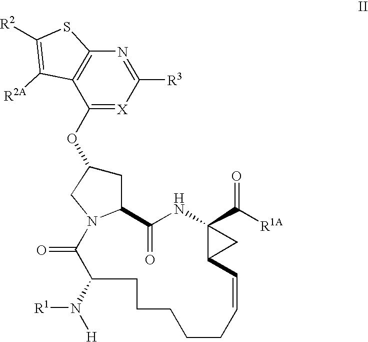
wherein:
R¹ is selected from C₁-C₁₀ alkyl, —OR⁵, C₃-C₁₀ cycloalkoxy, and C₃-C₁₀ cycloalkyl, wherein each of said C₁-C₁₀ alkyl, C₃-C₁₀ cycloalkoxy, and C₃-C₁₀ cycloalkyl moieties of said R¹ groups are optionally substituted with at least one R⁴ group, and wherein at least one carbon in each of said C₁-C₁₀ alkyl, C₃-C₁₀ cycloalkoxy, and C₃-C₁₀ cycloalkyl moieties of said R¹ groups is optionally replaced by —NH—, O or S, with the proviso that said C₁-C₁₀ alkyl, C₃-C₁₀ cycloalkoxy, and C₃-C₁₀ cycloalkyl moieties do not have O—O or S—S bonds;
R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁸, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said C₆-C₁₀ aryl, 4-10 membered heterocyclic, C₃-C₁₀ cycloalkoxy, and C₃-C₁₀ cycloalkyl moieties of said R^{1A} groups are optionally substituted with at least one R⁴ group;
R² and R^{2A}, which may be the same or different, are each independently selected from H, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein said —(CR⁷R⁸)ₜ(C₆-C₁₀ aryl) and heteroaryl are optionally substituted with at least one R⁴ group;
R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;
each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶, —NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;
each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);
each R⁷ and R⁸, which may be the same or different, is independently selected from H and C₁-C₆ alkyl;
each t is independently selected from 0, 1, 2, 3, 4, and 5; and
X is CH or N;
or pharmaceutically acceptable salts or solvates thereof.

12. A compound of Formula II 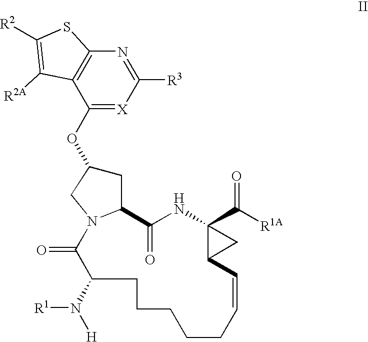
wherein: 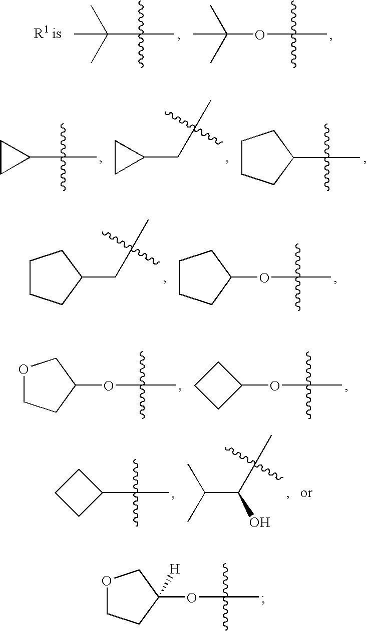
R^{1A} is selected from C₁-C₁₀ alkyl, C₂-C₆ alkenyl, C₂-C₆ alkynyl, —OR⁵, —C(O)R⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl, wherein each of said —(CR⁷R⁸)ₜ(C₆-C₁₀ aryl), —(CR⁷R⁸)ₜ(4-10 membered heterocyclic), C₃-C₁₀ cycloalkoxy, and C₃-C₁₀ cycloalkyl moieties of said R^{1A} groups are optionally substituted with at least one R⁴ group;
R² and R^{2A}, which may be the same or different, are each independently selected from H, halo, cyano, nitro, azido, C₁-C₆ alkyl, C₂-C₆ alkenyl, C₂-C₆ alkynyl, —C(O)NR⁵R⁶, —OR⁵, —C(O)R⁵, —C(O)OR⁵, —NR⁵C(O)R⁶, —NR⁵C(O)NR⁶, —NR⁵R⁶, —NR⁵OR⁶, —(CR⁷R⁸)ₜ(C₆-C₁₀ aryl), and heteroaryl, wherein each of said —(CR⁷R⁸)ₜ(C₆-C₁₀ aryl) and heteroaryl moieties of said R² and R^{2A} groups are optionally substituted with at least one R⁴ group;
R³ is selected from H, halo, cyano, nitro, azido, C₁-C₁₀ alkyl, C₂-C₆ alkenyl, C₂-C₆ alkynyl, —C(O)R⁵, —OR⁵, —C(O)OR⁵, —OC(O)R⁵, —NR⁵C(O)R⁶, —NR⁵C(O)NR⁶, —C(O)NR⁵R⁶, —NR⁵R⁶, —NR⁵OR⁶, —SO₂NR⁵R⁶, —NR⁵SO₂R⁶, —(CR⁷R⁸)ₜ(C₆-C₁₀ aryl), —(CR⁷R⁸)ₜ(4-10 membered heterocyclic), and C₃-C₁₀ cycloalkyl, wherein each of said —(CR⁷R⁸)ₜ(C₆-C₁₀ aryl), —(CR⁷R⁸)ₜ(4-10 membered heterocyclic), and C₃-C₁₀ cycloalkyl moieties of said R³ groups are optionally substituted with at least one R⁴ group;
each R⁴ is independently selected from C₁-C₆ alkyl, C₂-C₆ alkenyl, C₂-C₆ alkynyl, halo, nitro, —OR⁵, —NR⁵R⁶, —CF₃, —SO₂R⁵R⁶, —C(O)NR⁵R⁶, —C(O)R⁵, —NR⁵C(O)R⁶—NR⁵C(O)NR⁶, and —CN, wherein said C₁-C₆ alkyl, C₂-C₆ alkenyl, and C₂-C₆ alkynyl moieties of said R⁴ groups are optionally substituted with at least one NR⁵, O or S;
each R⁵ and R⁶, which may be the same or different, is independently selected from H, C₁-C₆ alkyl, C₃-C₁₀ cycloalkyl, C₂-C₆ alkenyl, C₂-C₆ alkynyl, —(CR⁷R⁸)ₜ(C₆-C₁₀ aryl), and —(CR⁷R⁸)ₜ(4-10 membered heterocyclic);
each R⁷ and R⁸, which may be the same or different, is independently selected from H and C₁-C₆ alkyl;
each t is independently selected from 0, 1, 2, 3, 4, and 5; and
X is CH or N;
or pharmaceutically acceptable salts or solvates thereof.

13. A compound selected from: 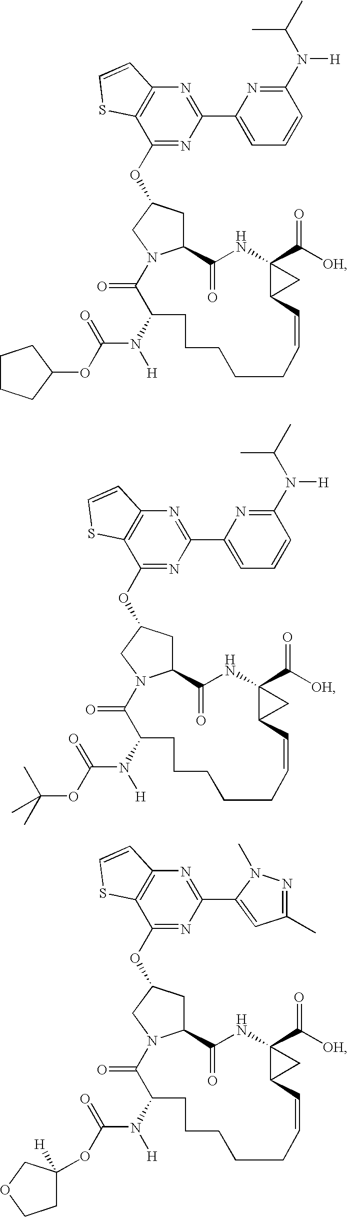 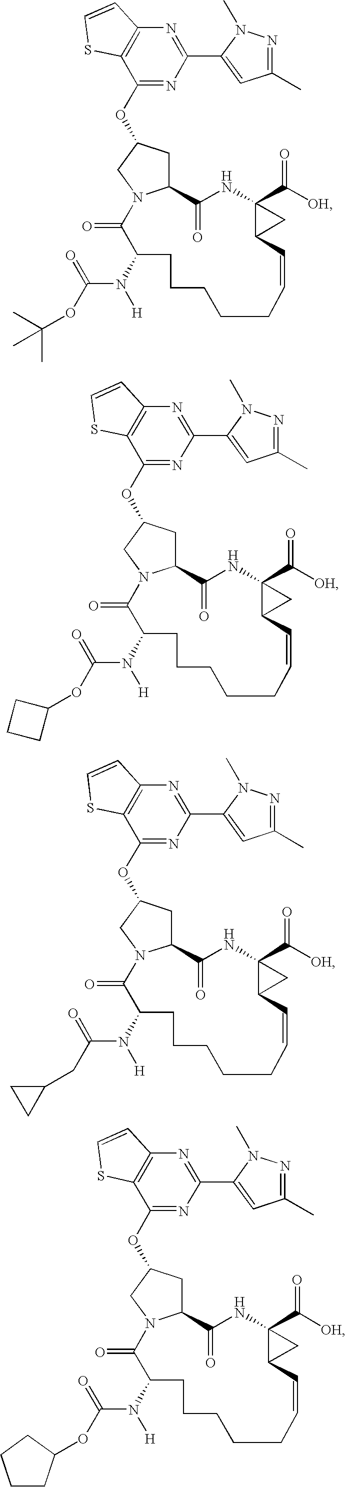 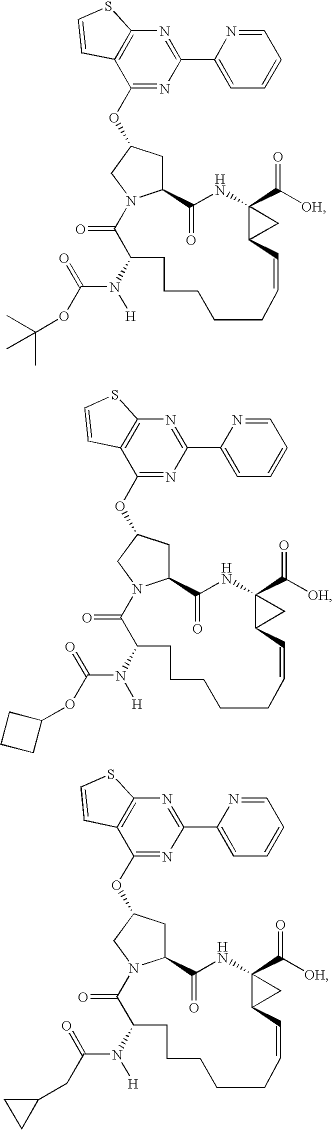 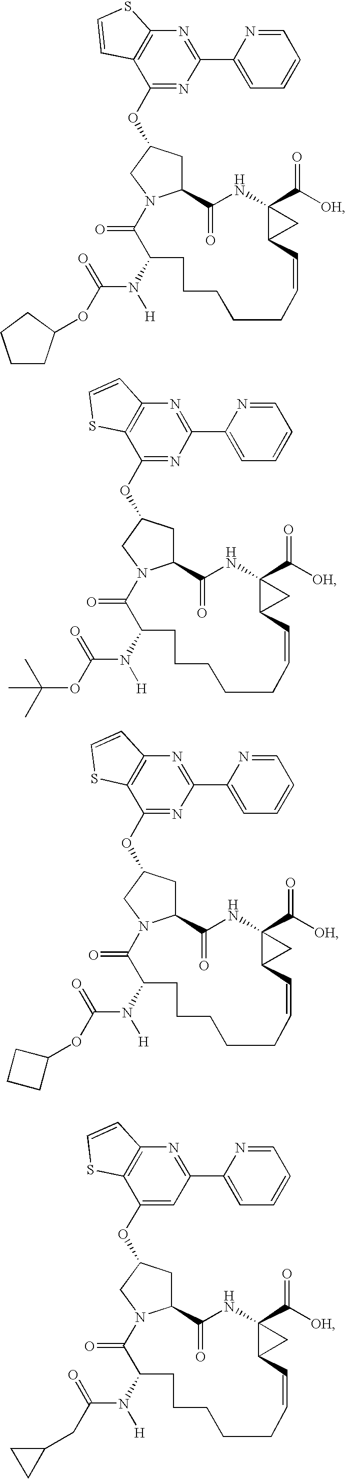 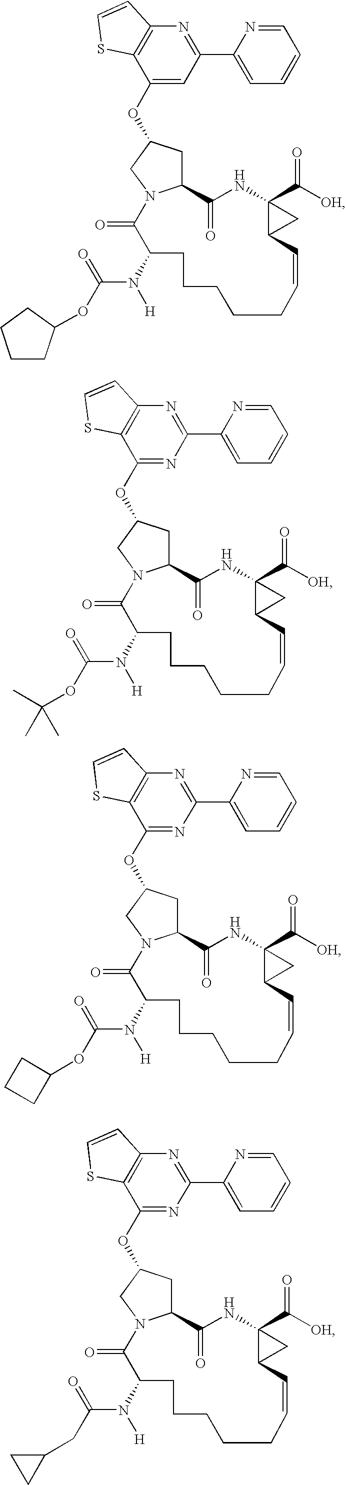 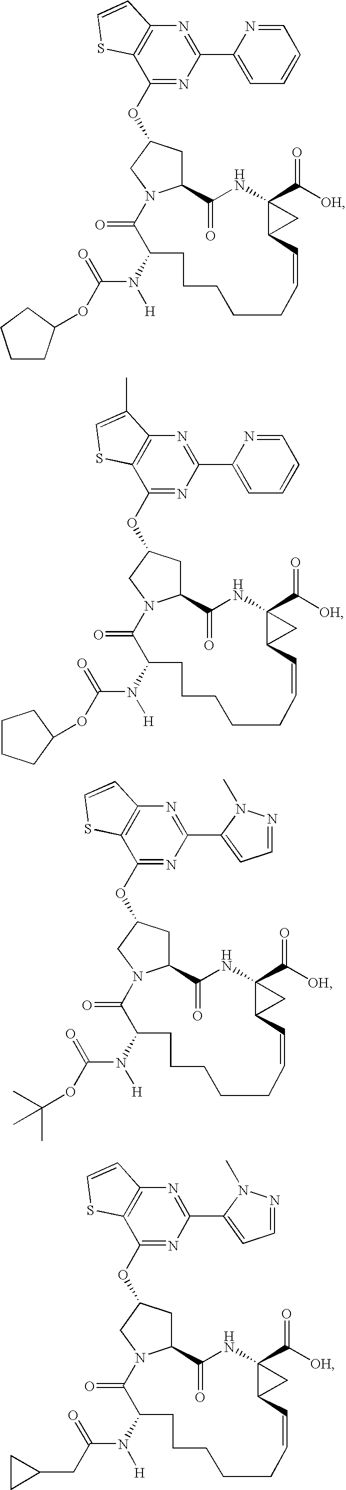 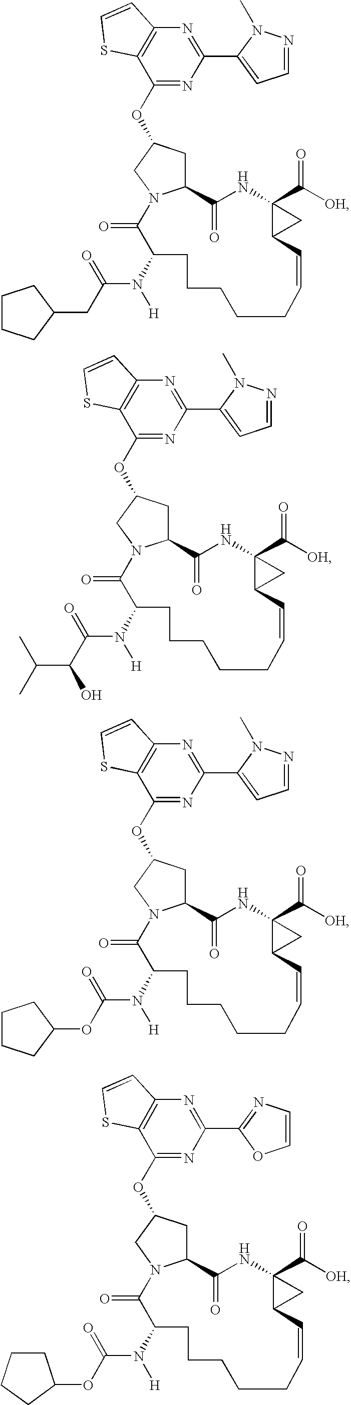 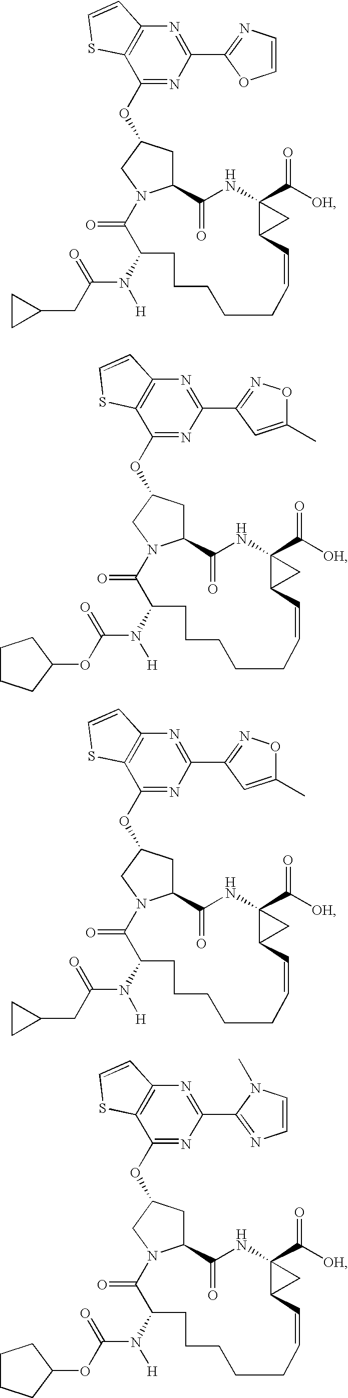 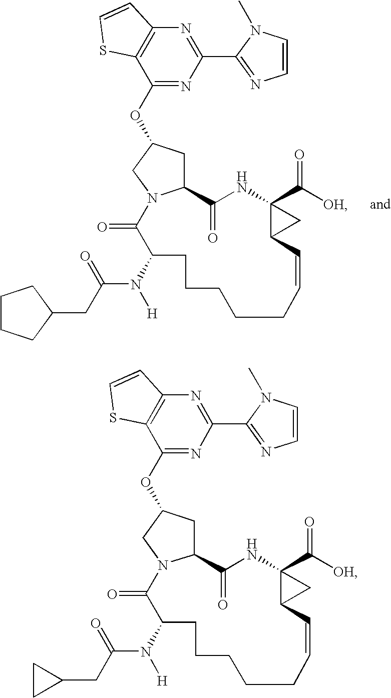
or pharmaceutically acceptable salts or solvates thereof.

14. A compound selected from: 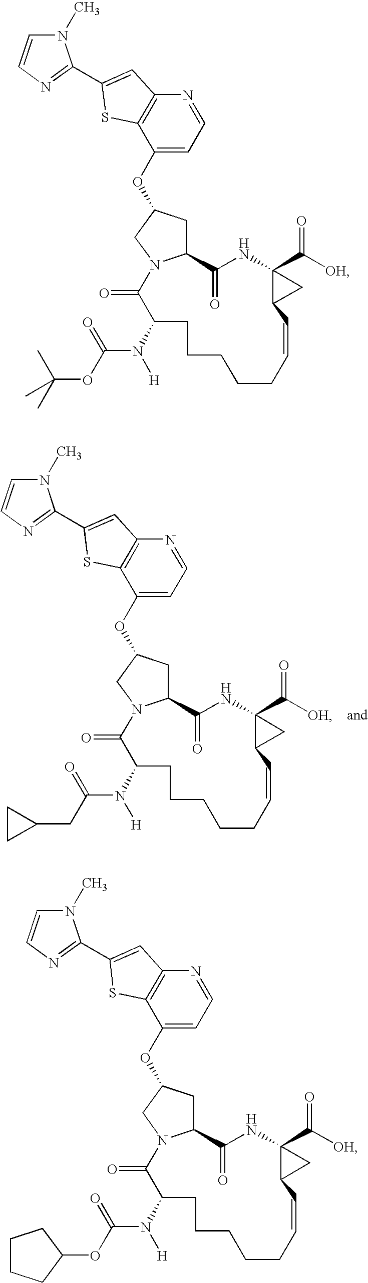
or pharmaceutically acceptable salts or solvates thereof.

15. A pharmaceutical composition comprising an amount of a compound according to any one of claims 1 to 14 that is effective in treating Hepatitis C virus in an infected mammal, and a pharmaceutically acceptable carrier.
